(19) United States
(12) Patent Application Publication
(10) Pub. No.: US 2026/0297530 A1
(43) Pub. Date: Oct. 1, 2026

(54) CELLS, ISLETS, AND ORGANOIDS THAT EVADE IMMUNE DETECTION AND AUTOIMMUNITY, METHODS OF PRODUCTION AND USE THEREOF

(71) Applicant: SALK INSTITUTE FOR BIOLOGICAL STUDIES, La Jolla, CA (US)

(72) Inventors: Eiji YOSHIHARA, La Jolla, CA (US); Ruth YU, La Jolla, CA (US); Michael DOWNES, La Jolla, CA (US); Ronald EVANS, La Jolla, CA (US); Annette ATKINS, La Jolla, CA (US)

(73) Assignee: SALK INSTITUTE FOR BIOLOGICAL STUDIES, La Jolla, CA (US)

(21) Appl. No.: 19/637,759

(22) Filed: Apr. 2, 2026

Related U.S. Application Data
(62) Division of application No. 17/284,355, filed on Oct. 11, 2019, now Pat. No. 12,637,662.
(60) Provisional application No. 62/745,086, filed on Oct. 12, 2018.
(60) Provisional application No. 62/795,284, filed on Jan. 22, 2019.

(51) Int. Cl. C12N 5/071; A61K 35/39

(52) U.S. Cl. CPC C12N 5/0677; A61K 35/39; C12N 2501/415; C12N 2502/1382; C12N 2502/28; C12N 2506/45; C12N 2510/00; C12N 2533/70; C12N 2533/90

(57) ABSTRACT

The invention features cells, islet-like cells, pancreatic islets and organoids (e.g., human islet-like organoids or HILOs), as well as cell cultures and methods that are useful for the rapid and reliable generation of cells and organoids, such as pancreatic islets and organoids, that are sustainable in vivo and that evade immune detection, rejection and autoimmunity. The invention also features methods of treating pancreatic diseases, such as type 2 diabetes, and pancreatic cancer, using the cells, islet-like cells, pancreatic islets and organoids (e.g., HILOs) that are designed to modulate the activity of immune cells that would otherwise react against them.

### CROSS-REFERENCE TO RELATED APPLICATIONS

This application is a divisional under 35 U.S.C. § 111(a) of U.S. patent application Ser. No. 17/284,355, filed Apr. 9, 2021, which is a U.S. national phase application under 35 USC § 371 of PCT International Patent Application No. PCT/US2019/055827, filed Oct. 11, 2019, designating the United States and published in English, which claims priority to and the benefit of U.S. Provisional Application No. 62/745,086, filed Oct. 12, 2018, and U.S. Provisional Application No. 62/795,284, filed Jan. 22, 2019, the entire contents of all of which are incorporated by reference herein.

### STATEMENT OF RIGHTS TO INVENTIONS MADE UNDER FEDERALLY SPONSORED RESEARCH

This invention was made with government support under Grant Nos. DK057978, DK090962, HL088093, HL105278 and ES010337 awarded by the National Institutes of Health, and Grant No. P30 014195 awarded by the National Institutes of Health and the National Cancer Institute. The government has certain rights in the invention.

### SEQUENCE LISTING

This application contains a Sequence Listing which has been submitted electronically in XML format and is hereby incorporated by reference in its entirety. The Sequence Listing XML file, created on Apr. 2, 2026, is named 167776-011316USDIV_SL.xml and is 222,684 bytes in size.

### BACKGROUND

For the treatment of insulin dependent diabetes, such as type 1 diabetes and late-stage type 2 diabetes, the shortage of human islets limits the number of patients who can benefit from this therapy. Despite progress in the field of in vitro differentiation of human induced pluripotent stem cells (hiPSCs) into β-like cells, the β-like cells generated in this manner typically exhibit impairments in glucose-stimulated insulin secretion (GSIS) and mitochondrial metabolic function, as well as detection and destruction by a recipient's immune system following administration. Thus, further improvements to the maturation process are required to fully capture pancreatic islet physiology and the generation of functional and lasting organoids.

Needed in the art are methods for generating functional human organs that survive transplant for the treatment of diseases, as well as new platforms for drug-screening and disease modeling to provide new treatment strategies and therapeutics for patients with organ failure.

### SUMMARY OF THE DESCRIBED EMBODIMENTS

Provided are compositions and methods for generating an immunoprotected cell, islet, organoid, or islet-like organoid, including, but not limited to, a human pancreatic islet organoid or a pancreatic organoid, in particular, a human islet-like organoid (abbreviated as “HILO” herein), that survives and evades detection by the immune system (autoimmunity) following administration to or transplant or implant in a subject. In an embodiment, the cell, islet, organoid, islet-like organoid (and cells therein) expresses interferon gamma (IFNγ)-receptors. In an embodiment, the cell, islet, organoid, or islet-like organoid (and cells therein) is human.

In an aspect, a method of increasing survival or reducing cell death of a transplanted donor cell is provided in which the method comprises contacting the donor cell with multiple intermittent exposures to interferon gamma (IFNγ) over a given time period, e.g., a time period of at least 24 hours, thereby increasing survival of the transplanted donor cell. In an embodiment, the transplanted donor cell is an organoid cell, an islet cell, an islet-like organoid cell, or a β-like islet cell. In an embodiment, the transplanted donor cell is syngeneic to the subject who receives the transplant. In an embodiment, the transplanted donor cell is autologous to the subject who receives the transplant. In an embodiment, the transplanted donor cell is allogeneic or xenogeneic to the subject who receives the transplant. In an embodiment, the transplanted donor cell is an interferon gamma (IFNγ) receptor-expressing cell. In an embodiment, the transplanted donor cell is a human cell.

In another aspect, a method of generating an immunoprotected cell, islet, or organoid that survives detection by immune system cells, e.g., T cells or B cell, is provided in which the method comprises subjecting an interferon gamma (IFNγ) receptor-expressing cell, islet, or organoid, or cells thereof, to multiple intermittent exposure to IFNγ over a given time period, e.g., a time period of at least 24 hours, thereby inducing expression of an immune checkpoint protein by the cell, islet, or organoid and allowing said cell, islet, or organoid to survive immune detection or autoimmunity.

In an aspect, the human islet-like organoid (HILO) and the cells comprising the HILO, namely, beta (β)-like cells, express or are induced to express following exposure to IFNγ one or more molecules involved in modulating the immune response or autoimmunity, such as an immune checkpoint protein, to overcome immune rejection or autoimmunity of “non-self” cells or HILOs introduced into, e.g., transplanted or implanted, into a subject. In an embodiment, the immune checkpoint protein is PD-L1. In an embodiment, the subject into whom HILOs are introduced, transplanted, or implanted has diabetes. In an embodiment, the subject into whom HILOs are introduced, transplanted, or implanted has type 1, type 2 diabetes, or late stage type 2 diabetes. In an embodiment, the subject into whom HILOs are introduced, transplanted, or implanted has type 1 diabetes. In an embodiment, the subject into whom HILOs are introduced, transplanted, or implanted is a human subject or patient. In an embodiment, the one or more immune checkpoint protein is recombinantly expressed in the introduced, transplanted, or implanted cells or HILOs. The terms “transplant” and “implant” may be used interchangeably herein to refer to cells, islets, or organoids (and cells therein) that are introduced or transferred into a subject by procedures practiced in the medical arts to effect or provide a function therein, especially a therapeutic function to treat a disease, disorder or pathology.

In one aspect, a method of generating a pancreatic islet organoid is provided in which induced pluripotent stem cell (iPSC)-derived beta (β)-like cells are cultured in a 3-dimensional matrix containing gellan gum, thereby generating a pancreatic islet organoid in which the organoid cells express one or more checkpoint proteins. Also provided is a cell culture including an iPSC-derived beta-like cell, which expresses one or more immune checkpoint proteins, in a three-dimensional matrix containing gellan gum. In an embodiment, the one or more immune checkpoint proteins is PD-L1.

In an aspect, a cell culture including a human iPSC-derived beta-like cell, a human adipose-derived stem cell (hADSC), and a human umbilical vein endothelial cell (HUVEC) in a three-dimensional matrix containing gellan gum is provided, in which the cells of the culture express one or more immune checkpoint proteins.

In various embodiments of any aspect delineated herein, the cell culture includes an adipose-derived stem cell and/or an endothelial cell.

In an aspect, a pancreatic islet-like organoid containing an iPSC-derived beta-like cell which expresses one or more immune checkpoint proteins is provided, wherein the organoid is vascularized and exhibits glucose-stimulated insulin secretion (GSIS) and wherein the cells of the organoid and the organoid express one or more immune checkpoint proteins. In an embodiment, the pancreatic islet-like organoid is a human pancreatic islet-like organoid. In an embodiment the one or more immune checkpoint proteins is PD-L1.

In an aspect, a pancreatic islet organoid containing an iPSC-derived beta (ß)-like cell, an iPSC-derived alpha (α) cell, an iPSC-derived delta (δ) cell, an iPSC-derived duct cell, an adipose-derived stem cell (hADSC), and an endothelial cell, wherein the iPSC cell expresses one or more immune checkpoint proteins, the organoid is vascularized and exhibits glucose-stimulated insulin secretion (GSIS), KCl-stimulated insulin secretion, GLP-1 stimulated insulin secretion, somatostatin secretion, and glucagon secretion is provided.

In a related aspect, a non-human organism transplanted or implanted with the organoid of any aspect delineated herein is provided.

In an aspect, a method of treating a pancreatic disease in a subject is provided, in which a pancreatic islet organoid, or HILO, is introduced or transplanted or implanted into the subject, wherein the pancreatic islet organoid, or HILO, contains iPSC-derived beta-like cells, which express one or more immune checkpoint proteins to evade immune detection; wherein the pancreatic islet organoid, or HILO, is vascularized and exhibits glucose-stimulated insulin secretion (GSIS). In an embodiment, the one or more immune checkpoint proteins is PD-L1. In an embodiment, the subject is human and the pancreatic islet organoid, or HILO, is generated from human tissue or cells.

In an aspect, a method of treating type 1 diabetes in a subject is provided, in which a pancreatic islet organoid, or HILO, is introduced, transplanted, or implanted into the subject, wherein the pancreatic islet organoid, or HILO, contains iPSC-derived beta-like cells, which express one or more immune checkpoint proteins to evade immune detection; wherein the pancreatic islet organoid, or HILO, is vascularized and exhibits glucose-stimulated insulin secretion (GSIS). In an embodiment, the pancreatic islet organoid, or HILO, expresses a checkpoint protein to evade immune detection. In an embodiment, the one or more immune checkpoint proteins is PD-L1. In an embodiment, the subject is human and the pancreatic islet organoid, or HILO, is generated from human tissue or cells.

In an aspect, a pancreatic islet organoid or HILO is provided, in which the pancreatic islet organoid or HILO is generated by culturing an induced pluripotent stem cell (iPSC)-derived beta-like cell in a 3-dimensional matrix containing gellan gum. In an embodiment, the pancreatic islet organoid, or HILO, expresses one or more immune checkpoint proteins to evade immune detection. In an embodiment, the subject is human and the pancreatic islet organoid, or HILO, is generated from human tissue or cells. In an embodiment, the one or more immune checkpoint proteins is PD-L1.

Provided in another aspect is a pancreatic organoid or HILO generated by culturing an induced pluripotent stem cell (iPSC)-derived beta-like cell and an iPSC-derived exocrine component cell in a 3-dimensional matrix containing gellan gum. In an embodiment, the pancreatic islet organoid, or HILO, expresses one or more immune checkpoint proteins to evade immune detection. In an embodiment, the one or more immune checkpoint proteins is PD-L1. In an embodiment, the subject is human and the pancreatic islet organoid, or HILO, is generated from human tissue or cells.

Provided in another aspect is a pancreatic organoid or HILO generated by culturing an induced pluripotent stem cell (iPSC)-derived beta-like cell and an iPSC-derived exocrine component cell in a culture medium, such as a 3-dimensional matrix containing gellan gum and an agent that stimulates expression and production of a checkpoint protein in the cells of the pancreatic organoid (B-cells) or HILO. Without wishing to be bound by theory, the PD-L1 is produced in the n-cells or HILO through the mechanism of transcriptional memory. In an embodiment, the culture medium or matrix comprises interferon gamma (IFNγ). In an embodiment, the pancreatic islet organoid, or HILO, expresses one or more immune checkpoint proteins to evade immune detection. In an embodiment, the one or more immune checkpoint proteins is PD-L1. In an embodiment, the subject is human and the pancreatic islet organoid, or HILO, is generated from human tissue or cells.

In another aspect, the invention provides a liver organoid generated by culturing an induced pluripotent stem cell (iPSC)-derived hepatocyte in a 3-dimensional matrix containing gellan gum; wherein the iPSC-derived hepatocyte expresses one or more immune checkpoint proteins such that the liver organoid evades immune detection. In an embodiment, the one or more immune checkpoint proteins is PD-L1.

In another aspect, the invention provides a heart organoid generated by culturing an induced pluripotent stem cell (iPSC)-derived cardiomyocyte in a 3-dimensional matrix containing gellan gum wherein the iPSC-derived cardiomyocyte expresses one or more immune checkpoint proteins such that the heart organoid evades immune detection. In an embodiment, the one or more immune checkpoint proteins is PD-L1.

In another aspect, the invention provides an intestinal organoid generated by culturing an induced pluripotent stem cell (iPSC)-derived intestinal cell in a 3-dimensional matrix containing gellan gum, wherein the iPSC-derived intestinal cell expresses one or more immune checkpoint proteins such that the intestinal organoid evades immune detection. In an embodiment, the one or more immune checkpoint proteins is PD-L1.

In various embodiments of any aspect delineated herein, the method involves culturing the iPSC-derived beta-like cell, which expresses one or more immune checkpoint proteins, with an adipose-derived stem cell and/or an endothelial cell. In an embodiment, the one or more immune checkpoint proteins is PD-L1. In various embodiments of any aspect delineated herein, the method involves culturing the iPSC-derived beta-like cell, which expresses one or more immune checkpoint proteins, with an iPSC-derived alpha-like cell, an iPSC-derived delta-like cell, and/or an iPSC-derived duct-like cell.

In various embodiments of any aspect delineated herein, the pancreatic islet organoid contains an iPSC-derived alpha-like cell, an iPSC-derived delta-like cell, and/or an iPSC-derived duct-like cell. In various embodiments of any aspect delineated herein, the pancreatic islet organoid includes an adipose-derived stem cell and/or an endothelial cell. In various embodiments of any aspect delineated herein, the pancreatic islet organoid exhibits KCl-stimulated insulin secretion, GLP-1 stimulated insulin secretion, somatostatin secretion, c-peptide expression, and/or glucagon secretion. In various embodiments of any aspect delineated herein, the pancreatic islet organoid expresses one or more of the beta cell transcription factors Pdx1, MafA, Pax4, Pax6, NeuroD1, Nkx6-1, Gata6, and Foxa2. In certain embodiments, the pancreatic islet organoid contains an iPSC-derived beta-like cell, which expresses one or more immune checkpoint proteins, an iPSC-derived alpha cell, an iPSC-derived delta cell, an iPSC-derived duct cell, an adipose-derived stem cell (hADSC), and an endothelial cell, where the organoid is vascularized and exhibits glucose-stimulated insulin secretion (GSIS), KCl-stimulated insulin secretion, GLP-1 stimulated insulin secretion, somatostatin secretion, and glucagon secretion. In an embodiment, the one or more immune checkpoint proteins is PD-L1. In various embodiments of any aspect delineated herein, the pancreatic islet organoid is surrounded by an iPSC-derived exocrine component. In various embodiments, the iPSC-derived exocrine component expresses one or more of the markers PDX1, Nkx6-1, and Ptf1.

In various embodiments of any aspect delineated herein, the liver organoid expresses one or more of the markers AFP, ALB, and Cyp3a7. In various embodiments of any aspect delineated herein, the liver organoid exhibits insulin signaling, insulin resistance by palmitic acids, and lipid accumulation.

In various embodiments of any aspect delineated herein, the heart organoid expresses one or more of the markers hMlc2a, hNkx2-5, alphaMHC and KCNQ1. In various embodiments of any aspect delineated herein, the heart organoid exhibits cardiac beating.

In various embodiments of any aspect delineated herein, the intestinal organoid expresses one or more of the markers CDX2, Muc2, and Lgr5. In various embodiments of any aspect delineated herein, the intestinal organoid exhibits budding in response to R-spondin.

In various embodiments of any aspect delineated herein, the iPSC-derived beta-like cell, iPSC-derived alpha-like cell, iPSC-derived delta-like cell, and/or iPSC-derived duct-like cell is human. In various embodiments of any aspect delineated herein, the iPSC-derived beta-like cell, iPSC-derived exocrine component cell, iPSC-derived hepatocyte, iPSC-derived cardiomyocyte, or iPSC-derived intestinal cell is human. In various embodiments, the adipose-derived stem cell is a human adipose-derived stem cell (hADSC). In various embodiments of any aspect delineated herein, the endothelial cell is a human umbilical vein endothelial cell (HUVEC). In various embodiments, the organoids are generated from human cells.

In various embodiments of any aspect delineated herein, the pancreatic islet organoid, pancreatic organoid, liver organoid, heart organoid, or intestinal organoid, contains an adipose-derived stem cell and/or an endothelial cell. In various embodiments of any aspect delineated herein, the pancreatic islet organoid, pancreatic organoid, liver organoid, heart organoid, or intestinal organoid is vascularized.

In another aspect, the invention provides a method of generating a pancreatic islet organoid of HILO, the method comprising culturing an induced pluripotent stem cell (iPSC)-derived beta-like cell, which expresses one or more immune checkpoint proteins, in a medium comprising Wnt4 or Wnt5a protein. In an embodiment, the one or more immune checkpoint proteins is PD-L1. In an embodiment, the induced pluripotent stem cell (iPSC)-derived beta-like cell is cultured in a 3-dimensional matrix. In an embodiment of the foregoing aspect, the Wnt4 or Wnt5a protein is a recombinant human Wnt4 or Wnt5a protein. In a particular embodiment, the medium comprises recombinant human Wnt4 protein. In another particular embodiment, the medium comprises recombinant human Wnt5a protein. In a particular embodiment, a Wnt4- or Wnt5-induced human islet organoid or HILO is a mature islet or a mature HILO.

In another aspect the invention provides a cell culture comprising a human iPSC-derived beta-like cell, which expresses one or more immune checkpoint proteins, and Wnt4 or Wnt5a protein. In an embodiment, the one or more immune checkpoint proteins is PD-L1. In an embodiment, the human iPSC-derived beta-like cell is in a three-dimensional matrix comprising gellan gum. In an embodiment, the Wnt4 or Wnt5a protein is a recombinant human Wnt4 or Wnt5a protein. In a particular embodiment, the medium comprises recombinant human Wnt4 protein. In another particular embodiment, the medium comprises recombinant human Wnt5a protein. In a particular embodiment, a Wnt4- or Wnt5-induced human islet organoid or HILO is a mature islet or a mature HILO.

In another aspect, the invention provides a pancreatic islet organoid comprising an iPSC-derived beta-like cell, which expresses one or more immune checkpoint proteins, cultured in medium comprising Wnt4 or Wnt5a protein, wherein the organoid is vascularized and exhibits glucose-stimulated insulin secretion (GSIS). In an embodiment, the one or more immune checkpoint proteins is PD-L1. In an embodiment, the organoid further exhibits KCl-stimulated insulin secretion or glucose stimulated insulin secretion. In an embodiment, the pancreatic islet organoid expresses Fltp and Esrrg genes. In an embodiment, the Wnt4 or Wnt5a protein is a recombinant human Wnt4 or Wnt5a protein. In a particular embodiment, the medium comprises recombinant human Wnt4 protein. In another particular embodiment, the medium comprises recombinant human Wnt5a protein. In a particular embodiment, a Wnt4- or Wnt5-induced human islet organoid or HILO is a mature islet or a mature HILO.

In another aspect, the invention provides a non-human organism transplanted or implanted with the organoid defined in the above described aspects.

In another aspect, the invention provides a method of enhancing self organization of adipose-derived stem cells (ADSCs) for generating an induced pluripotent stem cell (iPSC)-derived organoid, which evades immune surveillance and rejection, the method comprising culturing the ADSCs in a 3-dimensional (3-D) culture matrix medium comprising a Wnt5a protein. In an embodiment, the one or more immune checkpoint proteins is PD-L1. In an embodiment of the method, the ADSCs are cultured in a 3-D culture matrix comprising gellan gum. In an embodiment, the ADSCs are cultured in the 3-D culture matrix medium comprising a Wnt5 protein and an iPSC-derived cell selected from an iPSC-derived beta-like cell, an iPSC-derived exocrine component cell, an iPSC-derived hepatocyte, an iPSC-derived cardiomyocyte, or an iPSC-derived intestinal cell, which expresses one or more immune checkpoint inhibitor proteins. In an embodiment, the one or more immune checkpoint proteins is PD-L1. In an embodiment of the method, the iPSC-derived organoid is selected from a pancreatic islet organoid, pancreatic organoid, a liver organoid, a heart organoid, or an intestinal organoid. In an embodiment of the method, the induced pluripotent stem cell (iPSC)-derived organoid is a human induced pluripotent stem cell (hiPSC)-derived organoid. In an embodiment of the method, the Wnt5a protein is a recombinant human Wnt5a protein. In an embodiment of the method, the pancreatic islet organoid, pancreatic organoid, liver organoid, heart organoid, or intestinal organoid is derived from an iPSC-derived cell selected from an iPSC-derived beta-like cell, an iPSC-derived exocrine component cell, an iPSC-derived hepatocyte, an iPSC-derived cardiomyocyte, or an iPSC-derived intestinal cell, respectively. In an embodiment, of any of the above, the iPSC-derived cell is human.

In another aspect, the invention provides a method of enhancing self organization of adipose-derived stem cells (ADSCs) for generating a pancreatic islet or pancreatic organoid that evades immune rejection or autoimmunity, comprising culturing ADSCs, which express one or more immune checkpoint proteins, in medium comprising Wnt5a protein. In an embodiment, the one or more immune checkpoint proteins is PD-L1. In an embodiment, the ADSCs are cultured in a 3-dimensional matrix comprising gellan gum. In another embodiment, the Wnt5a protein a recombinant human Wnt5a protein.

In another aspect, the invention provides a pancreatic islet organoid, pancreatic organoid, a liver organoid, a heart organoid, or intestinal organoid produced by any of the above-delineated methods and embodiments thereof.

In various aspects of any of the foregoing embodiments, the immune checkpoint protein, or the one or more immune checkpoint proteins, or a fragment or portion of the immune checkpoint protein that binds to cognate ligand, is recombinantly expressed in or molecularly introduced into the cells of an organoid, (e.g., β-like cells that constitute HILOs) which express the one or more checkpoint proteins as membrane surface proteins that bind to a cognate ligand on an immune cell, e.g., a T cell, that is involved in autoimmunity, or that reacts against a foreign or ‘non-self’ cell, so as to suppress or block the T cell response (an allogeneic immune response or autoimmune response) and thus evade immune system surveillance and rejection in a recipient.

In embodiments, the cells of an organoid, (e.g., β-like cells that constitute HILOs) express one or more checkpoint proteins or molecules that bind to cognate ligands on the surface of an immune cell to suppress allogeneic immune activity or autoimmunity against the cells and the organoid. In a particular embodiment, the cells of an organoid, (e.g., a β-like cell) and the organoid (e.g., HILO) express the immune checkpoint protein PD-L1, programmed cell-death ligand 1, which binds to PD-1, programmed cell-death protein 1, which is expressed, for example, on T cells. PD-L2, programmed cell-death ligand 2, also binds to PD-1, but with a different Kd. In other embodiments, the cells of an organoid, (e.g., a β-like cell) and the organoid (e.g., HILO) are molecularly engineered to express a molecule that binds a checkpoint protein expressed on the surface of an immune cell, such as a T cell (e.g., an effector T cell), wherein the checkpoint protein expressed on the surface of an immune cell is CTLA-4 (cytotoxic T-lymphocyte protein 4, also called CD152); LAG-3, lymphocyte activation gene 3 protein; KIR, killer cell immunoglobulin-like receptor; IDO1, indoleamine 2,3-dioxygenase 1; 4-1BB, a tumor necrosis factor receptor superfamily member 9, (also known as CD137); GITR, “glucocorticoid-induced TNFR family related gene; TIM-3, “T-cell immunoglobulin domain and mucin domain;” OX40, tumor necrosis factor receptor superfamily member 4, (also known as CD134); A2AR, adenosine A2A receptor; B7-H3 (also called CD276); B7-H4 (also called VTCN1); B7-1/B7-2; BTLA (also called CD272); VISTA, “V-domain Ig suppressor of T cell activation;” or a combination of any of the foregoing.

In an aspect of any of the foregoing embodiments, the immune checkpoint protein comprises all, or a portion, e.g., the extracellular domain, of the checkpoint protein (also called a “checkpoint molecule” herein). In a particular embodiment, the immune checkpoint protein is PD-L1 or a binding portion thereof. In an embodiment, the checkpoint protein is the extracellular domain of the PD-L1 protein.

Another aspect provides a human induced pluripotent stem cell (hiPSC), human beta (β)-cell, or human islet-like organoid (HILO) generated therefrom, molecularly engineered to express one or more immune checkpoint proteins that bind to a cognate ligand on an immune cell, such as a T cell. In an embodiment, the one or more immune checkpoint proteins expressed by a hiPSC, human beta (β)-cell, or human islet-like organoid (HILO) binds to an immune cell-expressed cognate ligand selected from programmed cell-death protein 1 (PD-1); cytotoxic T-lymphocyte protein 4 (CTLA-4); lymphocyte activation gene 3 protein (LAG-3); killer cell immunoglobulin-like receptor (KIR); indoleamine 2,3-dioxygenase 1 (IDO1); tumor necrosis factor receptor superfamily member 9 (4-1BB); glucocorticoid-induced TNFR family related gene (GITR); T-cell immunoglobulin domain and mucin domain (TIM-3); tumor necrosis factor receptor superfamily member 4, (OX40); adenosine A2A receptor (A2AR); B7-H3; B7-H4; B7-1/B7-2; BTLA; V-domain Ig suppressor of T cell activation (VISTA); or a combination of any of the foregoing. In a particular embodiment, the hiPSC, human beta (β)-cell, or HILO expresses the immune checkpoint protein, programmed cell-death protein-ligand 1 (PD-L1), which binds to PD-1.

In another aspect, a method of generating cells, islets, organoids that survive and have reduced cell death following transplantation, implantation, or transfer is provided in which the method comprises: (a) contacting interferon gamma (IFNγ)-receptor expressing cells, islets, or organoids with interferon gamma (IFNγ) for at least 0.5 hour or at least one hour at a predetermined time point; and (b) repeating step (a) at least about two times during a time period of about or equal to 72-hours; wherein the cells, islets, or organoids are maintained in the absence of IFNγ between times of contact with IFNγ; and wherein steps (a) and (b) induce sustained expression of PD-L1 in the cells, islets, or organoids. In an embodiment of the method, the cells, islets, organoids or cells are contacted with IFNγ for a time period selected from about or equal to at least 0.5 hour, at least 1 hour, at least 2 hours, or more than 2 hours in step (a). In another embodiment of the method, the cells, islets, or organoids are contacted with IFNγ for a time period selected from about or equal to 0.5 hour, or about or equal to 1 hour, or about or equal to 2 hours, or about or equal to 12 hours in step (a). In another embodiment of the method, step (a) is repeated at least three times for at least about 0.5 hour each time, or for at least about 1 hour each time, or for at least about 2 hours each time in the about or equal to 72-hour time period of step (b). In another embodiment of the method, the cells, islets, or organoids are washed to remove the presence of IFNγ between step (a) and step (b). In another embodiment of the method, IFNγ is used in an amount of 1-25 ng/ml. In another embodiment of the method, IFNγ is used in an amount of 10 ng/ml. In another embodiment of the method, PD-L1 expression in the cells, islets, or organoids is maintained following step (b) for greater than about or equal to 7 days. In an embodiment, sustained expression of PD-L1 comprises about or equal to 3 days, 4 days, 5 days, 6 days, 7 days, 8 days, 9 days, 10 days, 11 days, 12 days, 13 days, 14 days, or longer, of PD-L1 expression in a cell.

In another aspect, a method of generating islets, or organoids and the cells thereof that survive and have reduced cell death following transplant, implant or transfer is provided, in which the method comprises: (a) contacting interferon gamma (IFNγ)-receptor expressing islets or organoids and the cells thereof with interferon gamma (IFNγ) in an amount of about 1 ng/ml to 25 ng/ml for greater than 1 hour at a first time point during a given time period, e.g., a time period of about or equal to 24-hours; and (b) contacting the islets or organoids and the cells thereof with IFNγ in an amount of about 1 ng/ml to 25 ng/ml for greater about 0.5-1 hour or longer at two or more additional time points during a following time period, e.g., a 48-hour time period, following step (a); wherein said islets or organoids are washed and rested in medium in the absence of IFNγ between being contacted with IFNγ; and wherein steps (a) and (b) induce sustained expression of PD-L1 in said islets or organoids. In an embodiment of the method, the islets or organoids are contacted with IFNγ in an amount of 10 ng/ml for at least 2 hours in step (a) and step (b). In another embodiment of the method, the islets or organoids are contacted with IFNγ for at least about 2 hours at 3 timepoints during the 72-hour timeperiod.

In an embodiment of any of the above-denoted methods, the cells, islets, or organoids are human cells, islets, or organoids. In another embodiment of the above methods, the organoids are HILOs or human HILOs. In another embodiment of the above methods, the islets are human cadaveric islets which are protected from destruction or clearance by the immune system.

In another aspect, a method of generating human cells, islets, or human islet like organoids (HILOs) that evade immune detection or autoimmunity is provided in which the method involves (a) contacting the human cells, islets or HILOs with interferon gamma (IFNγ) for greater than one hour at predetermined time point; repeating step (a) at least two times during a given time period, e.g., a 72-hour time period; wherein the human cells, islets, or HILOs are maintained in the absence of IFNγ between times of contact with IFNγ; and wherein steps (a) and (b) induce sustained expression of PD-L1 in the human islets or HILOs. In an embodiment of the method, the human cells, islets, or HILOs are contacted with IFNγ for 2 hours or more in step (a). In another embodiment of the method, the human cells, islets, or HILOs are contacted with IFNγ for 2 hours or 12 hours in step (a). In another embodiment of the method, step (a) is repeated three times for at least 2 hours each time in the given time period, i.e., a 72-hour time period. In another embodiment of the method, the human cells, islets, or HILOs are washed to remove IFNγ between step (a) and step (b). In another embodiment of the method, IFNγ is used in an amount of 1-25 ng/ml. In another embodiment of the method, IFNγ is used in an amount of 10 ng/ml. In another embodiment of the method, PD-L1 expression in the islets or HILOs is maintained or sustained following step (b) for greater than 7 days.

In another aspect, a method of generating human cells, islets or human islet like organoids (HILOs) that evade immune detection or autoimmunity is provided in which the method involves (a) contacting the human cells, islets or HILOs with interferon gamma (IFNγ) in an amount of about 1 ng/ml to 25 ng/ml for greater than 1 hour at a first time point during a given time period, e.g., a 24-hour time period; and (b) contacting the human cells, islets or HILOs with IFNγ in an amount of about 1 ng/ml to 25 ng/ml for greater than 1 hour at at least two additional time points during a next given time period, e.g., a 48-hour time period, following step (a); wherein the human cells, islets, or HILOs are washed and rested in medium in the absence of IFNγ between being contacted with IFNγ; and wherein steps (a) and (b) induce sustained expression of PD-L1 in the human islets or HILOs. In an embodiment of the method, the human islets or HILOs are contacted with interferon gamma (IFNγ) in an amount of 10 ng/ml for at least 2 hours in step (a) and step (b). In another embodiment of the method, the human islets or HILOs are contacted with interferon gamma (IFNγ) for at least 2 hours at 3 different intervals (time points) during a given time period, such as a 72-hour time period. In an embodiment of the method of the foregoing aspects, the human islets or HILOs are mature human islets or HILOs. In an embodiment, sustained expression of PD-L1 comprises about or equal to 3 days, 4 days, 5 days, 6 days, 7 days, 8 days, 9 days, 10 days, 11 days, 12 days, 13 days, 14 days, or longer, of PD-L1 expression in a cell. In embodiments of the method, the cells comprise cardiac cells, colon cells, kidney cells, bladder cells, liver cells (hepatocytes), esophageal cells, gastrointestinal cells, gastric (stomach) cells, lung cells, ovarian cells, cervical cells, uterine cells, testicular cells, pancreatic cells, pancreatic β cells, retinal cells, corneal cells, brain cells, muscle cells, hematopoietic cells, immune cells (B cells, T cells), chimeric antigen receptor-T cells (CAR-T cells), bone marrow cells, mononuclear cells, neurons, neuronal cells, insulin-producing pancreatic β cells derived from human skin cells, umbilical cord blood (UCB) cells, adipose derived mesenchymal stromal (stem) cells, cardiac stem cells, colon stem cells, kidney stem cells, liver (hepatocyte) stem cells, gastrointestinal stem cells, gastric stem cells, lung stem cells, pancreatic stem cells, pancreatic β stem cells, muscle stem cells, hematopoietic stem cells, immune cell (T cell or B cell) stem cells, bone marrow stem cells, CD133+ stem cells, CD34+ hematopoietic cells, CD34+ hematopoietic stem cells, mesenchymal stem cells, umbilical cord mesenchymal stem cells, retinal stem cells, neuronal stem cells, ectoderm-derived neuronal cells, immortalized dopaminergic neuronal precursor cells and organoids generated from or containing said cells. In an embodiment of the method, the organoids comprise cardiac organoids, intestinal/gastrointestinal organoids, colonic organoids, hepatic organoids, kidney organoids, bladder organoids, ovarian organoids, cervical organoids, neural organoids, or pulmonary (lung) organoids.

In an embodiment of the methods of any of the above-delineated aspects, the interferon gamma (IFNγ)-receptor expressing cells, islets, or organoids are contacted with IFNγ in culture medium or a physiologically acceptable solution, or in a three-dimensional matrix. In an embodiment, the the interferon gamma (IFNγ)-receptor expressing cells, islets, or organoids are contacted with IFNγ in a three-dimensional (3D) matrix, e.g., gellan gum, as described herein.

In another aspect, a method of generating an islet-like organoid that evades immune detection or autoimmunity is provided, in which the method comprises culturing endocrine progenitor cells in a three-dimensional matrix comprising Wnt4 or Wnt5a protein for a time sufficient to generate a multicellular islet-like organoid comprising two or more cell types selected from beta (β) cells, alpha (α) cells, delta (δ) cells, epsilon (ε) cells and duct-like cells; wherein the islet-like organoid secretes insulin in response to glucose; and subjecting the islet-like organoid to multiple intermittent exposure to interferon gamma (IFNγ) over a given time period, e.g., a time period of at least 24 hours; thereby inducing sustained expression of an immune checkpoint protein by the islet-like organoid and allowing the islet-like organoid to evade immune detection or autoimmunity. In an embodiment of the method, the islet-like organoid is exposed to IFNγ at least two times over at least a two-day time period. In another embodiment of the method, the islet-like organoid is exposed to IFNγ at least three times over a three-day time period. In another embodiment of the method, the islet-like organoid is exposed to IFNγ for greater than one hour at least two times over a two-day time period. In another embodiment of the method, the islet-like organoid is exposed to IFNγ for greater than one hour at least three times over a three-day time period. In another embodiment of the method, the islet-like organoid is exposed to IFNγ for two hours at least two times over a two-day time period. In another embodiment of the method, the islet-like organoid is exposed to IFNγ for two hours at least three times over a three-day time period. In embodiments of the method, the the islet-like organoid is intermittently exposed to IFNγ over a time period of 1, 2, 3, 4, 5, 6, 7, 8, 9, or 10 days, or longer.

In another aspect, a method of generating an islet-like organoid that evades immune detection or autoimmunity is provided, in which the method comprises culturing endocrine progenitor cells which recombinantly express an immune checkpoint protein in a three-dimensional matrix comprising Wnt4 or Wnt5a protein for a time sufficient to generate a multicellular islet-like organoid comprising two or more cell types selected from beta (β) cells, alpha (α) cells, delta (δ) cells, epsilon (ε) cells and duct-like cells; wherein the islet-like organoid secretes insulin in response to glucose and wherein the islet-like organoid evades immune detection and autoimmunity. In an embodiment, recombinant expression of the immune checkpoint protein results from transduction of islet-like organoid cells with a vector containing a polynucleotide encoding the immune checkpoint protein.

In an embodiment of the methods of the foregoing aspects, the three-dimensional matrix comprises a human Wnt4 protein, a recombinant human Wnt4 protein, a human Wnt5 protein, or a recombinant human Wnt5a protein. In a particular embodiment, the three-dimensional matrix comprises a recombinant human Wnt4 protein.

In an embodiment of the foregoing methods of generating an islet-like organoid that evades immune detection or autoimmunity, the three-dimensional matrix comprises gellan gum. In an embodiment, the three-dimensional matrix comprises recombinant human Wnt4 protein. In embodiments of the foregoing methods, the immune checkpoint protein binds to an immune cell-expressed cognate ligand selected from programmed cell-death protein 1 (PD-1); cytotoxic T-lymphocyte protein 4 (CTLA-4); lymphocyte activation gene 3 protein (LAG-3); killer cell immunoglobulin-like receptor (KIR); indoleamine 2,3-dioxygenase 1 (IDO1); tumor necrosis factor receptor superfamily member 9 (4-1BB); glucocorticoid-induced TNFR family related gene (GITR); T-cell immunoglobulin domain and mucin domain (TIM-3); tumor necrosis factor receptor superfamily member 4, (OX40); adenosine A2A receptor (A2AR); B7-H3; B7-H4; B7-1/B7-2; BTLA; V-domain Ig suppressor of T cell activation (VISTA); or a combination of any of the foregoing. In a particular embodiment, the immune checkpoint protein is programmed death ligand-1 (PD-L1).

In an embodiment of the methods of the foregoing aspects, the endocrine progenitor cells are selected from induced pluripotent stem cells (iPSCs), embryonic pluripotent stem cells (ePSCs), and/or pancreatic progenitor cells.

In an embodiment of the methods of the foregoing aspects, the the endocrine progenitor cells express at least one of neurogenin 3, neurod1, Nkx2.2 and Pax4 biomarkers.

In an embodiment of the methods of the foregoing aspects, the islet-like organoid is a human islet-like organoid (HILO). In a particular embodiment, the islet-like organoid is vascularized. In a particular embodiment, the islet-like organoid further comprises an adipose-derived stem cell and/or an endothelial cell. In an embodiment, the adipose-derived stem cell is a human adipose-derived stem cell (hADSC) and/or the endothelial cell is a human umbilical vein endothelial cell (HUVEC).

In an embodiment of the methods of the foregoing aspects, the islet-like organoid further exhibits at least one of KCl-stimulated insulin secretion, GLP-1 stimulated insulin secretion, somatostatin secretion, glucagon secretion.

In an embodiment of the methods of the foregoing aspects, the islet-like organoid expresses a beta cell lineage marker selected from the group consisting of NKX2-2, NEUROD1, RFX6, GCK, INS, NKX6-1, UCN3, MAFB and SYT4 and an ARX alpha cell lineage marker.

In an embodiment of the methods of the foregoing aspects, the islet-like organoid exhibits increased expression of Estrogen Related Receptor gamma (ERRγ).

In another embodiment of the methods of the foregoing aspects, the islet-like organoid exhibits increased oxidative metabolism characterized by increased oxygen consumption rate (OCR) and decreased cellular acidification rate (ECAR).

In an embodiment of the methods of the foregoing aspects, the islet-like organoid is a pancreatic islet organoid, a pancreatic organoid, a liver organoid, a heart organoid, or intestinal organoid. In a particular embodiment of the methods, the islet-like organoid is a human pancreatic islet organoid.

In another aspect, a method of generating a human islet like organoid (HILO) that evades immune detection or autoimmunity is provided, in which the method comprises (a) culturing endocrine progenitor cells in culture medium or a three-dimensional matrix comprising Wnt4 or Wnt5a protein for a time sufficient to generate a multicellular human islet-like organoid comprising two or more cell types selected from beta (β) cells, alpha (α) cells, delta (δ) cells, epsilon (ε) cells and duct-like cells; wherein the human islet-like organoid secretes insulin in response to glucose; (b) contacting the HILO of step (a) with interferon gamma (IFNγ) two or three times for greater than one hour each time over a total time period of at least 48-72 hours; wherein the human islets or HILOs are maintained in the absence of IFNγ between times of contact with IFNγ; and wherein steps (a) and (b) induce sustained expression of immune checkpoint protein programmed death ligand-1 (PD-L1) in the HILO. In an embodiment of the method, the HILO is contacted with IFNγ for 2 hours in step (b). In another embodiment of the method, the HILO is contacted with IFNγ two times for two hours each time, over at least 48 hours. In another embodiment of the method, the HILO is contacted with IFNγ three times for two hours each time, over at least 72 hours. In another embodiment of the method, the endocrine progenitor cells are selected from induced pluripotent stem cells (iPSCs), embryonic pluripotent stem cells (ePSCs), and/or pancreatic progenitor cells. In another embodiment of the method, the endocrine progenitor cells express at least one of neurogenin 3, neurod1, Nkx2.2 and Pax4 biomarkers. In another embodiment of the method, the HILO is vascularized and exhibits increased oxidative metabolism characterized by increased oxygen consumption rate (OCR) and decreased cellular acidification rate (ECAR).

In an embodiment of the methods of the foregoing aspects, IFNγ is used in an amount of 1-25 ng/ml. In an embodiment of the methods of the foregoing aspects, IFNγ is used in an amount of 10 ng/ml. In an embodiment of the methods of the foregoing aspects, PD-L1 expression in the islet-like organoid or HILO is maintained for greater than 7 days.

In an aspect, a human islet-like organoid or pancreatic islet organoid having sustained expression of an immune checkpoint protein is produced by the method as described in the above-delineated aspects. In an embodiment, the human islet-like organoid or pancreatic islet organoid exhibits sustained expression of the immune checkpoint protein PD-L1.

In another aspect is provided a human islet-like organoid (HILO) derived from endocrine progenitor cells cultured in culture medium or a three-dimensional matrix comprising Wnt4 or Wnt5 protein and comprising multi-lineage cells comprising at least two of beta (β) cells, alpha (α) cells, delta (δ) cells, epsilon (F) cells and duct-like cells, wherein the HILO is vascularized, exhibits glucose-stimulated insulin secretion (GSIS) and exhibits sustained expression of an immune checkpoint protein. In an embodiment, the human islet-like organoid (HILO) is a pancreatic islet-like organoid or a pancreatic organoid. In an embodiment, the human islet-like organoid (HILO) further exhibits KCl-stimulated insulin secretion or glucose stimulated insulin secretion. In another embodiment, the three-dimensional matrix for culturing the human islet-like organoid (HILO) comprises gellan gum. In another embodiment, the three-dimensional matrix for culturing the human islet-like organoid (HILO) comprises recombinant human Wnt4 protein. In an embodiment, the human islet-like organoid (HILO) is derived from endocrine progenitor cells which are selected from induced pluripotent stem cells (iPSCs), embryonic pluripotent stem cells (ePSCs), and/or pancreatic progenitor cells. In an embodiment, the endocrine progenitor cells express at least one of neurogenin 3, neurod1, Nkx2.2 and Pax4 biomarkers. In an embodiment, the human islet-like organoid (HILO) expresses FLTP and ESRR gamma genes. In an embodiment, the human islet-like organoid (HILO) further comprises an adipose-derived stem cell and/or an endothelial cell. In a particular embodiment, the adipose-derived stem cell is a human adipose-derived stem cell (hADSC) and/or the endothelial cell is a human umbilical vein endothelial cell (HUVEC). In another embodiment, the human islet-like organoid (HILO) further exhibits KCl-stimulated insulin secretion, GLP-1 stimulated insulin secretion, somatostatin secretion, or glucagon secretion. In another embodiment, the human islet-like organoid (HILO) expresses a beta cell lineage marker selected from the group consisting of NKX2-2, NEUROD1, RFX6, GCK, INS, NKX6-1, UCN3, MAFB and SYT4 and an ARX alpha cell lineage marker. In another embodiment, the human islet-like organoid (HILO) is a pancreatic HILO that expresses a beta cell transcription factor selected from the group consisting of Pdx1, MafA, Pax4, Pax6, NeuroDI, Nkx6-1, Gata6, and Foxa2. In embodiments, the human islet-like organoid (HILO) exhibit sustained expression of an immune checkpoint protein which binds to an immune cell-expressed cognate ligand selected from programmed cell-death protein 1 (PD-1); cytotoxic T-lymphocyte protein 4 (CTLA-4); lymphocyte activation gene 3 protein (LAG-3); killer cell immunoglobulin-like receptor (KIR); indoleamine 2,3-dioxygenase 1 (IDO1); tumor necrosis factor receptor superfamily member 9 (4-1BB); glucocorticoid-induced TNFR family related gene (GITR); T-cell immunoglobulin domain and mucin domain (TIM-3); tumor necrosis factor receptor superfamily member 4, (OX40); adenosine A2A receptor (A2AR); B7-H3; B7-H4; B7-1/B7-2; BTLA; V-domain Ig suppressor of T cell activation (VISTA); or a combination of any of the foregoing. In an embodiment, the human islet-like organoid (HILO) of any one of claims 40-54, wherein the immune checkpoint protein is programmed death ligand-1 (PD-L1).

In another aspect is provided a non-human organism transplanted or implanted with the human islet-like organoid, pancreatic islet organoid, or HILO as described in the foregoing aspects delineated above. In an embodiment, the non-human organism is a mammal. In an embodiment, the non-human organism is a mouse.

In another aspect, a method of treating a pancreatic disease in a subject is provided, in which the method comprises transplanting or implanting an islet-like organoid or a pancreatic islet organoid into the subject, wherein the islet-like organoid or a pancreatic islet organoid comprises endocrine progenitor cell-derived, multi-lineage cells including beta, alpha, delta, epsilon cells, duct-like cells, or a combination thereof, is vascularized, exhibits glucose-stimulated insulin secretion (GSIS) and exhibits sustained expression of an immune checkpoint protein to evade immune detection or autoimmunity.

In another aspect, a method of treating type 1 diabetes in a subject is provided, in which the method comprises transplanting or implanting an islet-like organoid or a pancreatic islet organoid into the subject, wherein the islet-like organoid or a pancreatic islet organoid comprises endocrine progenitor cell-derived multi-lineage cells including beta, alpha, delta, epsilon cells, duct-like cells, or a combination thereof, is vascularized, exhibits glucose-stimulated insulin secretion (GSIS) and exhibits sustained expression of an immune checkpoint protein to evade immune detection or autoimmunity.

In an embodiment of the methods delineated in the above-described aspects, the islet-like organoid or pancreatic islet organoid further exhibits KCl-stimulated insulin secretion, GLP-1 stimulated insulin secretion, somatostatin secretion, or glucagon secretion. In an embodiment of the methods delineated in the above-described aspects, the islet-like organoid or pancreatic islet organoid expresses a beta cell lineage marker selected from the group consisting of NKX2-2, NEUROD1, RFX6, GCK, INS, NKX6-1, UCN3, MAFB and SYT4 and an ARX alpha cell lineage marker. In an embodiment of the methods delineated in the above-described aspects, the endocrine progenitor cells are selected from induced pluripotent stem cells (iPSCs), embryonic pluripotent stem cells (ePSCs), and/or pancreatic progenitor cells. In an embodiment, the endocrine progenitor cells express at least one of neurogenin 3, neurod1, Nkx2.2 and Pax4 biomarkers. In an embodiment of the methods delineated in the above-described aspects, the islet-like organoid or pancreatic islet organoid expresses a beta cell transcription factor selected from the group consisting of Pdx1, MafA, Pax4, Pax6, NeuroD1, Nkx6-1, Gata6, and Foxa2. In an embodiment of the treatment methods as described in the above-delineated aspects, the immune checkpoint protein binds to an immune cell-expressed cognate ligand selected from programmed cell-death protein 1 (PD-1); cytotoxic T-lymphocyte protein 4 (CTLA-4); lymphocyte activation gene 3 protein (LAG-3); killer cell immunoglobulin-like receptor (KIR); indoleamine 2,3-dioxygenase 1 (IDO1); tumor necrosis factor receptor superfamily member 9 (4-1BB); glucocorticoid-induced TNFR family related gene (GITR); T-cell immunoglobulin domain and mucin domain (TIM-3); tumor necrosis factor receptor superfamily member 4, (OX40); adenosine A2A receptor (A2AR); B7-H3; B7-H4; B7-1/B7-2; BTLA; V-domain Ig suppressor of T cell activation (VISTA); or a combination of any of the foregoing. In a particular embodiment, the immune checkpoint protein is programmed death ligand-1 (PD-L1). In an embodiment of the treatment methods as described in the above-delineated aspects, the islet-like organoid or pancreatic islet organoid is produced by a method described in the aspects hereinabove. In an embodiment of the treatment methods as described in the above-delineated aspects, the islet-like organoid or pancreatic islet organoid is the organoid as described in the above-delineated aspects. In an embodiment of the treatment methods as described in the above-delineated aspects, an immunosuppressive agent is administered to the subject. In an embodiment of the treatment methods as described in the above-delineated aspects, the subject is human. In an embodiment of the treatment methods as described in the above-delineated aspects, the pancreatic disease is type 1 diabetes or type 2 diabetes.

In another aspect, a method of cell transplantation is provided, in which the method comprises administering to a subject in need thereof an immunoprotected cell, human islet-like organoid or pancreatic islet organoid as described in the above-delineated aspects. In an embodiment, the immunoprotected cell, human islet-like organoid or pancreatic islet organoid is syngeneic, autologous, allogeneic or xenogeneic to the subject receiving the transplant.

In another aspect, a kit containing an immunoprotected cell, human islet-like organoid or pancreatic islet organoid as described in the above-delineated aspects, or a pharmaceutically acceptable composition comprising the immunoprotected cell, human islet-like organoid or pancreatic islet organoid is provided. In an embodiment, the kit contains an immunoprotected cell, human islet-like organoid or pancreatic islet organoid that is syngeneic, autologous, allogeneic, or xenogeneic.

Other features and advantages will be apparent from the detailed description of the embodiments and from the claims.

### Definitions

Unless defined otherwise, all technical and scientific terms used herein have the meaning commonly understood by a person skilled in the art to which this invention pertains. The following references provide one of skill in the pertinent art with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “AFP polypeptide” or “alpha-fetoprotein” is meant a protein or fragment thereof having at least 85% amino acid sequence identity to the sequence provided at NCBI Accession No. NP_001125.1 and having a biological activity of an AFP polypeptide. Exemplary biological activities of an AFP polypeptide include binding to copper, nickel, fatty acids, and bilirubin. The amino acid sequence provided at NCBI Accession No. NP_001125.1 is shown below:

| | |
|---|---|
| 1 | MKWVESIFLI FLLNFTESRT LHRNEYGIAS ILDSYQCTAE ISLADLATIF FAQFVQEATY |
| 61 | KEVSKMVKDA LTAIEKPTGD EQSSGCLENQ LPAFLEELCH EKEILEKYGH SDCCSQSEEG |
| 121 | RHNCFLAHKK PTPASIPLFQ VPEPVTSCEA YEEDRETFMN KFIYEIARRH PFLYAPTILL |
| 181 | WAARYDKIIP SCCKAENAVE CFQTKAATVT KELRESSLLN QHACAVMKNF GTRTFQAITV |
| 241 | TKLSQKFTKV NFTEIQKLVL DVAHVHEHCC RGDVLDCLQD GEKIMSYICS QQDTLSNKIT |
| 301 | ECCKLTTLER GQCIIHAEND EKPEGLSPNL NRFLGDRDFN QFSSGEKNIF LASFVHEYSR |
| 361 | RHPQLAVSVI LRVAKGYQEL LEKCFQTENP LECQDKGEEE LQKYIQESQA LAKRSCGLFQ |
| 421 | KLGEYYLQNA FLVAYTKKAP QLTSSELMAI TRKMAATAAT CCQLSEDKLL ACGEGAADII |
| 481 | IGHLCIRHEM TPVNPGVGQC CTSSYANRRP CFSSLVVDET YVPPAFSDDK FIFHKDLCQA |
| 541 | QGVALQTMKQ EFLINLVKQK PQITEEQLEA VIADFSGLLE KCCQGQEQEV CFAEEGQKLI |
| 601 | SKTRAALGV |

By “AFP polynucleotide” is meant a polynucleotide encoding a AFP polypeptide or fragment thereof. An exemplary AFP polynucleotide sequence is provided at NCBI Ref NM_001134.2. The sequence provided at NCBI Ref NM_001134.2 is reproduced below:

| | |
|---|---|
| 1 | atattgtgct tccaccactg ccaataacaa aataactagc aaccatgaag tgggtggaat |
| 61 | caattttttt aattttccta ctaaatttta ctgaatccag aacactgcat agaaatgaat |
| 121 | atggaatagc ttccatattg gattcttacc aatgtactgc agagataagt ttagctgacc |
| 181 | tggctaccat attttttgcc cagtttgttc aagaagccac ttacaaggaa gtaagcaaaa |
| 241 | tggtgaaaga tgcattgact gcaattgaga aacccactgg agatgaacag tcttcagggt |
| 301 | gtttagaaaa ccagctacct gcctttctgg aagaactttg ccatgagaaa gaaattttgg |
| 361 | agaagtacgg acattcagac tgctgcagcc aaagtgaaga gggaagacat aactgttttc |
| 421 | ttgcacacaa aaagcccact ccagcatcga tcccactttt ccaagttcca gaacctgtca |
| 481 | caagctgtga agcatatgaa gaagacaggg agacattcat gaacaaattc atttatgaga |
| 541 | tagcaagaag gcatcccttc ctgtatgcac ctacaattct tctttgggct gctcgctatg |
| 601 | acaaaataat tccatcttgc tgcaaagctg aaaatgcagt tgaatgcttc caaacaaagg |
| 661 | cagcaacagt tacaaaagaa ttaagagaaa gcagcttgtt aaatcaacat gcatgtgcag |
| 721 | taatgaaaaa ttttgggacc cgaactttcc aagccataac tgttactaaa ctgagtcaga |
| 781 | agtttaccaa agttaatttt actgaaatcc agaaactagt cctggatgtg gcccatgtac |
| 841 | atgagcactg ttgcagagga gatgtgctgg attgtctgca ggatggggaa aaaatcatgt |
| 901 | cctacatatg ttctcaacaa gacactctgt caaacaaaat aacagaatgc tgcaaactga |
| 961 | ccacgctgga acgtggtcaa tgtataattc atgcagaaaa tgatgaaaaa cctgaaggtc |
| 1021 | tatctccaaa tctaaacagg tttttaggag atagagattt taaccaattt tcttcagggg |
| 1081 | aaaaaaatat cttcttggca agttttgttc atgaatattc aagaagacat cctcagcttg |
| 1141 | ctgtctcagt aattctaaga gttgctaaag gataccagga gttattggag aagtgtttcc |
| 1201 | agactgaaaa ccctcttgaa tgccaagata aaggagaaga agaattacag aaatacatcc |
| 1261 | aggagagcca agcattggca aagcgaagct gcggcctctt ccagaaacta ggagaatatt |
| 1321 | acttacaaaa tgcgtttctc gttgcttaca caaagaaagc cccccagctg acctcgtcgg |
| 1381 | agctgatggc catcaccaga aaaatggcag ccacagcagc cacttgttgc caactcagtg |
| 1441 | aggacaaact attggcctgt ggcgagggag cggctgacat tattatcgga cacttatgta |
| 1501 | tcagacatga aatgactcca gtaaaccctg gtgttggcca gtgctgcact tcttcatatg |
| 1561 | ccaacaggag gccatgcttc agcagcttgg tggtggatga aacatatgtc cctcctgcat |
| 1621 | tctctgatga caagttcatt ttccataagg atctgtgcca agctcagggt gtagcgctgc |
| 1681 | aaacgatgaa gcaagagttt ctcattaacc ttgtgaagca aaagccacaa ataacagagg |
| 1741 | aacaacttga ggctgtcatt gcagatttct caggcctgtt ggagaaatgc tgccaaggcc |
| 1801 | aggaacagga agtctgcttt gctgaagagg gacaaaaact gatttcaaaa actcgtgctg |
| 1861 | ctttgggagt ttaaattact tcaggggaag agaagacaaa acgagtcttt cattcggtgt |
| 1921 | gaacttttct ctttaatttt aactgattta acactttttg tgaattaatg aaatgataaa |
| 1981 | gacttttatg tgagatttcc ttatcacaga aataaaatat ctccaaatgt ttccttttca |
| 2041 | aaaaaaaaaa aaaaaaa |

By “ALB polypeptide” or “albumin” is meant a protein or fragment thereof having at least 85% amino acid sequence identity to the sequence provided at NCBI Accession No. NP_000468.1 and having a biological activity of ALB polypeptide. Exemplary biological activities of ALB polypeptide include binding to fatty acids, calcium ions, sodium ions, potassium ions, hormones, and bilirubin; stabilization of extracellular fluid volume; and, transport of plasma zinc. The amino acid sequence provided at NCBI Accession No. NP_000468.1 is shown below:

| | |
|---|---|
| 1 | MKWVTFISLL FLFSSAYSRG VFRRDAHKSE VAHRFKDLGE ENFKALVLIA FAQYLQQCPF |
| 61 | EDHVKLVNEV TEFAKTCVAD ESAENCDKSL HTLFGDKLCT VATLRETYGE MADCCAKQEP |
| 121 | ERNECFLQHK DDNPNLPRLV RPEVDVMCTA FHDNEETFLK KYLYEIARRH PYFYAPELLF |
| 181 | FAKRYKAAFT ECCQAADKAA CLLPKLDELR DEGKASSAKQ RLKCASLQKF GERAFKAWAV |
| 241 | ARLSQRFPKA EFAEVSKLVT DLTKVHTECC HGDLLECADD RADLAKYICE NQDSISSKLK |
| 301 | ECCEKPLLEK SHCIAEVEND EMPADLPSLA ADFVESKDVC KNYAEAKDVF LGMFLYEYAR |
| 361 | RHPDYSVVLL LRLAKTYETT LEKCCAAADP HECYAKVFDE FKPLVEEPQN LIKQNCELFE |
| 421 | QLGEYKFQNA LLVRYTKKVP QVSTPTLVEV SRNLGKVGSK CCKHPEAKRM PCAEDYLSVV |
| 481 | LNQLCVLHEK TPVSDRVTKC CTESLVNRRP CFSALEVDET YVPKEFNAET FTFHADICTL |
| 541 | SEKERQIKKQ TALVELVKHK PKATKEQLKA VMDDFAAFVE KCCKADDKET CFAEEGKKLV |
| 601 | AASQAALGL |

By “ALB polynucleotide” is meant a polynucleotide encoding a ALB polypeptide or fragment thereof. An exemplary AFP polynucleotide sequence is provided at NCBI Ref NM_000477.5. The sequence provided at NCBI Ref NM_000477.5 is reproduced below:

| | |
|---|---|
| 1 | agtatattag tgctaatttc cctccgtttg tcctagcttt tctcttctgt caaccccaca |
| 61 | cgcctttggc acaatgaagt gggtaacctt tatttccctt ctttttctct ttagctcggc |
| 121 | ttattccagg ggtgtgtttc gtcgagatgc acacaagagt gaggttgctc atcggtttaa |
| 181 | agatttggga gaagaaaatt tcaaagcctt ggtgttgatt gcctttgctc agtatcttca |
| 241 | gcagtgtcca tttgaagatc atgtaaaatt agtgaatgaa gtaactgaat ttgcaaaaac |
| 301 | atgtgttgct gatgagtcag ctgaaaattg tgacaaatca cttcataccc tttttggaga |
| 361 | caaattatgc acagttgcaa ctcttcgtga aacctatggt gaaatggctg actgctgtgc |
| 421 | aaaacaagaa cctgagagaa atgaatgctt cttgcaacac aaagatgaca acccaaacct |
| 481 | cccccgattg gtgagaccag aggttgatgt gatgtgcact gcttttcatg acaatgaaga |
| 541 | gacatttttg aaaaaatact tatatgaaat tgccagaaga catccttact tttatgcccc |
| 601 | ggaactcctt ttctttgcta aaaggtataa agctgctttt acagaatgtt gccaagctgc |
| 661 | tgataaagct gcctgcctgt tgccaaagct cgatgaactt cgggatgaag ggaaggcttc |
| 721 | gtctgccaaa cagagactca agtgtgccag tctccaaaaa tttggagaaa gagctttcaa |
| 781 | agcatgggca gtagctcgcc tgagccagag atttcccaaa gctgagtttg cagaagtttc |
| 841 | caagttagtg acagatctta ccaaagtcca cacggaatgc tgccatggag atctgcttga |
| 901 | atgtgctgat gacagggcgg accttgccaa gtatatctgt gaaaatcaag attcgatctc |
| 961 | cagtaaactg aaggaatgct gtgaaaaacc tctgttggaa aaatcccact gcattgccga |
| 1021 | agtggaaaat gatgagatgc ctgctgactt gccttcatta gctgctgatt ttgttgaaag |
| 1081 | taaggatgtt tgcaaaaact atgctgaggc aaaggatgtc ttcctgggca tgtttttgta |
| 1141 | tgaatatgca agaaggcatc ctgattactc tgtcgtgctg ctgctgagac ttgccaagac |
| 1201 | atatgaaacc actctagaga agtgctgtgc cgctgcagat cctcatgaat gctatgccaa |
| 1261 | agtgttcgat gaatttaaac ctcttgtgga agagcctcag aatttaatca aacaaaattg |
| 1321 | tgagcttttt gagcagcttg gagagtacaa attccagaat gcgctattag ttcgttacac |
| 1381 | caagaaagta ccccaagtgt caactccaac tcttgtagag gtctcaagaa acctaggaaa |
| 1441 | agtgggcagc aaatgttgta aacatcctga agcaaaaaga atgccctgtg cagaagacta |
| 1501 | tctatccgtg gtcctgaacc agttatgtgt gttgcatgag aaaacgccag taagtgacag |
| 1561 | agtcaccaaa tgctgcacag aatccttggt gaacaggcga ccatgctttt cagctctgga |
| 1621 | agtcgatgaa acatacgttc ccaaagagtt taatgctgaa acattcacct tccatgcaga |
| 1681 | tatatgcaca ctttctgaga aggagagaca aatcaagaaa caaactgcac ttgttgagct |
| 1741 | cgtgaaacac aagcccaagg caacaaaaga gcaactgaaa gctgttatgg atgatttcgc |
| 1801 | agcttttgta gagaagtgct gcaaggctga cgataaggag acctgctttg ccgaggaggg |
| 1861 | taaaaaactt gttgctgcaa gtcaagctgc cttaggctta taacatcaca tttaaaagca |
| 1921 | tctcagccta ccatgagaat aagagaaaga aaatgaagat caaaagctta ttcatctgtt |
| 1981 | tttctttttc gttggtgtaa agccaacacc ctgtctaaaa aacataaatt tctttaatca |
| 2041 | ttttgcctct tttctctgtg cttcaattaa taaaaaatgg aaagaatcta atagagtggt |
| 2101 | acagcactgt tatttttcaa agatgtgttg ctatcctgaa aattctgtag gttctgtgga |
| 2161 | agttccagtg ttctctctta ttccacttcg gtagaggatt tctagtttct tgtgggctaa |
| 2221 | ttaaataaat cattaatact cttctaaaaa aaaaaaaaaa aaaa |

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “altered” is meant an increase or decrease. An increase is any positive change, e.g., by at least about 5%, 10%, or 20%; by at least about 25%, 50%, 75%, or even by 100%, 200%, 300% or more. A decrease is a negative change, e.g., a decrease by at least about 5%, 10%, or 20%; by at least about 25%, 50%, 75%; or even an increase by 100%, 200%, 300% or more.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “CDX2 polypeptide” is meant a protein or fragment thereof having at least 85% amino acid sequence identity to the sequence provided at NCBI Accession No. NP_001256.3 and having transcription factor activity. The amino acid sequence provided at NCBI Accession No. NP_001256.3 is shown below:

| | |
|---|---|
| 1 | MYVSYLLDKD VSMYPSSVRH SGGLNLAPQN FVSPPQYPDY GGYHVAAAAA AAANLDSAQS |
| 61 | PGPSWPAAYG APLREDWNGY APGGAAAAAN AVAHGLNGGS PAAAMGYSSP ADYHPHHHPH |
| 121 | HHPHHPAAAP SCASGLLQTL NPGPPGPAAT AAAEQLSPGG QRRNLCEWMR KPAQQSLGSQ |
| 181 | VKTRTKDKYR VVYTDHQRLE LEKEFHYSRY ITIRRKAELA ATLGLSERQV KIWFQNRRAK |
| 241 | ERKINKKKLQ QQQQQQPPQP PPPPPQPPQP QPGPLRSVPE PLSPVSSLQA SVSGSVPGVL |
| 301 | GPTGGVLNPT VTQ |

By “CDX2 polynucleotide” is meant a polynucleotide encoding a CDX2 polypeptide or fragment thereof. An exemplary CDX2 polynucleotide sequence is provided at NCBI Ref: NM_001265.4. The sequence provided at NCBI Ref: NM_001265.4 is reproduced below:

| | |
|---|---|
| 1 | ctccaaccat tggtgtctgt gtcattacta atagagtctt gtaaacactc gttaatcacg |
| 61 | gaaggccgcc ggcctggggc tccgcacgcc agcctgtggc gggtcttccc cgcctctgca |
| 121 | gcctagtggg aaggaggtgg gaggaaagaa ggaagaaagg gagggaggga ggaggcaggc |
| 181 | cagagggagg gaccgcctcg gaggcagaag agccgcgagg agccagcgga gcaccgcggg |
| 241 | ctggggcgca gccacccgcc gctcctcgag tcccctcgcc cctttccctt cgtgcccccc |
| 301 | ggcagcctcc agcgtcggtc cccaggcagc atggtgaggt ctgctcccgg accctcgcca |
| 361 | ccatgtacgt gagctacctc ctggacaagg acgtgagcat gtaccctagc tccgtgcgcc |
| 421 | actctggcgg cctcaacctg gcgccgcaga acttcgtcag ccccccgcag tacccggact |
| 481 | acggcggtta ccacgtggcg gccgcagctg cagcggcagc gaacttggac agcgcgcagt |
| 541 | ccccggggcc atcctggccg gcagcgtatg gcgccccact ccgggaggac tggaatggct |
| 601 | acgcgcccgg aggcgccgcg gccgccgcca acgccgtggc tcacggcctc aacggtggct |
| 661 | ccccggccgc agccatgggc tacagcagcc ccgcagacta ccatccgcac caccacccgc |
| 721 | atcaccaccc gcaccacccg gccgccgcgc cttcctgcgc ttctgggctg ctgcaaacgc |
| 781 | tcaaccccgg ccctcctggg cccgccgcca ccgctgccgc cgagcagctg tctcccggcg |
| 841 | gccagcggcg gaacctgtgc gagtggatgc ggaagccggc gcagcagtcc ctcggcagcc |
| 901 | aagtgaaaac caggacgaaa gacaaatatc gagtggtgta cacggaccac cagcggctgg |
| 961 | agctggagaa ggagtttcac tacagtcgct acatcaccat ccggaggaaa gccgagctag |
| 1021 | ccgccacgct ggggctctct gagaggcagg ttaaaatctg gtttcagaac cgcagagcaa |
| 1081 | aggagaggaa aatcaacaag aagaagttgc agcagcaaca gcagcagcag ccaccacagc |
| 1141 | cgcctccgcc gccaccacag cctccccagc ctcagccagg tcctctgaga agtgtcccag |
| 1201 | agcccttgag tccggtgtct tccctgcaag cctcagtgtc tggctctgtc cctggggttc |
| 1261 | tggggccaac tgggggggtg ctaaacccca ccgtcaccca gtgacccacc gggttctgca |
| 1321 | gcggcagagc aattccaggc tgagccatga ggagcgtgga ctctgctaga ctcctcagga |
| 1381 | gagacccctc ccctcccacc cacagccata gacctacaga cctggctctc agaggaaaaa |
| 1441 | tgggagccag gagtaagaca agtgggattt ggggcctcaa gaaatatact ctcccagatt |
| 1501 | tttacttttt cccatctggc tttttctgcc actgaggaga cagaaagcct ccgctgggct |
| 1561 | tcattccgga ctggcagaag cattgcctgg actgaccaca ccaaccaggc cttcatcctc |
| 1621 | ctccccagct cttctcttcc tagatctgca ggctgcacct ctggctagag ccgaggggag |
| 1681 | agagggactc aagggaaagg caagcttgag gccaagatgg ctgctgcctg ctcatggccc |
| 1741 | tcggaggtcc agctgggcct cctgcctccg ggcaggcaag gtttacactg cggaagccaa |
| 1801 | aggcagctaa gatagaaagc tggactgacc aaagactgca gaacccccag gtggcctgcg |
| 1861 | tcttttttct cttcccttcc cagaccagga aaggcttggc tggtgtatgc acagggtgtg |
| 1921 | gtatgagggg gtggttattg gactccaggc ctgaccaggg ggcccgaaca gggacttgtt |
| 1981 | tagagagcct gtcaccagag cttctctggg ctgaatgtat gtcagtgcta taaatgccag |
| 2041 | agccaacctg gacttcctgt cattttcaca atcttggggc tgatgaagaa gggggtgggg |
| 2101 | ggagtttgtg ttgttgttgc tgctgtttgg gttgttggtc tgtgtaacat ccaagccaga |
| 2161 | gtttttaaag ccttctggat ccatgggggg agaagtgata tggtgaaggg aagtggggag |
| 2221 | tatttgaaca cagttgaatt ttttctaaaa agaaaaagag ataaatgagc tttccagatt |
| 2281 | tcagattctg tatttatctt cagattttgt ctgcaactat tttttatttt ttaaagaaat |
| 2341 | gaaatatctt caaaaaaaaa aaaaaaaaaa |

By “CYP3A7 polypeptide” or “cytochrome P450” is meant a protein or fragment thereof having at least 85% amino acid sequence identity to the sequence provided at NCBI Accession No. NP_000756.3 and having monooxygenase activity. The amino acid sequence provided at NCBI Accession No. NP_000756.3 is shown below:

| | |
|---|---|
| 1 | MDLIPNLAVE TWLLLAVSLI LLYLYGTRTH GLFKKLGIPG PTPLPFLGNA LSFRKGYWTF |
| 61 | DMECYKKYRK VWGIYDCQQP MLAITDPDMI KTVLVKECYS VFTNRRPFGP VGFMKNAISI |
| 121 | AEDEEWKRIR SLLSPTFTSG KLKEMVPIIA QYGDVLVRNL RREAETGKPV TLKHVFGAYS |
| 181 | MDVITSTSFG VSIDSLNNPQ DPFVENTKKL LRFNPLDPFV LSIKVFPFLT PILEALNITV |
| 241 | FPRKVISFLT KSVKQIKEGR LKETQKHRVD FLQLMIDSQN SKDSETHKAL SDLELMAQSI |
| 301 | IFIFAGYETT SSVLSFIIYE LATHPDVQQK VQKEIDTVLP NKAPPTYDTV LQLEYLDMVV |
| 361 | NETLRLFPVA MRLERVCKKD VEINGMFIPK GVVVMIPSYV LHHDPKYWTE PEKFLPERFS |
| 421 | KKNKDNIDPY IYTPFGSGPR NCIGMRFALV NMKLALVRVL QNFSFKPCKE TQIPLKLRFG |
| 481 | GLLLTEKPIV LKAESRDETV SGA |

By “CYP3A7 polynucleotide” is meant a polynucleotide encoding a CYP3A7 polypeptide or fragment thereof. An exemplary AFP polynucleotide sequence is provided at NCBI Ref: NM_000765.4. The sequence provided at NCBI Ref: NM_000765.4 is reproduced below:

| | |
|---|---|
| 1 | aatcactgct gtgcagggca ggaaagctcc acacacacag cccagcaaac agcagcacgc |
| 61 | tgctgaaaaa aagactcaga ggagagagat aaggaaggaa agtagtgatg gatctcatcc |
| 121 | caaacttggc cgtggaaacc tggcttctcc tggctgtcag cctgatactc ctctatctat |
| 181 | atggaacccg tacacatgga ctttttaaga agcttggaat tccagggccc acacctctgc |
| 241 | cttttttggg aaatgctttg tccttccgta agggctattg gacgtttgac atggaatgtt |
| 301 | ataaaaagta tagaaaagtc tggggtattt atgactgtca acagcctatg ctggctatca |
| 361 | cagatcccga catgatcaaa acagtgctag tgaaagaatg ttattctgtc ttcacaaacc |
| 421 | ggaggccttt cgggccagtg ggatttatga aaaatgccat ctctatagct gaggatgaag |
| 481 | aatggaagag aatacgatca ttgctgtctc caacattcac cagcggaaaa ctcaaggaga |
| 541 | tggtccctat cattgcccag tatggagatg tgttggtgag aaatctgagg cgggaagcag |
| 601 | agacaggcaa gcctgtcacc ttgaaacacg tctttggggc ctacagcatg gatgtgatca |
| 661 | ctagcacatc atttggagtg agcatcgact ctctcaacaa tccacaagac ccctttgtgg |
| 721 | aaaacaccaa gaagctttta agatttaatc cattagatcc attcgttctc tcaataaaag |
| 781 | tctttccatt ccttacccca attcttgaag cattaaatat cactgtgttt ccaagaaaag |
| 841 | ttataagttt tctaacaaaa tctgtaaaac agataaaaga aggtcgcctc aaagagacac |
| 901 | aaaagcaccg agtggatttc cttcagctga tgattgactc tcagaattca aaagactctg |
| 961 | agacccacaa agctctgtct gatctggagc tcatggccca atcaattatc tttatttttg |
| 1021 | ctggctatga aaccacgagc agtgttctct ccttcattat atatgaactg gccactcacc |
| 1081 | ctgatgtcca gcagaaagtg cagaaggaaa ttgatacagt tttacccaat aaggcaccac |
| 1141 | ccacctatga tactgtgcta cagttggagt atcttgacat ggtggtgaat gaaacactca |
| 1201 | gattattccc agttgctatg agacttgaga gggtctgcaa aaaagatgtt gaaatcaatg |
| 1261 | ggatgtttat tcccaaaggg gtggtggtga tgattccaag ctatgttctt catcatgacc |
| 1321 | caaagtactg gacagagcct gagaagttcc tccctgaaag gttcagtaaa aagaacaagg |
| 1381 | acaacataga tccttacata tacacaccct ttggaagtgg acccagaaac tgcattggca |
| 1441 | tgaggtttgc tctcgtgaac atgaaacttg ctctagtcag agtccttcag aacttctcct |
| 1501 | tcaaaccttg taaagaaaca cagatccccc tgaaattacg ctttggagga cttcttctaa |
| 1561 | cagaaaaacc cattgttcta aaggctgagt caagggatga gaccgtaagt ggagcctgat |
| 1621 | ttccctaagg acttctggtt tgctctttaa gaaagctgtg ccccagaaca ccagagacct |
| 1681 | caaattactt tacaaataga accctgaaat gaagacgggc ttcatccaat gtgctgcata |
| 1741 | aataatcagg gattctgtac gtgcattgtg ctctctcatg gtctgtatag agtgttatac |
| 1801 | ttggtaatat agaggagatg accaaatcag tgctggggaa gtagatttgg cttctctgct |
| 1861 | tctcatagga ctatctccac cacccccagt tagcaccatt aactcctcct gagctctgat |
| 1921 | aacataatta acatttctca ataatttcaa ccacaatcat taataaaaat aggaattatt |
| 1981 | ttgatggctc taacagtgac atttatatca tgtgttatat ctgtagtatt ctatagtaag |
| 2041 | ctttatatta agcaaatcaa taaaaacctc tttacaaaag taaaaaaaaa aaaaaaaaa |

“Autologous” refers to biological material, e.g., autologous cells, tissues, islets, organoids, or islet-like organoids, that are obtained or derived from the same individual, subject, or patient. By way of example, autologous transplants (e.g., donor cells, tissues, organs, islets, organoids, or islet-like organoids) involve one individual, subject, or patient as both donor and recipient. “Syngeneic” refers to cells, tissues, organs, islets, organoids, islet-like organoids, or organisms (or other biological material) that are genetically similar or identical, (and of the same species) and thus, are immunologically compatible. Syngeneic donor biological material is typically so closely related that transplantation does not provoke an immune response in the recipient. “Allogeneic” refers to biological material, e.g., donor allogeneic cells, tissues, organs, islets, organoids, or islet-like organoids, that is genetically dissimilar to the recipient. Allogeneic biological material is typically obtained or derived from individuals of the same species. In addition, allogeneic biological material may be from an unrelated donor or from a donor matched as to MHC or HLA histocompatibility antigen type(s) with that of the recipient. “Xenogeneic” refers to biological material (e.g., cells, tissues, organs, islets, organoids, or islet-like organoids) that are derived or obtained from individuals of a different species. By way of example, autologous, syngeneic, allogeneic, or xenogeneic cells, tissues, organs, islets, organoids, or islet-like organoids may be used for transplant or implant, particularly, those generated by the methods involving IFNγ treatment (e.g., MPS IFNγ treatment) as described herein to yield long-term, immune evasive, transplanted or implanted biological material. In an embodiment, such biological material is obtained or generated from a living donor (individual, subject, or organism). In an embodiment, such biological material is obtained or generated from a nonliving donor, e.g., cadaveric human islets or donor-matched cadaveric human islets.

As used herein, the term “carrier” refers to a physiologically acceptable diluent, excipient, buffer, or vehicle with which a composition (e.g., a physiologically acceptable or pharmaceutical composition), e.g., comprising a cell, islet, islet-like organoid, or organoid, may be administered to a subject or in which it may be stored. Pharmaceutical and pharmaceutically acceptable carriers include sterile liquids, such as medium, saline, buffers, and the like. In embodiments, the physiologically acceptable carriers are used in pharmaceutical compositions that are administered to or transplanted into a subject, including, but not limited to, a human subject or patient. In some embodiments, water or aqueous saline solutions and aqueous dextrose and glycerol solutions may be employed as carriers, particularly for injectable solutions. Suitable pharmaceutical carriers (and pharmaceutical compositions) are known and used by practitioners in the art and are described in Remington: The Science and Practice of Pharmacy (20th ed.), ed. A. R. Gennaro, Lippincott Williams & Wilkins, 2000, and later editions thereof.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, the term “immune response” refers to a subject's immune system response or reaction to one or more antigens, (e.g., an immunogenic protein or peptide), and/or the epitopes of the antigens, recognized by the immune system as foreign, allogeneic, or heterologous. Immune responses include both cell-mediated immune responses (i.e., responses mediated by effector T cells, such as antigen-specific or non-specific T-cells, such as CD8+ T-cells, Th1 cells, Th2 cells, and Th17 cells) as well as humoral immune responses (i.e., responses characterized by B-cell activation and the production of antigen-specific antibodies). The term “immune response” encompasses both the innate immune responses to an antigen or immunogen, as well as memory responses that are a result of acquired immunity and can involve either B cells or T cells, or both.

By “immune checkpoint protein” or “immune checkpoint molecule,” or simply, “checkpoint protein or molecule” is meant a protein or molecule that can either induce or hinder activation of T cells, or a particular process in a cellular or immune system pathway, e.g., to prevent errors or an abnormal or pathological activity or condition. In an immune response, the crucial interaction between antigen presenting cells (APCs) and T-cells is tightly regulated by a ‘three signal model’: (1) display of a surface complex consisting of an antigen bound on a major histocompatibility complex (MHC) protein class I or II (MHC I or II) molecule to a T-cell receptor (TCR) on a T-cell (CD8+ or CD4+); (2) costimulation by immune checkpoint proteins and (3) cytokines. Immune checkpoint proteins comprise costimulatory and inhibitory proteins that can either induce or inhibit activation of T-cells. Naive T-cells that only receive signal 1 without costimulatory signal 2 become anergic or die through apoptosis. The engagement of costimulatory ligand/receptor pairs triggers an accumulation of receptors and protein complexes at the center of the immunological synapse, which then amplifies and enhances the duration of TCR signaling (Wulfing, C. and Davis, M. M., 1998, Science, 282:2266-2269). The cytokine environment, signal 3, then induces naïve CD4+ T-cells to differentiate into various T-cell subsets, such as T helper (Th)1 cells, Th2 cells, Th17 cells and regulatory T-cells (Tregs), each of which produce and release a distinct set of cytokines upon activation. (Foks, A. C. and Kuiper, J., 2017, Br. J. Pharmacol., 174:3940-3955).

The immune system provides a large variety of stimulatory and inhibitory immune checkpoint proteins (signal 2), and each pathway has its own unique effect on the fate of individual immune cells. Signaling through stimulatory immune checkpoint proteins can promote cell survival, cell cycle progression and differentiation to effector and memory cells, while inhibitory immune checkpoint protein signaling can terminate these processes directly or indirectly by the induction of Tregs. Costimulation can be provided in cis, i.e., both signals 1 and 2 are provided by the same APC, or in trans, i.e., signal 2 is provided by a different or ‘bystander’ APC than signal 1 (Roska, A. K. and Lipsky, P. E., 1985, J Immunol., 135:2953-2961; Liu, Y. and Janeway, C. A., Jr., 1992, Proc. Natl. Acad. Sci. USA, 89:3845-3849; Ding, L. and Shevach, E. M., 1994, Eur. J Immunol., 24:859-866).

Checkpoint proteins are regulators of the immune system and frequently are bound by or interact with ligands (cognate ligands), which may cause a given effect, e.g., cell stimulation, anergy, or apoptosis. In an embodiment, the immune checkpoint protein is one which binds a cognate ligand (e.g., a receptor ligand) on an immune cell surface, e.g., a T cell surface receptor. In a specific embodiment, the immune checkpoint protein is PD-L1 or a binding portion thereof, where the cognate ligand of PD-L1 is PD-1 expressed on the surface of T cells. In an embodiment, the checkpoint protein is the extracellular domain of the checkpoint protein.

The term “cognate ligand” refers to the specific binding partner, binding member, or ligand with which an immune checkpoint protein specifically interacts or with which it specifically binds. For example, a specific ligand to which a receptor protein binds or with which it interacts is a “cognate ligand” for that receptor protein. Similarly, the receptor protein is a cognate ligand for a specific ligand molecule or protein.

By “constitutive expression” is meant expression of a gene that is transcribed continually compared to a facultative gene which is only transcribed as needed. Genes that are constitutively expressed are transcribed in an ongoing manner, with control limited to that which is directly associated with the metabolic state of a cell, tissue, or organism. The level of expression of a constitutively expressed gene may be modified, e.g., via post-transcriptional or post-translational modification. In an embodiment, the gene is PD-L1 that encodes the PD-L1 polypeptide.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

“Differentiation” refers to the developmental process of lineage commitment. Differentiation can be assayed by measuring an increase in one or more cell specific markers relative to their expression in a corresponding undifferentiated control cell. A “lineage” refers to a pathway of cellular development, in which precursor or “progenitor” cells undergo progressive physiological changes to become a specified cell type having a characteristic function. In some embodiments, the cell type is a beta cell. In some embodiments, the cell type is an alpha cell, delta cell, or duct cell. In some other embodiments, the cell type is a hepatocyte. In still other embodiments, the cell type is a cardiomyocyte. In some embodiments, the cell type is an intestinal cell. Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured). In some embodiments, an induced pluripotent stem cell (iPSC) is differentiated into a beta-like cell, an alpha-like cell, a delta-like cell, or a duct-like cell. In some other embodiments, an induced pluripotent stem cell (iPSC) is differentiated into a hepatocyte, cardiomyocyte, or intestinal cell.

A “de-differentiated cell” is a cell in which the process of differentiation has been, at least to some degree, reversed. De-differentiation can be assayed, for example, by identifying a reduction in the expression of one or more cell specific markers relative to their expression in a corresponding control cell. Alternatively, de-differentiation can be assayed by measuring an increase in one or more markers typically expressed in an embryonic stem cell, a pluripotent or multi-potent cell type, or expressed at an earlier stage of development. In some embodiments, the de-differentiated cell is an induced pluripotent stem cell (iPSC). In certain embodiments, the de-differentiated cell is a human induced pluripotent stem cell (iPSC).

By “disease” is meant any condition or disorder that adversely affects, damages or interferes with the normal function of a cell, tissue, or organ, or a part of the body, such as autoimmunity or autoimmune disease. Examples of diseases include type 1 diabetes, type 2 diabetes, and pancreatic cancer. An autoimmune disease is one in which the body produces immune cells (e.g., effector T cells or NK cells) and/or antibodies produced by B cells that immunologically react against (attack) its own tissues or organs (or tissue or organ transplants or implants), leading to the deterioration, and, in some cases, to the destruction of the tissue or organ (or tissue or organ transplant or implant).

By “effective amount” is meant the amount of a therapeutic agent or organoid required to ameliorate the symptoms of a disease in a subject relative to an untreated subject. The effective amount of a therapeutic used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, the therapeutic organoid is a pancreatic islet organoid. In some other embodiments, an effective amount of a pancreatic islet organoid is administered to a subject having type 1 or type 2 diabetes.

By “ESRRG polypeptide” is meant a protein or fragment thereof having at least 85% amino acid sequence identity to the sequence provided at NCBI Accession No. NP_001230448.1 and having nuclear hormone receptor activity. The amino acid sequence provided at NCBI Accession No. NP_001230448.1 is shown below:

| | |
|---|---|
| 1 | MSNKDRHIDS SCSSFIKTEP SSPASLTDSV NHHSPGGSSD ASGSYSSTMN GHQNGLDSPP |
| 61 | LYPSAPILGG SGPVRKLYDD CSSTIVEDPQ TKCEYMLNSM PKRLCLVCGD IASGYHYGVA |
| 121 | SCEACKAFFK RTIQGNIEYS CPATNECEIT KRRRKSCQAC RFMKCLKVGM LKEGVRLDRV |
| 181 | RGGRQKYKRR IDAENSPYLN PQLVQPAKKP YNKIVSHLLV AEPEKIYAMP DPTVPDSDIK |
| 241 | ALTTLCDLAD RELVVIIGWA KHIPGFSTLS LADQMSLLQS AWMEILILGV VYRSLSFEDE |
| 301 | LVYADDYIMD EDQSKLAGLL DLNNAILQLV KKYKSMKLEK EEFVTLKAIA LANSDSMHIE |
| 361 | DVEAVQKLQD VLHEALQDYE AGQHMEDPRR AGKMLMTLPL LRQTSTKAVQ HFYNIKLEGK |
| 421 | VPMHKLFLEM LEAKV |

By “ESRRG polynucleotide” is meant a polynucleotide encoding a ESRRG polypeptide or fragment thereof. An exemplary ESRRG polynucleotide sequence is provided at NCBI Ref: NM_001243519.1. The sequence provided at NCBI Ref: NM_001243519.1 is reproduced below:

| | |
|---|---|
| 1 | aagctccaat cggggcttta agtccttgat taggagagtg tgagagcttt ggtcccaact |
| 61 | ggctgtgcct ataggcttgt cactaggaga acatttgtgt taattgcact gtgctctgtc |
| 121 | aaggaaactt tgatttatag ctggggtgca caaataatgg ttgccggtcg cacatggatt |
| 181 | cggtagaact ttgccttcct gaatcttttt ccctgcacta cgaggaagag tagacttgaa |
| 241 | tgagacctgc ctcatcagtc atgggatcat agtgtcacag atggaaaagc aactatcagc |
| 301 | tgaattgtac tgaactacac acttggctaa ttcatcttat tgctctacac atctaaagga |
| 361 | aggctcattc tgttcttgga gtctagacag catcaggagt tgggctcagt gaacaaaact |
| 421 | ttaatgtcta gagcatttat gagggtttta atgattggaa aatctatcct gagaatgtgg |
| 481 | tcaccatatg tgacagcctt gctttctatc ttgtcttcag tttctggggc ttctctgcag |
| 541 | aatgtcaaac aaagatcgac acattgattc cagctgttcg tccttcatca agacggaacc |
| 601 | ttccagccca gcctccctga cggacagcgt caaccaccac agccctggtg gctcttcaga |
| 661 | cgccagtggg agctacagtt caaccatgaa tggccatcag aacggacttg actcgccacc |
| 721 | tctctaccct tctgctccta tcctgggagg tagtgggcct gtcaggaaac tgtatgatga |
| 781 | ctgctccagc accattgttg aagatcccca gaccaagtgt gaatacatgc tcaactcgat |
| 841 | gcccaagaga ctgtgtttag tgtgtggtga catcgcttct gggtaccact atggggtagc |
| 901 | atcatgtgaa gcctgcaagg cattcttcaa gaggacaatt caaggcaata tagaatacag |
| 961 | ctgccctgcc acgaatgaat gtgaaatcac aaagcgcaga cgtaaatcct gccaggcttg |
| 1021 | ccgcttcatg aagtgtttaa aagtgggcat gctgaaagaa ggggtgcgtc ttgacagagt |
| 1081 | acgtggaggt cggcagaagt acaagcgcag gatagatgcg gagaacagcc catacctgaa |
| 1141 | ccctcagctg gttcagccag ccaaaaagcc atataacaag attgtctcac atttgttggt |
| 1201 | ggctgaaccg gagaagatct atgccatgcc tgaccctact gtccccgaca gtgacatcaa |
| 1261 | agccctcact acactgtgtg acttggccga ccgagagttg gtggttatca ttggatgggc |
| 1321 | gaagcatatt ccaggcttct ccacgctgtc cctggcggac cagatgagcc ttctgcagag |
| 1381 | tgcttggatg gaaattttga tccttggtgt cgtataccgg tctctttcgt ttgaggatga |
| 1441 | acttgtctat gcagacgatt atataatgga cgaagaccag tccaaattag caggccttct |
| 1501 | tgatctaaat aatgctatcc tgcagctggt aaagaaatac aagagcatga agctggaaaa |
| 1561 | agaagaattt gtcaccctca aagctatagc tcttgctaat tcagactcca tgcacataga |
| 1621 | agatgttgaa gccgttcaga agcttcagga tgtcttacat gaagcgctgc aggattatga |
| 1681 | agctggccag cacatggaag accctcgtcg agctggcaag atgctgatga cactgccact |
| 1741 | cctgaggcag acctctacca aggccgtgca gcatttctac aacatcaaac tagaaggcaa |
| 1801 | agtcccaatg cacaaacttt ttttggaaat gttggaggcc aaggtctgac taaaagctcc |
| 1861 | ctgggccttc ccatccttca tgttgaaaaa gggaaaataa acccaagagt gatgtcgaag |
| 1921 | aaacttagag tttagttaac aacatcaaaa atcaacagac tgcactgata atttagcagc |
| 1981 | aagactatga agcagctttc agattcctcc ataggttcct gatgagtttc tttctacttt |
| 2041 | ctccatcatc ttctttcctc tttcttccca catttctctt tctctttatt ttttctcctt |
| 2101 | ttcttctttc acctccctta tttctttgct tctttcattc ctagttccca ttctccttta |
| 2161 | ttttcttccc gtctgcctgc cttctttctt ttctttacct actctcattc ctctcttttc |
| 2221 | tcatccttcc ccttttttct aaatttgaaa tagctttagt ttaaaaaaaa atcctccctt |
| 2281 | ccccctttcc tttccctttc tttccttttt ccctttcctt ttccctttcc tttcctttcc |
| 2341 | tcttgacctt ctttccatct ttctttttct tccttctgct gctgaacttt taaaagaggt |
| 2401 | ctctaactga agagagatgg aagccagccc tgccaaagga tggagatcca taatatggat |
| 2461 | gccagtgaac ttattgtgaa ccatactgtc cccaatgact aaggaatcaa agagagagaa |
| 2521 | ccaacgttcc taaaagtaca gtgcaacata tacaaattga ctgagtgcag tattagattt |
| 2581 | catgggagca gcctctaatt agacaactta agcaacgttg catcggctgc ttcttatcat |
| 2641 | tgcttttcca tctagatcag ttacagccat ttgattcctt aattgttttt tcaagtcttc |
| 2701 | caggtatttg ttagtttagc tactatgtaa ctttttcagg gaatagttta agctttattc |
| 2761 | attcatgcaa tactaaagag aaataagaat actgcaattt tgtgctggct ttgaacaatt |
| 2821 | acgaacaata atgaaggaca aatgaatcct gaaggaagat ttttaaaaat gttttgtttc |
| 2881 | ttcttacaaa tggagatttt tttgtaccag ctttaccact tttcagccat ttattaatat |
| 2941 | gggaatttaa cttactcaag caatagttga agggaaggtg catattatca cggatgcaat |
| 3001 | ttatgttgtg tgccagtctg gtcccaaaca tcaatttctt aacatgagct ccagtttacc |
| 3061 | taaatgttca ctgacacaaa ggatgagatt acacctacag tgactctgag tagtcacata |
| 3121 | tataagcact gcacatgaga tatagatccg tagaattgtc aggagtgcac ctctctactt |
| 3181 | gggaggtaca attgccatat gatttctagc tgccatggtg gttaggaatg tgatactgcc |
| 3241 | tgtttgcaaa gtcacagacc ttgcctcaga aggagctgtg agccagtatt catttaagag |
| 3301 | gcaataaggc aaatgccaga attaaaaaaa aaaatcatca aagacagaaa atgcctgacc |
| 3361 | aaattctaaa acctaatcca tataagttta ttcatttagg aatgttcgtt taaattaatc |
| 3421 | tgcagttttt accaagagct aagccaatat atgtgctttt caaccagtat tgtcacagca |
| 3481 | tgaaagtcaa gtcaggttcc agactgttaa gaggtgtaat ctaatgaaga aatcaattag |
| 3541 | atgccccgaa atctacagtc gctgaataac caataaacag taacctccat caaatgctat |
| 3601 | accaatggac cagtgttagt agctgctccc tgtattatgt gaacagtctt attctatgta |
| 3661 | cacagatgta attaaaattg taatcctaac aaacaaaaga aatgtagttc agcttttcaa |
| 3721 | tgtttcatgt ttgctgtgct tttctgaatt ttatgttgca ttcaaagact gttgtcttgt |
| 3781 | tcttgtggtg tttggattct tgtggtgtgt gcttttagac acagggtaga attagagaca |
| 3841 | atattggatg tacaattcct caggagacta cagtagtata ttctattcct taccagtaat |
| 3901 | aaggttcttc ctaataataa ttaagagatt gaaactccaa acaagtattc attatgaaca |
| 3961 | gatacacatc aaaatcataa taatattttc aaaacaagga ataatttctc taatggttta |
| 4021 | ttatagaata ccaatgtata gcttagaaat aaaactttga atatttcaag aatatagata |
| 4081 | agtctaattt ttaaatgctg tatatatggc tttcactcaa tcatctctca gatgttgtta |
| 4141 | ttaactcgct ctgtgttgtt gcaaaacttt ttggtgcaga ttcgtttcca aaactattgc |
| 4201 | tactttgtgt gctttaaaca aaataccttg ggttgatgaa acatcaaccc agtgctagga |
| 4261 | atactgtgta tctatcatta gctatatggg actatattgt agattgtggt ttctcagtag |
| 4321 | agaagtgact gtagtgtgat tctagataaa tcatcattag caattcattc agatggtcaa |
| 4381 | taacttgaaa tttatagctg tgataggagt tcagaaattg gcacatccct ttaaaaataa |
| 4441 | caacagaaaa tacaactcct gggaaaaaag gtgctgattc tataagatta tttatatatg |
| 4501 | taagtgttta aaaagattat tttccagaaa gtttgtgcag ggtttaagtt gctactattc |
| 4561 | aactacacta tatataaata aaatatatac aatatataca ttgttttcac tgtatcacat |
| 4621 | taaagtactt gggcttcaga agtaagagcc aaccaactga aaacctgaga tggagatatg |
| 4681 | ttcaaagaat gagatacaat tttttagttt tcagtttaag taactctcag cattacaaaa |
| 4741 | gagtaagtat ctcacaaata ggaaataaaa ctaaaacgtg gatttaaaaa gaactgcacg |
| 4801 | ggctttaggg taaatgctca tcttaaacct cactagaggg aagtcttctc aagtttcaag |
| 4861 | caagaccatt tacttaatgt gaagttttgg aaagttataa aggtgtatgt tttagccata |
| 4921 | tgattttaat tttaattttg cttcttttag gttcgttctt atttaaagca atatgattgt |
| 4981 | gtgactcctt gtagttacac ttgtgtttca atcagatcag attgttgtat ttattccact |
| 5041 | attttgcatt taaatgataa cataaaagat ataaaaaatt taaaactgct atttttctta |
| 5101 | tagaagagaa aatgggtgtt ggtgattgta ttttaattat ttaagcgtct ctgtttacct |
| 5161 | gcctaggaaa acattttatg gcagtcttat gtgcaaagat cgtaaaagga caaaaaattt |
| 5221 | aaactgctta taataatcca ggagttgcat tatagccagt agtaaaaata ataataataa |
| 5281 | taataaaacc atgtctatag ctgtagatgg gcttcacatc tgtaaagcaa tcaattgtat |
| 5341 | atttttgtga tgtgtaccat actgtgtgct ccagcaaatg tccatttgtg taaatgtatt |
| 5401 | tattttatat tgtatatatt gttaaatgca aaaaggagat atgattctgt aactccaatc |
| 5461 | agttcagatg tgtaactcaa attattatgc ctttcaggat gatggtagag caatattaaa |
| 5521 | caagcttcca cttttgactg ctaaaaaaaa aaaaaaaaa |

As used herein, “endocrine” refers to secretion of an agent (e.g., a hormone) into a bloodstream. “Exocrine” refers to secretion of an agent into an epithelial surface by way of a duct.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “FOXA2 polypeptide” is meant a protein or fragment thereof having at least 85% amino acid sequence identity to the sequence provided at NCBI Accession No. NP_068556.2 and having transcription factor activity. The amino acid sequence provided at NCBI Accession No. NP_068556.2 is shown below:

| | |
|---|---|
| 1 | MHSASSMLGA VKMEGHEPSD WSSYYAEPEG YSSVSNMNAG LGMNGMNTYM SMSAAAMGSG |
| 61 | SGNMSAGSMN MSSYVGAGMS PSLAGMSPGA GAMAGMGGSA GAAGVAGMGP HLSPSLSPLG |
| 121 | GQAAGAMGGL APYANMNSMS PMYGQAGLSR ARDPKTYRRS YTHAKPPYSY ISLITMAIQQ |
| 181 | SPNKMLTLSE IYQWIMDLFP FYRQNQQRWQ NSIRHSLSFN DCFLKVPRSP DKPGKGSFWT |
| 241 | LHPDSGNMFE NGCYLRRQKR FKCEKQLALK EAAGAAGSGK KAAAGAQASQ AQLGEAAGPA |
| 301 | SETPAGTESP HSSASPCQEH KRGGLGELKG TPAAALSPPE PAPSPGQQQQ AAAHLLGPPH |
| 361 | HPGLPPEAHL KPEHHYAFNH PFSINNLMSS EQQHHHSHHH HQPHKMDLKA YEQVMHYPGY |
| 421 | GSPMPGSLAM GPVTNKTGLD ASPLAADTSY YQGVYSRPIM NSS |

By “FOXA2 polynucleotide” is meant a polynucleotide encoding a FOXA2 polypeptide or fragment thereof. An exemplary FOXA2 polynucleotide sequence is provided at NCBI Ref: NM_021784.4. The sequence provided at NCBI Ref: NM_021784.4 is reproduced below:

| | |
|---|---|
| 1 | cccgcccact tccaactacc gcctccggcc tgcccaggga gagagaggga gtggagccca |
| 61 | gggagaggga gcgcgagaga gggagggagg aggggacggt gctttggctg actttttttt |
| 121 | aaaagagggt gggggtgggg ggtgattgct ggtcgtttgt tgtggctgtt aaattttaaa |
| 181 | ctgccatgca ctcggcttcc agtatgctgg gagcggtgaa gatggaaggg cacgagccgt |
| 241 | ccgactggag cagctactat gcagagcccg agggctactc ctccgtgagc aacatgaacg |
| 301 | ccggcctggg gatgaacggc atgaacacgt acatgagcat gtcggcggcc gccatgggca |
| 361 | gcggctcggg caacatgagc gcgggctcca tgaacatgtc gtcgtacgtg ggcgctggca |
| 421 | tgagcccgtc cctggcgggg atgtcccccg gcgcgggcgc catggcgggc atgggcggct |
| 481 | cggccggggc ggccggcgtg gcgggcatgg ggccgcactt gagtcccagc ctgagcccgc |
| 541 | tcggggggca ggcggccggg gccatgggcg gcctggcccc ctacgccaac atgaactcca |
| 601 | tgagccccat gtacgggcag gcgggcctga gccgcgcccg cgaccccaag acctacaggc |
| 661 | gcagctacac gcacgcaaag ccgccctact cgtacatctc gctcatcacc atggccatcc |
| 721 | agcagagccc caacaagatg ctgacgctga gcgagatcta ccagtggatc atggacctct |
| 781 | tccccttcta ccggcagaac cagcagcgct ggcagaactc catccgccac tcgctctcct |
| 841 | tcaacgactg tttcctgaag gtgccccgct cgcccgacaa gcccggcaag ggctccttct |
| 901 | ggaccctgca ccctgactcg ggcaacatgt tcgagaacgg ctgctacctg cgccgccaga |
| 961 | agcgcttcaa gtgcgagaag cagctggcgc tgaaggaggc cgcaggcgcc gccggcagcg |
| 1021 | gcaagaaggc ggccgccgga gcccaggcct cacaggctca actcggggag gccgccgggc |
| 1081 | cggcctccga gactccggcg ggcaccgagt cgcctcactc gagcgcctcc ccgtgccagg |
| 1141 | agcacaagcg agggggcctg ggagagctga aggggacgcc ggctgcggcg ctgagccccc |
| 1201 | cagagccggc gccctctccc gggcagcagc agcaggccgc ggcccacctg ctgggcccgc |
| 1261 | cccaccaccc gggcctgccg cctgaggccc acctgaagcc ggaacaccac tacgccttca |
| 1321 | accacccgtt ctccatcaac aacctcatgt cctcggagca gcagcaccac cacagccacc |
| 1381 | accaccacca accccacaaa atggacctca aggcctacga acaggtgatg cactaccccg |
| 1441 | gctacggttc ccccatgcct ggcagcttgg ccatgggccc ggtcacgaac aaaacgggcc |
| 1501 | tggacgcctc gcccctggcc gcagatacct cctactacca gggggtgtac tcccggccca |
| 1561 | ttatgaactc ctcttaagaa gacgacggct tcaggcccgg ctaactctgg caccccggat |
| 1621 | cgaggacaag tgagagagca agtgggggtc gagactttgg ggagacggtg ttgcagagac |
| 1681 | gcaagggaga agaaatccat aacaccccca ccccaacacc cccaagacag cagtcttctt |
| 1741 | cacccgctgc agccgttccg tcccaaacag agggccacac agatacccca cgttctatat |
| 1801 | aaggaggaaa acgggaaaga atataaagtt aaaaaaaagc ctccggtttc cactactgtg |
| 1861 | tagactcctg cttcttcaag cacctgcaga ttctgatttt tttgttgttg ttgttctcct |
| 1921 | ccattgctgt tgttgcaggg aagtcttact taaaaaaaaa aaaaaatttt gtgagtgact |
| 1981 | cggtgtaaaa ccatgtagtt ttaacagaac cagagggttg tactattgtt taaaaacagg |
| 2041 | aaaaaaaata atgtaagggt ctgttgtaaa tgaccaagaa aaagaaaaaa aaagcattcc |
| 2101 | caatcttgac acggtgaaat ccaggtctcg ggtccgatta atttatggtt tctgcgtgct |
| 2161 | ttatttatgg cttataaatg tgtattctgg ctgcaagggc cagagttcca caaatctata |
| 2221 | ttaaagtgtt atacccggtt ttatcccttg aatcttttct tccagatttt tcttttcttt |
| 2281 | acttggctta caaaatatac aggcttggaa attatttcaa gaaggaggga gggataccct |
| 2341 | gtctggttgc aggttgtatt ttattttggc ccagggagtg ttgctgtttt cccaacattt |
| 2401 | tattaataaa attttcagac ataaaaaa |

By “GATA6 polypeptide” is meant a protein or fragment thereof having at least 85% amino acid sequence identity to the sequence provided at NCBI Accession No. NP_005248.2 and having transcription factor activity. The amino acid sequence provided at NCBI Accession No. NP 005248.2 is shown below:

| | |
|---|---|
| 1 | MALTDGGWCL PKRFGAAGAD ASDSRAFPAR EPSTPPSPIS |
| | SSSSSCSRGG ERGPGGASNC |
| 61 | GTPQLDTEAA AGPPARSLLL SSYASHPFGA PHGPSAPGVA |
| | GPGGNLSSWE DLLLFTDLDQ |
| 121 | AATASKLLWS SRGAKLSPFA PEQPEEMYQT LAALSSQGPA |
| | AYDGAPGGFV HSAAAAAAAA |
| 181 | AAASSPVYVP TTRVGSMLPG LPYHLQGSGS GPANHAGGAG |
| | AHPGWPQASA DSPPYGSGGG |
| 241 | AAGGGAAGPG GAGSAAAHVS ARFPYSPSPP MANGAAREPG |
| | GYAAAGSGGA GGVSGGGSSL |
| 301 | AAMGGREPQY SSLSAARPLN GTYHHHHHHH HHHPSPYSPY |
| | VGAPLTPAWP AGPFETPVLH |
| 361 | SLQSRAGAPL PVPRGPSADL LEDLSESREC VNCGSIQTPL |
| | WRRDGTGHYL CNACGLYSKM |
| 421 | NGLSRPLIKP QKRVPSSRRL GLSCANCHTT TTTLWRRNAE |
| | GEPVCNACGL YMKLHGVPRP |
| 481 | LAMKKEGIQT RKRKPKNINK SKTCSGNSNN SIPMTPTSTS |
| | SNSDDCSKNT SPTTQPTASG |
| 541 | AGAPVMTGAG ESTNPENSEL KYSGQDGLYI GVSLASPAEV |
| | TSSVRPDSWC ALALA |

By “GATA6 polynucleotide” is meant a polynucleotide encoding a GATA6 polypeptide or fragment thereof. An exemplary KCNK3 polynucleotide sequence is provided at NCBI Ref NM_005257.5. The sequence provided at NCBI Ref: NM_005257.5 is reproduced below:

| | |
|---|---|
| 1 | agttccgacc cacagcctgg cacccttcgg cgagcgctgt |
| | ttgtttaggg ctcggtgagt |
| 61 | ccaatcagga gcccaggctg cagttttccg gcagagcagt |
| | aagaggcgcc tcctctctcc |
| 121 | tttttattca ccagcagcgc ggcgcagacc ccggactcgc |
| | gctcgcccgc tggcgccctc |
| 181 | ggcttctctc cgcgcctggg agcaccctcc gccgcggccg |
| | ttctccatgc gcagcgcccg |
| 241 | cccgaggagc tagacgtcag cttggagcgg cgccggaccg |
| | tggatggcct tgactgacgg |
| 301 | cggctggtgc ttgccgaagc gcttcggggc cgcgggtgcg |
| | gacgccagcg actccagagc |
| 361 | ctttccagcg cgggagccct ccacgccgcc ttcccccatc |
| | tcttcctcgt cctcctcctg |
| 421 | ctcccggggc ggagagcggg gccccggcgg cgccagcaac |
| | tgcgggacgc ctcagctcga |
| 481 | cacggaggcg gcggccggac ccccggcccg ctcgctgctg |
| | ctcagttcct acgcttcgca |
| 541 | tcccttcggg gctccccacg gaccttcggc gcctggggtc |
| | gcgggccccg ggggcaacct |
| 601 | gtcgagctgg gaggacttgc tgctgttcac tgacctcgac |
| | caagccgcga ccgccagcaa |
| 661 | gctgctgtgg tccagccgcg gcgccaagct gagccccttc |
| | gcacccgagc agccggagga |
| 721 | gatgtaccag accctcgccg ctctctccag ccagggtccg |
| | gccgcctacg acggcgcgcc |
| 781 | cggcggcttc gtgcactctg cggccgcggc ggcagcagcc |
| | gcggcggcgg ccagctcccc |
| 841 | ggtctacgtg cccaccaccc gcgtgggttc catgctgccc |
| | ggcctaccgt accacctgca |
| 901 | ggggtcgggc agtgggccag ccaaccacgc gggcggcgcg |
| | ggcgcgcacc ccggctggcc |
| 961 | tcaggcctcg gccgacagcc ctccatacgg cagcggaggc |
| | ggcgcggctg gcggcggggc |
| 1021 | cgcggggcct ggcggcgctg gctcagccgc ggcgcacgtc |
| | tcggcgcgct tcccctactc |
| 1081 | tcccagcccg cccatggcca acggcgccgc gcgggagccg |
| | ggaggctacg cggcggcggg |
| 1141 | cagtgggggc gcgggaggcg tgagcggcgg cggcagtagc |
| | ctggcggcca tgggcggccg |
| 1201 | cgagccccag tacagctcgc tgtcggccgc gcggccgctg |
| | aacgggacgt accaccacca |
| 1261 | ccaccaccac caccaccacc atccgagccc ctactcgccc |
| | tacgtggggg cgccactgac |
| 1321 | gcctgcctgg cccgccggac ccttcgagac cccggtgctg |
| | cacagcctgc agagccgcgc |
| 1381 | cggagccccg ctcccggtgc cccggggtcc cagtgcagac |
| | ctgctggagg acctgtccga |
| 1441 | gagccgcgag tgcgtgaact gcggctccat ccagacgccg |
| | ctgtggcggc gggacggcac |
| 1501 | cggccactac ctgtgcaacg cctgcgggct ctacagcaag |
| | atgaacggcc tcagccggcc |
| 1561 | cctcatcaag ccgcagaagc gcgtgccttc atcacggcgg |
| | cttggattgt cctgtgccaa |
| 1621 | ctgtcacacc acaactacca ccttatggcg cagaaacgcc |
| | gagggtgaac ccgtgtgcaa |
| 1681 | tgcttgtgga ctctacatga aactccatgg ggtgcccaga |
| | ccacttgcta tgaaaaaaga |
| 1741 | gggaattcaa accaggaaac gaaaacctaa gaacataaat |
| | aaatcaaaga cttgctctgg |
| 1801 | taatagcaat aattccattc ccatgactcc aacttccacc |
| | tcttctaact cagatgattg |
| 1861 | cagcaaaaat acttccccca caacacaacc tacagcctca |
| | ggggcgggtg ccccggtgat |
| 1921 | gactggtgcg ggagagagca ccaatcccga gaacagcgag |
| | ctcaagtatt cgggtcaaga |
| 1981 | tgggctctac ataggcgtca gtctcgcctc gccggccgaa |
| | gtcacgtcct ccgtgcgacc |
| 2041 | ggattcctgg tgcgccctgg ccctggcctg agcccacgcc |
| | gccaggaggc agggagggct |
| 2101 | ccgccgcggg cctcactcca ctcgtgtctg cttttgtgca |
| | gcggtccaga cagtggcgac |
| 2161 | tgcgctgaca gaacgtgatt ctcgtgcctt tattttgaaa |
| | gagatgtttt tcccaagagg |
| 2221 | cttgctgaaa gagtgagaga agatggaagg gaagggccag |
| | tgcaactggg cgcttgggcc |
| 2281 | actccagcca gcccgcctcc ggggcggacc ctgctccact |
| | tccagaagcc aggactagga |
| 2341 | cctgggcctt gcctgctatg gaatattgag agagattttt |
| | taaaaaagat tttgcatttt |
| 2401 | gtccaaaatc atgtgcttct tctgatcaat tttggttgtt |
| | ccagaatttc ttcatacctt |
| 2461 | ttccacatcc agatttcatg tgcgttcatg gagaagatca |
| | cttgaggcca tttggtacac |
| 2521 | atctctggag gctgagtcgg ttcatgaggt ctcttatcaa |
| | aaatattact cagtttgcaa |
| 2581 | gactgcattg taactttaac atacactgtg actgacgttt |
| | ctcaaagttc atattgtgtg |
| 2641 | gctgatctga agtcagtcgg aatttgtaaa cagggtagca |
| | aacaagatat ttttcttcca |
| 2701 | tgtatacaat aattttttta aaaagtgcaa tttgcgttgc |
| | agcaatcagt gttaaatcat |
| 2761 | ttgcataaga tttaacagca ttttttataa tgaatgtaaa |
| | cattttaact taatggtact |
| 2821 | taaaataatt taaaagaaaa atgttaactt agacattctt |
| | atgcttcttt tacaactaca |
| 2881 | tcccatttta tatttccaat tgttaaagaa aaatatttca |
| | agaacaaatc ttctctcagg |
| 2941 | aaaattgcct ttctctattt gttaagaatt tttatacaag |
| | aacaccaata tacccccttt |
| 3001 | attttactgt ggaatatgtg ctggaaaaat tgcaacaaca |
| | ctttactacc taacggatag |
| 3061 | catttgtaaa tactctaggt atctgtaaac actctgatga |
| | agtctgtata gtgtgactaa |
| 3121 | cccacaggca ggttggttta cattaatttt tttttttgaa |
| | tgggatgtcc tatggaaacc |
| 3181 | tatttcacca gagttttaaa aataaaaagg gtattgtttt |
| | gtcttctgta cagtgagttc |
| 3241 | cttccctttt caaagctttc tttttatgct gtatgtgact |
| | atagatattc atataaaaca |
| 3301 | agtgcacgtg aagtttgcaa aatgctttaa ggccttcctt |
| | tcaaagcata gtccttttgg |
| 3361 | agccgttttg taccttttat accttggctt atttgaagtt |
| | gacacatggg gttagttact |
| 3421 | actctccatg tgcattgggg acagttttta taagtgggaa |
| | ggactcagta ttattatatt |
| 3481 | tgagatgata agcattttgt ttgggaacaa tgcttaaaaa |
| | tattccagaa agttcagatt |
| 3541 | ttttttcttt gtgaatgaaa tatattctgg cccacgaaca |
| | gggcgatttc ctttcagttt |
| 3601 | tttccttttg caacgtgcct tgaagtctca aagctcacct |
| | gaggttgcag acgttacccc |
| 3661 | caacagaaga taggtagaaa tgattccagt ggcctctttg |
| | tattttcttc attgttgagt |
| 3721 | agatttcagg aaatcaggag gtgtttcaca atacagaatg |
| | atggccttta actgtgaaaa |
| 3781 | aaaaa |

By “gellan gum” is meant a polysaccharide having a straight chain with a repeating unit that has any one of the following molecular structures:

### Gellan Gum—High Acyl Form

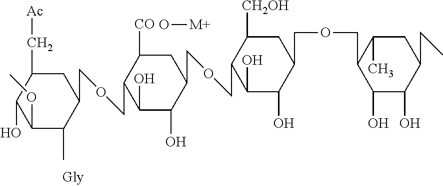

### Gellan Gum—Low Acyl Form

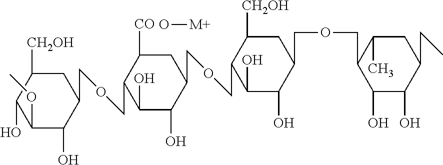

In the foregoing structures, “Ac” refers to an acetate group and “Gly” refers to a glycerate group and “M+” is a monovalent cation. In some embodiments, the gellan gum is KELCOGEL® gellan gum.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunosuppressive agent” or “immunosuppressant” is meant an agent that inhibits or prevents an immune reaction, such as rejection, of a transplanted or implanted organ, islet, or organoid in a subject. Examples of immunosuppressants include, but are not limited to, basilizimab, antithymocyte globulin, alemtuzumab, prednisone, azathioprine, mycophenolate, cyclosporine, sirolimus, methotrexate, interferon, and tacrolimus.

By “induced pluripotent stem cell” or “iPSC” is meant a differentiated somatic cell that acquires pluripotency by the exogenous expression of one or more transcription factors in the cell. An “iPSC-derived cell” is a cell derived from an induced pluripotent stem cell. An “iPSC-derived beta-like cell,” “iPSC-derived alpha-like cell,” “iPSC-derived delta-like cell,” or “iPSC-derived duct-like cell” is a cell derived from an induced pluripotent stem cell and has characteristics of a beta cell, alpha cell, delta cell, or duct cell, respectively.

“Interferon gamma (IFNγ) receptor-expressing” cells (e.g., donor cells), islets, organoids (and the cells therein) refer to cells, islets, organoids (and the cells therein) that express IFNγ receptor on their surface in an amount or level sufficient to respond to IFNγ following contact or exposure to IFNγ, e.g., MPS IFNγ exposure according to the methods described herein, and, in turn, to express or upregulate expression of a checkpoint protein-encoding gene or a checkpoint protein, e.g. PD-L1 (PD-L1 marker protein). In an embodiment, PD-L1 protein is expressed on the surface of the cells (cell membrane expression). In an embodiment, the expression or upregulation of the checkpoint protein, e.g., PD-L1 is sustained, e.g. for greater than or equal to 1, 2, 3, 4, 5, 6, or 7 days or longer. In an embodiment, the expression or upregulation of the checkpoint protein, e.g., PD-L1 is sustained, e.g. for greater than or equal to 7 days or longer. (e.g., more than 1, 2, 3, 4, 5, 6 weeks, or longer). The expression of PD-L1 or the level of expression of PD-L1 in or on cells, for example, may be detected or determined by any assay that is routinely known or used by those skilled in the art to detect or determine levels of proteins or polynucleotides, e.g., without limitation, enzymatic, fluorescent, chemiluminescent or electrochemiluminescent immunoassay, flow cytometry, spectrometry (mass spectrometry); PCR, or RNA or DNA detection methods.

Intermittent exposure as used herein refers to repeated exposure, e.g., short repeated exposure, of cells, islets, organoids (islet-like organoids, e.g., human islet-like organoids, and the cells therein), especially of interferon-gamma (IFNγ) receptor-expressing cells, islets, organoids (islet-like organoids and the cells therein), to multiple pulses, e.g., short repeated pulses, called multiple pulse stimulation (MPS), of IFNγ, as used in the described protocols to generate immunoprotected cells, islets, or organoids that survive and have reduced cell death, e.g., evade immune detection, following transplantation, implantation, or transfer, as described herein. The duration of each of the repeated pulses of IFNγ exposure is typically a short time period, such as minutes or a few hours, rather than a prolonged period of time. By way of example, the exposure to IFNγ may comprise a time period of 0.5 hour, 1 hour, 2 hours, or 3 hours, and the like, multiple times over a given or overall time period, e.g., hours (e.g., 2, 4, 6, 12, 24, 36, 48, 72, 144, or more hours, or intervals therebetween), days (1, 2, 3, 4, 5, 6, 7, 8, 9, 10, 11, 12, 13, 14, or more days), or weeks (1, 2, 3, 4, 5, 6, 7, 8, or more weeks), as described herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. The preparation can be at least 75%, at least 90%, and at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “KCNK3 polypeptide” is meant a protein or fragment thereof having at least 85%, at least 90%, at least 95%, at least 98%, or at least 99% amino acid sequence identity to the sequence provided at NCBI Accession No. NP_002237.1 and having potassium channel activity. The amino acid sequence provided at NCBI Accession No. NP_002237.1 is shown below:

| | |
|---|---|
| 1 | MKRQNVRTLA LIVCTFTYLL VGAAVFDALE SEPELIERQR |
| | LELRQQELRA RYNLSQGGYE |
| 61 | ELERVVLRLK PHKAGVQWRF AGSFYFAITV ITTIGYGHAA |
| | PSTDGGKVFC MFYALLGIPL |
| 121 | TLVMFQSLGE RINTLVRYLL HRAKKGLGMR RADVSMANMV |
| | LIGFFSCIST LCIGAAAFSH |
| 181 | YEHWTFFQAY YYCFITLTTI GFGDYVALQK DQALQTQPQY |
| | VAFSFVYILT GLTVIGAFLN |
| 241 | LVVLRFMTMN AEDEKRDAEH RALLTRNGQA GGGGGGGSAH |
| | TTDTASSTAA AGGGGFRNVY |
| 301 | AEVLHFQSMC SCLWYKSREK LQYSIPMIIP RDLSTSDTCV |
| | EQSHSSPGGG GRYSDTPSRR |
| 361 | CLCSGAPRSA ISSVSTGLHS LSTFRGLMKR RSSV |

By “KCNK3 polynucleotide” is meant a polynucleotide encoding a KCNK3 polypeptide or fragment thereof. An exemplary KCNK3 polynucleotide sequence is provided at NCBI Ref NM_002246.2. The sequence provided at NCBI Ref NM_002246.2 is reproduced below:

| | |
|---|---|
| 1 | ggcggcggcg gcggcggcgg ccccgggcgc tgagcgggtg |
| | cccggcgcgg agagcggcga |
| 61 | gcgcagccat gccccaggcc gcctccgggg cagcagcagc |
| | ggcggccggg gccgaggcgc |
| 121 | gggccggggg cgccgggggg ccggcggcgg cccgggcggg |
| | acgatgaagc ggcagaacgt |
| 181 | gcgcacgctg gcgctcatcg tgtgcacctt cacctacctg |
| | ctggtgggcg ccgcggtctt |
| 241 | cgacgcgctg gagtcggagc ccgagctgat cgagcggcag |
| | cggctggagc tgcggcagca |
| 301 | ggagctgcgg gcgcgctaca acctcagcca gggcggctac |
| | gaggagctgg agcgcgtcgt |
| 361 | gctgcgcctc aagccgcaca aggccggcgt gcagtggcgc |
| | ttcgccggct ccttctactt |
| 421 | cgccatcacc gtcatcacca ccatcggcta cgggcacgcg |
| | gcacccagca cggatggcgg |
| 481 | caaggtgttc tgcatgttct acgcgctgct gggcatcccg |
| | ctcacgctcg tcatgttcca |
| 541 | gagcctgggc gagcgcatca acaccttggt gaggtacctg |
| | ctgcaccgcg ccaagaaggg |
| 601 | gctgggcatg cggcgcgccg acgtgtccat ggccaacatg |
| | gtgctcatcg gcttcttctc |
| 661 | gtgcatcagc acgctgtgca tcggcgccgc cgccttctcc |
| | cactacgagc actggacctt |
| 721 | cttccaggcc tactactact gcttcatcac cctcaccacc |
| | atcggcttcg gcgactacgt |
| 781 | ggcgctgcag aaggaccagg ccctgcagac gcagccgcag |
| | tacgtggcct tcagcttcgt |
| 841 | ctacatcctt acgggcctca cggtcatcgg cgccttcctc |
| | aacctcgtgg tgctgcgctt |
| 901 | catgaccatg aacgccgagg acgagaagcg cgacgccgag |
| | caccgcgcgc tgctcacgcg |
| 961 | caacgggcag gcgggcggcg gcggaggggg tggcagcgcg |
| | cacactacgg acaccgcctc |
| 1021 | atccacggcg gcagcgggcg gcggcggctt ccgcaacgtc |
| | tacgcggagg tgctgcactt |
| 1081 | ccagtccatg tgctcgtgcc tgtggtacaa gagccgcgag |
| | aagctgcagt actccatccc |
| 1141 | catgatcatc ccgcgggacc tctccacgtc cgacacgtgc |
| | gtggagcaga gccactcgtc |
| 1201 | gccgggaggg ggcggccgct acagcgacac gccctcgcga |
| | cgctgcctgt gcagcggggc |
| 1261 | gccacgctcc gccatcagct cggtgtccac gggtctgcac |
| | agcctgtcca ccttccgcgg |
| 1321 | cctcatgaag cgcaggagct ccgtgtgact gccccgaggg |
| | gcctggagca cctgggggcg |
| 1381 | cgggcggggg acccctgctg ggaggccagg agactgcccc |
| | tgctgccttc tgcccagtgg |
| 1441 | gaccccgcac aacatccctc accactctcc cccagcaccc |
| | ccatctccga ctgtgcctgc |
| 1501 | ttgcaccagc cggcaggagg ccgggctctg aggacccctg |
| | gggcccccat cggagccctg |
| 1561 | caaattccga gaaatgtgaa acttggtggg gtcagggagg |
| | aaaggcagaa gctgggagcc |
| 1621 | tcccttccct ttgaaaatct aagaagctcc cagtcctcag |
| | agaccctgct ggtacccaga |
| 1681 | cccccacctt cggaggggac ttcatgttcc gtgtacgttt |
| | gcatctctat ttatacctct |
| 1741 | gtcctgctag gtctcccacc ttcccttggt tccaaaagcc |
| | agggtgtcta tgtccaagtc |
| 1801 | acccctactc agccccactc cccttcctca tccccagctg |
| | tgtctcccaa cctcccttcg |
| 1861 | tgttgttttg catggctttg cagttatgga gaaagtggaa |
| | acccagcagt ccctaaagct |
| 1921 | ggtccccaga aagcaggaca gaaagaagga gggacaggca |
| | ggcagcagga ggggcgagct |
| 1981 | gggaggcagg aggcagcggc ctgtcagtct gcagaatggt |
| | cgcactggag gttcaagcta |
| 2041 | actggcctcc agccacattc tcatagcagg taggacttca |
| | gccttccaga cactgccctt |
| 2101 | agaatctgga acagaagact tcagactcac cataattgct |
| | gataattacc cactcttaaa |
| 2161 | tttgtcgagt gatttttagc ctctgaaaac tctatgctgg |
| | ccactgattc ctttgagtct |
| 2221 | cacaaaaccc tacttaggtc atcagggcag gagttctcac |
| | tcccatttta cagatgagaa |
| 2281 | tactgaggcc tggacaggtg aagtgaccag agagcaaaag |
| | gcaaaggggt gggggctggg |
| 2341 | tgcagtggct cacacctgta ttcccaacac ttttggaggc |
| | tgaggttgga ggattgcttg |
| 2401 | agcccaggaa tttgagacca gcctaggtga catagtgaga |
| | ccccatctct acaaaaaata |
| 2461 | aaaaattaac caggtgtggt ggcacgtgcc tgggagtccc |
| | agcgacttgg gaggctgagg |
| 2521 | tgggaggatt gtttgagcct gggaggtcga ggctgtagtg |
| | agccctgatt gcaccactgt |
| 2581 | actccagcct gggtgacagg gcaagaccct gtctcaaaaa |
| | aaaaaaaaaa aatggcaaag |
| 2641 | ggagacaaga gcccagcctg cttgttgcta gccaaagtgt |
| | tctttccttc cagcttggcc |
| 2701 | tgctcttaaa agcaaagctc ctgcagtgta catcctggca |
| | ttgtgtggct acctgggttt |
| 2761 | taaaccagaa tcagaagtcc cggatcagag ggcactgctg |
| | aggttcagcc tcttctcttc |
| 2821 | ttggccagga ggcagcagct ctgaatgggc ccctgaggct |
| | gcacaggggc ctttgtcact |
| 2881 | ggggcgcatg cttacaaaca gtgcagttct tgggaccgag |
| | gtaagcaggg ctgggtctca |
| 2941 | tggcagaaag gccaggatct ggggctctag gaatttggga |
| | attgggcaga gtggccaaga |
| 3001 | aagctggcag gcatatccta tgggacatca cacctggcac |
| | cattgtcatt gttggtgcct |
| 3061 | gtgtcccaag tagctagtga taagctgagg ctgcagcaag |
| | aaacaccctt cccaggtggg |
| 3121 | ggagtttgga ccagaggtgc cctctgccca ccacacctgc |
| | aacccagaag cccagatgga |
| 3181 | acgcagctga cgaaggtgat gcttgaggct cacttttggg |
| | gccccacagc tggagccggt |
| 3241 | ataatgactg ggacaacatc aaggggtgga tgaggggcct |
| | ctcctcccgc aacactgcct |
| 3301 | tcccatgctg ttcccctgcc agctccttaa cactgccgac |
| | caaggccagc cctggcattc |
| 3361 | agggaaattg gagggcagca cccgtagggt ggccagcctc |
| | aggccccacc ccagctgtgt |
| 3421 | cctctagtct ctggggaccc ctggggggaa gaagtctacc |
| | ctgcttgtga gtcccgtctc |
| 3481 | agtgtggagg aactggctgc acgtgggacc tgaaggtgcc |
| | ctctgtgttt atgttggggg |
| 3541 | tgggggggca gtgctggctg cctctgtcct gtgtgtgacc |
| | ctgccctcga agggtcctgt |
| 3601 | cctgtcagtc ccgagggagc cacaaccaaa gctgcggaga |
| | gaaggtgggg aagggtgcag |
| 3661 | aatggccgtg gggcacagcg tggcagactg ttcagtctct |
| | gctgggtctt tcctagggac |
| 3721 | ctggaaggcc agtgttgctt ccccctcact ccctttcact |
| | gcaggcagcc tctctgcttc |
| 3781 | cccaatgcct tatgcctggg cacactgcca cagaatatgc |
| | aatatgtgtg ggtgaccatg |
| 3841 | ccctcacgac cacaccccca ccccgggcag cccccggact |
| | ccaaaggtcg tggctgccac |
| 3901 | agcctccctc agctcttcct gcctatctgt cttcacactg |
| | agaatggcgc ccaataaatg |
| 3961 | ctatccacgg agaccagg |

By “KCNQ1 polypeptide” is meant a protein or fragment thereof having at least 85%, at least 90%, at least 95%, at least 98%, or at least 99% amino acid sequence identity to the sequence provided at NCBI Accession No. NP_000209.2 (isoform 1) or NP_861463.1 (isoform 2) and having potassium channel activity. The amino acid sequence provided at NCBI Accession No. NP_000209.2 is shown below:

| | |
|---|---|
| 1 | MAAASSPPRA ERKRWGWGRL PGARRGSAGL AKKCPFSLEL |
| | AEGGPAGGAL YAPIAPGAPG |
| 61 | PAPPASPAAP AAPPVASDLG PRPPVSLDPR VSIYSTRRPV |
| | LARTHVQGRV YNFLERPTGW |
| 121 | KCFVYHFAVF LIVLVCLIFS VLSTIEQYAA LATGTLFWME |
| | IVLVVFFGTE YVVRLWSAGC |
| 181 | RSKYVGLWGR LRFARKPISI IDLIVVVASM VVLCVGSKGQ |
| | VFATSAIRGI RFLQILRMLH |
| 241 | VDRQGGTWRL LGSVVFIHRQ ELITTLYIGF LGLIFSSYFV |
| | YLAEKDAVNE SGRVEFGSYA |
| 301 | DALWWGVVTV TTIGYGDKVP QTWVGKTIAS CFSVFAISFF |
| | ALPAGILGSG FALKVQQKQR |
| 361 | QKHFNRQIPA AASLIQTAWR CYAAENPDSS TWKIYIRKAP |
| | RSHTLLSPSP KPKKSVVVKK |
| 421 | KKFKLDKDNG VTPGEKMLTV PHITCDPPEE RRLDHFSVDG |
| | YDSSVRKSPT LLEVSMPHFM |
| 481 | RTNSFAEDLD LEGETLLTPI THISQLREHH RATIKVIRRM |
| | QYFVAKKKFQ QARKPYDVRD |
| 541 | VIEQYSQGHL NLMVRIKELQ RRLDQSIGKP SLFISVSEKS |
| | KDRGSNTIGA RLNRVEDKVT |
| 601 | QLDQRLALIT DMLHQLLSLH GGSTPGSGGP PREGGAHITQ |
| | PCGSGGSVDP ELFLPSNTLP |
| 661 | TYEQLTVPRR GPDEGS |

By “KCNQ1 polynucleotide” is meant a polynucleotide encoding a KCNQ1 polypeptide or fragment thereof. An exemplary KCNQ1 polynucleotide sequence is provided at NCBI Ref NM_000218.2. The sequence provided at NCBI Ref: NM_000218.2 is reproduced below:

| | |
|---|---|
| 1 | gcggcggggc tggcagcagt ggctgcccgc actgcgcccg |
| | ggcgctcgcc ttcgctgcag |
| 61 | ctcccggtgc cgccgctcgg gccggccccc cggcaggccc |
| | tcctcgttat ggccgcggcc |
| 121 | tcctccccgc ccagggccga gaggaagcgc tggggttggg |
| | gccgcctgcc aggcgcccgg |
| 181 | cggggcagcg cgggcctggc caagaagtgc cccttctcgc |
| | tggagctggc ggagggcggc |
| 241 | ccggcgggcg gcgcgctcta cgcgcccatc gcgcccggcg |
| | ccccaggtcc cgcgccccct |
| 301 | gcgtccccgg ccgcgcccgc cgcgccccca gttgcctccg |
| | accttggccc gcggccgccg |
| 361 | gtgagcctag acccgcgcgt ctccatctac agcacgcgcc |
| | gcccggtgtt ggcgcgcacc |
| 421 | cacgtccagg gccgcgtcta caacttcctc gagcgtccca |
| | ccggctggaa atgcttcgtt |
| 481 | taccacttcg ccgtcttcct catcgtcctg gtctgcctca |
| | tcttcagcgt gctgtccacc |
| 541 | atcgagcagt atgccgccct ggccacgggg actctcttct |
| | ggatggagat cgtgctggtg |
| 601 | gtgttcttcg ggacggagta cgtggtccgc ctctggtccg |
| | ccggctgccg cagcaagtac |
| 661 | gtgggcctct gggggcggct gcgctttgcc cggaagccca |
| | tttccatcat cgacctcatc |
| 721 | gtggtcgtgg cctccatggt ggtcctctgc gtgggctcca |
| | aggggcaggt gtttgccacg |
| 781 | tcggccatca ggggcatccg cttcctgcag atcctgagga |
| | tgctacacgt cgaccgccag |
| 841 | ggaggcacct ggaggctcct gggctccgtg gtcttcatcc |
| | accgccagga gctgataacc |
| 901 | accctgtaca tcggcttcct gggcctcatc ttctcctcgt |
| | actttgtgta cctggctgag |
| 961 | aaggacgcgg tgaacgagtc aggccgcgtg gagttcggca |
| | gctacgcaga tgcgctgtgg |
| 1021 | tggggggtgg tcacagtcac caccatcggc tatggggaca |
| | aggtgcccca gacgtgggtc |
| 1081 | gggaagacca tcgcctcctg cttctctgtc tttgccatct |
| | ccttctttgc gctcccagcg |
| 1141 | gggattcttg gctcggggtt tgccctgaag gtgcagcaga |
| | agcagaggca gaagcacttc |
| 1201 | aaccggcaga tcccggcggc agcctcactc attcagaccg |
| | catggaggtg ctatgctgcc |
| 1261 | gagaaccccg actcctccac ctggaagatc tacatccgga |
| | aggccccccg gagccacact |
| 1321 | ctgctgtcac ccagccccaa acccaagaag tctgtggtgg |
| | taaagaaaaa aaagttcaag |
| 1381 | ctggacaaag acaatggggt gactcctgga gagaagatgc |
| | tcacagtccc ccatatcacg |
| 1441 | tgcgaccccc cagaagagcg gcggctggac cacttctctg |
| | tcgacggcta tgacagttct |
| 1501 | gtaaggaaga gcccaacact gctggaagtg agcatgcccc |
| | atttcatgag aaccaacagc |
| 1561 | ttcgccgagg acctggacct ggaaggggag actctgctga |
| | cacccatcac ccacatctca |
| 1621 | cagctgcggg aacaccatcg ggccaccatt aaggtcattc |
| | gacgcatgca gtactttgtg |
| 1681 | gccaagaaga aattccagca agcgcggaag ccttacgatg |
| | tgcgggacgt cattgagcag |
| 1741 | tactcgcagg gccacctcaa cctcatggtg cgcatcaagg |
| | agctgcagag gaggctggac |
| 1801 | cagtccattg ggaagccctc actgttcatc tccgtctcag |
| | aaaagagcaa ggatcgcggc |
| 1861 | agcaacacga tcggcgcccg cctgaaccga gtagaagaca |
| | aggtgacgca gctggaccag |
| 1921 | aggctggcac tcatcaccga catgcttcac cagctgctct |
| | ccttgcacgg tggcagcacc |
| 1981 | cccggcagcg gcggcccccc cagagagggc ggggcccaca |
| | tcacccagcc ctgcggcagt |
| 2041 | ggcggctccg tcgaccctga gctcttcctg cccagcaaca |
| | ccctgcccac ctacgagcag |
| 2101 | ctgaccgtgc ccaggagggg ccccgatgag gggtcctgag |
| | gaggggatgg ggctggggga |
| 2161 | tgggcctgag tgagagggga ggccaagagt ggccccacct |
| | ggccctctct gaaggaggcc |
| 2221 | acctcctaaa aggcccagag agaagagccc cactctcaga |
| | ggccccaata ccccatggac |
| 2281 | catgctgtct ggcacagcct gcacttgggg gctcagcaag |
| | gccacctctt cctggccggt |
| 2341 | gtgggggccc cgtctcaggt ctgagttgtt accccaagcg |
| | ccctggcccc cacatggtga |
| 2401 | tgttgacatc actggcatgg tggttgggac ccagtggcag |
| | ggcacagggc ctggcccatg |
| 2461 | tatggccagg aagtagcaca ggctgagtgc aggcccaccc |
| | tgcttggccc agggggcttc |
| 2521 | ctgaggggag acagagcaac ccctggaccc cagcctcaaa |
| | tccaggaccc tgccaggcac |
| 2581 | aggcagggca ggaccagccc acgctgacta cagggccgcc |
| | ggcaataaaa gcccaggagc |
| 2641 | ccatttggag ggcctgggcc tggctccctc actctcagga |
| | aatgctgacc catgggcagg |
| 2701 | agactgtgga gactgctcct gagcccccag cttccagcag |
| | gagggacagt ctcaccattt |
| 2761 | ccccagggca cgtggttgag tggggggaac gcccacttcc |
| | ctgggttaga ctgccagctc |
| 2821 | ttcctagctg gagaggagcc ctgcctctcc gcccctgagc |
| | ccactgtgcg tggggctccc |
| 2881 | gcctccaacc cctcgcccag tcccagcagc cagccaaaca |
| | cacagaaggg gactgccacc |
| 2941 | tccccttgcc agctgctgag ccgcagagaa gtgacggttc |
| | ctacacagga caggggttcc |
| 3001 | ttctgggcat tacatcgcat agaaatcaat aatttgtggt |
| | gatttggatc tgtgttttaa |
| 3061 | tgagtttcac agtgtgattt tgattattaa ttgtgcaagc |
| | ttttcctaat aaacgtggag |
| 3121 | aatcacaggc tgggctgggc actgctctca ccttggttcc |
| | tggggcatcc atggggtctc |
| 3181 | tcacagacag gacccctgca gttcccctgg aagcagtgcc |
| | caggtggctg tggaatagga |
| 3241 | acgctaaaaa aaaaaaaaaa aa |

By “LGR5 polypeptide” is meant a protein or fragment thereof having at least 85%, at least 90%, at least 95%, at least 98%, or at least 99% amino acid sequence identity to the sequence provided at NCBI Accession No. NP_003658.1 (isoform 1), NP_001264155.1 (isoform 2), or NP_001264156.1 (isoform 3) and having transmembrane signaling receptor activity or G-protein coupled receptor activity. The amino acid sequence provided at NCBI Accession No. NP_003658.1 is shown below:

| | |
|---|---|
| 1 | MDTSRLGVLL SLPVLLQLAT GGSSPRSGVL LRGCPTHCHC |
| | EPDGRMLLRV DCSDLGLSEL |
| 61 | PSNLSVFTSY LDLSMNNISQ LLPNPLPSLR FLEELRLAGN |
| | ALTYIPKGAF TGLYSLKVLM |
| 121 | LQNNQLRHVP TEALQNLRSL QSLRLDANHI SYVPPSCFSG |
| | LHSLRHLWLD DNALTEIPVQ |
| 181 | AFRSLSALQA MTLALNKIHH IPDYAFGNLS SLVVLHLHNN |
| | RIHSLGKKCF DGLHSLETLD |
| 241 | LNYNNLDEFP TAIRTLSNLK ELGFHSNNIR SIPEKAFVGN |
| | PSLITIHFYD NPIQFVGRSA |
| 301 | FQHLPELRTL TLNGASQITE FPDLTGTANL ESLILTGAQI |
| | SSLPQTVCNQ LPNLQVLDLS |
| 361 | YNLLEDLPSF SVCQKLQKID LRHNEIYEIK VDTFQQLLSL |
| | RSLNLAWNKI AIIHPNAFST |
| 421 | LPSLIKLDLS SNLLSSFPIT GLHGLTHLKL TGNHALQSLI |
| | SSENFPELKV IEMPYAYQCC |
| 481 | AFGVCENAYK ISNQWNKGDN SSMDDLHKKD AGMFQAQDER |
| | DLEDFLLDFE EDLKALHSVQ |
| 541 | CSPSPGPFKP CEHLLDGWLI RIGVWTIAVL ALTCNALVTS |
| | TVFRSPLYIS PIKLLIGVIA |
| 601 | AVNMLTGVSS AVLAGVDAFT FGSFARHGAW WENGVGCHVI |
| | GFLSIFASES SVFLLTLAAL |
| 661 | ERGFSVKYSA KFETKAPFSS LKVIILLCAL LALTMAAVPL |
| | LGGSKYGASP LCLPLPFGEP |
| 721 | STMGYMVALI LLNSLCFLMM TIAYTKLYCN LDKGDLENIW |
| | DCSMVKHIAL LLFTNCILNC |
| 781 | PVAFLSFSSL INLTFISPEV IKFILLVVVP LPACLNPLLY |
| | ILFNPHFKED LVSLRKQTYV |
| 841 | WTRSKHPSLM SINSDDVEKQ SCDSTQALVT FTSSSITYDL |
| | PPSSVPSPAY PVTESCHLSS |
| 901 | VAFVPCL |

By “LGR5 polynucleotide” is meant a polynucleotide encoding a LGR5 polypeptide or fragment thereof. An exemplary LGR5 polynucleotide sequence is provided at NCBI Ref: NM_003667.3. The sequence provided at NCBI Ref NM_003667.3 is reproduced below:

| | |
|---|---|
| 1 | aaaaaacgag cgtgcaagca gagatgctgc tccacaccgc |
| | tcaggccgcg agcagcagca |
| 61 | aggcgcaccg ccactgtcgc cgctgcagcc agggctgctc |
| | cgaaggccgg cgtggcggca |
| 121 | accggcacct ctgtccccgc cgcgcttctc ctcgccgccc |
| | acgccgtggg gtcaggaacg |
| 181 | cggcgtctgg cgctgcagac gcccgctgag ttgcagaagc |
| | ccacggagcg gcgcccggcg |
| 241 | cgccacggcc cgtagcagtc cggtgctgct ctccgcccgc |
| | gtccggctcg tggcccccta |
| 301 | cttcgggcac catggacacc tcccggctcg gtgtgctcct |
| | gtccttgcct gtgctgctgc |
| 361 | agctggcgac cgggggcagc tctcccaggt ctggtgtgtt |
| | gctgaggggc tgccccacac |
| 421 | actgtcattg cgagcccgac ggcaggatgt tgctcagggt |
| | ggactgctcc gacctggggc |
| 481 | tctcggagct gccttccaac ctcagcgtct tcacctccta |
| | cctagacctc agtatgaaca |
| 541 | acatcagtca gctgctcccg aatcccctgc ccagtctccg |
| | cttcctggag gagttacgtc |
| 601 | ttgcgggaaa cgctctgaca tacattccca agggagcatt |
| | cactggcctt tacagtctta |
| 661 | aagttcttat gctgcagaat aatcagctaa gacacgtacc |
| | cacagaagct ctgcagaatt |
| 721 | tgcgaagcct tcaatccctg cgtctggatg ctaaccacat |
| | cagctatgtg cccccaagct |
| 781 | gtttcagtgg cctgcattcc ctgaggcacc tgtggctgga |
| | tgacaatgcg ttaacagaaa |
| 841 | tccccgtcca ggcttttaga agtttatcgg cattgcaagc |
| | catgaccttg gccctgaaca |
| 901 | aaatacacca cataccagac tatgcctttg gaaacctctc |
| | cagcttggta gttctacatc |
| 961 | tccataacaa tagaatccac tccctgggaa agaaatgctt |
| | tgatgggctc cacagcctag |
| 1021 | agactttaga tttaaattac aataaccttg atgaattccc |
| | cactgcaatt aggacactct |
| 1081 | ccaaccttaa agaactagga tttcatagca acaatatcag |
| | gtcgatacct gagaaagcat |
| 1141 | ttgtaggcaa cccttctctt attacaatac atttctatga |
| | caatcccatc cagtttgttg |
| 1201 | ggagatctgc ttttcaacat ttacctgaac taagaacact |
| | gactctgaat ggtgcctcac |
| 1261 | aaataactga atttcctgat ttaactggaa ctgcaaacct |
| | ggagagtctg actttaactg |
| 1321 | gagcacagat ctcatctctt cctcaaaccg tctgcaatca |
| | gttacctaat ctccaagtgc |
| 1381 | tagatctgtc ttacaaccta ttagaagatt tacccagttt |
| | ttcagtctgc caaaagcttc |
| 1441 | agaaaattga cctaagacat aatgaaatct acgaaattaa |
| | agttgacact ttccagcagt |
| 1501 | tgcttagcct ccgatcgctg aatttggctt ggaacaaaat |
| | tgctattatt caccccaatg |
| 1561 | cattttccac tttgccatcc ctaataaagc tggacctatc |
| | gtccaacctc ctgtcgtctt |
| 1621 | ttcctataac tgggttacat ggtttaactc acttaaaatt |
| | aacaggaaat catgccttac |
| 1681 | agagcttgat atcatctgaa aactttccag aactcaaggt |
| | tatagaaatg ccttatgctt |
| 1741 | accagtgctg tgcatttgga gtgtgtgaga atgcctataa |
| | gatttctaat caatggaata |
| 1801 | aaggtgacaa cagcagtatg gacgaccttc ataagaaaga |
| | tgctggaatg tttcaggctc |
| 1861 | aagatgaacg tgaccttgaa gatttcctgc ttgactttga |
| | ggaagacctg aaagcccttc |
| 1921 | attcagtgca gtgttcacct tccccaggcc ccttcaaacc |
| | ctgtgaacac ctgcttgatg |
| 1981 | gctggctgat cagaattgga gtgtggacca tagcagttct |
| | ggcacttact tgtaatgctt |
| 2041 | tggtgacttc aacagttttc agatcccctc tgtacatttc |
| | ccccattaaa ctgttaattg |
| 2101 | gggtcatcgc agcagtgaac atgctcacgg gagtctccag |
| | tgccgtgctg gctggtgtgg |
| 2161 | atgcgttcac ttttggcagc tttgcacgac atggtgcctg |
| | gtgggagaat ggggttggtt |
| 2221 | gccatgtcat tggttttttg tccatttttg cttcagaatc |
| | atctgttttc ctgcttactc |
| 2281 | tggcagccct ggagcgtggg ttctctgtga aatattctgc |
| | aaaatttgaa acgaaagctc |
| 2341 | cattttctag cctgaaagta atcattttgc tctgtgccct |
| | gctggccttg accatggccg |
| 2401 | cagttcccct gctgggtggc agcaagtatg gcgcctcccc |
| | tctctgcctg cctttgcctt |
| 2461 | ttggggagcc cagcaccatg ggctacatgg tcgctctcat |
| | cttgctcaat tccctttgct |
| 2521 | tcctcatgat gaccattgcc tacaccaagc tctactgcaa |
| | tttggacaag ggagacctgg |
| 2581 | agaatatttg ggactgctct atggtaaaac acattgccct |
| | gttgctcttc accaactgca |
| 2641 | tcctaaactg ccctgtggct ttcttgtcct tctcctcttt |
| | aataaacctt acatttatca |
| 2701 | gtcctgaagt aattaagttt atccttctgg tggtagtccc |
| | acttcctgca tgtctcaatc |
| 2761 | cccttctcta catcttgttc aatcctcact ttaaggagga |
| | tctggtgagc ctgagaaagc |
| 2821 | aaacctacgt ctggacaaga tcaaaacacc caagcttgat |
| | gtcaattaac tctgatgatg |
| 2881 | tcgaaaaaca gtcctgtgac tcaactcaag ccttggtaac |
| | ctttaccagc tccagcatca |
| 2941 | cttatgacct gcctcccagt tccgtgccat caccagctta |
| | tccagtgact gagagctgcc |
| 3001 | atctttcctc tgtggcattt gtcccatgtc tctaattaat |
| | atgtgaagga aaatgttttc |
| 3061 | aaaggttgag aacctgaaaa tgtgagattg agtatatcag |
| | agcagtaatt aataagaaga |
| 3121 | gctgaggtga aactcggttt aaaaaccaaa aaagaatctc |
| | tcagttagta agaaaaggct |
| 3181 | gaaaacctct tgatacttga gagtgaatat aagtctaaat |
| | gctgctttgt ataatttgtt |
| 3241 | cagctaaggg atagatcgat cacactattt aagtgagccc |
| | agatcaaaaa agcagattga |
| 3301 | aattttcttt agaaaagatt ctccatgatt tgaattgcat |
| | tctctttaaa ctcaccaatg |
| 3361 | taatcatttt gggaggaggg agaacccact tgctttccaa |
| | atgggtttat ttaaacccac |
| 3421 | aaactcaaga ggttgttggg ggaattagga aaataagggt |
| | tttcaatgac ctacattgct |
| 3481 | aggtagaggc tgtgatccat gggatttcat tctaatgacc |
| | atgtgaagat gtttgagtcc |
| 3541 | tcctttgcct ttcctcagaa agaatccttc taaggcacaa |
| | atcccttaga tggataatgt |
| 3601 | aaggtattgt taactcactc atattgagat catttttaga |
| | gataccaggt tttatgtatc |
| 3661 | agcactagat ggttccaccc tcatgggata aaactgctta |
| | caagtatttt gaaagaaaaa |
| 3721 | ctgaccaaaa ttcttaaatt gttactaagg caatcatgca |
| | caggtgacgt atgtcttatc |
| 3781 | tgatttgttt ttaactcctt ggtgcccaaa gctcagaagg |
| | gaattccact gccagcaatg |
| 3841 | aacatacctg gaaaagaaag taagcaatct gggatttttt |
| | ttctgggtta gtaaagaatt |
| 3901 | tttgcaataa gttttatcag ttgattcaaa ctgatgtgca |
| | tcttaatgat caaatgtgca |
| 3961 | cattacataa attaagtcca ctgatacaac ttcttacaca |
| | tgtatctcta gtagctctgg |
| 4021 | caaacccaat atctgacacc actttggact caagagactc |
| | agtaacgtat tatcctgttt |
| 4081 | atttagcttg gttttagctg tgttctctct ggataaccca |
| | cttgatgtta ggaacattac |
| 4141 | ttctctgctt attccatatt aatactgtgt taggtatttt |
| | aagaagcaag ttattaaata |
| 4201 | agaaaagtca aagtattaat tcttaccttc tattatccta |
| | tattagcttc aatacatcca |
| 4261 | aaccaaatgg ctgttaggta gatttatttt tatataagca |
| | tgtttatttt gatcagatgt |
| 4321 | tttaacttgg atttgaaaaa atacatttat gagatgtttt |
| | ataagatgtg taaatataga |
| 4381 | actgtattta ttactatagt aaaggttcag taacattaag |
| | gaccatgata atgataataa |
| 4441 | accttgtaca gtggcatatt ctttgattta tattgtgttt |
| | ctctgcccat tttctttaaa |
| 4501 | ttcattaact gtatatatgt aaatatatag tacttgtaaa |
| | tagattccaa atttgctttt |
| 4561 | ctattgggta aaaaataaat ttgtaataaa atgtgtgact |
| | atgaaacaaa aaaaaaaaaa |
| 4621 | aaaaa |

By “LDHA polypeptide” or “lactate dehydrogenase A polypeptide” is meant a protein or fragment thereof having at least 85%, at least 90%, at least 95%, at least 98%, or at least 99% amino acid sequence identity to the sequence provided at NCBI Accession 50 No. NP_005557.1 (isoform 1), NP_001128711.1 (isoform 2), NP_001158886.1 (isoform 3), NP_001158887.1 (isoform 4), or NP_001158888.1 (isoform 5) and having dehydrogenase activity. The amino acid sequence provided at NCBI Accession No. NP_005557.1 is shown below:

| | |
|---|---|
| 1 | MATLKDQLIY NLLKEEQTPQ NKITVVGVGA VGMACAISIL |
| | MKDLADELAL VDVIEDKLKG |
| 61 | EMMDLQHGSL FLRTPKIVSG KDYNVTANSK LVIITAGARQ |
| | QEGESRLNLV QRNVNIFKFI |
| 121 | IPNVVKYSPN CKLLIVSNPV DILTYVAWKI SGFPKNRVIG |
| | SGCNLDSARF RYLMGERLGV |
| 181 | HPLSCHGWVL GEHGDSSVPV WSGMNVAGVS LKTLHPDLGT |
| | DKDKEQWKEV HKQVVESAYE |
| 241 | VIKLKGYTSW AIGLSVADLA ESIMKNLRRV HPVSTMIKGL |
| | YGIKDDVFLS VPCILGQNGI |
| 301 | SDLVKVTLTS EEEARLKKSA DTLWGIQKEL QF |

By “LDHA polynucleotide” or “lactate dehydrogenase A polynucleotide” is meant a polynucleotide encoding a LDHA polypeptide or fragment thereof. An exemplary LDHA polynucleotide sequence is provided at NCBI Ref: NM_005566.3. The sequence provided at NCBI Ref: NM_005566.3 is reproduced below:

| | |
|---|---|
| 1 | gtctgccggt cggttgtctg gctgcgcgcg ccacccgggc |
| | ctctccagtg ccccgcctgg |
| 61 | ctcggcatcc acccccagcc cgactcacac gtgggttccc |
| | gcacgtccgc cggccccccc |
| 121 | cgctgacgtc agcatagctg ttccacttaa ggcccctccc |
| | gcgcccagct cagagtgctg |
| 181 | cagccgctgc cgccgattcc ggatctcatt gccacgcgcc |
| | cccgacgacc gcccgacgtg |
| 241 | cattcccgat tccttttggt tccaagtcca atatggcaac |
| | tctaaaggat cagctgattt |
| 301 | ataatcttct aaaggaagaa cagacccccc agaataagat |
| | tacagttgtt ggggttggtg |
| 361 | ctgttggcat ggcctgtgcc atcagtatct taatgaagga |
| | cttggcagat gaacttgctc |
| 421 | ttgttgatgt catcgaagac aaattgaagg gagagatgat |
| | ggatctccaa catggcagcc |
| 481 | ttttccttag aacaccaaag attgtctctg gcaaagacta |
| | taatgtaact gcaaactcca |
| 541 | agctggtcat tatcacggct ggggcacgtc agcaagaggg |
| | agaaagccgt cttaatttgg |
| 601 | tccagcgtaa cgtgaacatc tttaaattca tcattcctaa |
| | tgttgtaaaa tacagcccga |
| 661 | actgcaagtt gcttattgtt tcaaatccag tggatatctt |
| | gacctacgtg gcttggaaga |
| 721 | taagtggttt tcccaaaaac cgtgttattg gaagcggttg |
| | caatctggat tcagcccgat |
| 781 | tccgttacct aatgggggaa aggctgggag ttcacccatt |
| | aagctgtcat gggtgggtcc |
| 841 | ttggggaaca tggagattcc agtgtgcctg tatggagtgg |
| | aatgaatgtt gctggtgtct |
| 901 | ctctgaagac tctgcaccca gatttaggga ctgataaaga |
| | taaggaacag tggaaagagg |
| 961 | ttcacaagca ggtggttgag agtgcttatg aggtgatcaa |
| | actcaaaggc tacacatcct |
| 1021 | gggctattgg actctctgta gcagatttgg cagagagtat |
| | aatgaagaat cttaggcggg |
| 1081 | tgcacccagt ttccaccatg attaagggtc tttacggaat |
| | aaaggatgat gtcttcctta |
| 1141 | gtgttccttg cattttggga cagaatggaa tctcagacct |
| | tgtgaaggtg actctgactt |
| 1201 | ctgaggaaga ggcccgtttg aagaagagtg cagatacact |
| | ttgggggatc caaaaggagc |
| 1261 | tgcaatttta aagtcttctg atgtcatatc atttcactgt |
| | ctaggctaca acaggattct |
| 1321 | aggtggaggt tgtgcatgtt gtccttttta tctgatctgt |
| | gattaaagca gtaatatttt |
| 1381 | aagatggact gggaaaaaca tcaactcctg aagttagaaa |
| | taagaatggt ttgtaaaatc |
| 1441 | cacagctata tcctgatgct ggatggtatt aatcttgtgt |
| | agtcttcaac tggttagtgt |
| 1501 | gaaatagttc tgccacctct gacgcaccac tgccaatgct |
| | gtacgtactg catttgcccc |
| 1561 | ttgagccagg tggatgttta ccgtgtgtta tataacttcc |
| | tggctccttc actgaacatg |
| 1621 | cctagtccaa cattttttcc cagtgagtca catcctggga |
| | tccagtgtat aaatccaata |
| 1681 | tcatgtcttg tgcataattc ttccaaagga tcttattttg |
| | tgaactatat cagtagtgta |
| 1741 | cattaccata taatgtaaaa agatctacat acaaacaatg |
| | caaccaacta tccaagtgtt |
| 1801 | ataccaacta aaacccccaa taaaccttga acagtgacta |
| | ctttggttaa ttcattatat |
| 1861 | taagatataa agtcataaag ctgctagtta ttatattaat |
| | ttggaaatat taggctattc |
| 1921 | ttgggcaacc ctgcaacgat tttttctaac agggatatta |
| | ttgactaata gcagaggatg |
| 1981 | taatagtcaa ctgagttgta ttggtaccac ttccattgta |
| | agtcccaaag tattatatat |
| 2041 | ttgataataa tgctaatcat aattggaaag taacattcta |
| | tatgtaaatg taaaatttat |
| 2101 | ttgccaactg aatataggca atgatagtgt gtcactatag |
| | ggaacacaga tttttgagat |
| 2161 | cttgtcctct ggaagctggt aacaattaaa aacaatctta |
| | aggcagggaa aaaaaaaaaa |
| 2221 | aaaaaa |

By “MAFA polypeptide” is meant a protein or fragment thereof having at least 85%, at least 90%, at least 95%, at least 98%, or at least 99% amino acid sequence identity to the sequence provided at NCBI Accession No. NP_963883.2 and having transcription factor activity. The amino acid sequence provided at NCBI Accession No. NP_963883.2 is shown below:

| | |
|---|---|
| 1 | MAAELAMGAE LPSSPLAIEY VNDFDLMKFE VKKEPPEAER |
| | FCHRLPPGSL SSTPLSTPCS |
| 61 | SVPSSPSFCA PSPGTGGGGG AGGGGGSSQA GGAPGPPSGG |
| | PGAVGGTSGK PALEDLYWMS |
| 121 | GYQHHLNPEA LNLTPEDAVE ALIGSGHHGA HHGAHHPAAA |
| | AAYEAFRGPG FAGGGGADDM |
| 181 | GAGHHHGAHH AAHHHHAAHH HHHHHHHHGG AGHGGGAGHH |
| | VRLEERFSDD QLVSMSVREL |
| 241 | NRQLRGFSKE EVIRLKQKRR TLKNRGYAQS CRFKRVQQRH |
| | ILESEKCQLQ SQVEQLKLEV |
| 301 | GRLAKERDLY KEKYEKLAGR GGPGSAGGAG FPREPSPPQA |
| | GPGGAKGTAD FFL |

By “MAFA polynucleotide” is meant a polynucleotide encoding a MAFA polypeptide or fragment thereof. An exemplary MAFA polynucleotide sequence is provided at NCBI Ref NM_201589.3. The sequence provided at NCBI Ref NM_201589.3 is reproduced below:

| | |
|---|---|
| 1 | gcgcggccgg gcgcgggccc cgggcgatgg ccgcggagct |
| | ggcgatgggc gccgagctgc |
| 61 | ccagcagccc gctggccatc gagtacgtca acgacttcga |
| | cctgatgaag ttcgaggtga |
| 121 | agaaggagcc tcccgaggcc gagcgcttct gccaccgcct |
| | gccgccaggc tcgctgtcct |
| 181 | cgacgccgct cagcacgccc tgctcctccg tgccctcctc |
| | gcccagcttc tgcgcgccca |
| 241 | gcccgggcac cggcggcggc ggcggcgcgg ggggcggcgg |
| | cggctcgtct caggccgggg |
| 301 | gcgcccccgg gccgccgagc gggggccccg gcgccgtcgg |
| | gggcacctcg gggaagccgg |
| 361 | cgctggagga tctgtactgg atgagcggct accagcatca |
| | cctcaacccc gaggcgctca |
| 421 | acctgacgcc cgaggacgcg gtggaggcgc tcatcggcag |
| | cggccaccac ggcgcgcacc |
| 481 | acggcgcgca ccacccggcg gccgccgcag cctacgaggc |
| | tttccgcggc ccgggcttcg |
| 541 | cgggcggcgg cggagcggac gacatgggcg ccggccacca |
| | ccacggcgcg caccacgccg |
| 601 | cccaccatca ccacgccgcc caccaccacc accaccacca |
| | ccaccaccat ggcggcgcgg |
| 661 | gacacggcgg tggcgcgggc caccacgtgc gcctggagga |
| | gcgcttctcc gacgaccagc |
| 721 | tggtgtccat gtcggtgcgc gagctgaacc ggcagctccg |
| | cggcttcagc aaggaggagg |
| 781 | tcatccggct caagcagaag cggcgcacgc tcaagaaccg |
| | cggctacgcg cagtcctgcc |
| 841 | gcttcaagcg ggtgcagcag cggcacattc tggagagcga |
| | gaagtgccaa ctccagagcc |
| 901 | aggtggagca gctgaagctg gaggtggggc gcctggccaa |
| | agagcgggac ctgtacaagg |
| 961 | agaaatacga gaagctggcg ggccggggcg gccccgggag |
| | cgcgggcggg gccggtttcc |
| 1021 | cgcgggagcc ttcgccgccg caggccggtc ccggcggggc |
| | caagggcacg gccgacttct |
| 1081 | tcctgtaggc gccggacccc gagcccgcgc cgccgtcgcc |
| | ggggacaagt tcgcgcaggc |
| 1141 | ctctcggggc ctcggctcgg actccgcggt acaggacgtg |
| | gacaccaggc ccggcccggc |
| 1201 | cgtgctggcc ccggtgccaa gtctgcgggc gcggggctgg |
| | aggccccttc gctcccggtc |
| 1261 | cccgttcgcg cgcgtcggcc cgggtcgccg tcctgaggtt |
| | gagcggagaa cggtgatttc |
| 1321 | taaggaaact tgagccaggt ctaacttctt tccaagcgtc |
| | cgcttgtaca tacgttgaac |
| 1381 | gtggttctcc gttcccacct tcgccctgcc agcctagagg |
| | gaccgcgctg ccgtcccttc |
| 1441 | ccgggtggcc cctgcctgcc cccgccctcc ttcgttctct |
| | tctcagcctc cctttccttg |
| 1501 | ccttttttaa cttcccctcc ccgttttaaa atcggtctta |
| | ttttcgaagt atttataatt |
| 1561 | attatgcttg gtgattagaa aagaaaacct tggaggaagc |
| | cccttctttc cccagccggg |
| 1621 | gtccgccctc agtcgcgagt cacagcatga gtcgctcgcc |
| | aggaggggcc cggcccctgc |
| 1681 | ctgccccctc cccgcttgcc cccgaccctg ctaccggcgt |
| | tccttggagg tcgaagccag |
| 1741 | ggacgtcacc cgtgctgtgt ccaggcctgc tgtcctacta |
| | tgctcaaccg ggggtggggg |
| 1801 | gaggggggtg agtcctgtgc tcagtcgggt gggggctggc |
| | ccggatcccg agctgctgtc |
| 1861 | tctctatgca ccagaacata tctgtaactc ctggggaaat |
| | acatcttgtt ttaaccttca |
| 1921 | agagaagtga aagaaaaaag taatgcacag tatttctagc |
| | agaaaatttt tttttttaag |
| 1981 | aggaggcttg ggccagagcc ttctggcatg gggcgggtgg |
| | agaaagtgtt tttattttaa |
| 2041 | tttaaattgt gtttcgtttt gtttgtggaa tctttcttta |
| | atgcttcgtc gctctttgga |
| 2101 | ctagccggga gagagggcga ggaggcgggt gctccaggcc |
| | ctgtaggctg ggccaggcgc |
| 2161 | ctgggggatc tgcccgtttt cggaggccct caggggccat |
| | cagtgggatt ccagccgctc |
| 2221 | cacacccctc ccctgagcac tcggagtgga aggcgcgccg |
| | actcgttgaa agttttgttg |
| 2281 | tgtagttggt tttcgttgag ttcttttttc atttgctacg |
| | aaactgagaa aaagaaaaaa |
| 2341 | atacacaaaa taaatctgtt cagatccaag tca |

As used herein, a “marker” is meant any protein or polynucleotide having an alteration in expression level or activity that is associated with a disease or disorder or that is associated with a particular cell type. In some embodiments, a marker for a beta cell is Pdx1, MafA, Pax4, Pax6, NeuroDI, Nkx6-1, Gata6, or Foxa2. In some embodiments, a marker for a hepatocyte is AFP, ALB, or Cyp3a7. In some other embodiments, a marker for a cardiomyocyte is hMlc2a, hNkx2-5, alphaMHC or KCNQ1. In still other embodiments, a marker for a small intestine cell is CDX2, Muc2, or Lgr5.

By “alphaMHC polypeptide” or “myosin heavy chain (MHC) alpha polypeptide” is meant a protein or fragment thereof having at least 85%, at least 90%, at least 95%, at least 98%, or at least 99% amino acid sequence identity to the sequence provided at NCBI Accession No. NP_002462.2 and having actin binding activity. The amino acid sequence provided at NCBI Accession No. NP_002462.2 is shown below:

| | |
|---|---|
| 1 | MTDAQMADFG AAAQYLRKSE KERLEAQTRP FDIRTECFVP |
| | DDKEEFVKAK ILSREGGKVI |
| 61 | AETENGKTVT VKEDQVLQQN PPKFDKIEDM AMLTFLHEPA |
| | VLFNLKERYA AWMIYTYSGL |
| 121 | FCVTVNPYKW LPVYNAEVVA AYRGKKRSEA PPHIFSISDN |
| | AYQYMLTDRE NQSILITGES |
| 181 | GAGKTVNTKR VIQYFASIAA IGDRGKKDNA NANKGTLEDQ |
| | IIQANPALEA FGNAKTVRND |
| 241 | NSSRFGKFIR IHFGATGKLA SADIETYLLE KSRVIFQLKA |
| | ERNYHIFYQI LSNKKPELLD |
| 301 | MLLVTNNPYD YAFVSQGEVS VASIDDSEEL MATDSAFDVL |
| | GFTSEEKAGV YKLTGAIMHY |
| 361 | GNMKFKQKQR EEQAEPDGTE DADKSAYLMG LNSADLLKGL |
| | CHPRVKVGNE YVTKGQSVQQ |
| 421 | VYYSIGALAK AVYEKMFNWM VTRINATLET KQPRQYFIGV |
| | LDIAGFEIFD FNSFEQLCIN |
| 481 | FTNEKLQQFF NHHMFVLEQE EYKKEGIEWT FIDFGMDLQA |
| | CIDLIEKPMG IMSILEEECM |
| 541 | FPKATDMIFK AKLYDNHLGK SNNFQKPRNI KGKQEAHFSL |
| | IHYAGTVDYN ILGWLEKNKD |
| 601 | PLNETVVALY QKSSLKLMAT LFSSYATADT GDSGKSKGGK |
| | KKGSSFQTVS ALHRENLNKL |
| 661 | MTNLRTTHPH FVRCIIPNER KAPGVMDNPL VMHQLRCNGV |
| | LEGIRICRKG FPNRILYGDF |
| 721 | RQRYRILNPV AIPEGQFIDS RKGTEKLLSS LDIDHNQYKF |
| | GHTKVFFKAG LLGLLEEMRD |
| 781 | ERLSRIITRM QAQARGQLMR IEFKKIVERR DALLVIQWNI |
| | RAFMGVKNWP WMKLYFKIKP |
| 841 | LLKSAETEKE MATMKEEFGR IKETLEKSEA RRKELEEKMV |
| | SLLQEKNDLQ LQVQAEQDNL |
| 901 | NDAEERCDQL IKNKIQLEAK VKEMNERLED EEEMNAELTA |
| | KKRKLEDECS ELKKDIDDLE |
| 961 | LTLAKVEKEK HATENKVKNL TEEMAGLDEI IAKLTKEKKA |
| | LQEAHQQALD DLQVEEDKVN |
| 1021 | SLSKSKVKLE QQVDDLEGSL EQEKKVRMDL ERAKRKLEGD |
| | LKLTQESIMD LENDKLQLEE |
| 1081 | KLKKKEFDIN QQNSKIEDEQ VLALQLQKKL KENQARIEEL |
| | EEELEAERTA RAKVEKLRSD |
| 1141 | LSRELEEISE RLEEAGGATS VQIEMNKKRE AEFQKMRRDL |
| | EEATLQHEAT AAALRKKHAD |
| 1201 | SVAELGEQID NLQRVKQKLE KEKSEFKLEL DDVTSNMEQI |
| | IKAKANLEKV SRTLEDQANE |
| 1261 | YRVKLEEAQR SLNDFTTQRA KLQTENGELA RQLEEKEALI |
| | SQLTRGKLSY TQQMEDLKRQ |
| 1321 | LEEEGKAKNA LAHALQSARH DCDLLREQYE EETEAKAELQ |
| | RVLSKANSEV AQWRTKYETD |
| 1381 | AIQRTEELEE AKKKLAQRLQ DAEEAVEAVN AKCSSLEKTK |
| | HRLQNEIEDL MVDVERSNAA |
| 1441 | AAALDKKQRN FDKILAEWKQ KYEESQSELE SSQKEARSLS |
| | TELFKLKNAY EESLEHLETF |
| 1501 | KRENKNLQEE ISDLTEQLGE GGKNVHELEK VRKQLEVEKL |
| | ELQSALEEAE ASLEHEEGKI |
| 1561 | LRAQLEFNQI KAEIERKLAE KDEEMEQAKR NHQRVVDSLQ |
| | TSLDAETRSR NEVLRVKKKM |
| 1621 | EGDLNEMEIQ LSHANRMAAE AQKQVKSLQS LLKDTQIQLD |
| | DAVRANDDLK ENIAIVERRN |
| 1681 | NLLQAELEEL RAVVEQTERS RKLAEQELIE TSERVQLLHS |
| | QNTSLINQKK KMESDLTQLQ |
| 1741 | SEVEEAVQEC RNAEEKAKKA ITDAAMMAEE LKKEQDTSAH |
| | LERMKKNMEQ TIKDLQHRLD |
| 1801 | EAEQIALKGG KKQLQKLEAR VRELEGELEA EQKRNAESVK |
| | GMRKSERRIK ELTYQTEEDK |
| 1861 | KNLLRLQDLV DKLQLKVKAY KRQAEEAEEQ ANTNLSKFRK |
| | VQHELDEAEE RADIAESQVN |
| 1921 | KLRAKSRDIG AKQKMHDEE |

By “alphaMHC polynucleotide” is meant a polynucleotide encoding a alphaMHC polypeptide or fragment thereof. An exemplary alphaMHC polynucleotide sequence is provided at NCBI Ref: NM_002471.3. The sequence provided at NCBI Ref: NM_002471.3 is reproduced below:

| | |
|---|---|
| 1 | agatagagag actcctgcgg cccagattct tcaggattct |
| | ccgtgaaggg ataaccaggg |
| 61 | gaagcaccaa gatgaccgat gcccagatgg ctgactttgg |
| | ggcagcggcc cagtacctcc |
| 121 | gcaagtcaga gaaggagcgt ctagaggccc agacccggcc |
| | ctttgacatt cgcactgagt |
| 181 | gcttcgtgcc cgatgacaag gaagagtttg tcaaagccaa |
| | gattttgtcc cgggagggag |
| 241 | gcaaggtcat tgctgaaacc gagaatggga agacggtgac |
| | tgtgaaggag gaccaggtgt |
| 301 | tgcagcagaa cccacccaag ttcgacaaga ttgaggacat |
| | ggccatgctg accttcctgc |
| 361 | acgagcccgc ggtgcttttc aacctcaagg agcgctacgc |
| | ggcctggatg atatatacct |
| 421 | actcgggcct cttctgtgtc actgtcaacc cctacaagtg |
| | gctgccggtg tacaatgccg |
| 481 | aggtggtggc cgcctaccgg ggcaagaaga ggagtgaggc |
| | cccgccccac atcttctcca |
| 541 | tctccgacaa cgcctatcag tacatgctga cagatcggga |
| | gaaccagtcc atcctcatca |
| 601 | cgggagaatc cggggcgggg aagactgtga acaccaagcg |
| | tgtcatccag tactttgcca |
| 661 | gcattgcagc cataggtgac cgtggcaaga aggacaatgc |
| | caatgcgaac aagggcaccc |
| 721 | tggaggacca gatcatccag gccaaccccg ctctggaggc |
| | cttcggcaat gccaagactg |
| 781 | tccggaacga caactcctcc cgctttggga aattcattag |
| | gatccacttt ggggccactg |
| 841 | gaaagctggc ttctgcagac atagagacct acctgctgga |
| | gaagtcccgg gtgatcttcc |
| 901 | agctgaaagc tgagagaaac taccacatct tctaccagat |
| | tctgtccaac aagaagccgg |
| 961 | agttgctgga catgctgctg gtcaccaaca atccctacga |
| | ctacgccttc gtgtctcagg |
| 1021 | gagaggtgtc cgtggcctcc attgatgact ccgaggagct |
| | catggccacc gatagtgcct |
| 1081 | ttgacgtgct gggcttcact tcagaggaga aagctggcgt |
| | ctacaagctg acgggagcca |
| 1141 | tcatgcacta cgggaacatg aagttcaagc agaagcagcg |
| | ggaggagcag gcggagccag |
| 1201 | acggcaccga agatgctgac aagtcggcct acctcatggg |
| | gctgaactca gctgacctgc |
| 1261 | tcaaggggct gtgccaccct cgggtgaaag tgggcaacga |
| | gtatgtcacc aaggggcaga |
| 1321 | gcgtgcagca ggtgtactac tccatcgggg ctctggccaa |
| | ggcagtgtat gagaagatgt |
| 1381 | tcaactggat ggtgacgcgc atcaacgcca ccctggagac |
| | caagcagcca cgccagtact |
| 1441 | tcataggagt cctggacatc gctggcttcg agatcttcga |
| | cttcaacagc tttgagcagc |
| 1501 | tctgcatcaa cttcaccaac gagaagctgc agcagttctt |
| | caaccaccac atgttcgtgc |
| 1561 | tggagcagga ggagtacaag aaggagggca ttgagtggac |
| | attcattgac tttggcatgg |
| 1621 | acctgcaggc ctgcattgac ctcatcgaga agcccatggg |
| | catcatgtcc atcctggagg |
| 1681 | aggagtgcat gttccccaag gccactgaca tgaccttcaa |
| | ggccaagctg tacgacaacc |
| 1741 | acctgggcaa gtccaacaat ttccagaagc cacgcaacat |
| | caaggggaag caggaagccc |
| 1801 | acttctccct gatccactac gccggcactg tggactacaa |
| | catcctgggc tggctggaaa |
| 1861 | aaaacaagga tcctctcaac gagactgttg tggccctgta |
| | ccagaagtcc tccctcaagc |
| 1921 | tcatggccac tctcttctcc tcctacgcaa ctgccgatac |
| | tggggacagt ggtaaaagca |
| 1981 | aaggaggcaa gaaaaagggc tcatccttcc agacggtgtc |
| | ggctctccac cgggaaaatc |
| 2041 | tcaacaagct aatgaccaac ctgaggacca cccatcctca |
| | ctttgtgcgt tgcatcatcc |
| 2101 | ccaatgagcg gaaggctcca ggggtgatgg acaaccccct |
| | ggtcatgcac cagctgcgct |
| 2161 | gcaatggcgt gctggagggc atccgcatct gcaggaaggg |
| | cttccccaac cgcatcctct |
| 2221 | acggggactt ccggcagagg tatcgcatcc tgaacccagt |
| | ggccatccct gagggacagt |
| 2281 | tcattgatag caggaagggg acagagaagc tgctcagctc |
| | tctggacatt gatcacaacc |
| 2341 | agtacaagtt tggccacacc aaggtgttct tcaaggcagg |
| | gctgcttggg ctgctggagg |
| 2401 | agatgcggga tgagaggctg agccgcatca tcacgcgcat |
| | gcaggcccaa gcccggggcc |
| 2461 | agctcatgcg cattgagttc aagaagatag tggaacgcag |
| | ggatgccctg ctggtaatcc |
| 2521 | agtggaacat tcgggccttc atgggggtca agaattggcc |
| | ctggatgaag ctctacttca |
| 2581 | agatcaagcc gctgctgaag agcgcagaga cggagaagga |
| | gatggccacc atgaaggaag |
| 2641 | agttcgggcg catcaaagag acgctggaga agtccgaggc |
| | tcgccgcaag gagctggagg |
| 2701 | agaagatggt gtccctgctg caggagaaga atgacctgca |
| | gctccaagtg caggcggaac |
| 2761 | aagacaacct caatgatgct gaggagcgct gcgaccagct |
| | gatcaaaaac aagattcagc |
| 2821 | tggaggccaa agtaaaggag atgaatgaga ggctggagga |
| | tgaggaggag atgaacgcgg |
| 2881 | agctcactgc caagaagcgc aagctggaag acgagtgctc |
| | agagctcaag aaggacattg |
| 2941 | atgacctgga gctgacactg gccaaggtgg agaaggagaa |
| | gcatgcaaca gagaacaagg |
| 3001 | tgaagaacct aacagaggag atggctgggc tggatgaaat |
| | catcgctaag ctgaccaagg |
| 3061 | agaagaaagc tctacaagag gcccatcagc aggccctgga |
| | tgaccttcag gttgaggaag |
| 3121 | acaaggtcaa cagcctgtcc aagtctaagg tcaagctgga |
| | gcagcaggtg gatgatctgg |
| 3181 | agggatccct agagcaagag aagaaggtgc gcatggacct |
| | ggagcgagca aagcggaaac |
| 3241 | tggagggcga cctgaagctg acccaggaga gcatcatgga |
| | cctggaaaat gataaactgc |
| 3301 | agctggaaga aaagcttaag aagaaggagt ttgacattaa |
| | tcagcagaac agtaagattg |
| 3361 | aggatgagca ggtgctggcc cttcaactac agaagaaact |
| | gaaggaaaac caggcacgca |
| 3421 | tcgaggagct ggaggaggag ctggaggccg agcgcaccgc |
| | cagggctaag gtggagaagc |
| 3481 | tgcgctcaga cctgtctcgg gagctggagg agatcagcga |
| | gcggctggaa gaggccggcg |
| 3541 | gggccacgtc cgtgcagatc gagatgaaca agaagcgcga |
| | ggccgagttc cagaagatgc |
| 3601 | ggcgggacct ggaggaggcc acgctgcagc acgaggccac |
| | tgccgcggcc ctgcgcaaga |
| 3661 | agcacgccga cagcgtggcc gagctgggcg agcagatcga |
| | caacctgcag cgggtgaagc |
| 3721 | agaagctgga gaaggagaag agcgagttca agctggagct |
| | ggatgacgtc acctccaaca |
| 3781 | tggagcagat catcaaggcc aaggcaaacc tggagaaagt |
| | gtctcggacg ctggaggacc |
| 3841 | aggccaatga gtaccgcgtg aagctagaag aggcccaacg |
| | ctccctcaat gatttcacca |
| 3901 | cccagcgagc caagctgcag accgagaatg gagagttggc |
| | ccggcagcta gaggaaaagg |
| 3961 | aggcgctaat ctcgcagctg acccggggga agctctctta |
| | tacccagcaa atggaggacc |
| 4021 | tcaaaaggca gctggaggag gagggcaagg cgaagaacgc |
| | cctggcccat gcactgcagt |
| 4081 | cggcccggca tgactgcgac ctgctgcggg agcagtacga |
| | ggaggagaca gaggccaagg |
| 4141 | ccgagctgca gcgcgtcctg tccaaggcca actcggaggt |
| | ggcccagtgg aggaccaagt |
| 4201 | atgagacgga cgccattcag cggactgagg agctcgaaga |
| | ggccaaaaag aagctggccc |
| 4261 | agcggctgca ggatgccgag gaggccgtgg aggctgttaa |
| | tgccaagtgc tcctcactgg |
| 4321 | agaagaccaa gcaccggcta cagaatgaga tagaggactt |
| | gatggtggac gtagagcgct |
| 4381 | ccaatgctgc tgctgcagcc ctggacaaga agcagagaaa |
| | ctttgacaag atcctggccg |
| 4441 | agtggaagca gaagtatgag gagtcgcagt ctgagctgga |
| | gtcctcacag aaggaggctc |
| 4501 | gctccctcag cacagagctc ttcaagctca agaacgccta |
| | cgaggagtcc ctggagcacc |
| 4561 | tagagacctt caagcgggag aacaagaacc ttcaggagga |
| | aatctcggac cttactgagc |
| 4621 | agctaggaga aggaggaaag aatgtgcatg agctggagaa |
| | ggtccgcaaa cagctggagg |
| 4681 | tggagaagct ggagctgcag tcagccctgg aggaggcaga |
| | ggcctccctg gagcacgagg |
| 4741 | agggcaagat cctccgggcc cagctagagt tcaaccagat |
| | caaggcagag atcgagcgga |
| 4801 | agctggcaga gaaggacgag gagatggaac aggccaagcg |
| | caaccaccag cgggtggtgg |
| 4861 | actcgctgca gacctccctg gatgcagaga cacgcagccg |
| | caacgaggtc ctgagggtga |
| 4921 | agaagaagat ggaaggagac ctcaatgaga tggagatcca |
| | gctcagccac gccaaccgca |
| 4981 | tggctgccga ggcccagaag caagtcaaga gcctccagag |
| | cttgctgaag gacacccaga |
| 5041 | tccagctgga cgatgcggtc cgtgccaacg acgacctgaa |
| | ggagaacatc gccatcgtgg |
| 5101 | agcggcgcaa caacctgctg caggctgagc tggaggagct |
| | gcgtgccgtg gtggagcaga |
| 5161 | cagagcggtc ccggaagctg gcggagcagg agctgattga |
| | gaccagcgag cgggtgcagc |
| 5221 | tgctgcattc ccagaacacc agcctcatca accagaagaa |
| | gaagatggag tcggatctga |
| 5281 | cccagctcca gtcggaagtg gaggaggcag tgcaggagtg |
| | cagaaacgcc gaggagaagg |
| 5341 | ccaagaaggc catcacggat gccgccatga tggcagagga |
| | gctgaagaag gagcaggaca |
| 5401 | ccagcgccca cctggagcgc atgaagaaga acatggagca |
| | gaccattaag gacctgcagc |
| 5461 | accggctgga cgaggccgag cagatcgccc tcaagggagg |
| | caagaagcag ctgcagaagc |
| 5521 | tggaagcgcg ggtgcgggag ctggagggtg agctggaggc |
| | cgagcagaag cgcaacgcag |
| 5581 | agtcggtgaa gggcatgagg aagagcgagc ggcgcatcaa |
| | ggagctcacc taccagacag |
| 5641 | aggaagacaa aaagaacctg ctgcggctac aggacctggt |
| | ggacaagctg caactgaagg |
| 5701 | tcaaggccta caagcgccag gccgaggagg cggaggagca |
| | agccaacacc aacctgtcca |
| 5761 | agttccgcaa ggtgcagcat gagctggatg aggcagagga |
| | gcgggcggac atcgctgagt |
| 5821 | cccaggtcaa caagcttcga gccaagagcc gtgacattgg |
| | tgccaagcaa aaaatgcacg |
| 5881 | atgaggagtg acactgcctc gggaacctca ctcttgccaa |
| | cctgtaataa atatgagtgc |
| 5941 | c |

By “MLC2A polypeptide” or “human MLSC2A (hMLC2A) polypeptide” is meant a protein or fragment thereof having at least 85%, at least 90%, at least 95%, at least 98%, or at least 99% amino acid sequence identity to the sequence provided at NCBI Accession No. NP_067046.1 and having calcium binding activity. The amino acid sequence provided at NCBI Accession No. NP_067046.1 is shown below:

| | |
|---|---|
| 1 | MASRKAGTRG KVAATKQAQR GSSNVFSMFE QAQIQEFKEA |
| | FSCIDQNRDG IICKADLRET |
| 61 | YSQLGKVSVP EEELDAMLQE GKGPINFTVF LTLFGEKLNG |
| | TDPEEAILSA FRMFDPSGKG |
| 121 | VVNKDEFKQL LLTQADKFSP AEVEQMFALT PMDLAGNIDY |
| | KSLCYIITHG DEKEE |

By “MLC2A polynucleotide” is meant a polynucleotide encoding a MLC2A polypeptide or fragment thereof. An exemplary MLC2A polynucleotide sequence is provided at NCBI Ref: NM_021223.2. The sequence provided at NCBI Ref: NM_021223.2 is reproduced below:

(SEQ ID NO: 28)

| | |
|---|---|
| 1 | tctgcagaga gaatggccag caggaaggcg |
| | gggacccggg gcaaggtggc agccaccaag |
| 61 | caggcccaac gtggttcttc caacgtcttt |
| | tccatgtttg aacaagccca gatacaggag |
| 121 | ttcaaagaag ccttcagctg tatcgaccag |
| | aatcgtgatg gcatcatctg caaggcagac |
| 181 | ctgagggaga cctactccca gctggggaag |
| | gtgagtgtcc cagaggagga gctggacgcc |
| 241 | atgctgcaag agggcaaggg ccccatcaac |
| | ttcaccgtct tcctcacgct ctttggggag |
| 301 | aagctcaatg ggacagaccc cgaggaagcc |
| | atcctgagtg ccttccgcat gtttgacccc |
| 361 | agcggcaaag gggtggtgaa caaggatgag |
| | ttcaagcagc ttctcctgac ccaggcagac |
| 421 | aagttctctc cagctgaggt ggagcagatg |
| | ttcgccctga cacccatgga cctggcgggg |
| 481 | aacatcgact acaagtcact gtgctacatc |
| | atcacccatg gagacgagaa agaggaatga |
| 541 | ggggcagggc caggcccacg ggggggcacc |
| | tcaataaact ctgttgcaaa attggaaaaa |
| 601 | aaaaaaaaaa aaaaaaaaa |

By “MUC2 polypeptide” is meant a protein or fragment thereof having at least 85%, at least 90%, at least 95%, at least 98%, or at least 99% amino acid sequence identity to the sequence provided at NCBI Accession No. NP_002448.3 and having and having a biological activity of a MUC2 polypeptide. Exemplary biological activities of a MUC2 polypeptide include polymerization into a gel and coating of epithelia of the intestines and other mucus membrane-containing organs. The amino acid sequence provided at NCBI Accession No. NP_002448.3 is shown below:

| (SEQ ID NO: 29) | |
|---|---|
| 1 | MGLPLARLAA VCLALSLAGG SELQTEGRTR NHGHNVCSTW GNFHYKTFDG DVFRFPGPCD |
| 61 | YNFASDCRGS YKEFAVHLKR GPGQAEAPAG VESILLTIKD DTIYLTRHLA VLNGAVVSTP |
| 121 | HYSPGLLIEK SDAYTKVYSR AGLTLMWNRE DALMLELDTK FRNHTCGLCG DYNGLQSYSE |
| 181 | FLSDGVLFSP LEFGNMQKIN QPDVVCEDPE EEVAPASCSE HRAECERLLT AEAFADCQDL |
| 241 | VPLEPYLRAC QQDRCRCPGG DTCVCSTVAE FSRQCSHAGG RPGNWRTATL CPKTCPGNLV |
| 301 | YLESGSPCMD TCSHLEVSSL CEEHRMDGCF CPEGTVYDDI GDSGCVPVSQ CHCRLHGHLY |
| 361 | TPGQEITNDC EQCVCNAGRW VCKDLPCPGT CALEGGSHIT TFDGKTYTFH GDCYYVLAKG |
| 421 | DHNDSYALLG ELAPCGSTDK QTCLKTVVLL ADKKKNVVVF KSDGSVLLNE LQVNLPHVTA |
| 481 | SFSVFRPSSY HIMVSMAIGV RLQVQLAPVM QLFVTLDQAS QGQVQGLCGN FNGLEGDDFK |
| 541 | TASGLVEATG AGFANTWKAQ STCHDKLDWL DDPCSLNIES ANYAEHWCSL LKKTETPFGR |
| 601 | CHSAVDPAEY YKRCKYDTCN CQNNEDCLCA ALSSYARACT AKGVMLWGWR EHVCNKDVGS |
| 661 | CPNSQVFLYN LTTCQQTCRS LSEADSHCLE GFAPVDGCGC PDHTFLDEKG RCVPLAKCSC |
| 721 | YHRGLYLEAG DVVVRQEERC VCRDGRLHCR QIRLIGQSCT APKIHMDCSN LTALATSKPR |
| 781 | ALSCQTLAAG YYHTECVSGC VCPDGLMDDG RGGCVVEKEC PCVHNNDLYS SGAKIKVDCN |
| 841 | TCTCKRGRWV CTQAVCHGTC SIYGSGHYIT FDGKYYDFDG HCSYVAVQDY CGQNSSLGSF |
| 901 | SIITENVPCG TTGVTCSKAI KIFMGRTELK LEDKHRVVIQ RDEGHHVAYT TREVGQYLVV |
| 961 | ESSTGIIVIW DKRTTVFIKL APSYKGTVCG LCGNFDHRSN NDFTTRDHMV VSSELDFGNS |
| 1021 | WKEAPTCPDV STNPEPCSLN PHRRSWAEKQ CSILKSSVFS ICHSKVDPKP FYEACVHDSC |
| 1081 | SCDTGGDCEC FCSAVASYAQ ECTKEGACVF WRTPDLCPIF CDYYNPPHEC EWHYEPCGNR |
| 1141 | SFETCRTING IHSNISVSYL EGCYPRCPKD RPIYEEDLKK CVTADKCGCY VEDTHYPPGA |
| 1201 | SVPTEETCKS CVCTNSSQVV CRPEEGKILN QTQDGAFCYW EICGPNGTVE KHFNICSITT |
| 1261 | RPSTLTTFTT ITLPTTPTTF TTTTTTTTPT SSTVLSTTPK LCCLWSDWIN EDHPSSGSDD |
| 1321 | GDRETFDGVC GAPEDIECRS VKDPHLSLEQ LGQKVQCDVS VGFICKNEDQ FGNGPFGLCY |
| 1381 | DYKIRVNCCW PMDKCITTPS PPTTTPSPPP TSTTTLPPTT TPSPPTTTTT TPPPTTTPSP |
| 1441 | PITTTTTPPP TTTPSPPIST TTTPPPTTTP SPPTTTPSPP TTTPSPPTTT TTTPPPTTTP |
| 1501 | SPPTTTPITP PASTTTLPPT TTPSPPTTTT TTPPPTTTPS PPTTTPITPP TSTTTLPPTT |
| 1561 | TPSPPPTTTT TPPPTTTPSP PTTTTPSPPT ITTTTPPPTT TPSPPTTTTT TPPPTTTPSP |
| 1621 | PTTTPITPPT STTTLPPTTT PSPPPTTTTT PPPTTTPSPP TTTTPSPPIT TTTTPPPTTT |
| 1681 | PSSPITTTPS PPTTTMTTPS PTTTPSSPIT TTTTPSSTTT PSPPPTTMTT PSPTTTPSPP |
| 1741 | TTTMTTLPPT TTSSPLTTTP LPPSITPPTF SPFSTTTPTT PCVPLCNWTG WLDSGKPNFH |
| 1801 | KPGGDTELIG DVCGPGWAAN ISCRATMYPD VPIGQLGQTV VCDVSVGLIC KNEDQKPGGV |
| 1861 | IPMAFCLNYE INVQCCECVT QPTTMTTTTT ENPTPPTTTP ITTTTTVTPT PTPTGTQTPT |
| 1921 | TTPITTTTTV TPTPTPTGTQ TPTTTPITTT TTVTPTPTPT GTQTPTTTPI TTTTTVTPTP |
| 1981 | TPTGTQTPTT TPITTTTTVT PTPTPTGTQT PTTTPITTTT TVTPTPTPTG TQTPTTTPIT |
| 2041 | TTTTVTPTPT PTGTQTPTTT PITTTTTVTP TPTPTGTQTP TTTPITTTTT VTPTPTPTGT |
| 2101 | QTPTTTPITT TTTVTPTPTP TGTQTPTTTP ITTTTTVTPT PTPTGTQTPT TTPITTTTTV |
| 2161 | TPTPTPTGTQ TPTTTPITTT TTVTPTPTPT GTQTPTTTPI TTTTTVTPTP TPTGTQTPTT |
| 2221 | TPITTTTTVT PTPTPTGTQT PTTTPITTTT TVTPTPTPTG TQTPTTTPIT TTTTVTPTPT |
| 2281 | PTGTQTPTTT PITTTTTVTP TPTPTGTQTP TTTPITTTTT VTPTPTPTGT QTPTTTPITT |
| 2341 | TTTVTPTPTP TGTQTPTTTP ITTTTTVTPT PTPTGTQTPT TTPITTTTTV TPTPTPTGTQ |
| 2401 | TPTTTPITTT TTVTPTPTPT GTQTPTTTPI TTTTTVTPTP TPTGTQTPTT TPITTTTTVT |
| 2461 | PTPTPTGTQT PTTTPITTTT TVTPTPTPTG TQTPTTTPIT TTTTVTPTPT PTGTQTPTTT |
| 2521 | PITTTTTVTP TPTPTGTQTP TTTPITTTTT VTPTPTPTGT QTPTTTPITT TTTVTPTPTP |
| 2581 | TGTQTPTTTP ITTTTTVTPT PTPTGTQTPT TTPITTTTTV TPTPTPTGTQ TPTTTPITTT |
| 2641 | TTVTPTPTPT GTQTPTTTPI TTTTTVTPTP TPTGTQTPTT TPITTTTTVT PTPTPTGTQT |
| 2701 | PTTTPITTTT TVTPTPTPTG TQTPTTTPIT TTTTVTPTPT PTGTQTPTTT PITTTTTVTP |
| 2761 | TPTPTGTQTP TTTPITTTTT VTPTPTPTGT QTPTTTPITT TTTVTPTPTP TGTQTPTTTP |
| 2821 | ITTTTTVTPT PTPTGTQTPT TTPITTTTTV TPTPTPTGTQ TPTTTPITTT TTVTPTPTPT |
| 2881 | GTQTPTTTPI TTTTTVTPTP TPTGTQTPTT TPITTTTTVT PTPTPTGTQT PTTTPITTTT |
| 2941 | TVTPTPTPTG TQTPTTTPIT TTTTVTPTPT PTGTQTPTTT PITTTTTVTP TPTPTGTQTP |
| 3001 | TTTPITTTTT VTPTPTPTGT QTPTTTPITT TTTVTPTPTP TGTQTPTTTP ITTTTTVTPT |
| 3061 | PTPTGTQTPT TTPITTTTTV TPTPTPTGTQ TPTTTPITTT TTVTPTPTPT GTQTPTTTPI |
| 3121 | TTTTTVTPTP TPTGTQTPTT TPITTTTTVT PTPTPTGTQT PTTTPITTTT TVTPTPTPTG |
| 3181 | TQTPTTTPIT TTTTVTPTPT PTGTQTPTTT PITTTTTVTP TPTPTGTQTP TTTPITTTTT |
| 3241 | VTPTPTPTGT QTPTTTPITT TTTVTPTPTP TGTQTPTTTP ITTTTTVTPT PTPTGTQTPT |
| 3301 | TTPITTTTTV TPTPTPTGTQ TPTTTPITTT TTVTPTPTPT GTQTPTTTPI TTTTTVTPTP |
| 3361 | TPTGTQTPTT TPITTTTTVT PTPTPTGTQT PTTTPITTTT TVTPTPTPTG TQTPTTTPIT |
| 3421 | TTTTVTPTPT PTGTQTPTTT PITTTTTVTP TPTPTGTQTP TTTPITTTTT VTPTPTPTGT |
| 3481 | QTPTTTPITT TTTVTPTPTP TGTQTPTTTP ITTTTTVTPT PTPTGTQTPT TTPITTTTTV |
| 3541 | TPTPTPTGTQ TPTTTPITTT TTVTPTPTPT GTQTPTTTPI TTTTTVTPTP TPTGTQTPTT |
| 3601 | TPITTTTTVT PTPTPTGTQT PTTTPITTTT TVTPTPTPTG TQTPTTTPIT TTTTVTPTPT |
| 3661 | PTGTQTPTTT PITTTTTVTP TPTPTGTQTP TTTPITTTTT VTPTPTPTGT QTPTTTPITT |
| 3721 | TTTVTPTPTP TGTQTPTTTP ITTTTTVTPT PTPTGTQTPT TTPITTTTTV TPTPTPTGTQ |
| 3781 | TPTTTPITTT TTVTPTPTPT GTQTPTTTPI TTTTTVTPTP TPTGTQTPTT TPITTTTTVT |
| 3841 | PTPTPTGTQT PTTTPITTTT TVTPTPTPTG TQTPTTTPIT TTTTVTPTPT PTGTQTPTTT |
| 3901 | PITTTTTVTP TPTPTGTQTP TTTPITTTTT VTPTPTPTGT QTPTTTPITT TTTVTPTPTP |
| 3961 | TGTQTPTTTP ITTTTTVTPT PTPTGTQTPT TTPITTTTTV TPTPTPTGTQ TPTTTPITTT |
| 4021 | TTVTPTPTPT GTQTPTTTPI TTTTTVTPTP TPTGTQTPTT TPITTTTTVT PTPTPTGTQT |
| 4081 | PTTTPITTTT TVTPTPTPTG TQTPTTTPIT TTTTVTPTPT PTGTQTPTTT PITTTTTVTP |
| 4141 | TPTPTGTQTP TTTPITTTTT VTPTPTPTGT QTPTTTPITT TTTVTPTPTP TGTQTGPPTH |
| 4201 | TSTAPIAELT TSNPPPESST PQTSRSTSSP LTESTTLLST LPPAIEMTST APPSTPTAPT |
| 4261 | TTSGGHTLSP PPSTTTSPPG TPTRGTTTGS SSAPTPSTVQ TTTTSAWTPT PTPLSTPSII |
| 4321 | RTTGLRPYPS SVLICCVLND TYYAPGEEVY NGTYGDTCYF VNCSLSCTLE FYNWSCPSTP |
| 4381 | SPTPTPSKST PTPSKPSSTP SKPTPGTKPP ECPDFDPPRQ ENETWWLCDC FMATCKYNNT |
| 4441 | VEIVKVECEP PPMPTCSNGL QPVRVEDPDG CCWHWECDCY CTGWGDPHYV TFDGLYYSYQ |
| 4501 | GNCTYVLVEE ISPSVDNFGV YIDNYHCDPN DKVSCPRTLI VRHETQEVLI KTVHMMPMQV |
| 4561 | QVQVNRQAVA LPYKKYGLEV YQSGINYVVD IPELGVLVSY NGLSFSVRLP YHRFGNNTKG |
| 4621 | QCGTCTNTTS DDCILPSGEI VSNCEAAADQ WLVNDPSKPH CPHSSSTTKR PAVTVPGGGK |
| 4681 | TTPHKDCTPS PLCQLIKDSL FAQCHALVPP QHYYDACVFD SCFMPGSSLE CASLQAYAAL |
| 4741 | CAQQNICLDW RNHTHGACLV ECPSHREYQA CGPAEEPTCK SSSSQQNNTV LVEGCFCPEG |
| 4801 | TMNYAPGFDV CVKTCGCVGP DNVPREFGEH FEFDCKNCVC LEGGSGIICQ PKRCSQKPVT |
| 4861 | HCVEDGTYLA TEVNPADTCC NITVCKCNTS LCKEKPSVCP LGFEVKSKMV PGRCCPFYWC |
| 4921 | ESKGVCVHGN AEYQPGSPVY SSKCQDCVCT DKVDNNTLLN VIACTHVPCN TSCSPGFELM |
| 4981 | EAPGECCKKC EQTHCIIKRP DNQHVILKPG DFKSDPKNNC TFFSCVKIHN QLISSVSNIT |
| 5041 | CPNFDASICI PGSITFMPNG CCKTCTPRNE TRVPCSTVPV TTEVSYAGCT KTVLMNHCSG |
| 5101 | SCGTFVMYSA KAQALDHSCS CCKEEKTSQR EVVLSCPNGG SLTHTYTHIE SCQCQDTVCG |
| 5161 | LPTGTSRRAR RSPRHLGSG |

By “MUC2 polynucleotide” is meant a polynucleotide encoding a MUC2 polypeptide or fragment thereof. An exemplary MUC2 polynucleotide sequence is provided at NCBI Ref NM_002457.3. The sequence provided at NCBI Ref NM_002457.3 is reproduced below:

| (SEQ ID NO: 30) | |
|---|---|
| 1 | caacccacac cgcccctgcc agccaccatg gggctgccac tagcccgcct ggcggctgtg |
| 61 | tgcctggccc tgtctttggc agggggctcg gagctccaga cagagggcag aacccgaaac |
| 121 | cacggccaca acgtctgcag cacctggggc aacttccact acaagacctt cgacggggac |
| 181 | gtcttccgct tccccggccc ctgcgactac aacttcgcct ccgactgccg aggctcctac |
| 241 | aaggaatttg ctgtgcacct gaagcggggt ccgggccagg ctgaggcccc cgccggggtg |
| 301 | gagtccatcc tgctgaccat caaggatgac accatctacc tcacccgcca cctggctgtg |
| 361 | cttaacgggg ccgtggtcag caccccgcac tacagccccg ggctgctcat tgagaagagc |
| 421 | gatgcctaca ccaaagtcta ctcccgcgcc ggcctcaccc tcatgtggaa ccgggaggat |
| 481 | gcactcatgc tggagctgga cactaagttc cggaaccaca cctgtggcct ctgcggggac |
| 541 | tacaacggcc tgcagagcta ttcagaattc ctctctgacg gcgtgctctt cagtcccctg |
| 601 | gagtttggga acatgcagaa gatcaaccag cccgatgtgg tgtgtgagga tcccgaggag |
| 661 | gaggtggccc ccgcatcctg ctccgagcac cgcgccgagt gtgagaggct gctgaccgcc |
| 721 | gaggccttcg cggactgtca ggacctggtg ccgctggagc cgtatctgcg cgcctgccag |
| 781 | caggaccgct gccggtgccc gggcggtgac acctgcgtct gcagcaccgt ggccgagttc |
| 841 | tcccgccagt gctcccacgc cggcggccgg cccgggaact ggaggaccgc cacgctctgc |
| 901 | cccaagacct gccccgggaa cctggtgtac ctggagagcg gctcgccctg catggacacc |
| 961 | tgctcacacc tggaggtgag cagcctgtgc gaggagcacc gcatggacgg ctgtttctgc |
| 1021 | ccagaaggca ccgtatatga cgacatcggg gacagtggct gcgttcctgt gagccagtgc |
| 1081 | cactgcaggc tgcacggaca cctgtacaca ccgggccagg agatcaccaa tgactgcgag |
| 1141 | cagtgtgtct gtaacgctgg ccgctgggtg tgcaaagacc tgccctgccc cggcacctgt |
| 1201 | gccctggaag gcggctccca catcaccacc ttcgatggga agacgtacac cttccacggg |
| 1261 | gactgctact atgtcctggc caagggtgac cacaacgatt cctacgctct cctgggcgag |
| 1321 | ctggccccct gtggctccac agacaagcag acctgcctga agacggtggt gctgctggct |
| 1381 | gacaagaaga agaatgtggt ggtcttcaag tccgatggca gtgtactgct caacgagctg |
| 1441 | caggtgaacc tgccccacgt gaccgcgagc ttctctgtct tccgcccgtc ttcctaccac |
| 1501 | atcatggtga gcatggccat tggcgtccgg ctgcaggtgc agctggcccc agtcatgcaa |
| 1561 | ctctttgtga cactggacca ggcctcccag gggcaggtgc agggcctctg cgggaacttc |
| 1621 | aacggcctgg aaggtgacga cttcaagacg gccagcgggc tggtggaggc cacgggggcc |
| 1681 | ggctttgcca acacctggaa ggcacagtca acctgccatg acaagctgga ctggttggac |
| 1741 | gatccctgct ccctgaacat cgagagcgcc aactacgccg agcactggtg ctccctcctg |
| 1801 | aagaagacag agaccccctt tggcaggtgc cactcggctg tggaccctgc tgagtattac |
| 1861 | aagaggtgca aatatgacac gtgtaactgt cagaacaatg aggactgcct gtgcgccgcc |
| 1921 | ctgtcctcct acgcgcgcgc ctgcaccgcc aagggcgtca tgctgtgggg ctggcgggag |
| 1981 | catgtctgca acaaggatgt gggctcctgc cccaactcgc aggtcttcct gtacaacctg |
| 2041 | accacctgcc agcagacctg ccgctccctc tccgaggccg acagccactg tctcgagggc |
| 2101 | tttgcgcctg tggacggctg cggctgccct gaccacacct tcctggacga gaagggccgc |
| 2161 | tgcgtacccc tggccaagtg ctcctgttac caccgcggtc tctacctgga ggcgggggac |
| 2221 | gtggtcgtca ggcaggaaga acgatgtgtg tgccgggatg ggcggctgca ctgtaggcag |
| 2281 | atccggctga tcggccagag ctgcacggcc ccaaagatcc acatggactg cagcaacctg |
| 2341 | actgcactgg ccacctcgaa gccccgagcc ctcagctgcc agacgctggc cgccggctat |
| 2401 | taccacacag agtgtgtcag tggctgtgtg tgccccgacg ggctgatgga tgacggccgg |
| 2461 | ggtggctgcg tggtggagaa ggaatgccct tgcgtccata acaacgacct gtattcttcc |
| 2521 | ggcgccaaga tcaaggtgga ctgcaatacc tgcacctgca agagaggacg ctgggtgtgc |
| 2581 | acccaggctg tgtgccatgg cacctgctcc atttacggga gtggccacta catcaccttt |
| 2641 | gacgggaagt actacgactt tgacggacac tgctcctacg tggctgttca ggactactgc |
| 2701 | ggccagaact cctcactggg ctcattcagc atcatcaccg agaacgtccc ctgtggcact |
| 2761 | acgggcgtca cctgctccaa ggccatcaag atcttcatgg ggaggacgga gctgaagttg |
| 2821 | gaagacaagc accgtgtggt gatccagcgt gatgagggtc accacgtggc ctacaccacg |
| 2881 | cgggaggtgg gccagtacct ggtggtggag tccagcacgg gcatcatcgt catctgggac |
| 2941 | aagaggacca ccgtgttcat caagctggct ccctcctaca agggcaccgt gtgtggcctg |
| 3001 | tgtgggaact ttgaccaccg ctccaacaac gacttcacca cgcgggacca catggtggtg |
| 3061 | agcagcgagc tggacttcgg gaacagctgg aaggaggccc ccacctgccc agatgtgagc |
| 3121 | accaaccccg agccctgcag cctgaacccg caccgccgct cctgggccga gaagcagtgc |
| 3181 | agcatcctca aaagcagcgt gttcagcatc tgccacagca aggtggaccc caagcccttc |
| 3241 | tacgaggcct gtgtgcacga ctcgtgctcc tgtgacacgg gtggggactg tgagtgcttc |
| 3301 | tgctctgccg tggcctccta cgcccaggag tgtaccaaag agggggcctg cgtgttctgg |
| 3361 | aggacgccgg acctgtgccc catattctgc gactactaca accctccgca tgagtgtgag |
| 3421 | tggcactatg agccatgtgg gaaccggagc ttcgagacct gcaggaccat caatggcatc |
| 3481 | cactccaaca tctccgtgtc ctacctggag ggctgctacc cccggtgccc caaggacagg |
| 3541 | cccatctatg aggaggatct gaagaagtgt gtcactgcag acaagtgtgg ctgctatgtc |
| 3601 | gaggacaccc actacccacc tggagcatcg gttcccaccg aggagacctg caagtcctgc |
| 3661 | gtgtgtacca actcctccca agtcgtctgc aggccggagg aaggaaagat tcttaaccag |
| 3721 | acccaggatg gcgccttctg ctactgggag atctgtggcc ccaacgggac ggtggagaag |
| 3781 | cacttcaaca tctgttccat tacgacacgc ccgtccaccc tgaccacctt caccaccatc |
| 3841 | accctcccca ccacccccac caccttcacc actaccacca ccaccaccac cccgacctcc |
| 3901 | agcacagttt tatcaacaac tccgaagctg tgctgcctct ggtctgactg gatcaatgag |
| 3961 | gaccacccca gcagtggcag cgacgacggt gaccgagaaa catttgatgg ggtctgcggg |
| 4021 | gcccctgagg acatcgagtg caggtcggtc aaggatcccc acctcagctt ggagcagcta |
| 4081 | ggccagaagg tgcagtgtga tgtctctgtt gggttcattt gcaagaatga agaccagttt |
| 4141 | ggaaatggac catttggact gtgttacgac tacaagatac gtgtcaattg ttgctggccc |
| 4201 | atggataagt gtatcaccac tcccagccct ccaactacca ctcccagccc tccaccaacc |
| 4261 | agcacgacca cccttccacc aaccaccacc cccagccctc caaccaccac cacaaccacc |
| 4321 | cctccaccaa ccaccacccc cagccctcca ataaccacca cgaccacccc tccaccaacc |
| 4381 | accactccca gccctccaat aagcaccaca accacccctc caccaaccac cactcccagc |
| 4441 | cctccaacca ccactcccag ccctccaacc accactccca gccctccaac aaccaccaca |
| 4501 | accacccctc caccaaccac cactcccagc cctccaacga ctacgcccat cactccacca |
| 4561 | gccagcacta ccacccttcc accaaccacc actcccagcc ctccaacaac caccacaacc |
| 4621 | acccctccac caaccaccac tcccagtcct ccaacgacta cgcccatcac tccaccaacc |
| 4681 | agcactacta cccttccacc aaccaccact cccagccctc caccaaccac cacaaccacc |
| 4741 | cctccaccaa ccaccactcc cagccctcca acaaccacca ctcccagtcc tccaacaatc |
| 4801 | accacaacca cccctccacc aaccaccact cccagccctc caacaacgac cacaaccacc |
| 4861 | cctccaccaa ccaccactcc cagccctcca acgactacac ccatcactcc accaaccagc |
| 4921 | actaccaccc ttccaccaac caccactccc agccctccac caaccaccac aaccacccct |
| 4981 | ccaccaacca ccactcccag ccctccaaca accaccactc ccagccctcc aataaccacc |
| 5041 | acaaccaccc ctccaccaac caccactccc agctctccaa taaccaccac tcccagccct |
| 5101 | ccaacaacca ccatgaccac cccttcacca accaccaccc ccagctctcc aataaccacc |
| 5161 | acaaccaccc cttcctcaac taccactccc agccctccac caaccaccat gaccacccct |
| 5221 | tcaccaacca ccactcccag ccctccaaca accaccatga ccacccttcc accaaccacc |
| 5281 | acttccagcc ctctaacaac tactcctcta cctccatcaa taactcctcc tacattttca |
| 5341 | ccattctcaa cgacaacccc tactacccca tgcgtgcctc tctgcaattg gactggctgg |
| 5401 | ctggattctg gaaaacccaa ctttcacaaa ccaggtggag acacagaatt gattggagac |
| 5461 | gtctgtggac caggctgggc agctaacatc tcttgcagag ccaccatgta tcctgatgtt |
| 5521 | cccattggac agcttggaca aacagtggtg tgtgatgtct ctgtggggct gatatgcaaa |
| 5581 | aatgaagacc aaaagccagg tggggtcatc cctatggcct tctgcctcaa ctacgagatc |
| 5641 | aacgttcagt gctgtgagtg tgtcacccaa cccaccacca tgacaaccac caccacagag |
| 5701 | aacccaactc cgccaaccac gacacccatc accaccacca ctacggtgac cccaacccca |
| 5761 | acacccaccg gcacacagac cccaaccacg acacccatca ccaccaccac tacggtgacc |
| 5821 | ccaaccccaa cacccaccgg cacacagacc ccaaccacga cacccatcac caccaccact |
| 5881 | acggtgaccc caaccccaac acccaccggc acacagaccc caaccacgac acccatcacc |
| 5941 | accaccacta cggtgacccc aaccccaaca cccaccggca cacagacccc aaccacgaca |
| 6001 | cccatcacca ccaccactac ggtgacccca accccaacac ccaccggcac acagacccca |
| 6061 | accacgacac ccatcaccac caccactacg gtgaccccaa ccccaacacc caccggcaca |
| 6121 | cagaccccaa ccacgacacc catcaccacc accactacgg tgaccccaac cccaacaccc |
| 6181 | accggcacac agaccccaac cacgacaccc atcaccacca ccactacggt gaccccaacc |
| 6241 | ccaacaccca ccggcacaca gaccccaacc acgacaccca tcaccaccac cactacggtg |
| 6301 | accccaaccc caacacccac cggcacacag accccaacca cgacacccat caccaccacc |
| 6361 | actacggtga ccccaacccc aacacccacc ggcacacaga ccccaaccac gacacccatc |
| 6421 | accaccacca ctacggtgac cccaacccca acacccaccg gcacacagac cccaaccacg |
| 6481 | acacccatca ccaccaccac tacggtgacc ccaaccccaa cacccaccgg cacacagacc |
| 6541 | ccaaccacga cacccatcac caccaccact acggtgaccc caaccccaac acccaccggc |
| 6601 | acacagaccc caaccacgac acccatcacc accaccacta cggtgacccc aaccccaaca |
| 6661 | cccaccggca cacagacccc aaccacgaca cccatcacca ccaccactac ggtgacccca |
| 6721 | accccaacac ccaccggcac acagacccca accacgacac ccatcaccac caccactacg |
| 6781 | gtgaccccaa ccccaacacc caccggcaca cagaccccaa ccacgacacc catcaccacc |
| 6841 | accactacgg tgaccccaac cccaacaccc accggcacac agaccccaac cacgacaccc |
| 6901 | atcaccacca ccactacggt gaccccaacc ccaacaccca ccggcacaca gaccccaacc |
| 6961 | acgacaccca tcaccaccac cactacggtg accccaaccc caacacccac cggcacacag |
| 7021 | accccaacca cgacacccat caccaccacc actacggtga ccccaacccc aacacccacc |
| 7081 | ggcacacaga ccccaaccac gacacccatc accaccacca ctacggtgac cccaacccca |
| 7141 | acacccaccg gcacacagac cccaaccacg acacccatca ccaccaccac tacggtgacc |
| 7201 | ccaaccccaa cacccaccgg cacacagacc ccaaccacga cacccatcac caccaccact |
| 7261 | acggtgaccc caaccccaac acccaccggc acacagaccc caaccacgac acccatcacc |
| 7321 | accaccacta cggtgacccc aaccccaaca cccaccggca cacagacccc aaccacgaca |
| 7381 | cccatcacca ccaccactac ggtgacccca accccaacac ccaccggcac acagacccca |
| 7441 | accacgacac ccatcaccac caccactacg gtgaccccaa ccccaacacc caccggcaca |
| 7501 | cagaccccaa ccacgacacc catcaccacc accactacgg tgaccccaac cccaacaccc |
| 7561 | accggcacac agaccccaac cacgacaccc atcaccacca ccactacggt gaccccaacc |
| 7621 | ccaacaccca ccggcacaca gaccccaacc acgacaccca tcaccaccac cactacggtg |
| 7681 | accccaaccc caacacccac cggcacacag accccaacca cgacacccat caccaccacc |
| 7741 | actacggtga ccccaacccc aacacccacc ggcacacaga ccccaaccac gacacccatc |
| 7801 | accaccacca ctacggtgac cccaacccca acacccaccg gcacacagac cccaaccacg |
| 7861 | acacccatca ccaccaccac tacggtgacc ccaaccccaa cacccaccgg cacacagacc |
| 7921 | ccaaccacga cacccatcac caccaccact acggtgaccc caaccccaac acccaccggc |
| 7981 | acacagaccc caaccacgac acccatcacc accaccacta cggtgacccc aaccccaaca |
| 8041 | cccaccggca cacagacccc aaccacgaca cccatcacca ccaccactac ggtgacccca |
| 8101 | accccaacac ccaccggcac acagacccca accacgacac ccatcaccac caccactacg |
| 8161 | gtgaccccaa ccccaacacc caccggcaca cagaccccaa ccacgacacc catcaccacc |
| 8221 | accactacgg tgaccccaac cccaacaccc accggcacac agaccccaac cacgacaccc |
| 8281 | atcaccacca ccactacggt gaccccaacc ccaacaccca ccggcacaca gaccccaacc |
| 8341 | acgacaccca tcaccaccac cactacggtg accccaaccc caacacccac cggcacacag |
| 8401 | accccaacca cgacacccat caccaccacc actacggtga ccccaacccc aacacccacc |
| 8461 | ggcacacaga ccccaaccac gacacccatc accaccacca ctacggtgac cccaacccca |
| 8521 | acacccaccg gcacacagac cccaaccacg acacccatca ccaccaccac tacggtgacc |
| 8581 | ccaaccccaa cacccaccgg cacacagacc ccaaccacga cacccatcac caccaccact |
| 8641 | acggtgaccc caaccccaac acccaccggc acacagaccc caaccacgac acccatcacc |
| 8701 | accaccacta cggtgacccc aaccccaaca cccaccggca cacagacccc aaccacgaca |
| 8761 | cccatcacca ccaccactac ggtgacccca accccaacac ccaccggcac acagacccca |
| 8821 | accacgacac ccatcaccac caccactacg gtgaccccaa ccccaacacc caccggcaca |
| 8881 | cagaccccaa ccacgacacc catcaccacc accactacgg tgaccccaac cccaacaccc |
| 8941 | accggcacac agaccccaac cacgacaccc atcaccacca ccactacggt gaccccaacc |
| 9001 | ccaacaccca ccggcacaca gaccccaacc acgacaccca tcaccaccac cactacggtg |
| 9061 | accccaaccc caacacccac cggcacacag accccaacca cgacacccat caccaccacc |
| 9121 | actacggtga ccccaacccc aacacccacc ggcacacaga ccccaaccac gacacccatc |
| 9181 | accaccacca ctacggtgac cccaacccca acacccaccg gcacacagac cccaaccacg |
| 9241 | acacccatca ccaccaccac tacggtgacc ccaaccccaa cacccaccgg cacacagacc |
| 9301 | ccaaccacga cacccatcac caccaccact acggtgaccc caaccccaac acccaccggc |
| 9361 | acacagaccc caaccacgac acccatcacc accaccacta cggtgacccc aaccccaaca |
| 9421 | cccaccggca cacagacccc aaccacgaca cccatcacca ccaccactac ggtgacccca |
| 9481 | accccaacac ccaccggcac acagacccca accacgacac ccatcaccac caccactacg |
| 9541 | gtgaccccaa ccccaacacc caccggcaca cagaccccaa ccacgacacc catcaccacc |
| 9601 | accactacgg tgaccccaac cccaacaccc accggcacac agaccccaac cacgacaccc |
| 9661 | atcaccacca ccactacggt gaccccaacc ccaacaccca ccggcacaca gaccccaacc |
| 9721 | acgacaccca tcaccaccac cactacggtg accccaaccc caacacccac cggcacacag |
| 9781 | accccaacca cgacacccat caccaccacc actacggtga ccccaacccc aacacccacc |
| 9841 | ggcacacaga ccccaaccac gacacccatc accaccacca ctacggtgac cccaacccca |
| 9901 | acacccaccg gcacacagac cccaaccacg acacccatca ccaccaccac tacggtgacc |
| 9961 | ccaaccccaa cacccaccgg cacacagacc ccaaccacga cacccatcac caccaccact |
| 10021 | acggtgaccc caaccccaac acccaccggc acacagaccc caaccacgac acccatcacc |
| 10081 | accaccacta cggtgacccc aaccccaaca cccaccggca cacagacccc aaccacgaca |
| 10141 | cccatcacca ccaccactac ggtgacccca accccaacac ccaccggcac acagacccca |
| 10201 | accacgacac ccatcaccac caccactacg gtgaccccaa ccccaacacc caccggcaca |
| 10261 | cagaccccaa ccacgacacc catcaccacc accactacgg tgaccccaac cccaacaccc |
| 10321 | accggcacac agaccccaac cacgacaccc atcaccacca ccactacggt gaccccaacc |
| 10381 | ccaacaccca ccggcacaca gaccccaacc acgacaccca tcaccaccac cactacggtg |
| 10441 | accccaaccc caacacccac cggcacacag accccaacca cgacacccat caccaccacc |
| 10501 | actacggtga ccccaacccc aacacccacc ggcacacaga ccccaaccac gacacccatc |
| 10561 | accaccacca ctacggtgac cccaacccca acacccaccg gcacacagac cccaaccacg |
| 10621 | acacccatca ccaccaccac tacggtgacc ccaaccccaa cacccaccgg cacacagacc |
| 10681 | ccaaccacga cacccatcac caccaccact acggtgaccc caaccccaac acccaccggc |
| 10741 | acacagaccc caaccacgac acccatcacc accaccacta cggtgacccc aaccccaaca |
| 10801 | cccaccggca cacagacccc aaccacgaca cccatcacca ccaccactac ggtgacccca |
| 10861 | accccaacac ccaccggcac acagacccca accacgacac ccatcaccac caccactacg |
| 10921 | gtgaccccaa ccccaacacc caccggcaca cagaccccaa ccacgacacc catcaccacc |
| 10981 | accactacgg tgaccccaac cccaacaccc accggcacac agaccccaac cacgacaccc |
| 11041 | atcaccacca ccactacggt gaccccaacc ccaacaccca ccggcacaca gaccccaacc |
| 11101 | acgacaccca tcaccaccac cactacggtg accccaaccc caacacccac cggcacacag |
| 11161 | accccaacca cgacacccat caccaccacc actacggtga ccccaacccc aacacccacc |
| 11221 | ggcacacaga ccccaaccac gacacccatc accaccacca ctacggtgac cccaacccca |
| 11281 | acacccaccg gcacacagac cccaaccacg acacccatca ccaccaccac tacggtgacc |
| 11341 | ccaaccccaa cacccaccgg cacacagacc ccaaccacga cacccatcac caccaccact |
| 11401 | acggtgaccc caaccccaac acccaccggc acacagaccc caaccacgac acccatcacc |
| 11461 | accaccacta cggtgacccc aaccccaaca cccaccggca cacagacccc aaccacgaca |
| 11521 | cccatcacca ccaccactac ggtgacccca accccaacac ccaccggcac acagacccca |
| 11581 | accacgacac ccatcaccac caccactacg gtgaccccaa ccccaacacc caccggcaca |
| 11641 | cagaccccaa ccacgacacc catcaccacc accactacgg tgaccccaac cccaacaccc |
| 11701 | accggcacac agaccccaac cacgacaccc atcaccacca ccactacggt gaccccaacc |
| 11761 | ccaacaccca ccggcacaca gaccccaacc acgacaccca tcaccaccac cactacggtg |
| 11821 | accccaaccc caacacccac cggcacacag accccaacca cgacacccat caccaccacc |
| 11881 | actacggtga ccccaacccc aacacccacc ggcacacaga ccccaaccac gacacccatc |
| 11941 | accaccacca ctacggtgac cccaacccca acacccaccg gcacacagac cccaaccacg |
| 12001 | acacccatca ccaccaccac tacggtgacc ccaaccccaa cacccaccgg cacacagacc |
| 12061 | ccaaccacga cacccatcac caccaccact acggtgaccc caaccccaac acccaccggc |
| 12121 | acacagaccc caaccacgac acccatcacc accaccacta cggtgacccc aaccccaaca |
| 12181 | cccaccggca cacagacccc aaccacgaca cccatcacca ccaccactac ggtgacccca |
| 12241 | accccaacac ccaccggcac acagacccca accacgacac ccatcaccac caccactacg |
| 12301 | gtgaccccaa ccccaacacc caccggcaca cagaccccaa ccacgacacc catcaccacc |
| 12361 | accactacgg tgaccccaac cccaacaccc accggcacac agaccccaac cacgacaccc |
| 12421 | atcaccacca ccactacggt gaccccaacc ccaacaccca ccggcacaca gaccccaacc |
| 12481 | acgacaccca tcaccaccac cactacggtg accccaaccc caacacccac cggcacacag |
| 12541 | accccaacca cgacacccat caccaccacc actacggtga ccccaacccc aacacccacc |
| 12601 | ggcacacaga ccgggccccc cacccacaca agcacagcac cgattgctga gttgaccaca |
| 12661 | tccaatcctc cgcctgagtc ctcaacccct cagacctctc ggtccacctc ttcccctctc |
| 12721 | acggagtcaa ccacccttct gagtacccta ccacctgcca ttgagatgac cagcacggcc |
| 12781 | ccaccctcca cacccacggc acccacgacc acgagcggag gccacacact gtctccaccg |
| 12841 | cccagcacca ccacgtcccc tccaggcacc cccactcgcg gtaccacgac tgggtcatct |
| 12901 | tcagccccca cccccagcac tgtgcagacg accaccacca gtgcctggac ccccacgccg |
| 12961 | accccactct ccacacccag catcatcagg accacaggcc tgaggcccta cccttcctct |
| 13021 | gtgcttatct gctgtgtcct gaacgacacc tactacgcac caggtgagga ggtgtacaac |
| 13081 | ggcacatacg gagacacctg ttatttcgtc aactgctcac tgagctgtac gttggagttc |
| 13141 | tataactggt cctgcccatc cacgccctcc ccaacaccca cgccctccaa gtcgacgccc |
| 13201 | acgccttcca agccatcgtc cacgccctcc aagccgacgc ccggcaccaa gccccccgag |
| 13261 | tgcccagact ttgatcctcc cagacaggag aacgagactt ggtggctgtg cgactgcttc |
| 13321 | atggccacgt gcaagtacaa caacacggtg gagatcgtga aggtggagtg tgagccgccg |
| 13381 | cccatgccca cctgctccaa cggcctccaa cccgtgcgcg tcgaggaccc cgacggctgc |
| 13441 | tgctggcact gggagtgcga ctgctactgc acgggctggg gcgacccgca ctatgtcacc |
| 13501 | ttcgacggac tctactacag ctaccagggc aactgcacct acgtgctggt ggaggagatc |
| 13561 | agcccctccg tggacaactt cggagtttac atcgacaact accactgcga tcccaacgac |
| 13621 | aaggtgtcct gcccccgcac cctcatcgtg cgccacgaga cccaggaggt gctgatcaag |
| 13681 | accgtgcata tgatgcccat gcaggtgcag gtgcaggtga acaggcaggc ggtggcactg |
| 13741 | ccctacaaga agtacgggct ggaggtgtac cagtctggca tcaactacgt ggtggacatc |
| 13801 | cccgagctgg gtgtcctcgt ctcctacaat ggcctgtcct tctccgtcag gctgccctac |
| 13861 | caccggtttg gcaacaacac caagggccag tgtggcacct gcaccaacac cacctccgac |
| 13921 | gactgcattc tgcccagcgg ggagatcgtc tccaactgtg aggctgcggc tgaccagtgg |
| 13981 | ctggtgaacg acccctccaa gccacactgc ccccacagca gctccacgac caagcgcccg |
| 14041 | gccgtcactg tgcccggggg cggtaaaacg accccacaca aggactgcac cccatctccc |
| 14101 | ctctgccagc tcatcaagga cagcctgttt gcccagtgcc acgcactggt gcccccgcag |
| 14161 | cactactacg atgcctgcgt gttcgacagc tgcttcatgc cgggctcgag cctggagtgc |
| 14221 | gccagtctgc aggcctacgc agccctctgt gcccagcaga acatctgcct cgactggcgg |
| 14281 | aaccacacgc atggggcctg cttggtggag tgcccatctc acagggagta ccaggcctgt |
| 14341 | ggccctgcag aagagcccac gtgcaaatcc agctcctccc agcagaacaa cacagtcctg |
| 14401 | gtggaaggct gcttctgtcc tgagggcacc atgaactacg ctcctggctt tgatgtctgc |
| 14461 | gtgaagacct gcggctgtgt gggacctgac aatgtgccca gagagtttgg ggagcacttc |
| 14521 | gagttcgact gcaagaactg tgtctgcctg gagggtggaa gtggcatcat ctgccaaccc |
| 14581 | aagaggtgca gccagaagcc cgttacccac tgcgtggaag acggcaccta cctcgccacg |
| 14641 | gaggtcaacc ctgccgacac ctgctgcaac attaccgtct gcaagtgcaa caccagcctg |
| 14701 | tgcaaagaga agccctccgt gtgcccgctg ggattcgaag tgaagagcaa gatggtgcct |
| 14761 | ggaaggtgct gtcctttcta ctggtgtgag tccaaggggg tgtgtgttca cgggaatgct |
| 14821 | gagtaccagc ccggttctcc agtttattcc tccaagtgcc aggactgcgt gtgcacggac |
| 14881 | aaggtggaca acaacaccct gctcaacgtc atcgcctgca cccacgtgcc ctgcaacacc |
| 14941 | tcctgcagcc ctggcttcga actcatggag gcccccgggg agtgctgtaa gaagtgtgaa |
| 15001 | cagacgcact gtatcatcaa acggcccgac aaccagcacg tcatcctgaa gcccggggac |
| 15061 | ttcaagagcg acccgaagaa caactgcaca ttcttcagct gcgtgaagat ccacaaccag |
| 15121 | ctcatctcgt ccgtctccaa catcacctgc cccaactttg atgccagcat ttgcatcccg |
| 15181 | ggctccatca cattcatgcc caatggatgc tgcaagacct gcacccctcg caatgagacc |
| 15241 | agggtgccct gctccaccgt ccccgtcacc acggaggttt cgtacgccgg ctgcaccaag |
| 15301 | accgtcctca tgaatcattg ctccgggtcc tgcgggacat ttgtcatgta ctcggccaag |
| 15361 | gcccaggccc tggaccacag ctgctcctgc tgcaaagagg agaaaaccag ccagcgtgag |
| 15421 | gtggtcctga gctgccccaa tggcggctcg ctgacacaca cctacaccca catcgagagc |
| 15481 | tgccagtgcc aggacaccgt ctgcgggctc cccaccggca cctcccgccg ggcccggcgc |
| 15541 | tcccctaggc atctggggag cgggtgagcg gggtgggcac agcccccttc actgccctcg |
| 15601 | acagctttac ctcccccgga ccctctgagc ctcctaagct cggcttcctc tcttcagata |
| 15661 | tttattgtct gagtctttgt tcagtccttg ctttccaata ataaactcag ggggacatgc |

By “NKX2-5 polypeptide” or “human NKX2-5 (hNKX2-5) polypeptide” is meant a protein or fragment thereof having at least 85%, at least 90%, at least 95%, at least 98%, or at least 99% amino acid sequence identity to the sequence provided at NCBI Accession No. NP_004378.1 (isoform 1), NP_001159647.1 (isoform 2), or NP_001159648.1 (isoform 3) and having transcription factor activity. The amino acid sequence provided at NCBI Accession No. NP_004378.1 is shown below:

(SEQ ID NO: 31)

| | |
|---|---|
| 1 | MFPSPALTPT PFSVKDILNL EQQQRSLAAA |
| | GELSARLEAT LAPSSCMLAA FKPEAYAGPE |
| 61 | AAAPGLPELR AELGRAPSPA KCASAFPAAP |
| | AFYPRAYSDP DPAKDPRAEK KELCALQKAV |
| 121 | ELEKTEADNA ERPRARRRRK PRVLFSQAQV |
| | YELERRFKQQ RYLSAPERDQ LASVLKLTST |
| 181 | QVKIWFQNRR YKCKRQRQDQ TLELVGLPPP |
| | PPPPARRIAV PVLVRDGKPC LGDSAPYAPA |
| 241 | YGVGLNPYGY NAYPAYPGYG GAACSPGYSC |
| | TAAYPAGPSP AQPATAAANN NEVNEGVGDL |
| 301 | NAVQSPGIPQ SNSGVSTLHG IRAW |

By “NKX2-5 polynucleotide” is meant a polynucleotide encoding a NKX2-5 polypeptide or fragment thereof. An exemplary NKX2-5 polynucleotide sequence is provided at NCBI Ref: NM_004387.3. The sequence provided at NCBI Ref: NM_004387.3 is reproduced below:

(SEQ ID NO: 32)

| | |
|---|---|
| 1 | gctcctgtca tcgaggcccc tggcccaatg |
| | gcaggctgag tccccctcct ctggcctggt |
| 61 | cccgcctctc ctgccccttg tgctcagcgc |
| | tacctgctgc ccggacacat ccagagctgg |
| 121 | ccgacgggtg cgcgggcggg cggcggcacc |
| | atgcagggaa gctgccaggg gccgtgggca |
| 181 | gcgccgcttt ctgccgccca cctggcgctg |
| | tgagactggc gctgccacca tgttccccag |
| 241 | ccctgctctc acgcccacgc ccttctcagt |
| | caaagacatc ctaaacctgg aacagcagca |
| 301 | gcgcagcctg gctgccgccg gagagctctc |
| | tgcccgcctg gaggcgaccc tggcgccctc |
| 361 | ctcctgcatg ctggccgcct tcaagccaga |
| | ggcctacgct gggcccgagg cggctgcgcc |
| 421 | gggcctccca gagctgcgcg cagagctggg |
| | ccgcgcgcct tcaccggcca agtgtgcgtc |
| 481 | tgcctttccc gccgcccccg ccttctatcc |
| | acgtgcctac agcgaccccg acccagccaa |
| 541 | ggaccctaga gccgaaaaga aagagctgtg |
| | cgcgctgcag aaggcggtgg agctggagaa |
| 601 | gacagaggcg gacaacgcgg agcggccccg |
| | ggcgcgacgg cggaggaagc cgcgcgtgct |
| 661 | cttctcgcag gcgcaggtct atgagctgga |
| | gcggcgcttc aagcagcagc ggtacctgtc |
| 721 | ggcccccgaa cgcgaccagc tggccagcgt |
| | gctgaaactc acgtccacgc aggtcaagat |
| 781 | ctggttccag aaccggcgct acaagtgcaa |
| | gcggcagcgg caggaccaga ctctggagct |
| 841 | ggtggggctg cccccgccgc cgccgccgcc |
| | tgcccgcagg atcgcggtgc cagtgctggt |
| 901 | gcgcgatggc aagccatgcc taggggactc |
| | ggcgccctac gcgcctgcct acggcgtggg |
| 961 | cctcaatccc tacggttata acgcctaccc |
| | cgcctatccg ggttacggcg gcgcggcctg |
| 1021 | cagccctggc tacagctgca ctgccgctta |
| | ccccgccggg ccttccccag cgcagccggc |
| 1081 | cactgccgcc gccaacaaca acttcgtgaa |
| | cttcggcgtc ggggacttga atgcggttca |
| 1141 | gagccccggg attccgcaga gcaactcggg |
| | agtgtccacg ctgcatggta tccgagcctg |
| 1201 | gtagggaagg gacccgcgtg gcgcgaccct |
| | gaccgatccc acctcaacag ctccctgact |
| 1261 | ctcgggggga gaaggggctc ccaacatgac |
| | cctgagtccc ctggattttg cattcactcc |
| 1321 | tgcggagacc taggaacttt ttctgtccca |
| | cgcgcgtttg ttcttgcgca cgggagagtt |
| 1381 | tgtggcggcg attatgcagc gtgcaatgag |
| | tgatcctgca gcctggtgtc ttagctgtcc |
| 1441 | ccccaggagt gccctccgag agtccatggg |
| | cacccccggt tggaactggg actgagctcg |
| 1501 | ggcacgcagg gcctgagatc tggccgccca |
| | ttccgcgagc cagggccggg cgcccgggcc |
| 1561 | tttgctatct cgccgtcgcc cgcccacgca |
| | cccacccgta tttatgtttt tacctattgc |
| 1621 | tgtaagaaat gacgatcccc ttcccattaa |
| | agagagtgcg ttgaccccg |

By “NEUROD1 polypeptide” is meant a protein or fragment thereof having at least 85%, at least 90%, at least 95%, at least 98%, or at least 99% amino acid sequence identity to the sequence provided at NCBI Accession No. NP_002491.2 and having transcription factor activity. The amino acid sequence provided at NCBI Accession No. NP_002491.2 is shown below:

(SEQ ID NO: 33)

| | |
|---|---|
| 1 | MTKSYSESGL MGEPQPQGPP SWTDECLSSQ |
| | DEEHEADKKE DDLEAMNAEE DSLRNGGEEE |
| 61 | DEDEDLEEEE EEEEEDDDQK PKRRGPKKKK |
| | MTKARLERFK LRRMKANARE RNRMHGLNAA |
| 121 | LDNLRKVVPC YSKTQKLSKI ETLRLAKNYI |
| | WALSEILRSG KSPDLVSFVQ TLCKGLSQPT |
| 181 | TNLVAGCLQL NPRTFLPEQN QDMPPHLPTA |
| | SASFPVHPYS YQSPGLPSPP YGTMDSSHVF |
| 241 | HVKPPPHAYS AALEPFFESP LTDCTSPSFD |
| | GPLSPPLSIN GNFSFKHEPS AEFEKNYAFT |
| 301 | MHYPAATLAG AQSHGSIFSG TAAPRCEIPI |
| | DNIMSFDSHS HHERVMSAQL NAIFHD |

By “NEUROD1 polynucleotide” is meant a polynucleotide encoding a NEUROD1 polypeptide or fragment thereof. An exemplary NEUROD1 polynucleotide sequence is provided at NCBI Ref: NM_002500.4. The sequence provided at NCBI Ref: NM_002500.4 is reproduced below:

(SEQ ID NO: 34)

| | |
|---|---|
| 1 | ggggaggagg ggagaacggg gagcgcacag |
| | cctggacgcg tgcgcaggcg tcaggcgcat |
| 61 | agacctgcta gcccctcagc tagcggcccc |
| | gcccgcgctt agcatcacta actgggctat |
| 121 | ataacctgag cgcccgcgcg gccacgacac |
| | gaggaattcg cccacgcagg aggcgcggcg |
| 181 | tccggaggcc ccagggttat gagactatca |
| | ctgctcagga cctactaaca acaaaggaaa |
| 241 | tcgaaacatg accaaatcgt acagcgagag |
| | tgggctgatg ggcgagcctc agccccaagg |
| 301 | tcctccaagc tggacagacg agtgtctcag |
| | ttctcaggac gaggagcacg aggcagacaa |
| 361 | gaaggaggac gacctcgaag ccatgaacgc |
| | agaggaggac tcactgagga acgggggaga |
| 421 | ggaggaggac gaagatgagg acctggaaga |
| | ggaggaagaa gaggaagagg aggatgacga |
| 481 | tcaaaagccc aagagacgcg gccccaaaaa |
| | gaagaagatg actaaggctc gcctggagcg |
| 541 | ttttaaattg agacgcatga aggctaacgc |
| | ccgggagcgg aaccgcatgc acggactgaa |
| 601 | cgcggcgcta gacaacctgc gcaaggtggt |
| | gccttgctat tctaagacgc agaagctgtc |
| 661 | caaaatcgag actctgcgct tggccaagaa |
| | ctacatctgg gctctgtcgg agatcctgcg |
| 721 | ctcaggcaaa agcccagacc tggtctcctt |
| | cgttcagacg ctttgcaagg gcttatccca |
| 781 | acccaccacc aacctggttg cgggctgcct |
| | gcaactcaat cctcggactt ttctgcctga |
| 841 | gcagaaccag gacatgcccc cccacctgcc |
| | gacggccagc gcttccttcc ctgtacaccc |
| 901 | ctactcctac cagtcgcctg ggctgcccag |
| | tccgccttac ggtaccatgg acagctccca |
| 961 | tgtcttccac gttaagcctc cgccgcacgc |
| | ctacagcgca gcgctggagc ccttctttga |
| 1021 | aagccctctg actgattgca ccagcccttc |
| | ctttgatgga cccctcagcc cgccgctcag |
| 1081 | catcaatggc aacttctctt tcaaacacga |
| | accgtccgcc gagtttgaga aaaattatgc |
| 1141 | ctttaccatg cactatcctg cagcgacact |
| | ggcaggggcc caaagccacg gatcaatctt |
| 1201 | ctcaggcacc gctgcccctc gctgcgagat |
| | ccccatagac aatattatgt ccttcgatag |
| 1261 | ccattcacat catgagcgag tcatgagtgc |
| | ccagctcaat gccatatttc atgattagag |
| 1321 | gcacgccagt ttcaccattt ccgggaaacg |
| | aacccactgt gcttacagtg actgtcgtgt |
| 1381 | ttacaaaagg cagccctttg ggtactactg |
| | ctgcaaagtg caaatactcc aagcttcaag |
| 1441 | tgatatatgt atttattgtc attactgcct |
| | ttggaagaaa caggggatca aagttcctgt |
| 1501 | tcaccttatg tattattttc tatagctctt |
| | ctatttaaaa aataaaaaaa tacagtaaag |
| 1561 | tttaaaaaat acaccacgaa tttggtgtgg |
| | ctgtattcag atcgtattaa ttatctgatc |
| 1621 | gggataacaa aatcacaagc aataattagg |
| | atctatgcaa tttttaaact agtaatgggc |
| 1681 | caattaaaat atatataaat atatattttt |
| | caaccagcat tttactactt gttacctttc |
| 1741 | ccatgctgaa ttattttgtt gtgattttgt |
| | acagaatttt taatgacttt ttataatgtg |
| 1801 | gatttcctat tttaaaacca tgcagcttca |
| | tcaattttta tacatatcag aaaagtagaa |
| 1861 | ttatatctaa tttatacaaa ataatttaac |
| | taatttaaac cagcagaaaa gtgcttagaa |
| 1921 | agttattgtg ttgccttagc acttctttcc |
| | tctccaattg taaaaaaaaa aaaaaaaaaa |
| 1981 | aaaaaaaaaa aaaaattgca caatttgagc |
| | aattcatttc actttaaagt ctttccgtct |
| 2041 | ccctaaaata aaaaccagaa tcataatttt |
| | caagagaaga aaaaattaag agatacattc |
| 2101 | cctatcaaaa catatcaatt caacacatta |
| | cttgcacaag cttgtatata catattataa |
| 2161 | ataaatgcca acataccctt ctttaaatca |
| | aaagctgctt gactatcaca tacaatttgc |
| 2221 | actgttactt tttagtcttt tactcctttg |
| | cattccatga ttttacagag aatctgaagc |
| 2281 | tattgatgtt tccagaaaat ataaatgcat |
| | gattttatac atagtcacaa aaatggtggt |
| 2341 | ttgtcatata ttcatgtaat aaatctgagc |
| | ctaaatctaa tcaggttgtt aatgttggga |
| 2401 | tttatatcta tagtagtcaa ttagtacagt |
| | agcttaaata aattcaaacc atttaattca |
| 2461 | taattagaac aatagctatt gcatgtaaaa |
| | tgcagtccag aataagtgct gtttgagatg |
| 2521 | tgatgctggt accactggaa tcgatctgta |
| | ctgtaatttt gtttgtaatc ctgtatatta |
| 2581 | tggtgtaatg cacaatttag aaaacattca |
| | tccagttgca ataaaatagt attgaaagtg |
| 2641 | agagcaattg ttgcatttct tcttaaaggg |
| | attctgtttt tatttttggg gaaagtagtt |
| 2701 | gcttttttgc tgagttaaaa aatactaaac |
| | actatatgta gaataaaaga aaagaaaaaa |
| 2761 | gtttaccttg gcatatgctc ttgtctgttt |
| | atcttgcaca gggagtcacc agttctatgt |
| 2821 | agataatgaa aagacctaac tgatatttca |
| | ttatttggaa tatgggactg gacggcagta |
| 2881 | caaacagtgt gtttttttct ttgttttaag |
| | tggcttagcc tttaggtttt ttatttccat |
| 2941 | ttttaaaaat gattgttaca tgttttcttc |
| | tatttctttt tttaaaaggt ggattttaat |
| 3001 | aa |

By “NKX6-1 polypeptide” is meant a protein or fragment thereof having at least 85%, at least 90%, at least 95%, at least 98%, or at least 99% amino acid sequence identity to the sequence provided at NCBI Accession No. NP_006159.2 and having transcription factor activity. The amino acid sequence provided at NCBI Accession No. NP_006159.2 is shown below:

(SEQ ID NO: 35)

| | |
|---|---|
| 1 | MLAVGAMEGT RQSAFLLSSP PLAALHSMAE |
| | MKTPLYPAAY PPLPAGPPSS SSSSSSSSSP |
| 61 | SPPLGTHNPG GLKPPATGGL SSLGSPPQQL |
| | SAATPHGIND ILSRPSMPVA SGAALPSASP |
| 121 | SGSSSSSSSS ASASSASAAA AAAAAAAAAA |
| | SSPAGLLAGL PRFSSLSPPP PPPGLYFSPS |
| 181 | AAAVAAVGRY PKPLAELPGR TPIFWPGVMQ |
| | SPPWRDARLA CTPHQGSILL DKDGKRKHTR |
| 241 | PTFSGQQIFA LEKTFEQTKY LAGPERARLA |
| | YSLGMTESQV KVWFQNRRTK WRKKHAAEMA |
| 301 | TAKKKQDSET ERLKGASENE EEDDDYNKPL |
| | DPNSDDEKIT QLLKKHKSSS GGGGGLLLHA |
| 361 | SEPESSS |

By “NKX6-1 polynucleotide” is meant a polynucleotide encoding a NKX6-1 polypeptide or fragment thereof. An exemplary NKX6-1 polynucleotide sequence is provided at NCBI Ref: NM_006168.2. The sequence provided at NCBI Ref: NM_006168.2 is reproduced below:

(SEQ ID NO: 36)

| | |
|---|---|
| 1 | cgtgggatgt tagcggtggg ggcaatggag |
| | ggcacccggc agagcgcatt cctgctcagc |
| 61 | agccctcccc tggccgccct gcacagcatg |
| | gccgagatga agaccccgct gtaccctgcc |
| 121 | gcgtatcccc cgctgcctgc cggccccccc |
| | tcctcctcgt cctcgtcgtc gtcctcctcg |
| 181 | tcgccctccc cgcctctggg cacccacaac |
| | ccaggcggcc tgaagccccc ggccacgggg |
| 241 | gggctctcat ccctcggcag ccccccgcag |
| | cagctctcgg ccgccacccc acacggcatc |
| 301 | aacgatatcc tgagccggcc ctccatgccc |
| | gtggcctcgg gggccgccct gccctccgcc |
| 361 | tcgccctccg gttcctcctc ctcctcttcc |
| | tcgtccgcct ctgcctcctc cgcctctgcc |
| 421 | gccgccgcgg ctgctgccgc ggccgcagcc |
| | gccgcctcat ccccggcggg gctgctggcc |
| 481 | ggactgccac gctttagcag cctgagcccg |
| | ccgccgccgc cgcccgggct ctacttcagc |
| 541 | cccagcgccg cggccgtggc cgccgtgggc |
| | cggtacccca agccgctggc tgagctgcct |
| 601 | ggccggacgc ccatcttctg gcccggagtg |
| | atgcagagcc cgccctggag ggacgcacgc |
| 661 | ctggcctgta cccctcatca aggatccatt |
| | ttgttggaca aagacgggaa gagaaaacac |
| 721 | acgagaccca ctttttccgg acagcagatc |
| | ttcgccctgg agaagacttt cgaacaaaca |
| 781 | aaatacttgg cggggcccga gagggctcgt |
| | ttggcctatt cgttggggat gacagagagt |
| 841 | caggtcaagg tctggttcca gaaccgccgg |
| | accaagtgga ggaagaagca cgctgccgag |
| 901 | atggccacgg ccaagaagaa gcaggactcg |
| | gagacagagc gcctcaaggg ggcctcggag |
| 961 | aacgaggaag aggacgacga ctacaataag |
| | cctctggatc ccaactcgga cgacgagaaa |
| 1021 | atcacgcagc tgttgaagaa gcacaagtcc |
| | agcagcggcg gcggcggcgg cctcctactg |
| 1081 | cacgcgtccg agccggagag ctcatcctga |
| | acgccg |

By “NDUFA4 polypeptide” is meant a protein or fragment thereof having at least 85%, at least 90%, at least 95%, at least 98%, or at least 99% amino acid sequence identity to the sequence provided at NCBI Accession No. NP_002480.1 and having NADH dehydrogenase activity and oxidoreductase activity. The amino acid sequence provided at NCBI Accession No. NP_002480.1 is shown below:

(SEQ ID NO: 37)

| | |
|---|---|
| 1 | MAAELAMGAE LPSSPLAIEY VNDFDLMKFE |
| | VKKEPPEAER FCHRLPPGSL SSTPLSTPCS |
| 61 | SVPSSPSFCA PSPGTGGGGG AGGGGGSSQA |
| | GGAPGPPSGG PGAVGGTSGK PALEDLYWMS |
| 121 | GYQHHLNPEA LNLTPEDAVE ALIGSGHHGA |
| | HHGAHHPAAA AAYEAFRGPG FAGGGGADDM |
| 181 | GAGHHHGAHH AAHHHHAAHH HHHHHHHHGG |
| | AGHGGGAGHH VRLEERFSDD QLVSMSVREL |
| 241 | NRQLRGFSKE EVIRLKQKRR TLKNRGYAQS |
| | CRFKRVQQRH ILESEKCQLQ SQVEQLKLEV |
| 301 | GRLAKERDLY KEKYEKLAGR GGPGSAGGAG |
| | FPREPSPPQA GPGGAKGTAD FFL |

By “NDUFA4 polynucleotide” is meant a polynucleotide encoding a NDUFA4 polypeptide or fragment thereof. An exemplary NDUFA4 polynucleotide sequence is 40 provided at NCBI Ref: NM_002489.3. The sequence provided at NCBI Ref: NM_002489.3 is reproduced below:

(SEQ ID NO: 38)

| | |
|---|---|
| 1 | gggtccttca ggtaggaggt cctgggtgac |
| | tttggaagtc cgtagtgtct cattgcagat |
| 61 | aatttttagc ttagggcctg gtggctaggt |
| | cggttctctc ctttccagtc ggagacctct |
| 121 | gccgcaaaca tgctccgcca gatcatcggt |
| | caggccaaga agcatccgag cttgatcccc |
| 181 | ctctttgtat ttattggaac tggagctact |
| | ggagcaacac tgtatctctt gcgtctggca |
| 241 | ttgttcaatc cagatgtttg ttgggacaga |
| | aataacccag agccctggaa caaactgggt |
| 301 | cccaatgatc aatacaagtt ctactcagtg |
| | aatgtggatt acagcaagct gaagaaggaa |
| 361 | cgtccagatt tctaaatgaa atgtttcact |
| | ataacgctgc tttagaatga aggtcttcca |
| 421 | gaagccacat ccgcacaatt ttccacttaa |
| | ccaggaaata tttctcctct aaatgcatga |
| 481 | aatcatgttg gagatctcta ttgtaatctc |
| | tattggagat tacaatgatt aaatcaataa |
| 541 | ataactgaaa cttgatatgt gtcacttttt |
| | tatgctgaaa gtatgctctg aactttagag |
| 601 | tataggaaat taactattag aatttaaaga |
| | atttcttgaa tttctgtagt ttgaaaatac |
| 661 | gactttaagc tgctttagta aaacacttcc |
| | attttgtgta tagactgttg gtaacttcac |
| 721 | tagagcatac ataacaactg gaactggaaa |
| | ttatacaaaa gtaaattggg aaggatactc |
| 781 | cagcatctga cactggcaaa atggaaacct |
| | ttgagtttct cttactggct gttgaagtgt |
| 841 | gtgcagtttt taacaatggt ttttacttgg |
| | catctctttg ttgtgatttt caaggttata |
| 901 | agttgctttg gtcctaggat tgaagttgaa |
| | atctgagttt atcagtgcta accatggtgc |
| 961 | tagtagtcaa gagatcttga gaattttggc |
| | tgctgagtct tggtgcaggg tgcaggtttt |
| 1021 | cttttctttt ttcttttttt tttttttgag |
| | atagtctctg tcacccaggc tggagtgcag |
| 1081 | tggtacaaac atggatcact gcagcctcta |
| | cctcccgggc ttaagtgatc ctcctgcctc |
| 1141 | agcccctaag tagccgggac tacaggtatg |
| | tgccaccatg cccagttaat ttttgtaatt |
| 1201 | ttttttagag acagggtttt gccatgttgc |
| | ccaggctggt ctcaaactct tgagctcaag |
| 1261 | cgatccattc tcctcagcct cccagggtgc |
| | tgggattaca ggcgtgagcc attgcgctta |
| 1321 | gccatggtgc aggttttcaa aggccaggaa |
| | gtatattcat aattttaaga tggggaatat |
| 1381 | agcaagtttt cacataggtg tgtgtaagtc |
| | atcacatcat agaaacttga ggaattcagt |
| 1441 | gacattaatt ttggattttc atacgtaagt |
| | atacaattaa atgtttacag ggtagtagaa |
| 1501 | gcacatttta aatgtcagga actgaactaa |
| | gtatttgaat tacgtggatt atctcaaaaa |
| 1561 | ttttgaaatt gttaaacgag ttgaattact |
| | tgaattcatt ctgttagtca aatggtggat |
| 1621 | atttacaccc atgtagtttt gaatttagag |
| | tgtgtagagt gttttcagtt accagactcc |
| 1681 | atgcttttac ctcctatgtg tcaggtataa |
| | tttgaacctc taagaacagg gtttctcaac |
| 1741 | cttgccactg ttgactattt ctgaaagaca |
| | gtttggttta gcagaccatc ccatgcgctt |
| 1801 | tagcttgttt agtagctaac ttgggctctg |
| | ccactacaga caaaaagcac tctttccctc |
| 1861 | caattcccac aggctatgag aagaatggag |
| | acattaccaa atgtccattg gtgggcaaaa |
| 1921 | ttgcttcatt cctacctctg ttgagaatta |
| | ctctagatcc tttggcacaa attacctcaa |
| 1981 | agtttaaaat tgtgtaaaca aacagtgtgt |
| | catgtaattg aaaaacatta agcaactcca |
| 2041 | aataaatgct acattaag |

As used herein, “obtaining” as in “obtaining an agent” includes synthesizing, purchasing, procuring, deriving, or otherwise acquiring the agent.

By “organ” is meant a collection of cells that perform a biological function. In one embodiment, an organ includes, but is not limited to, bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. The biological function of an organ can be assayed using standard methods known to the skilled artisan.

By “organoid” is meant an in vitro generated body that mimics organ structure and function. “Organoid” and “mini organ” are used interchangeably herein. An “islet-like organoid,” “pancreatic islet organoid,” “pancreatic islet,” or “pancreatic organoid” is an in vitro generated cell cluster that mimics the structure and function of a pancreatic islet. Exemplary functions of a pancreatic islet include, without limitation, glucose-stimulated insulin secretion (GSIS), potassium chloride (KCl)-stimulated insulin secretion, GLP-1 stimulated insulin secretion, somatostatin secretion, or glucagon secretion. “Pancreatic islet organoid,” “islet-like organoid,” “pancreatic organoid” and “mini pancreatic islet” are used interchangeably herein. In an embodiment, a “pancreatic organoid” is an in vitro generated body that mimics structure and function of a pancreas. Exemplary functions of a pancreas include, without limitation, endocrine secretion of hormones, such as glucose and glucagon, that regulate glucose metabolism and blood glucose concentration, and exocrine secretion of digestive enzymes that help break down carbohydrates, proteins, and lipids. “Pancreatic organoid” and “mini pancreas” are also used interchangeably herein. In an embodiment, an organoid is a human islet-like organoid (“HILO”) as described herein. In an embodiment, a HILO is generated from induced pluripotent stem cells (iPSCs). In an embodiment, the HILO is functionally mature and contains endocrine-like cell types that, upon transplantation, effectively re-establish glucose homeostasis, e.g., in a diabetic mouse model (NOD-SCID mouse). In an embodiment, the HILO is a WNT4-treated HILO (wHILOs). In an embodiment, overexpression of the checkpoint protein PD-L1 in HILOs allowed the HILOs to evade an immune reaction or surveillance by T cells such that they were able to maintain glucose homeostasis in immune-competent diabetic mice (NOD-SCID mice) for a long time period, e.g., at least 50 days. In an embodiment, induction of endogenous PD-L1 expression in HILOs following multiple intermittent ex vivo exposures to interferon gamma (IFNγ) over a given time period, e.g., at least 24 hours, restricts T cell activation and graft rejection. In embodiments, multiple intermittent exposure of cells or HILOs and the cells therein to IFNγ encompasses exposure (e.g., in culture, such as liquid culture or 3D matrix culture) of cells or HILOs and the cells therein to an amount (e.g., low levels) of IFNγ for multiple times, e.g., 2, 3, 4, 5, 6, 7, 8, 9, 10, or more times, over a given time period, with periods of no IFNγ exposure in between. In an embodiment, HILOs that have undergone multiple intermittent exposure to IFNγ so as to express PD-L1 polypeptide as described herein may be referred to as immune evasive HILOs, wHILOs or wHILO^{ic} herein.

By “PD-L1 polypeptide” (also called CD274) is meant a protein or fragment thereof having at least 85%, at least 90%, at least 95%, at least 98%, or at least 99% amino acid sequence identity to the sequence provided at UniProt Accession No. Q9NZQ7-1 and having transcription factor activity. The amino acid sequence is provided at NCBI Accession No. NP_006184.2 is shown below:

(SEQ ID NO: 39)
MRIFAVFIFMTYWHLLNAFTVTVPKDLYVV
EYGSNMTIECKFPVEKQLDLAALIVYWEME
DKNIIQFVHGEEDLKVQHSSYRQRARLLKD
QLSLGNAALQITDVKLQDAGVYRCMISYGG
ADYKRITVKVNAPYNKINQRILVVDPVTSE
HELTCQAEGYPKAEVIWTSSDHQVLSGKTT
TTNSKREEKLFNVTSTLRINTTTNEIFYCT
FRRLDPEENHTAELVIPELPLAHPPNERTH
LVILGAILLCLGVALTFIFRLRKGRMMDVK
KCGIQDTNSKKQSDTHLEET

By “PD-L1 polynucleotide” is meant a polynucleotide encoding a PD-L1 polypeptide or fragment thereof. An exemplary PD-L1 polynucleotide sequence is provided at NCBI Accession No.: CCDS59118.1. The sequence provided at NCBI Accession No.: CCDS59118.1 is reproduced below:

### Nucleotide Sequence (531 nt):

(SEQ ID NO: 40)
atgaggatatttgctgtctttatattcatgacctactggcatttgct
gaacgccccatacaacaaaatcaaccaaagaattttggttgtggatc
cagtcacctctgaacatgaactgacatgtcaggctgagggctacccc
aaggccgaagtcatctggacaagcagtgaccatcaagtcctgagtgg
taagaccaccaccaccaattccaagagagaggagaagcttttcaatg
tgaccagcacactgagaatcaacacaacaactaatgagattttctac
tgcacttttaggagattagatcctgaggaaaaccatacagctgaatt
ggtcatcccagaactacctctggcacatcctccaaatgaaaggactc
acttggtaattctgggagccatcttattatgccttggtgtagcactg
acattcatcttccgtttaagaaaagggagaatgatggatgtgaaaaa
atgtggcatccaagatacaaactcaaagaagcaaagtgatacacatt
tggaggagacgtaa

By “PAX4 polypeptide” is meant a protein or fragment thereof having at least 85%, at least 90%, at least 95%, at least 98%, or at least 99% amino acid sequence identity to the sequence provided at NCBI Accession No. NP_006184.2 and having transcription factor activity. The amino acid sequence is provided at NCBI Accession No. NP_006184.2 is shown below:

(SEQ ID NO: 41)

| | |
|---|---|
| 1 | MNQLGGLFVN GRPLPLDTRQ QIVRLAVSGM |
| | RPCDISRILK VSNGCVSKIL GRYYRTGVLE |
| 61 | PKGIGGSKPR LATPPVVARI AQLKGECPAL |
| | FAWEIQRQLC AEGLCTQDKT PSVSSINRVL |
| 121 | RALQEDQGLP CTRLRSPAVL APAVLTPHSG |
| | SETPRGTHPG TGHRNRTIFS PSQAEALEKE |
| 181 | FQRGQYPDSV ARGKLATATS LPEDTVRVWF |
| | SNRRAKWRRQ EKLKWEMQLP GASQGLTVPR |
| 241 | VAPGIISAQQ SPGSVPTAAL PALEPLGPSC |
| | YQLCWATAPE RCLSDTPPKA CLKPCWGHLP |
| 301 | PQPNSLDSGL LCLPCPSSHC HLASLSGSQA |
| | LLWPGCPLLY GLE |

By “PAX4 polynucleotide” is meant a polynucleotide encoding a PAX4 polypeptide or fragment thereof. An exemplary PAX4 polynucleotide sequence is provided at NCBI Ref NM_006193.2. The sequence provided at NCBI Ref NM_006193.2 is reproduced below:

(SEQ ID NO: 42)

| | |
|---|---|
| 1 | caaagactca cccgtgagcc agctctcaaa |
| | gaaagcagct tgcgttgaca gcctgggggc |
| 61 | agcaaggatg cagtctccca ggagaggatg |
| | cactcggtgg tgggaagcca ggctggaggg |
| 121 | gcctgagtga ccctctccac aggcgggcag |
| | ggcagtggga gaggtggtgt gtggatacct |
| 181 | ctgtctcacg cccagggatc agcagcatga |
| | accagcttgg ggggctcttt gtgaatggcc |
| 241 | ggcccctgcc tctggatacc cggcagcaga |
| | ttgtgcggct agcagtcagt ggaatgcggc |
| 301 | cctgtgacat ctcacggatc cttaaggtat |
| | ctaatggctg tgtgagcaag atcctagggc |
| 361 | gttactaccg cacaggtgtc ttggagccaa |
| | agggcattgg gggaagcaag ccacggctgg |
| 421 | ctacaccccc tgtggtggct cgaattgccc |
| | agctgaaggg tgagtgtcca gccctctttg |
| 481 | cctgggaaat ccaacgccag ctttgtgctg |
| | aagggctttg cacccaggac aagactccca |
| 541 | gtgtctcctc catcaaccga gtcctgcggg |
| | cattacagga ggaccaggga ctaccgtgca |
| 601 | cacggctcag gtcaccagct gttttggctc |
| | cagctgtcct cactccccat agtggctctg |
| 661 | agactccccg gggtacccac ccagggaccg |
| | gccaccggaa tcggactatc ttctccccaa |
| 721 | gccaagcaga ggcactggag aaagagttcc |
| | agcgtgggca gtatcctgat tcagtggccc |
| 781 | gtggaaagct ggctactgcc acctctctgc |
| | ctgaggacac ggtgagggtc tggttttcca |
| 841 | acagaagagc caaatggcgt cggcaagaga |
| | agctcaagtg ggaaatgcag ctgccaggtg |
| 901 | cttcccaggg gctgactgta ccaagggttg |
| | ccccaggaat catctctgca cagcagtccc |
| 961 | ctggcagtgt gcccacagca gccctgcctg |
| | ccctggaacc actgggtccc tcctgctatc |
| 1021 | agctgtgctg ggcaacagca ccagaaaggt |
| | gtctgagtga caccccacct aaagcctgtc |
| 1081 | tcaagccctg ctggggccac ttgcccccac |
| | agccgaattc cctggactca ggactgcttt |
| 1141 | gccttccttg cccttcctcc cactgtcacc |
| | tggccagtct tagtggctct caggccctgc |
| 1201 | tctggcctgg ctgcccacta ctgtatggct |
| | tggaatgagg caggagtggg aaggagatgg |
| 1261 | catagagaag atctaatacc atcctgccca |
| | ttgtccttac cgtcctgccc atacagactg |
| 1321 | tggctccttc ctccttcctg tgattgctcc |
| | ctcctgtgtg gacgttgcct ggccctgcct |
| 1381 | cgatgcctct ctggcgcatc acctgattgg |
| | aggggctggt aaagcaacac ccacccactt |
| 1441 | ctcacactag ccttaagagg cctccactca |
| | gcagtaataa aagctgtttt tattagcagt |
| 1501 | agttctgttg tccatcatgt tttccctatg |
| | agcaccccta tgcccactct aatattcaac |
| 1561 | aattatagac aatttgccct atcatttatt |
| | tacatctatg tatctaccat ctaatctatg |
| 1621 | catgtatgta ggcaatacat gtatctaaac |
| | aatgtatttg tcaatgcatc aatttaccta |
| 1681 | ctctatgtat gcatctatat gtgtattatg |
| | tatgcgtgca tgcgtgcgcg cacacacaca |
| 1741 | cacacacaca cacactgaca ttatatcatg |
| | gcattttatt cctaaatctt ccagcatgca |
| 1801 | tccccaaaaa acaagaaact tgtcttacat |
| | aatcacaata atatatccac atctaagaaa |
| 1861 | atttactgta acttcttaat ctaagaaaat |
| | tatgtatttt tgtcatatgt attttgtcat |
| 1921 | atgtattttg tatttgcata tgtattttgt |
| | atttgcatat gtatttttgt catagcagca |
| 1981 | aacagagtga aatgccattt ttcatattct |

By “PAX6 polypeptide” is meant a protein or fragment thereof having at least 85%, at least 90%, at least 95%, at least 98%, or at least 99% amino acid sequence identity to the sequence provided at NCBI Accession No. NP_001297090.1 and having transcription factor activity. The amino acid sequence provided at NCBI Accession No. NP_001297090.1 is shown below:

| | |
|---|---|
| 1 | MGADGMYDKL RMLNGQTGSW GTRPGWYPGT |
| | SVPGQPTQDG CQQQEGGGEN TNSISSNGED |
| 61 | SDEAQMRLQL KRKLQRNRTS FTQEQIEALE |
| | KEFERTHYPD VFARERLAAK IDLPEARIQV |
| 121 | WFSNRRAKWR REEKLRNQRR QASNTPSHIP |
| | ISSSFSTSVY QPIPQPTTPV SSFTSGSMLG |
| 181 | RTDTALTNTY SALPPMPSFT MANNLPMQPP |
| | VPSQTSSYSC MLPTSPSVNG RSYDTYTPPH |
| 241 | MQTHMNSQPM GTSGTTSTGL ISPGVSVPVQ |
| | VPGSEPDMSQ YWPRLQ |

By “PAX6 polynucleotide” is meant a polynucleotide encoding a PAX6 polypeptide or fragment thereof. An exemplary PAX6 polynucleotide sequence is provided at NCBI Ref: NM_001310161.1. The sequence provided at NCBI Ref: NM_001310161.1 is reproduced below:

| | |
|---|---|
| 1 | cttttcaatt agccttccat gcatgatccg gagcgacttc cgcctatttc cagaaattaa |
| 61 | gctcaaactt gacgtgcagc tagttttatt ttaaagacaa atgtcagaga ggctcatcat |
| 121 | attttccccc ctcttctata tttggagctt atttattgct aagaagctca ggctcctggc |
| 181 | gtcaatttat cagtaggctc caaggagaag agaggagagg agaggagagc tgaacaggga |
| 241 | gccacgtctt ttcctgggag ggctgctatc taagtcgggg ctgcaggtca cagcggagtg |
| 301 | aatcagctcg gtggtgtctt tgtcaacggg cggccactgc cggactccac ccggcagaag |
| 361 | attgtagagc tagctcacag cggggcccgg ccgtgcgaca tttcccgaat tctgcagacc |
| 421 | catgcagatg caaaagtcca agtgctggac aatcaaaacg tgtccaacgg atgtgtgagt |
| 481 | aaaattctgg gcaggtatta cgagactggc tccatcagac ccagggcaat cggtggtagt |
| 541 | aaaccgagag tagcgactcc agaagttgta agcaaaatag cccagtataa gcgggagtgc |
| 601 | ccgtccatct ttgcttggga aatccgagac agattactgt ccgagggggt ctgtaccaac |
| 661 | gataacatac caagcgtgtc atcaataaac agagttcttc gcaacctggc tagcgaaaag |
| 721 | caacagatgg gcgcagacgg catgtatgat aaactaagga tgttgaacgg gcagaccgga |
| 781 | agctggggca cccgccctgg ttggtatccg gggacttcgg tgccagggca acctacgcaa |
| 841 | gatggctgcc agcaacagga aggaggggga gagaatacca actccatcag ttccaacgga |
| 901 | gaagattcag atgaggctca aatgcgactt cagctgaagc ggaagctgca aagaaataga |
| 961 | acatccttta cccaagagca aattgaggcc ctggagaaag agtttgagag aacccattat |
| 1021 | ccagatgtgt ttgcccgaga aagactagca gccaaaatag atctacctga agcaagaata |
| 1081 | caggtatggt tttctaatcg aagggccaaa tggagaagag aagaaaaact gaggaatcag |
| 1141 | agaagacagg ccagcaacac acctagtcat attcctatca gcagtagttt cagcaccagt |
| 1201 | gtctaccaac caattccaca acccaccaca ccggtttcct ccttcacatc tggctccatg |
| 1261 | ttgggccgaa cagacacagc cctcacaaac acctacagcg ctctgccgcc tatgcccagc |
| 1321 | ttcaccatgg caaataacct gcctatgcaa cccccagtcc ccagccagac ctcctcatac |
| 1381 | tcctgcatgc tgcccaccag cccttcggtg aatgggcgga gttatgatac ctacaccccc |
| 1441 | ccacatatgc agacacacat gaacagtcag ccaatgggca cctcgggcac cacttcaaca |
| 1501 | ggactcattt cccctggtgt gtcagttcca gttcaagttc ccggaagtga acctgatatg |
| 1561 | tctcaatact ggccaagatt acagtaaaaa aaaaaaaaaa aaaaaaaagg aaaggaaata |
| 1621 | ttgtgttaat tcagtcagtg actatgggga cacaacagtt gagctttcag gaaagaaaga |
| 1681 | aaaatggctg ttagagccgc ttcagttcta caattgtgtc ctgtattgta ccactgggga |
| 1741 | aggaatggac ttgaaacaag gacctttgta tacagaaggc acgatatcag ttggaacaaa |
| 1801 | tcttcatttt ggtatccaaa cttttattca ttttggtgta ttatttgtaa atgggcattt |
| 1861 | gtatgttata atgaaaaaaa gaacaatgta gactggatgg atgtttgatc tgtgttggtc |
| 1921 | atgaagttgt tttttttttt tttaaaaaga aaaccatgat caacaagctt tgccacgaat |
| 1981 | ttaagagttt tatcaagata tatcgaatac ttctacccat ctgttcatag tttatggact |
| 2041 | gatgttccaa gtttgtatca ttcctttgca tataattaaa cctggaacaa catgcactag |
| 2101 | atttatgtca gaaatatctg ttggttttcc aaaggttgtt aacagatgaa gtttatgtgc |
| 2161 | aaaaaagggt aagatataaa ttcaaggaag aaaaaaagtt gatagctaaa aggtagagtg |
| 2221 | tgtcttcgat ataatccaat ttgttttatg tcaaaatgta agtatttgtc ttccctagaa |
| 2281 | atcctcagaa tgatttctat aataaagtta atttcattta tatttgacaa gaatatagat |
| 2341 | gttttataca cattttcatg caatcatacg tttctttttt ggccagcaaa agttaattgt |
| 2401 | tcttagatat agttgtatta ctgttcacgg tccaatcatt ttgtgcatct agagttcatt |
| 2461 | cctaatcaat taaaagtgct tgcaagagtt ttaaacttaa gtgttttgaa gttgttcaca |
| 2521 | actacatatc aaaattaacc attgttgatt gtaaaaaacc atgccaaagc ctttgtattt |
| 2581 | cctttattat acagttttct ttttaacctt atagtgtggt gttacaaatt ttatttccat |
| 2641 | gttagatcaa cattctaaac caatggttac tttcacacac actctgtttt acatcctgat |
| 2701 | gatccttaaa aaataatcct tatagatacc ataaatcaaa aacgtgttag aaaaaaattc |
| 2761 | cacttacagc agggtgtaga tctgtgccca tttataccca caacatatat acaaaatggt |
| 2821 | aacatttccc agttagccat ttaattctaa agctcaaagt ctagaaataa tttaaaaatg |
| 2881 | caacaagcga ttagctagga attgtttttt gaattaggac tggcattttc aatctgggca |
| 2941 | gatttccatt gtcagcctat ttcaacaatg atttcactga agtatattca aaagtagatt |
| 3001 | tcttaaagga gactttctga aagctgttgc ctttttcaaa taggccctct cccttttctg |
| 3061 | tctccctccc ctttgcacaa gaggcatcat ttcccattga accactacag ctgttcccat |
| 3121 | ttgaatcttg ctttctgtgc ggttgtggat ggttggaggg tggagggggg atgttgcatg |
| 3181 | tcaaggaata atgagcacag acacatcaac agacaacaac aaagcagact gtgactggcc |
| 3241 | ggtgggaatt aaaggccttc agtcattggc agcttaagcc aaacattccc aaatctatga |
| 3301 | agcagggccc attgttggtc agttgttatt tgcaatgaag cacagttctg atcatgttta |
| 3361 | aagtggaggc acgcagggca ggagtgcttg agcccaagca aaggatggaa aaaaataagc |
| 3421 | ctttgttggg taaaaaagga ctgtctgaga ctttcatttg ttctgtgcaa catataagtc |
| 3481 | aatacagata agtcttcctc tgcaaacttc actaaaaagc ctgggggttc tggcagtcta |
| 3541 | gattaaaatg cttgcacatg cagaaacctc tggggacaaa gacacacttc cactgaatta |
| 3601 | tactctgctt taaaaaaatc cccaaaagca aatgatcaga aatgtagaaa ttaatggaag |
| 3661 | gatttaaaca tgaccttctc gttcaatatc tactgttttt tagttaagga attacttgtg |
| 3721 | aacagataat tgagattcat tgctccggca tgaaatatac taataatttt attccaccag |
| 3781 | agttgctgca catttggaga caccttccta agttgcagtt tttgtatgtg tgcatgtagt |
| 3841 | tttgttcagt gtcagcctgc actgcacagc agcacatttc tgcaggggag tgagcacaca |
| 3901 | tacgcactgt tggtacaatt gccggtgcag acatttctac ctcctgacat tttgcagcct |
| 3961 | acattccctg agggctgtgt gctgagggaa ctgtcagaga agggctatgt gggagtgcat |
| 4021 | gccacagctg ctggctggct tacttcttcc ttctcgctgg ctgtaatttc caccacggtc |
| 4081 | aggcagccag ttccggccca cggttctgtt gtgtagacag cagagacttt ggagacccgg |
| 4141 | atgtcgcacg ccaggtgcaa gaggtgggaa tgggagaaaa ggagtgacgt gggagcggag |
| 4201 | ggtctgtatg tgtgcacttg ggcacgtata tgtgtgctct gaaggtcagg attgccaggg |
| 4261 | caaagtagca cagtctggta tagtctgaag aagcggctgc tcagctgcag aagccctctg |
| 4321 | gtccggcagg atgggaacgg ctgccttgcc ttctgcccac accctaggga catgagctgt |
| 4381 | ccttccaaac agagctccag gcactctctt ggggacagca tggcaggctc tgtgtggtag |
| 4441 | cagtgcctgg gagttggcct tttactcatt gttgaaataa tttttgttta ttatttattt |
| 4501 | aacgatacat atatttatat atttatcaat ggggtatctg cagggatgtt ttgacaccat |
| 4561 | cttccaggat ggagattatt tgtgaagact tcagtagaat cccaggacta aacgtctaaa |
| 4621 | ttttttctcc aaacttgact gacttgggaa aaccaggtga atagaataag agctgaatgt |
| 4681 | tttaagtaat aaacgttcaa actgctctaa gtaaaaaaat gcattttact gcaatgaatt |
| 4741 | tctagaatat ttttccccca aagctatgcc tcctaaccct taaatggtga acaactggtt |
| 4801 | tcttgctaca gctcactgcc atttcttctt actatcatca ctaggtttcc taagattcac |
| 4861 | tcatacagta ttatttgaag attcagcttt gttctgtgaa tgtcatctta ggattgtgtc |
| 4921 | tatattcttt tgcttatttc tttttactct gggcctctca tactagtaag attttaaaaa |
| 4981 | gccttttctt ctctgtatgt ttggctcacc aaggcgaaat atatattctt ctctttttca |
| 5041 | tttctcaaga ataaacctca tctgcttttt tgtttttctg tgttttggct tggtactgaa |
| 5101 | tgactcaact gctcggtttt aaagttcaaa gtgtaagtac ttagggttag tactgcttat |
| 5161 | ttcaataatg ttgacggtga ctatctttgg aaagcagtaa catgctgtct tagaaatgac |
| 5221 | attaataatg ggcttaaaca aatgaatagg ggggtccccc cactctcctt ttgtatgcct |
| 5281 | atgtgtgtct gatttgttaa aagatggaca gggaattgat tgcagagtgt cgcttccttc |
| 5341 | taaagtagtt ttattttgtc tactgttagt atttaaagat cctggaggtg gacataagga |
| 5401 | ataaatggaa gagaaaagta gatattgtat ggtggctact aaaaggaaat tcaaaaagtc |
| 5461 | ttagaacccg agcacctgag caaactgcag tagtcaaaat atttatctca tgttaaagaa |
| 5521 | aggcaaatct agtgtaagaa atgagtacca tatagggttt tgaagttcat atactagaaa |
| 5581 | cacttaaaag atatcatttc agatattacg tttggcattg ttcttaagta tttatatctt |
| 5641 | tgagtcaagc tgataattaa aaaaaatctg ttaatggagt gtatatttca taatgtatca |
| 5701 | aaatggtgtc tatacctaag gtagcattat tgaagagaga tatgtttatg tagtaagtta |
| 5761 | ttaacataat gagtaacaaa taatgtttcc agaagaaagg aaaacacatt ttcagagtgc |
| 5821 | gtttttatca gaggaagaca aaaatacaca cccctctcca gtagcttatt tttacaaagc |
| 5881 | cggcccagtg aattagaaaa acaaagcact tggatatgat ttttggaaag cccaggtaca |
| 5941 | cttattattc aaaatgcact tttactgagt ttgaaaagtt tcttttatat ttaaaataag |
| 6001 | ggttcaaata tgcatattca atttttatag tagttatcta tttgcaaagc atatattaac |
| 6061 | tagtaattgg ctgttaattt tatagacatg gtagccaggg aagtatatca atgacctatt |
| 6121 | aagtattttg acaagcaatt tacatatctg atgacctcgt atctcttttt cagcaagtca |
| 6181 | aatgctatgt aattgttcca ttgtgtgttg tataaaatga atcaacacgg taagaaaaag |
| 6241 | gttagagtta ttaaaataat aaactgacta aaatactcat ttgaatttat tcagaatgtt |
| 6301 | cataatgctt tcaaaggaca tagcagagct tttgtggagt atccgcacaa cattatttat |
| 6361 | tatctatgga ctaaatcaat tttttgaagt tgctttaaaa tttaaaagca cctttgctta |
| 6421 | atataaagcc ctttaatttt aactgacaga tcaattctga aactttattt tgaaaagaaa |
| 6481 | atggggaaga atctgtgtct ttagaattaa aagaaatgaa aaaaataaac ccgacattct |
| 6541 | aaaaaaatag aataagaaac ctgattttta gtactaatga aatagcgggt gacaaaatag |
| 6601 | ttgtcttttt gattttgatc acaaaaaata aactggtagt gacaggatat gatggagaga |
| 6661 | tttgacatcc tggcaaatca ctgtcattga ttcaattatt ctaattctga ataaaagctg |
| 6721 | tatacagtaa aa |

By “PDX1 polypeptide” is meant a protein or fragment thereof having at least 85%, at least 90%, at least 95%, at least 98%, or at least 99% amino acid sequence identity to the sequence provided at NCBI Accession No. NP_000200.1 and having transcription factor activity. The amino acid sequence provided at NCBI Accession No. NP_000200.1 is shown below:

| | |
|---|---|
| 1 | MNGEEQYYAA TQLYKDPCAF QRGPAPEFSA |
| | SPPACLYMGR QPPPPPPHPF PGALGALEQG |
| 61 | SPPDISPYEV PPLADDPAVA HLHHHLPAQL |
| | ALPHPPAGPF PEGAEPGVLE EPNRVQLPFP |
| 121 | WMKSTKAHAW KGQWAGGAYA AEPEENKRTR |
| | TAYTRAQLLE LEKEFLFNKY ISRPRRVELA |
| 181 | VMLNLTERHI KIWFQNRRMK WKKEEDKKRG |
| | GGTAVGGGGV AEPEQDCAVT SGEELLALPP |
| 241 | PPPPGGAVPP AAPVAAREGR LPPGLSASPQ |
| | PSSVAPRRPQ EPR |

By “PDX1 polynucleotide” is meant a polynucleotide encoding a PDX1 polypeptide or fragment thereof. An exemplary PDX1 polynucleotide sequence is provided at NCBI Ref: NM_000209.3. The sequence provided at NCBI Ref NM_000209.3 is reproduced below:

| | |
|---|---|
| 1 | gggtggcgcc gggagtggga acgccacaca gtgccaaatc cccggctcca gctcccgact |
| 61 | cccggctccc ggctcccggc tcccggtgcc caatcccggg ccgcagccat gaacggcgag |
| 121 | gagcagtact acgcggccac gcagctttac aaggacccat gcgcgttcca gcgaggcccg |
| 181 | gcgccggagt tcagcgccag cccccctgcg tgcctgtaca tgggccgcca gcccccgccg |
| 241 | ccgccgccgc acccgttccc tggcgccctg ggcgcgctgg agcagggcag ccccccggac |
| 301 | atctccccgt acgaggtgcc ccccctcgcc gacgaccccg cggtggcgca ccttcaccac |
| 361 | cacctcccgg ctcagctcgc gctcccccac ccgcccgccg ggcccttccc ggagggagcc |
| 421 | gagccgggcg tcctggagga gcccaaccgc gtccagctgc ctttcccatg gatgaagtct |
| 481 | accaaagctc acgcgtggaa aggccagtgg gcaggcggcg cctacgctgc ggagccggag |
| 541 | gagaacaagc ggacgcgcac ggcctacacg cgcgcacagc tgctagagct ggagaaggag |
| 601 | ttcctattca acaagtacat ctcacggccg cgccgggtgg agctggctgt catgttgaac |
| 661 | ttgaccgaga gacacatcaa gatctggttc caaaaccgcc gcatgaagtg gaaaaaggag |
| 721 | gaggacaaga agcgcggcgg cgggacagct gtcgggggtg gcggggtcgc ggagcctgag |
| 781 | caggactgcg ccgtgacctc cggcgaggag cttctggcgc tgccgccgcc gccgcccccc |
| 841 | ggaggtgctg tgccgcccgc tgcccccgtt gccgcccgag agggccgcct gccgcctggc |
| 901 | cttagcgcgt cgccacagcc ctccagcgtc gcgcctcggc ggccgcagga accacgatga |
| 961 | gaggcaggag ctgctcctgg ctgaggggct tcaaccactc gccgaggagg agcagagggc |
| 1021 | ctaggaggac cccgggcgtg gaccacccgc cctggcagtt gaatggggcg gcaattgcgg |
| 1081 | ggcccacctt agaccgaagg ggaaaacccg ctctctcagg cgcatgtgcc agttggggcc |
| 1141 | ccgcgggtag atgccggcag gccttccgga agaaaaagag ccattggttt ttgtagtatt |
| 1201 | ggggccctct tttagtgata ctggattggc gttgtttgtg gctgttgcgc acatccctgc |
| 1261 | cctcctacag cactccacct tgggacctgt ttagagaagc cggctcttca aagacaatgg |
| 1321 | aaactgtacc atacacattg gaaggctccc taacacacac agcggggaag ctgggccgag |
| 1381 | taccttaatc tgccataaag ccattcttac tcgggcgacc cctttaagtt tagaaataat |
| 1441 | tgaaaggaaa tgtttgagtt ttcaaagatc ccgtgaaatt gatgccagtg gaatacagtg |
| 1501 | agtcctcctc ttcctcctcc tcctcttccc cctccccttc ctcctcctcc tcttcttttc |
| 1561 | cctcctcttc ctcttcctcc tgctctcctt tcctccccct cctcttttcc ctcctcttcc |
| 1621 | tcttcctcct gctctccttt cctccccctc ctctttctcc tcctcctcct cttcttcccc |
| 1681 | ctcctctccc tcctcctctt cttccccctc ctctccctcc tcctcttctt ctccctcctc |
| 1741 | ttcctcttcc tcctcttcca cgtgctctcc tttcctcccc ctcctcttgc tccccttctt |
| 1801 | ccccgtcctc ttcctcctcc tcctcttctt ctccctcctc ttcctcctcc tctttcttcc |
| 1861 | tgacctcttt ctttctcctc ctcctccttc tacctcccct tctcatccct cctcttcctc |
| 1921 | ttctctagct gcacacttca ctactgcaca tcttataact tgcacccctt tcttctgagg |
| 1981 | aagagaacat cttgcaaggc agggcgagca gcggcagggc tggcttagga gcagtgcaag |
| 2041 | agtccctgtg ctccagttcc acactgctgg cagggaaggc aaggggggac gggcctggat |
| 2101 | ctgggggtga gggagaaaga tggacccctg ggtgaccact aaaccaaaga tattcggaac |
| 2161 | tttctattta ggatgtggac gtaattcctg ttccgaggta gaggctgtgc tgaagacaag |
| 2221 | cacagtggcc tggtgcgcct tggaaaccaa caactattca cgagccagta tgaccttcac |
| 2281 | atctttagaa attatgaaaa cgtatgtgat tggagggttt ggaaaaccag ttatcttatt |
| 2341 | taacatttta aaaattacct aacagttatt tacaaacagg tctgtgcatc ccaggtctgt |
| 2401 | cttcttttca aggtctgggc cttgtgctcg ggttatgttt gtgggaaatg cttaataaat |
| 2461 | actgataata tgggaagaga tgaaaactga ttctcctcac tttgtttcaa acctttctgg |
| 2521 | cagtgggatg attcgaattc acttttaaaa ttaaattagc gtgttttgtt ttg |

By “PTF1 polypeptide” is meant a protein or fragment thereof having at least 85%, at least 90%, at least 95%, at least 98%, or at least 99% amino acid sequence identity to the sequence provided at NCBI Accession No. NP_835455.1 and having transcription factor activity. The amino acid sequence provided at NCBI Accession No. NP_835455.1 is shown below:

| | |
|---|---|
| 1 | MDAVLLEHFP GGLDAFPSSY FDEDDFFTDQ |
| | SSRDPLEDGD ELLADEQAEV EFLSHQLHEY |
| 61 | CYRDGACLLL QPAPPAAPLA LAPPSSGGLG |
| | EPDDGGGGGY CCETGAPPGG FPYSPGSPPS |
| 121 | CLAYPCAGAA VLSPGARLRG LSGAAAAAAR |
| | RRRRVRSEAE LQQLRQAANV RERRRMQSIN |
| 181 | DAFEGLRSHI PTLPYEKRLS KVDTLRLAIG |
| | YINFLSELVQ ADLPLRGGGA GGCGGPGGGG |
| 241 | RLGGDSPGSQ AQKVIICHRG TRSPSPSDPD |
| | YGLPPLAGHS LSWTDEKQLK EQNIIRTAKV |
| 301 | WTPEDPRKLN SKSSFNNIEN EPPFEFVS |

By “PTF1 polynucleotide” is meant a polynucleotide encoding a PTF1 polypeptide or fragment thereof. An exemplary PTF1 polynucleotide sequence is provided at NCBI Ref: NM_178161.2. The sequence provided at NCBI Ref: NM_178161.2 is reproduced below:

| | |
|---|---|
| 1 | atggacgcgg tgttgctgga gcacttcccc gggggcctag acgcctttcc ttcttcgtac |
| 61 | ttcgacgagg acgacttctt caccgaccag tcttcacggg accccctgga ggacggcgat |
| 121 | gagctgctgg cggacgagca ggccgaggtg gagttcctta gccaccagct ccacgagtac |
| 181 | tgctaccgcg acggggcgtg cctgctgctg cagcccgcgc ccccggccgc cccgctagcg |
| 241 | ctcgccccgc cgtcctcggg gggcctcggt gagccagacg acggcggcgg cggcggctac |
| 301 | tgctgcgaga cgggggcgcc cccaggcggc ttcccctact cgcccggctc gccgccctcg |
| 361 | tgcctggcct acccgtgcgc cggggcggca gtactgtctc ccggggcgcg gctgcgcggc |
| 421 | ctgagcggag cggcggctgc ggcggcgcgg cgccggcggc gggtgcgctc cgaggcggag |
| 481 | ctgcagcagc tgcggcaggc ggccaacgtg cgcgagcggc ggcgcatgca gtccatcaac |
| 541 | gacgccttcg aggggctgcg ctcgcacatc cccacgctgc cctacgagaa gcgcctctcc |
| 601 | aaggtggaca cgctgcgcct ggccatcggc tacatcaact tcctcagcga gctcgtgcag |
| 661 | gccgacctgc ccttgcgcgg cggtggcgcg ggcggctgcg gggggccggg cggcggcggg |
| 721 | cgcctgggcg gggacagccc gggcagccag gcccagaagg tcatcatctg ccatcggggc |
| 781 | acccggtccc cctcccccag cgaccctgat tatggcctcc ctcccctagc aggacactct |
| 841 | ctctcatgga ctgatgaaaa acaactcaag gaacaaaata ttatccgaac agccaaagtc |
| 901 | tggaccccag aggaccccag aaaactcaac agcaaatctt ccttcaacaa catagaaaac |
| 961 | gaaccaccat ttgagtttgt gtcctgagaa gtcccagact cggctgaaga tctgattatg |
| 1021 | tctctgtgca tattgtacat gtaaatatct ataatgtaaa tgtaatttaa gaatcaaatt |
| 1081 | tttcgaatgg caatcaactg tttattattt atctatttat tatcctgttg agttgatgaa |
| 1141 | atagatgatt tctttttaaa tatataattt atataactta tcctgatttt ctgaaaatat |
| 1201 | gcaatagcct atgattttcc tgaactctgt gttgttggga gaactctggc cagaaaacgt |
| 1261 | cctgcttatt tattgccaga tatggtttat ttctaagcgt tgtcaataaa tgctatttac |
| 1321 | accttttcct gaaaaaaaa |

By “Wnt3a polynucleotide” is meant a polynucleotide encoding a Wnt3a polypeptide or a fragment thereof. An exemplary human Wnt3a polynucleotide sequence is provided at NCBI GenBank Accession No. AB060284.1. The polynucleotide sequence provided at NCBI GenBank Accession No. AB060284.1 is reproduced below:

| | |
|---|---|
| 1 | cggcgatggc cccactcgga tacttcttac tcctctgcag cctgaagcag gctctgggca |
| 61 | gctacccgat ctggtggtcg ctggctgttg ggccacagta ttcctccctg ggctcgcagc |
| 121 | ccatcctgtg tgccagcatc ccgggcctgg tccccaagca gctccgcttc tgcaggaact |
| 181 | acgtggagat catgcccagc gtggccgagg gcatcaagat tggcatccag gagtgccagc |
| 241 | accagttccg cggccgccgg tggaactgca ccaccgtcca cgacagcctg gccatcttcg |
| 301 | ggcccgtgct ggacaaagct accagggagt cggcctttgt ccacgccatt gcctcagccg |
| 361 | gtgtggcctt tgcagtgaca cgctcatgtg cagaaggcac ggccgccatc tgtggctgca |
| 421 | gcagccgcca ccagggctca ccaggcaagg gctggaagtg gggtggctgt agcgaggaca |
| 481 | tcgagtttgg tgggatggtg tctcgggagt tcgccgacgc ccgggagaac cggccagatg |
| 541 | cccgctcagc catgaaccgc cacaacaacg aggctgggcg ccaggccatc gccagccaca |
| 601 | tgcacctcaa gtgcaagtgc cacgggctgt cgggcagctg cgaggtgaag acatgctggt |
| 661 | ggtcgcaacc cgacttccgc gccatcggtg acttcctcaa ggacaagtac gacagcgcct |
| 721 | cggagatggt ggtggagaag caccgggagt cccgcggctg ggtggagacc ctgcggccgc |
| 781 | gctacaccta cttcaaggtg cccacggagc gcgacctggt ctactacgag gcctcgccca |
| 841 | acttctgcga gcccaaccct gagacgggct ccttcggcac gcgcgaccgc acctgcaacg |
| 901 | tcagctcgca cggcatcgac ggctgcgacc tgctgtgctg cggccgcggc cacaacgcgc |
| 961 | gagcggagcg gcgccgggag aagtgccgct gcgtgttcca ctggtgctgc tacgtcagct |
| 1021 | gccaggagtg cacgcgcgtc tacgacgtgc acacctgcaa gtaggcaccg gccgcggctc |
| 1081 | cccctggacg gggcgggccc tgcctgaggg tgggcttttc cctgggtgga gcaggactcc |
| 1141 | cacctaaacg gggcagtact cctccctggg ggcgggactc ctccctgggg gtggggctcc |
| 1201 | tacctggggg cagaactcct acctgaaggc agggctcctc cctggagcta gtgtctcctc |
| 1261 | tctggtggct gggctgctcc tgaatgaggc ggagctccag gatggggagg ggctctgcgt |
| 1321 | tggcttctcc ctggggacgg ggctcccctg gacagaggcg gggctacaga ttgggcgggg |
| 1381 | cttctcttgg gtgggacagg gcttctcctg cgggggcgag gcccctccca gtaagggcgt |
| 1441 | ggctctgggt gggcggggca ctaggtaggc ttctacctgc aggcggggct cctcctgaag |
| 1501 | gaggcggggc tctaggatgg ggcacggctc tggggtaggc tgctccctga gggcg |

By “Wnt3a polypeptide” is meant a Wnt3a polypeptide or a fragment thereof, or a polypeptide having at least 85%, at least 90%, at least 95%, at least 98%, or at least 99% amino acid sequence identity to the human Wnt3a polypeptide sequence. An exemplary human Wnt3a polypeptide sequence is provided at NCBI GenBank: AAI03924.1. The sequence provided at GenBank: AAI03924.1 is reproduced below:

| | |
|---|---|
| 1 | MAPLGYFLLL CSLKQALGSY PIWWSLAVGP |
| | QYSSLGSQPI LCASIPGLVP KQLRFCRNYV |
| 61 | EIMPSVAEGI KIGIQECQHQ FRGRRWNCTT |
| | VHDSLAIFGP VLDKATRESA FVHAIASAGV |
| 121 | AFAVTRSCAE GTAAICGCSS RHQGSPGKGW |
| | KWGGCSEDIE FGGMVSREFA DARENRPDAR |
| 181 | SAMNRHNNEA GRQAIASHMH LKCKCHGLSG |
| | SCEVKTCWWS QPDFRAIGDF LKDKYDSASE |
| 241 | MVVEKHRESR GWVETLRPRY TYFKVPTERD |
| | LVYYEASPNF CEPNPETGSF GTRDRTCNVS |
| 301 | SHGIDGCDLL CCGRGHNARA ERRREKCRCV |
| | FHWCCYVSCQ ECTRVYDVHT CKNPGSRAGN |
| 361 | SAHQPPHPQP PVRFHPPLRR AGKVP |

By “Wnt4 polynucleotide” is meant a polynucleotide encoding Wnt4 polypeptide or a fragment thereof. An exemplary human Wnt4 polynucleotide sequence is provided at NCBI GenBank Accession No. AY009398.1. Accession number NCBI Ref NG_008974.1 is a reference standard Wnt4a polynucleotide sequence. The polynucleotide sequence provided at NCBI GenBank Accession No. AY009398.1 is reproduced below:

| | |
|---|---|
| 1 | atgagtcccc gctcgtgcct gcgttcgctg cgcctcctcg tcttcgccgt cttctcagcc |
| 61 | gccgcgagca actggctgta cctggccaag ctgtcgtcgg tggggagcat ctcagaggag |
| 121 | gagacgtgcg agaaactcaa gggcctgatc cagaggcagg tgcagatgtg caagcggaac |
| 181 | ctggaagtca tggactcggt gcgccgcggt gcccagctgg ccattgagga gtgccagtac |
| 241 | cagttccgga accggcgctg gaactgctcc acactcgact ccttgcccgt cttcggcaag |
| 301 | gtggtgacgc aagggattcg ggaggcggcc ttggtgtacg ccatctcttc ggcaggtgtg |
| 361 | gcctttgcag tgacgcgggc gtgcagcagt ggggagctgg agaagtgcgg ctgtgacagg |
| 421 | acagtgcatg gggtcagccc acagggcttc cagtggtcag gatgctctga caacatcgcc |
| 481 | tacggtgtgg ccttctcaca gtcgtttgtg gatgtgcggg agagaagcaa gggggcctcg |
| 541 | tccagcagag ccctcatgaa cctccacaac aatgaggccg gcaggaaggc catcctgaca |
| 601 | cacatgcggg tggaatgcaa gtgccacggg gtgtcaggct cctgtgaggt aaagacgtgc |
| 661 | tggcgagccg tgccgccctt ccgccaggtg ggtcacgcac tgaaggagaa gtttgatggt |
| 721 | gccactgagg tggagccacg ccgcgtgggc tcctccaggg cactggtgcc acgcaacgca |
| 781 | cagttcaagc cgcacacaga tgaggacttg gtgtacttgg agcctagccc cgacttctgt |
| 841 | gagcaggaca tgcgcagcgg cgtgctgggc acgaggggcc gcacatgcaa caagacgtcc |
| 901 | aaggccatcg acggctgtga gctgctgtgc tgtggccgcg gcttccacac ggcgcaggtg |
| 961 | gagctggctg aacgctgcag ctgcaaattc cactggtgct gcttcgtcaa gtgccggcag |
| 1021 | tgccagcggc tcgtggagtt gcacacgtgc cgatga |

By “Wnt4 polypeptide” is meant a Wnt4 polypeptide or a fragment thereof, or a polypeptide having at least 85%, at least 90%, at least 95%, at least 98%, or at least 99% amino acid sequence identity to the human Wnt4 polypeptide sequence. An exemplary human Wnt4 polypeptide sequence is provided at NCBI GenBank Accession No.: AAG38658.1. The sequence provided at GenBank Accession No.: AAG38658.1 is reproduced below:

| | |
|---|---|
| 1 | MSPRSCLRSL RLLVFAVFSA AASNWLYLAK |
| | LSSVGSISEE ETCEKLKGLI QRQVQMCKRN |
| 61 | LEVMDSVRRG AQLAIEECQY QFRNRRWNCS |
| | TLDSLPVFGK VVTQGIREAA LVYAISSAGV |
| 121 | AFAVTRACSS GELEKCGCDR TVHGVSPQGF |
| | QWSGCSDNIA YGVAFSQSFV DVRERSKGAS |
| 181 | SSRALMNLHN NEAGRKAILT HMRVECKCHG |
| | VSGSCEVKTC WRAVPPFRQV GHALKEKFDG |
| 241 | ATEVEPRRVG SSRALVPRNA QFKPHTDEDL |
| | VYLEPSPDFC EQDMRSGVLG TRGRTCNKTS |
| 301 | KAIDGCELLC CGRGFHTAQV ELAERCSCKF |
| | HWCCFVKCRQ CQRLVELHTC R |

By “Wnt5a polynucleotide” is meant a polynucleotide encoding Wnt5a polypeptide or a fragment thereof. An exemplary polynucleotide sequence coding for human Wnt5a is provided at NCBI Ref: GenBank NM_003392, a reference standard sequence. Nucleotides 658-1800 of the Wnt5a genomic sequence having 6194 nucleotides codes for a human Wnt5a polypeptide. The polynucleotide sequence of the human Wnt5a coding sequence provided at bases 658-1800 of NCBI Ref: GenBank NM 003392 is reproduced below:

| | |
|---|---|
| 658 | atg |
| 661 | aagaagtcca ttggaatatt aagcccagga |
| | gttgctttgg ggatggctgg aagtgcaatg |
| 721 | tcttccaagt tcttcctagt ggctttggcc |
| | atatttttct ccttcgccca ggttgtaatt |
| 781 | gaagccaatt cttggtggtc gctaggtatg |
| | aataaccctg ttcagatgtc agaagtatat |
| 841 | attataggag cacagcctct ctgcagccaa |
| | ctggcaggac tttctcaagg acagaagaaa |
| 901 | ctgtgccact tgtatcagga ccacatgcag |
| | tacatcggag aaggcgcgaa gacaggcatc |
| 961 | aaagaatgcc agtatcaatt ccgacatcga |
| | aggtggaact gcagcactgt ggataacacc |
| 1021 | tctgtttttg gcagggtgat gcagataggc |
| | agccgcgaga cggccttcac atacgcggtg |
| 1081 | agcgcagcag gggtggtgaa cgccatgagc |
| | cgggcgtgcc gcgagggcga gctgtccacc |
| 1141 | tgcggctgca gccgcgccgc gcgccccaag |
| | gacctgccgc gggactggct ctggggcggc |
| 1201 | tgcggcgaca acatcgacta tggctaccgc |
| | tttgccaagg agttcgtgga cgcccgcgag |
| 1261 | cgggagcgca tccacgccaa gggctcctac |
| | gagagtgctc gcatcctcat gaacctgcac |
| 1321 | aacaacgagg ccggccgcag gacggtgtac |
| | aacctggctg atgtggcctg caagtgccat |
| 1381 | ggggtgtccg gctcatgtag cctgaagaca |
| | tgctggctgc agctggcaga cttccgcaag |
| 1441 | gtgggtgatg ccctgaagga gaagtacgac |
| | agcgcggcgg ccatgcggct caacagccgg |
| 1501 | ggcaagttgg tacaggtcaa cagccgcttc |
| | aactcgccca ccacacaaga cctggtctac |
| 1561 | atcgacccca gccctgacta ctgcgtgcgc |
| | aatgagagca ccggctcgct gggcacgcag |
| 1621 | ggccgcctgt gcaacaagac gtcggagggc |
| | atggatggct gcgagctcat gtgctgcggc |
| 1681 | cgtggctacg accagttcaa gaccgtgcag |
| | acggagcgct gccactgcaa gttccactgg |
| 1741 | tgctgctacg tcaagtgcaa gaagtgcacg |
| | gagatcgtgg accagtttgt gtgcaagtag |

By “Wnt5a polypeptide” is meant a Wnt5a polypeptide or a fragment thereof, or a polypeptide having at least 85%, at least 90%, at least 95%, at least 98%, or at least 99% amino acid sequence identity to the human Wnt5a polypeptide sequence. An exemplary human Wnt5a (isoform 1) polypeptide sequence is provided at UniProtKB Identifier: P41221-1. The sequence provided at UniProtKB Identifier: P41221-1 is reproduced below:

| | |
|---|---|
| 1 | MKKSIGILSP GVALGMAGSA MSSKFFLVAL |
| | AIFFSFAQVV IEANSWWSLG |
| 51 | MNNPVQMSEV YIIGAQPLCS QLAGLSQGQK |
| | KLCHLYQDHM QYIGEGAKTG |
| 101 | IKECQYQFRH RRWNCSTVDN TSVFGRVMQI |
| | GSRETAFTYA VSAAGVVNAM |
| 151 | SRACREGELS TCGCSRAARP KDLPRDWLWG |
| | GCGDNIDYGY RFAKEFVDAR |
| 201 | ERERIHAKGS YESARILMNL HNNEAGRRTV |
| | YNLADVACKC HGVSGSCSLK |
| 251 | TCWLQLADFR KVGDALKEKY DSAAAMRLNS |
| | RGKLVQVNSR FNSPTTQDLV |
| 301 | YIDPSPDYCV RNESTGSLGT QGRLCNKTSE |
| | GMDGCELMCC GRGYDQFKTV |
| 351 | QTERCHCKFH WCCYVKCKKC TEIVDQFVCK |

An “immune checkpoint protein or molecule” or “immune checkpoint” refers to a specific subtype of transmembrane protein molecule that provides fine-tuning of the immune response. In normal tissues, immune checkpoints are inhibitory signals and play an important role in immune cell function by preventing autoimmunity. In a subject with a tumor or cancer, up-regulation of immune checkpoint proteins on the tumor or cancer cells allows tumors and cancers to escape immune surveillance and evade anti-tumor immunity. Nonlimiting examples of immune checkpoint proteins that have been the focus of clinical immunotherapeutics are cytotoxic T-lymphocyte-associated antigen 4 (CTLA-4), programmed cell death protein 1 (PD-1), and programmed cell death protein ligand 1 (PD-L1). CTLA-4, also known as CD152, is essential for the activation of CD4⁺ T cells and the priming phase of the immune response. PD-1, also known as CD279 and formerly as B7.1, is a key immune checkpoint receptor expressed by activated T cells, B cells and myeloid cells, and mediates immunosuppression. PD-L1, also known as CD274 and formerly as B7-H1, is an immune regulatory protein that plays a significant role in suppressing the immune system during certain disease states, including cancer and autoimmune disease. PD-L1 is the cognate ligand that binds to PD-1 to modulate activation or inhibition of immune cells. Under normal circumstances, the immune system reacts to foreign antigens that are associated with exogenous or endogenous agents, e.g., microorganisms or cells, which triggers the proliferation of antigen-specific cytotoxic CD8+ T cells and/or CD4+ helper T cells. The binding of PD-L1 to PD-1 transmits an inhibitory signal that reduces the proliferation of the antigen-specific T cells in lymph nodes, while simultaneously reducing apoptosis in regulatory T cells (anti-inflammatory, suppressive T cells).

The K_{d} (dissociation constant), which reflects the binding affinity between PD-L1 and PD-1, is 770 nM. PD-L1 also has an appreciable affinity for the costimulatory molecule CD80 (B7-1), but not for CD86 (B7-2). The affinity of PD-L1 of CD80 is 1.4 μM, which is a value that is intermediate between the affinity of PD-L1 for CD28 and CTLA-4 (4.0 μM and 400 nM, respectively). The related molecule PD-L2 does not have affinity for CD80 or CD86, but shares PD-1 as a receptor (with a stronger K_{d} of 140 nM). PD-1 is up-regulated on activated CD4 T-cells and can bind to PD-L1-expressing monocytes to induce the production of IL-10. (E. A. Said et al., 2010, Nature Medicine, 16(4):452-459). The interaction of PD-L1 with its receptor PD-1 on T cells delivers a signal that inhibits T cell receptor (TCR)-mediated activation of IL-2 production and T cell proliferation. The PD-1/PD-L1 interaction has been implicated in autoimmunity. By way of example, NOD mice, an animal model for autoimmunity, exhibit a susceptibility to spontaneous development of type I diabetes and other autoimmune diseases and have been shown to develop a precipitated onset of diabetes from the blockade of PD-1 or PD-L1 (but not PD-L2), (M. J. Ansari et al., 2003, J. Exp. Med., 198(1):63-69).

By “immune surveillance” or “immunological surveillance” is meant a monitoring process by cells of the immune system to detect and destroy cells that are recognized as non-self, other, or allogeneic in the tissues and organs of the body. For example, such non-self cells may be virally-infected, mutated, neoplastically transformed, or may express a cell surface molecule that is not recognized as a self or autologous molecule by cells of the immune system.

By “progenitor cell” is meant a cell that a multipotent stem cell that is capable of generating (e.g., by differentiation or division) an endothelial cell. A progenitor cell that is capable of generating an endothelial cell may express this capability when grown under appropriate in vitro or in vivo conditions, such as those described herein.

By “progeny” is meant a cell derived from a multipotent stem cell of the invention. Progeny include without limitation progenitor cells, differentiated cells, and terminally differentiated cells.

By “derived from” is meant “obtained from” or the process of obtaining a progeny cell.

By “reduces” is meant a negative alteration of at least 10%, 25%, 50%, 75%, or 100%.

By “reference” or “control” is meant a standard condition. For example, an untreated or healthy (nondiseased) cell, tissue, or organ that is used as a reference.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, or at least about 25 amino acids. The length of the reference polypeptide sequence can be about 35 amino acids, about 50 amino acids, or about 100 amino acids. For nucleic acids, the length of the reference nucleic acid sequence will generally be at least about 50 nucleotides, at least about 60 nucleotides, or at least about 75 nucleotides. The length of the reference nucleic acid sequence can be about 100 nucleotides, about 300 nucleotides or any integer thereabout or therebetween.

A “somatic” cell refers to a cell that is obtained from a tissue of a subject. Such subjects are at a post-natal stage of development (e.g., adult, infant, child). In contrast, an “embryonic cell” or “embryonic stem cell” is derived from an embryo at a pre-natal stage of development.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., at least about 37° C., and at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., and at least about 68° C. In one embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, at least 80%, at least 85%, at least 90%, at least 95% or even at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “self-renewal” as used herein refers to the process by which a stem cell divides to generate one (asymmetric division) or two (symmetric division) daughter cells with development potentials that are indistinguishable from those of the mother cell. Self renewal involves both proliferation and the maintenance of an undifferentiated state.

The term “stem cell” is meant a pluripotent cell or multipotent stem cell having the capacity to self-renew and to differentiate into multiple cell lineages.

By “subject” is meant a mammal, including, but not limited to, a human or non-human mammal, such as a non-human primate, bovine, equine, canine, ovine, rodent, or feline. In a particular embodiment, a subject is a human subject, such as a human patient.

Ranges provided herein are understood to be shorthand for all of the values within the range, inclusive of the first and last values. By way of nonlimiting example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “tissue” is meant a collection of cells having a similar morphology and function.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “vascularized” is meant having a blood vessel. In some embodiments, the pancreatic islet organoid or pancreatic organoid is vascularized.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided and described herein.

### BRIEF DESCRIPTION OF THE DRAWINGS

FIGS. 1A-1G provide images, a schematic diagram, and graphs related to enhancement of the functionality of hiPSC-derived β-like cells via cellular crosstalk in polymer-based cultures. FIG. 1A (top) shows the results of a Principal Component analysis of transcriptomes from human iPSCs (hiPSCs), primary human pancreatic epithelial cells (hPanc Epithelial), human adipose-derived stem cells (hADSCs), human pancreatic fibroblasts (hPanc Fibroblast), human umbilical vein endothelial cells (HUVECs) and human pancreatic microvascular endothelial cells (hPanc Endothelial). FIG. 1A (bottom) shows a time course of human adipose-derived stem cell (hADSC) culture in Matrigel (1:1 dilution in hADSC medium, 2 million cells in 300 μl) showing intrinsic self-organization (Scale bar 1 mm). FIG. 1B shows a schematic of the generation of multicellular islet-like spheroids (MCS) and islet-like spheroid (IS). hiPSC-derived endocrine progenitor cells (EP) were co-cultured with hADSC and endothelial cells (ECs, HUVECs) in gellan gum-based 3D culture system (left). EPs are multipotent cells that differentiate into endocrine cells including α, β, δ, ε, pancreatic polypeptide and G cells, as defined by the expression of neurogenin 3, neurod1, Nkx2.2 and Pax4 biomarkers (Rezania, A. et al., 2014, Nature Biotechnology, 32:1121-1133). MCS generated in the matrigel environment show the incorporation of ECs (mCherry expression) and insulin expression as detected by Green Fluorescent Protein (GFP) expression, right). (Scale bar 100 μm). FIG. 1C illustrates multicellular islet-like spheroids (MCS) cultured in the 3D gellan gum system showing insulin expression (GFP, upper panel). Electron microscopy images of MCS showing insulin granules (lower right) and lipid droplets in hADSC (lower right). FIG. 1D presents graphs of gene expression in sorted insulin-expressing cells (GFP⁺) in islet-like spheroids (IS; hiPSC derived β-like cells generated in the absence of hADSCs and ECs), MCSs, or human islets (hislets). FIG. 1E presents a graph demonstrating human c-peptide secretion in response to 3 mM (G3) or 20 mM (G20) glucose from IS, MCS and hislets. FIG. 1F presents a graph demonstrating random fed blood glucose levels in STZ-induced diabetic NOD-SCID mice after sham treatment or transplantation of MCS (500) or human islets. FIG. 1G presents a graph demonstrating serum human c-peptide levels during feeding, fasting, and refeeding cycles in mice from 4 weeks after transplantation. Error bars represent±SEM. *p<0.05, **p<0.01, ***p<0.001.

FIGS. 2A-2F provide a heat map, graphs and plots demonstrating the expression of non-canonical Wnts in endocrine and supportive cells in human islets. FIG. 2A presents a heatmap of expression changes during hADSC culture in Matrigel. A significantly affected gene ontology category is presented at the right, namely, Wnt5a and downstream signaling (5.1e-03). FIG. 2B presents a graph showing tSNE clustering of temporal expression of WNTs during hADSC self organization as shown in FIG. 2A. FIG. 2C presents a graph and heatmap showing relative expression of WNTs in human islets (n=5). FIG. 2D shows t-SNE clustering of human islet single cell transcriptomes (n=3245). Annotated cell types are assigned based on known marker gene expression. FIGS. 2E and 2F show a single cell plot and violin plots, respectively, of WNT2B, WNT4, WNT5A, WNT7A, WNT7B and WNT9A expression in human islets. Error bar represents±SEM.

FIGS. 3A-3K provide schematics, images, heatmaps and graphs related to the generation of human islets like organoids (HILOs) and the induction of functional maturation of HILOs by WNT4. FIG. 3A presents a schematic of human islet-like organoid (HILO) generation. FIG. 3B shows representative images of HILOs in 3D culture (left) and insulin expression (human insulin promoter driven GFP (right, scale bar 100 μm). FIG. 3C depicts electron microscopy images showing insulin and glucagon granules in β and α cells, respectively, in WNT4-treated HILOs (“wHILOs”) and human islets. Scale bar, 1 μm. FIG. 3D-1 presents a heatmap of relative expression of key islets genes in hiPSCs, HILOs treated with PBS (P) or WNT4 (W), and in human islets (log 2 expression with Z-score). FIG. 3D-2 presents plots showing the relative expression of ISL1, SYT4, PDX1, GCK, NEUROD1, NKX2-2, INSULIN, NKX6-1, MAFA, MAFB and UCN3 in wHILOs and human islets as determined by qPCR (n=8 per sample type). FIG. 3E is a gene ontology map of genes that are up- and down-regulated in HILOs by treatment with WNT4 (100 ng/ml from day26 to day33). FIG. 3F shows the relative expression of ERRγ, NDUFA7 and COX7A2 in HILOs treated with increasing concentrations of WNT4 (0, 10, 25, 50, 200 ng/ml) for 5 days. FIG. 3G presents a heatmap of relative expressions of oxidative phosphorylation genes in 3D cultured hiPSCs, HILOs with PBS and HILOs with WNT4 treatment (wHILOs), and human islets (Z-Score). FIG. 3H is a graph demonstrating oxygen consumption rates (OCRs) measured in hiPSC spheroids on day 0 (upside down triangle), PBS treated HILOs (upright triangle), WNT4 treated HILOs (square) and human islets (circle). FIG. 3I presents a graph showing in vitro human c-peptide secretion in response to 3 mM (G3) or 20 mM (G20) glucose or 20 mM KCl (K₂O) from HILOs generated with and without WNT4 treatment. FIG. 3J presents a cartoon schematic depicting culture conditions for commercially available hiPSC-derived β-like cells (left) and light microscopy images of cultured cells (right). FIG. 3K presents a bar graph showing in vitro c-peptide secretion in response to 3 mM (G3) and 20 mM (G20) glucose from cultures described in FIG. 7D-2.

FIGS. 4A-4M provide plots, graphs, a microscopy image, flow cytometry results and a schematic related to studies of PD-L1-expressing wHILOs extended functionality and glucose control in immune competent mice and immune profiling of wHILO grafts in C57BL6J mice. FIG. 4A shows tSNE clustering of single cell transcriptomes from WNT4 treated HILOs (wHILOs, n=4840). FIG. 4B is a graph showing relative cell type populations in HILOs and human islets. FIG. 4C presents a graph demonstrating random fed blood glucose levels after transplantation of wHILOs with or without PD-L1 expression (in kidney/kidney capsule of induced diabetic C57BL6J mice (n=11 and 9 mice, respectively). The top plot on the graph represents wHILOs (−); the middle plot on the graph represents wHILOs (PD-L1 expression); the bottom plot on the graph represents mislets. FIG. 4D presents flow cytometric analysis of insulin-expressing and mouse immune (CD45+) cells recovered from kidney capsule grafts 27 days after transplantation of wHILOs with and without PD-L1 expression. Grafts containing HILOs expressing PD-L1, which can potentially bind to PD-1 on T cells (e.g., CD45+ cells), thereby suppressing T cell activation and killing activity, show fewer infiltrating CD45+ T cells compared with grafts containing HILOs that do not express PD-L1. FIG. 4E shows the quantification of the analysis of blood glucose levels in STZ-induced diabetic mice after transplantation of wHILOs with or without PD-L1 expression, as shown in FIG. 4D (Error bars represent±SEM. *p<0.05, **p<0.01, ***p<0.001). FIG. 4F presents a flow cytometry analysis of insulin expressing and mouse immune (CD45⁺) cells recovered from kidney capsule grafts 27 days after transplantation of wHILOs with and without PD-L1 expression. CD45⁺ cells were further categorized as B cells (CD19⁺), T cells (CD3⁺) and NK cells (NK1.1⁺). FIG. 4G shows dot plots of the quantification of the analysis described for FIG. 4F (n=6 and 6). FIG. 4H shows an image of wHILO (PD-L1) cells in a kidney graft 27 days after transplantation (insulin promoter driven GFP expression). Scale bar, 100 μm Error bars represent±SEM. *p<0.05. FIG. 4I presents a schematic showing transplantation of wHILOs with and without PD-L1 overexpression (500 HILOs per mouse) into multi low dose streptozotocin (MLD-STZ, 50 mg/kg/day for 5 days) induced diabetic Hu-PBMC-NSG mice. FIG. 4J presents a flow cytometric analysis of human T cells (CD4⁺ and CD8⁺ cells in CD45⁺/CD3⁺ population) in PBMC from Hu-PBMC-NSG mice (n=15 mice) 3 weeks after human PBMC transplantation. FIG. 4K shows a graph of random fed blood glucose levels in MLD-STZ induced diabetic Hu-PBMC-NSG mice after transplantation of wHILOs with or without PD-L1 expression (n=6 and 6 mice). FIG. 4L shows a graph of serum human c-peptide levels in mice described in FIG. 4K. FIG. 4M presents a flow cytometric analysis of insulin-expressing and human CD45⁺ immune cells recovered from kidney capsule grafts 27 days after transplantation of wHILOs, with and without PD-L1 expression. FIG. 4N presents dot plot graphs that quantify the results of analyses shown in FIG. 4M. (Error bars represent±SEM. *p<0.05, **p<0.01, ***p<0.001).

FIGS. 5A-5K provide graphs and schematic diagrams demonstrating that immune tolerance is induced by epigenetic memory. FIG. 5A presents a graph showing PD-L1 expression in islet (wHILOs) cells sorted by flow cytometry based on insulin expression (GFP+ and GFP−, respectively) after IFNγ treatment (10 ng/ml, 12 hours). The GFP+ cells comprise β-like cells; the GFP-cells comprise alpha (α), delta (δ) and epsilon (ε) cells. FIG. 5B presents a graph showing temporal PD-L1 expression in wHILOs after a single IFNγ treatment (10 ng/ml, 2 hours). FIG. 5C is a schematic illustrating IFNγ (10 ng/ml) pulse treatment of wHILOs. (MPS treatment). FIG. 5D presents a graph showing PD-L1 expression induced by indicated cycles of IFNγ treatment, 7 days after last treatment. FIG. 5E presents a graph of PD-L1 protein levels 1 and 7 days after indicated IFNγ (10 ng/ml) treatments. PD-L1 overexpressing wHILOs (PDL1OE) and a single 12 h exposure to IFNγ was used as a positive control. FIG. 5F presents a dot plot showing human c-peptide secretion from IFNγ treated wHILOs in response to 3 mM (G3) or 20 mM (G20) glucose. FIG. 5G is a schematic illustrating IFNγ treatment in combination with an IL-1β treatment challenge (10 ng/ml for 24 hours) to induce β cell dedifferentiation. FIG. 5H presents a graph showing INS and UCN3 expression after the indicated IFNγ and IL-1 treatments (10 ng/ml, 24 hours) of wHILOs. FIG. 5I presents a schematic of an experimental protocol for in vivo transplantation of wHILOs into induced diabetic animals. High dose streptozotocin (HD-STZ, 180 mg/kg) induced diabetic C57BL6J mice received transplants of wHILOs that had or had not been subjected to the IFNγ treatment protocol shown in FIG. 5C, (n=6 and 6, 500 wHILOs/mouse). FIG. 5J presents a graph showing blood glucose levels in recipient mice (STZ-treated (180 mg/kg) diabetic C57BL6J mice) at day 17 following kidney capsule transplantation of wHILOs and IFNγ pulse stimulated wHILO (“immune evasive wHILOs” or “wHILO^{ie}”). FIG. 5K presents a graph showing serum human c-peptide levels in mice treated as described in FIG. 5I. Error bars represent±SEM. *p<0.05, **p<0.01.

FIGS. 6A-6F provide images, graphs and results related to multicellular spheroids (MCSs) as described herein. FIG. 6A shows a 3D gellan gum suspension of multicellular spheroids (MCS, top), light microscopy images of single MCS (lower left) and hislets (lower right). FIG. 6B shows images of insulin promoter driven GPF expression, and endothelial cells (EC, marked by mCherry expression) in MCS. FIG. 6C presents images showing the progressive development of vascular-like structures in MCSs that were cultured with endothelial growth media in the Matrigel system. FIG. 6D is a schematic for single cell RNA-seq analyses. FIG. 6E presents a heatmap of expression of the top 10 signature genes in human islet cell clusters from FIG. 2D. FIG. 6F present plots showing t-SNE_2 single cell expression of signature hormonal and cell type specific genes in human islets. Relative expression scale: low (0.5, least intense), to high (5, most intense).

FIGS. 7A-7F provide a schematic, graphs, images, and data related to the characterization of mature HILOs. FIG. 7A depicts a diagram of CRISPR-Cas9-based knockin for human insulin promoter driven GFP expression in hiPSC. FIG. 7B presents a differential interference contrast (DIC) image of wHILOs with insulin-GFP and UCN3-RFP expression (scale bar, 100 μm). FIG. 7C presents a Seahorse analysis of extracellular acidification rate (ECAR) measured in day 0 hiPSC spheroids (open square), HILOs (Vehicle/PBS-treated, filled triangle), wHILOs (Wnt4 treated, filled circle) and human islets (open circle). 20 mM glucose (Glu), oligomycin (Olig), Fccp, antimycin+Rotenon (Ant+Rot) were treated in order. FIG. 7D-1 presents a graph showing the kinetics of human c-peptide secretion from WNT4 treated HILOs in response to progressive exposure of the HILOs to 3 mM glucose, 20 mM glucose, 20 mM glucose+100 mM GLP-1, 3 mM glucose, and 3 mM glucose+20 mM KCl over time. FIG. 7D-2 presents a bar graph showing glucose stimulated human c-peptide secretion from wHILOs treated with and without XAV939 to promote β-catenin degradation (XAV939, 1 μM for 3 days). FIG. 7E presents data illustrating motif enrichment in chromatin regions with enhanced accessibility upon WNT4 treatment. FIG. 7F depicts chromatin accessibility at ERRγ target genes (determined by ATAC-Seq) in insulin expressing cells sorted from HILO treated with PBS or WNT4 for 7 days (fold change>1.5).

FIGS. 8A-8H provide images, graphs, a schematic and a diagram showing results related to WNT4 mediated insulin-GFP expression and WNT4-driven metabolic maturation. FIG. 8A presents representative images of mitochondrial content, indicated by MitoTracker (red) staining, in PBS and WNT4 treated HILOs (scale bar, 100 μm). FIG. 8B presents graphs of flow cytometry quantification of insulin expression (GFP) and mitochondrial content in HILOs treated with recombinant human WNT4 (rhWNT4), WNT5A (rhWNT5A), or conditioned medium (CM) from control or WNT5A overexpressing fibroblasts (n=3). Error bars represent±SEM. *p<0.05. FIG. 8C presents a gene ontology of transcriptional changes induced by WNT4 treatment (100 ng/ml WNT4 from day26 to day33) in HILOs. FIG. 8D presents a graph demonstrating blood glucose levels in STZ-induced diabetic NOD-SCID mice after transplantation (TP) of 500 wHILOs or hislets, or sham surgery was performed at day 3 (n=7, wHILOs; n=6, hislets; n=3, Sham). Error bars represent±SEM. *p<0.05. FIG. 8E presents a Venn diagram showing overlap between WNT4-induced increases in chromatin accessibility in GFP⁺ cells and increases in HILO gene expression (upper panel), and gene ontology pathways enriched in the intersection gene set. FIG. 8F shows motifs that are enriched in the intersection gene set shown in FIG. 8E. FIGS. 8G and 8H demonstrate the results of experiments in which postnatal islets (day P11-14) from WT and β cell specific ERRγKO mice were cultured with or without rhWNT4 (100 ng/ml) for >5 days. FIG. 8G shows relative gene expression measured by qPCR, and FIG. 8H shows insulin secretion in response to 3 mM and 20 mM glucose. *p<0.05, ***p<0.001. For FIGS. 8G and 8H, postnatal islets (day P11-14) from WT and β cell specific ERRγKO mice were cultured with or without rhWNT4 (100 ng/ml) for >5 days.

FIGS. 9A-9M provide microscopy (confocal) images, plots, heatmaps and graphs demonstrating immunofluorescent characterization of wHILOs, flow cytometry analysis of HILOs, and single cell analysis of wHILOs. FIGS. 9AB, 9C and 9D present confocal images of wHILOs stained for C-peptide. FIG. 9A shows representative immunofluorescent staining results for glucagon, somatostatin and pancreatic polypeptide (PP) in wHILOs. FIG. 9B presents confocal images of wHILOs stained for C-peptide. FIG. 9C presents confocal images of wHILOs stained for β cell enriched markers NKX2-2, NKX6-1, MAFA, MAFB, PDX1. Images are representative of three independent experiments. FIG. 9D presents confocal images of wHILOs stained for endocrine markers chromogranin A (CHGA), Synaptophysin (red, middle images) with Insulin-GFP (green, left images) visualization. Hoechst nuclei staining is shown in the right (Merge) panels. Scale bar, 100 μm. Images are representative of three independent experiments. FIG. 9E shows representative flow cytometry results for ß cell and endocrine marker co-staining in HILOs with and without WNT4 treatment. FIG. 9F graphically depicts the quantification of results presented in FIG. 9E (n=6 and 6). FIG. 9G shows tSNE clustering of single cell transcriptomes from WNT4 treated HILOs (wHILOs, n=4840). FIGS. 9H and 9I show Violin Plots (9H) and single cell expression (9I) of INS, CHGA, SOX9, HES1 in wHILOs. FIG. 9J shows expression of ß cell-enriched (INS, PDX1, NKX6-1, NKX2-2, NEUROD1, NPTX2, ITGA1, PCSK1, MAFA, MAFB, UCN3, CHGA), a cell-enriched (GCG, ARX) and 6 cell-enriched genes (SST, RBP4) overlaid on tSNE clustering. FIG. 9K presents a heatmap of the top 10 differentially-expressed genes in each cell cluster. FIG. 9L presents tSNE clusters according to cell type (Panc P=pancreatic progenitor, Rep=replicating, UK=unknown). FIG. 9M presents tSNE clustering of combined HILOs and wHILO single cell data sets (right panel) and clustering analysis-defined cell types.

FIGS. 10A-10C provide plots showing quality analyses of scRNA-seq. FIG. 10A shows plots illustrating a correlation of number of detected genes and UMIs in HILO, wHILO and human islets. FIG. 10B presents tSNE clustering of combined wHILO (blue dots, n=4840) and human islet (red dots, n=3245) single cell transcriptomes (left panel) and clustering analysis-defined cell types (left). FIG. 10C shows the expression of endocrine specific genes (INS, NKX2-2, GCG, SST, PPY), duct marker (KRT19) and stellate cell marker (ACTA2) in tSNE visualization of merged single cell data sets for wHILO and hislets.

FIGS. 11A-11D provide a schematic depiction, graphs and plots related to plate based scRNA-seq analysis. FIG. 11A is a scheme of plate based scRNA-seq. Dissociated single cells from wHILO were sorted by FACS into 96 well tissue culture plate (microplate). FIGS. 11B and 11C: A box plot showing average gene counts per cells (FIG. 11B) and identification of 45 single cells with high quality gene detection (FIG. 11C). FIG. 11D illustrates that single cell RNA-seq revealed single hormone expressing insulin, glucagon, somatostatin cells in wHILOs.

FIGS. 12A-12F provide graphs and images related to PD-L1 gene and protein expression in R cells and HILOs. FIG. 12A (left) shows tSNE endogenous expression of PD-L1 in human islet cells (β cells are outlined), and (right) a heatmap of the top differentially expressed genes between PD-L1+ and PD-L1− R cells. FIG. 12B presents immunohistochemistry results overlap of lentiviral-driven PD-L1 expression and insulin promoter-driven GFP expression in wHILOs (scale bar, 100 μm). FIG. 12C presents bar graphs showing human PD-L1 expression (left) and human insulin expression (right) in wHILOs, with and without lentiviral PD-L1 overexpression, as measured by qPCR. FIG. 12D (top) presents a schematic depiction of an in vivo experimental study conducted in induced diabetic C57BL6J mice. High dose streptozotocin (HD-STZ, 180 mg/kg) induced diabetic C57BL6J mice received transplants of wHILOs with and without PD-L1 overexpression (n=500), or mouse islets; FIG. 12D (bottom) shows results following transplantation of PD-L1-overexpressing wHILOs into the kidney capsule of STZ-induced diabetic mice. FIG. 12E presents a bar graph showing PD-L1 expression in wHILOs 12 hours after indicated IFNγ stimulation. Error bars represent±SEM. ***p<0.001. FIG. 12F presents a bar graph showing PD-L1 gene expression in human islets 12 hours after INFγ (ng/ml) stimulation. Error bars represent±SEM. ***p<0.001.

FIG. 13 provides a schematic diagram of the strategy for generation of mature, immune evasive wHILOs (wHILO^{ie}s).

FIGS. 14A-14D present a Venn diagram, heatmap, gene ontology chart and browser track related to studies investigating IFNγ-induced changes in wHILOs. FIG. 14A shows a Venn diagram of differentially regulated genes upon acute (12 h at 10 ng/ml) and multi pulse stimulated (MPS), (2 h at 10 ng/ml for 3 days) IFNγ treatment of wHILOs. In the diagram, the leftmost circle represents “MPS IFNγ treatment” and the rightmost circle represents “acute IFNγ treatment.” FIG. 14B shows a heatmap of differentially expressed genes upon acute and MPS IFNγ stimulation. Sustainable PD-L1 genes expression by MPS are highlighted. FIG. 14C shows gene ontology of selectively regulated genes upon MPS-IFNγ (top panel) and acute IFNγ (bottom panel) treatments. FIG. 14D shows panels of browser tracks indicating chromatin accessibility at selected genes 7 days after the last IFNγ treatment in the MPS method, or 12 hours after acute IFNγ stimulation in wHILOs.

FIGS. 15A-15C present a schematic, graph and flow cytometry plots related to studies demonstrating the immune evasiveness of wHILOs by enhanced endogenous PD-L1 expression. FIG. 15A shows a schematic of a treatment regimen involving multi low dose streptozotocin treatment (MLD-STZ, 50 mg/kg/day for 5 days) of Hu-PBMC-NSG mice to produce an immune competent diabetic animal model. MPS induced PD-L1 expressed wHILOs (n=500) were transplanted under kidney capsule. FIG. 15B shows a graph of random fed blood glucose levels in STZ-induced diabetic Hu-PBMC-NSG mice after transplantation of wHILOs that had undergone or had not undergone MPS (n=6 mice, respectively). wHILOs (−) data from FIG. 4K and FIG. 4G are replicated, since those experiments were performed parallely. FIG. 15C shows a flow cytometry analysis of insulin-expressing and human immune (CD45⁺) cells recovered from kidney capsule grafts 27 days after transplantation of wHILOs with or without MPS. Error bars represent±SEM. *p<0.05, **p<0.01, ***p<0.001.

### DETAILED DESCRIPTION OF THE EMBODIMENTS

Featured herein are methods and systems for the generation and utilization of stem cell-derived human islets and human islet-like organoids, which provide a promising strategy for the therapeutic treatment of diseases and pathologies, such as pancreatic diseases and insulin dependent diabetes, a disease caused by the loss of endogenous insulin-producing R cells. Advantageously, the methods and systems as described can generate biological products, e.g., cells, human islet-like organoids and cells thereof, as therapeutics that can alleviate the shortage of donor-matched cadaveric human islets, which are currently being used to treat patients.

As described herein, functional human islet-like organoids (HILOs) are generated from human pluripotent stem cells, such as induced pluripotent stem cells (iPSCs). In an embodiment, a culture system which allows for non-canonical WNT4 signaling is employed to generate HILOs. Without wishing to be bound by theory, WNT4 signaling in cells such as iPSCs, human islet and HILO cells drives metabolic maturation necessary for robust glucose stimulated insulin secretion (GSIS). The stem-cell derived islets and HILOs as described herein achieve functional maturity and exhibit robust, glucose-stimulated insulin secretion (GSIS) through enhanced glucose-responsive oxidative capacity, which is regulated by the WNT4-ERR (Estrogen-Related Receptor) metabolic pathway. The functionally mature HILOs contain endocrine-like cell types that, upon transplantation, rapidly re-establish glucose homeostasis in diabetic NOD-SCID mice (e.g., Examples 4 and 5). In an embodiment and as described herein, the HILOs and cells thereof avoid rejection by immune cells under immune-competent conditions.

In an aspect, single cell RNA (scRNA)-sequencing analysis of functional HILOs, as well as human cadaveric islets, revealed transcriptional heterogeneity of HILO-derived cells, including a small population of immune-evasive β cells. As described in an aspect herein, HILOs were molecularly engineered to express a checkpoint protein, e.g., PD-L1, in order to mimic the transcriptional program of immune-evasive β cells. When the PD-L1-expressing HILOs were assessed, it was found that PD-L1 expression overcame autoimmune rejection of the HILOs, which had been transplanted in immune-competent mice with type 1 diabetes. Thus, the generation, in a scalable fashion, of functional β cells and HILOs that can avoid immune detection, autoimmune activation, and transplant or implant rejection afford advantageous and beneficial treatments and therapies for diabetes, in particular, type 1 diabetes and late stage type 2 diabetes. In an embodiment, β cells, human HILOs and human islets are molecularly engineered (e.g., transduced or transfected) to express a checkpoint protein such as PD-L1. In an embodiment, β cells, human HILOs and human islets are induced to express the PD-L1 protein as described herein.

Methods of Protecting Islets, Organoids and the Cells Therein from Immune Surveillance and Immune Cell Killing and Clearance

In an aspect, methods, particularly in vitro or ex vivo methods, are provided for generating islets and organoids, including the cells therein, (e.g., donor cells, islet and organoid cells) that survive, have reduced cell death and/or can better evade immune detection by cells of the immune system, especially after transplantation, implantation, or transfer into a subject, such as a recipient individual. In an embodiment, the transplantation, implantation, or transfer involves allogeneic cells, islets, and/or organoids that survive and have reduced killing and detection by immune cells, e.g., T cells, β cells, monocytes, macrophages and the like, subsequent to the practice of the methods described herein.

In an aspect, the expression (or upregulated expression) of a checkpoint protein-encoding gene and/or its encoded product, in particular, PD-L1 and/or the PD-L1 protein, in or by IFNγ receptor-expressing islets, organoids (e.g., HILOs), or cells (e.g., β cells of HILOs) following multiple intermittent exposures to interferon gamma (IFNγ) over a given time period (such as at least 24 hours) allows the HILOs to maintain glucose homeostasis, e.g., in immune-competent diabetic mice for a long time period, e.g., at least 50 days, as well as to evade an immune response by activated T cells and/or graft rejection. In an embodiment, the islets, organoids, or cells are human islets, organoids, or cells. In embodiments, such islets, organoids, or cells express IFNγ receptors and/or are responsive to treatment with IFN γ. In an embodiment, the islets, organoids, or cells naturally express IFNγ receptors. In an embodiment, IFNγ receptors may be introduced into the islets, organoids, or cells, for example, without limitation, by recombinant, viral, or molecular biology techniques as known and practiced in the art. In an embodiment, PD-L1 gene and/or protein expression (or upregulated expression) in the IFNγ receptor-expressing islets, organoids, and cells constitutes a detectable marker, which is indicative of the response of the islets, organoids, and cells to IFNγ exposure. PD-L1 expression or upregulated expression of PD-L1 as a marker of IFNγ responsiveness following exposure of islets, organoids, and cells to IFNγ may be assayed by polynucleotide and/or protein detection methods routinely used and known in the art, and are not intended to be limiting.

In embodiments, the method comprises stimulating the cells with interferon gamma (IFNγ) in low amounts or doses, e.g., 0.5-100 ng/ml, 1-50 ng/ml, 1-25 ng/ml, 1-20 ng/ml, 1-10 ng/ml, 10 ng/ml or 20 ng/ml. In an embodiment, this is achieved by subjecting the islets, organoids, and/or cells, e.g., HILOs, to IFNγ for discrete time periods, e.g., 1, 2, 3, 4, 5, 6, 7, 8, 9, 10, 11, 12, 13, 14, or 15 hours, or more, in particular, for about or equal to 2 hours or 12 hours, for example, multiple times, e.g., 2 times, 3 times, 4 times, 5 times, 6 times or more, over a given time period. In some embodiments, the multiple exposures or pulses are performed over at least a 24-hour period of time (about 1 day), a 48-hour period, a 72-hour period, or over the course of 4, 5, 6, 7, 8, 9, or 10 days. In some embodiments, the cells are exposed to IFNγ for a total of 0.5-3 hours, 0.5-4 hours, 0.5-5 hours, 0.5-6 hours, 0.5-7 hours, or 0.5-10 hours. Between IFNγ exposures or pulses the cells are allowed to ‘rest,’ e.g., in culture medium or 3D matrix, in the absence of IFNγ between the time periods of exposure to IFNγ. In some embodiments, the cells are allowed to ‘rest’ in the absence of IFNγ for at least about 0.5, 1, 2, 3, 4, 5, 6, 7, 8, 9, 10, 11, 12, 13, 14, 15, 16, 17, 18, 19, 20, 21, 22, 23, or 24 hours between exposure to IFNγ. In other embodiments, the cells are allowed to ‘rest’ in the absence of IFNγ for about 1, 2, 3, 4 or 5 days. In one embodiment, the IFNγ treatment causes a constitutive (prolonged) upregulation and expression (and maintenance) of PD-L1 expression in the islets, organoids, and/or cells, e.g., HILOs. This procedure involves multiple pulse stimulation (MPS), also referred to as intermittent exposure, of cells, islets, organoids, e.g., HILOs or islets and the cells therein, to IFNγ. Expression of PD-L1 by the cells, islets, and/or organoids, is long-lasting following MPS, particularly, if the islets, organoids, and/or cells (e.g., HILOs) experience at least 3 pulses or intermittent exposures to IFNγ (e.g., 10 ng/ml) for about or equal to a 2-hour time period per pulse of IFNγ. For example, by this regimen, sustained expression of PD-L1 is found in the islets, organoids and/or cells, e.g., HILOs, for at least 7 days following subjecting the islets, organoids and/or cells, e.g., HILOs, to the MPS procedure. In an embodiment, islets, organoids, (e.g., HILOs), or cells generated by the method survive in a recipient subject following transplantation, implantation, or transfer for at least about or equal to 50 days.

Without wishing or intending to be bound by theory, the MPS IFNγ exposure procedure results in PD-L1 expression (or upregulation of PD-L1 expression) in islets, organoids and/or cells (e.g., HILOs and the cells therein (e.g., β cells)), which involves a mechanism of transcriptional memory. The described procedure comprising MPS IFNγ exposure of cells, islets, and/or organoids may stimulate or create an intracellular signaling cascade in which the de-differentiation of the cells, islets and/or organoids is inhibited or blocked. The short pulses of IFNγ (MPS IFNγ) to which the cells, islets or organoids are exposed in the methods may ultimately involve an alteration of chromatin structure, thereby protecting the cells, islets or organoids from de-differentiation and affording the MPS IFNγ exposed cells, islets, or organoids, with the ability to survive (e.g. by reduced cell death by cells of the immune system), as well as to be immune to the effects of inflammatory cytokines and chemokines, e.g., Interleukin-1B (IL-1B) as described infra, so as to provide an anti-inflammatory effect for the cells, islets, or organoids. The absence or reduction of inflammation associated with MPS IFNγ exposed cells, islets, or organoids generated from the described methods may enhance their potential for survival and reduction in killing by immune cells post transplantation, implantation, or transfer into a subject. The described methods thus generate donor cells, islets and organoids that have improved survival and retain their functionalities following transplant, implant, or transfer into a subject and offer a number of beneficial advantages in their use as therapeutics.

In a particular embodiment, a method is provided for generating human islets, organoids (e.g., HILOs) and various primary or differentiated cells (of different lineages) that survive, have reduced cell death, and can better evade immune detection or autoimmunity in which the method involves (a) contacting the human islets, organoids (e.g., HILOs), or cells with interferon gamma (IFNγ) for greater than one hour at a predetermined time point; repeating step (a) at least about two times during a given time period, e.g., a time period of about or equal to 72-hours; wherein the human islets, organoids (e.g., HILOs), or cells are maintained in the absence of IFNγ between times of contact with IFNγ; and wherein steps (a) and (b) induce sustained expression of PD-L1 in the human islets, organoids (e.g., HILOs), or cells. In an embodiment of the method, the human islets, organoids (e.g., HILOs), or cells are contacted with IFNγ for a time period of about or equal to at least 1 hour, or at least 2 hours, or more than 2 hours in step (a). In a particular embodiment of the method, the human islets, organoids (e.g., HILOs), or cells are contacted with IFNγ for a time period of about or equal to 2 hours or about or equal to 12 hours in step (a). In another particular embodiment of the method, step (a) is repeated three times for at least about or equal to 2 hours each time in the given time period, e.g., an about or equal to 72-hour time period. In another embodiment of the method, the human islets, organoids (e.g., HILOs), or cells are washed to remove the presence of IFNγ between step (a) and step (b). In another embodiment of the method, IFNγ is used in an amount of 1-25 ng/ml. In another embodiment of the method, IFNγ is used in an amount of 10 ng/ml. In another embodiment of the method, PD-L1 expression in the human islets, organoids (e.g., HILOs), or cells is maintained following step (b) for greater than about or equal to 7 days. In an embodiment, the method generates human cadaveric islets (e.g., syngeneic or allogeneic) that are protected from destruction or clearance by the immune system.

In another particular aspect, a method of generating various cells, islets, or organoids (e.g., HILOs), including human cells, islets, or organoids, that survive, have reduced cell death, and/or evade immune detection or autoimmunity is provided in which the method involves (a) contacting the cells, human islets, or organoids (e.g., HILOs) with interferon gamma (IFNγ) in an amount of about 1 ng/ml to 25 ng/ml for greater than 1 hour at a first time point during a given time period, e.g., a time period of about or equal to 24-hours; and (b) contacting the cells, human islets, or HILOs with IFNγ in an amount of about 1 ng/ml to 25 ng/ml for greater than about or equal to 0.5 hours or more, or about or equal to 1 hour at at least two additional time points during a following time period, e.g., a 48-hour time period, following step (a); wherein the islets or organoids (e.g., HILOs) are washed and rested in medium in the absence of IFNγ between being contacted with IFNγ; and wherein steps (a) and (b) induce sustained expression of PD-L1 in the islets or organoids (e.g., HILOs). In a particular embodiment of the method, the cells, islets, or organoids (e.g., HILOs) are contacted with IFNγ in an amount of 10 ng/ml for at least 2 hours in step (a) and step (b). In another particular embodiment of the method, the cells, islets, or organoids (e.g., HILOs) are contacted with IFNγ for at least about or equal to 2 hours at 3 time points (different time points) during a 72-hour time period.

The practice of the above-described methods for immune evasion of IFNγ receptor-expressing islets, organoids, and cells provide advantages for such islets, organoids and cells, particularly, human cells, islets and organoids, used for transplants, implants, or transfer from one subject to another as therapeutics and therapeutic treatment of diseases, disorders and pathologies. The practice of the described methods provides immunoprotection and enhanced survival of islets, organoids and cells that are transplanted, implant, or transferred into a recipient subject (e.g., an adoptive recipient, transplant recipient, and the like), such that the transplanted, implanted, or transferred islets, organoids, or cells are maintained and are functional in the recipient for several days, or weeks, or longer, for example, for about 2 days or longer to 1, 2, 3, 4, or more weeks, or longer.

The methods and systems described herein are suitable for use with a variety of cells and cell types, or donor cells for transplantation, particularly, IFNγ receptor-expressing cells, derived from different lineages, as well as islets, and organoids, e.g., to provide immune protection after transplant, implant, administration or transfer into a recipient subject. In general, by way of nonlimiting example, stem cells, primary cells, differentiated cells of various lineages and types, or cells of one type derived from cells of a different source may be used. In embodiments, such suitable cells express IFNγ receptors and/or are responsive to treatment with IFN γ may be used in accordance with the above-described methods. Responsiveness to IFNγ treatment in the described methods may be determined or identified by assaying for detectable expression of PD-L1 or the PD-L1 protein by the IFNγ receptor-expressing cells, islets, or organoids (and cells therein).

By way of particular, yet nonlimting, example, the methods described herein, which involve induction of sustained PD-L1 expression by IFNγ MPS, may be suitable or applicable for use with a variety of cells and cell types, or donor cells for transplantation, including, without limitation, cardiac cells, colon cells, kidney cells, bladder cells, liver cells (hepatocytes), gastrointestinal cells, gastric (stomach) cells, lung cells, ovarian cells, cervical cells, uterine cells, testicular cells, pancreatic cells, pancreatic β cells, muscle cells, hematopoietic cells, immune cells (β cells, T cells), retinal cells, corneal cells, brain cells, chimeric antigen receptor-T cells (CAR-T cells), bone marrow cells, e.g., mononuclear cells, neurons, neuronal cells, insulin-producing pancreatic β cells derived from human skin cells (e.g., as reported by Li, K. et al., 2014, Cell Stem Cell, 14(2):228-236); umbilical cord blood (UCB) cells, adipose derived mesenchymal stromal (stem) cells, cardiac stem cells, colon stem cells, kidney stem cells, liver (hepatocyte) stem cells, gastrointestinal stem cells, gastric (stomach) stem cells, lung stem cells, pancreatic stem cells, pancreatic β stem cells, muscle stem cells, hematopoietic stem cells, immune cell (T cell or B cell) stem cells, bone marrow stem cells, CD133+ stem cells, CD34+ hematopoietic cells, CD34+ stem cells, mesenchymal stem cells, umbilical cord mesenchymal stem cells, retinal stem cells, neuronal stem cells, and the like, as well as islets and organoids generated from or containing such cells. By way of example, the following types of organoids are suitable for use in the methods: intestinal organoids, hepatic organoids, neural organoids, pulmonary organoids, for example, as may be produced using art-described procedures, or commercially available, e.g., Stemcell™ Technology, Cambridge, MA.

Other suitable cells are those derived from embryonic stem cells which give rise to various differentiated cell types, for example, ectoderm-derived cells, such as neuronal cells, dopaminergic neuronal cells (e.g., immortalized dopaminergic neuronal precursor cells (LUHMES) commercially available from abm, Vancouver, British Columbia); comeal-derived cells (e.g., normal human comeal epithelial cells, commercially available from LifeLine Cell Technology, Oceanside, CA); endoderm-derived cells, such as liver cells (e.g., human hepatocytes wild type, available from DefiniGEN, Cambridge, UK); and mesoderm-derived cells, such as muscle cells, bone marrow cells, kidney cells and skeletal muscle cells (e.g., human skeletal muscle cells (skMDC), commercially available from Cook MyoSite®, Pittsburgh, PA). Nonlimiting examples of β cells (e.g., having pancreatic β-cell characteristics/function) or islets which may be used in the described methods may be found, for example, in WO 2016/100898, WO 2016/100909, WO 2016/100921, WO 2016/100925, WO 2016/100930, WO 2014/145625.

Accordingly, the methods, systems and compositions as featured and described herein are useful and applicable for generating cells, tissues and organoids, which exhibit long-lasting viability and functional activity following administration, e.g., via transplantation, implantation, injection, and the like, to a subject in need thereof, based on the sustained expression of a checkpoint protein, such as PD-L1 by the cells, tissues and organoids, and their resultant evasion of and protection from immune surveillance and destruction by cells of the immune system, e.g., as occurs in graft versus host reaction.

In a particular aspect, the methods, systems and compositions as featured and described herein are useful for generating in vitro scalable, functional, vascularized organoids, particularly human pancreatic or pancreatic islet organoids (HILOs), that can evade immune detection following transplantation or implantation. In an embodiment, the culturing of iPSC-derived beta-like cells, which express an immune checkpoint protein, with human adipose-derived stem cells (hADSC) and human umbilical vein endothelial cells (Huvec) in a three-dimensional matrix containing gellan gum generated functional pancreatic and pancreatic islet organoids is also provided.

The HILOs generated in accordance with the described methods were vascularized and exhibited functional properties, such as glucose-stimulated insulin secretion (GSIS). While recent studies have reported the possibility of generating glucose-responsive, insulin-producing, beta-like cells from human Pluripotent Stem Cells (PSCs), the generation of functional, vascularized pancreatic islets organoids from PSCs that secrete insulin, glucagon and somatostatin in response to nutrients and that are capable of evading immune detection and graft or transplantation or implantation rejection by cells of the immune system for substantial periods of time is advantageously provided herein.

As described herein, the self-organizing function of human adipose-derived stem cells (hADSC), HUVEC, and human iPSC-derived beta-like cells allows for the in vitro generation of glucose-responsive insulin secreting islet-like organoids with the ability to form functional vasculature. In addition, successful scaling of islet-like organoids production through the use of Gellan gum based 3D culture systems is achieved. Using a Gaussia luciferase reporter to measure insulin secretion, the functional heterogeneity in hiPSC-derived islet-like organoids was characterized. Without intending to be bound by theory, results herein suggest that the human islet-like organoids (HILOs) which express a checkpoint protein may offer a beneficial therapeutic treatment for diabetes and a new treatment for organ failure, as well as a platform for drug screening, genome editing, and the modeling of organogenesis and pathogenesis of diabetes.

### Immune Checkpoint Proteins

Maintaining immune homeostasis is critical for host survival. Overt or uncontrolled immune responses to pathogens or to mutated, modified, or over-expressed self-antigens can cause inflammatory tissue damage and autoimmune diseases. To prevent this, the breadth and magnitude of the immune response is regulated by a balance between co-stimulatory and inhibitory signals. These signals are collectively referred to as immune checkpoints, which are necessary for maintaining self-tolerance and protecting a subject from tissue damage.

Activated T cells are the primary mediators of immune effector functions and as such, they express multiple co-inhibitory receptors such as, e.g., lymphocyte-activation gene 3 (LAG-3), programmed cell death protein 1 (PD-1) and cytotoxic T-lymphocyte-associated protein 4 (CTLA-4). These immune checkpoint molecules have been shown to modulate T cell responses to ‘self’ proteins, as well as to chronic infections and tumor antigens. Of note, the pathways utilized by these checkpoint proteins are unique and non-redundant, thus, reflecting the important role of immune checkpoints in regulating immune homeostasis,

As noted supra, an immune checkpoint protein” or “immune checkpoint molecule,” or simply, “checkpoint protein or molecule” is a protein or molecule that regulates the immune system and frequently binds to or interact with ligands (cognate ligands), which may cause a given effect, e.g., cell stimulation, anergy, or apoptosis. In an embodiment, the immune checkpoint protein is one that binds a cognate ligand (e.g., a receptor ligand) on the membrane surface of an immune cell, e.g., a T cell surface receptor. In a specific embodiment, an immune checkpoint protein is PD-L1 or a binding portion thereof, where the cognate ligand of PD-L1 is PD-1, e.g., as expressed on the surface of T cells. In an embodiment, the checkpoint protein is the extracellular domain of the protein.

In an aspect, a checkpoint protein binds to its cognate ligand, which may also be a checkpoint protein receptor on an immune cell, such as a T cell, and blocks or interrupts signaling, activity, or function of the cell that expresses the cognate ligand or receptor. Alternatively, immune checkpoint inhibitors, which include antibodies and fragments of the antibodies that retain binding to checkpoint proteins, can bind to checkpoint proteins on cells, such as immune cells (e.g., effector T cells) and block or interrupt signaling, activity, or function of the cell. The binding of a checkpoint protein inhibitor to a checkpoint protein expressed on a cell can cause inactivation of the normal activity of the cell expressing the checkpoint protein. In embodiments, a checkpoint protein inhibitor is an antibody, such as a monoclonal antibody, a humanized antibody, a human antibody, a single chain antibody, etc., or a fragment thereof that binds to a checkpoint protein (cognate ligand).

Nonlimiting examples of immune checkpoint proteins, or cognate ligand binding portions thereof, that may be expressed in a cell, an iPSC, beta-cell, and the like, or an organoid, e.g., HILOs and other organoids as described herein, include PD-1, programmed cell-death protein 1, PD-L1, programmed cell-death ligand 1, which is the cognate binding ligand of PD-1; PD-L2, programmed cell-death ligand 2, which also binds PD-1; CTLA-4 (cytotoxic T-lymphocyte protein 4, also called CD152); LAG-3, lymphocyte activation gene 3 protein; KIR, killer cell immunoglobulin-like receptor; IDO1, indoleamine 2,3-dioxygenase 1; 4-1BB, a tumor necrosis factor receptor superfamily member 9, (also known as CD137); 4-1BBL (binds to 4-1BB); GITR, “glucocorticoid-induced TNFR family related gene; TIM-3, “T-cell immunoglobulin domain and mucin domain;” OX40, tumor necrosis factor receptor superfamily member 4, (also known as CD134); OX40L (binds to OX40), CD40, CD40L, A2AR, adenosine A2A receptor; B7-H3 (also called CD276); B7-H4 (also called VTCN1); B7-1/B7-2; BTLA (also called CD272); VISTA, “V-domain Ig suppressor of T cell activation;” and the like.

In embodiments, the immune checkpoint protein molecule is, without limitation, PD-L1 or the extracellular domain of PD-L1, which binds to PD-1 expressed by T cells. In an embodiment, a polynucleotide encoding an immune checkpoint protein is utilized to molecularly engineer a cell to express a checkpoint protein, or one or more checkpoint proteins, such as by infecting the cell with a viral or bacterial vector containing the checkpoint protein-encoding polynucleotide. In some embodiments, a cell (e.g., a beta-cell, or HILO cell) expresses more than one immune checkpoint protein, or a ligand binding portion thereof. In some embodiments, the cell is molecularly engineered to contain one, or more than one immune checkpoint protein, or ligand binding portion thereof, which is expressed by the cell. In an embodiment, the cell is infected with a viral vector, e.g., a lentiviral vector or adeno-associated viral vector, or more than one viral vector, that contains one or more polynucleotide(s) that encode(s) one or more immune checkpoint proteins or a ligand binding portion thereof, using procedures and methods that are well-known in the art. In an embodiment, the cell is transformed or transfected with a plasmid vector, or more than one plasmid vector, that contains one or more polynucleotide(s) that encode(s) one or more immune checkpoint proteins or a ligand binding portion thereof, using procedures and methods that are well-known in the art.

PD-1, the Programmed Death 1 (PD-1) protein, is a key immune checkpoint protein (receptor protein) that is expressed by activated T cells, as well as β cells, antigen presenting cells (APCs) and natural killer cells (NK cells) and mediates immunosuppression. PD-1 functions mainly in peripheral tissues where T cells may encounter the immunosuppressive PD-1 ligands PD-L1 (B7-H1) and PD-L2 that are expressed by other cells, such as cells molecularly engineered to express PD-L1, as well as, e.g., tumor cells, stromal cells, or both. Without intending to be limited by theory and by way of particular, nonlimiting example, PD-L1 expressed by transplanted, implanted, or engrafted beta(β)-cells, organoid cells, including HILO cells as described herein, binds to PD-1 expressed by effector T cells, thus effectively suppressing a T cell response directed against the beta-cells, organoid cells, or HILO cells and mediating the normal T cell response so as to tamp down or block autoimmunity and inactivate the immune response against the beta-cells, organoid cells, or HILOs. In an embodiment, the beta-cells, organoid cells, or HILOs express the immune checkpoint protein in situ, in the localized area of a transplant, implant, or graft; therefore, the ability of the cells and HILOs to evade autoimmunity occurs in and around the localized area of the transplant, implant, or graft and results in less risk of a systemic or more widespread modulation of immune cell activity in a recipient subject.

### Pancreas

In some aspects, a pancreatic organoid or a pancreatic islet organoid, also called a human islet-like organoid, or HILO, herein, is provided. The pancreas is an organ that lies in the abdomen and has endocrine and exocrine functions. The portion of the pancreas having an endocrine role are cell clusters called “pancreatic islets” (also known as islets of Langerhans). Pancreatic endocrine secretions include hormones that regulate glucose metabolism and blood glucose concentration. Four main cell types are present in the islets: alpha cells, which secrete glucagon (a hormone that increases blood glucose concentration); beta cells, which secrete insulin (a hormone that decreases blood glucose concentration); delta cells, which secrete somatostatin (a hormone that regulates alpha and beta cells), and gamma cells, which secrete pancreatic polypeptide.

The portion of the pancreas that has an exocrine role is referred to as the exocrine component. The exocrine pancreatic secretions contain digestive enzymes that pass into the small intestine and help break down carbohydrates, proteins, and lipids. The exocrine component has ducts arranged in clusters called pancreatic acini. Pancreatic exocrine secretions are secreted into the lumen of the acinus; the secretions accumulate and drain into the pancreatic duct and duodenum.

Pancreatic islet organoids, pancreatic organoids and HILOs as described herein mimic the structure of a pancreatic islet and a pancreas, respectively. In some embodiments, the pancreatic islet organoid or pancreatic organoid contains any one or more of the following cells: an iPSC-derived beta-like cell, an iPSC-derived alpha-like cell, an iPSC derived delta-like cell, and an iPSC-derived duct-like cell. In some embodiments, the pancreatic organoid contains an iPSC-derived exocrine component. In some embodiments, the iPSC is a human iPSC (hiPSC). Human embryonic stem cells and human induced pluripotent stem cells are commercially available (e.g., from WiCell, which provides iPS(IMR-90)-1, iPS(IMR-90)-4 and iPS(Foreskin)-1). Human induced pluripotent stem cells can also be generated using methods known in the art from a variety of somatic cell types (Yu, J., K. Hu, et al. (2009). “Human induced pluripotent stem cells free of vector and transgene sequences.” Science, 324(5928):797-801).

Pancreatic islet organoids, pancreatic organoids and HILOs as described herein also exhibit function(s) of a pancreatic islet and a pancreas. In certain embodiments, the pancreatic islet organoid or pancreatic organoid exhibits any one or more of the following functions: glucose-stimulated insulin secretion (GSIS), KCl-stimulated insulin secretion, GLP-1 stimulated insulin secretion, somatostatin secretion, and glucagon secretion. In some embodiments, the pancreatic islet or pancreatic organoid expresses any one or more of the transcription factors Pdx1, MafA, Pax4, Pax6, NeuroDI, Nkx6-1, Gata6, and Foxa2. In some embodiments, the HILOs express a checkpoint protein, or a functional portion thereof, that functions to allow the HILOs to evade immune detection and destruction by cells of the immune system. In some embodiments, the HILOs express more than one type of checkpoint protein or molecule, or a functional portion thereof.

### Generation of Pancreatic and Pancreatic Islet Organoids

In other aspects, methods of generating a pancreatic or pancreatic islet organoid are described. Recent studies have shown that while it was possible to generate glucose-responsive, insulin-producing, beta-like cells, efforts to generate pancreatic islets which are capable of secreting insulin, glucagon and somatostatin in response to nutrients, as well as efforts to obtain vascularization from stem cells, have not succeeded. Described herein are results demonstrating that using the self-organizing function of human adipose-derived stem cells (hADSCs), human umbilical vein endothelial cells (HUVECs), and human iPSC-derived beta-like cells, glucose responsive insulin secreting islet-like organoids (HILOs) capable of functional vascularization are successfully generated in vitro. Further, islet-like organoid generation methods were successfully scaled up using gellan gum based 3D culture systems. The functional heterogeneity in hiPSC-derived human islet-like organoids was also investigated using a Gaussia luciferase reporter to measure insulin secretion.

Generation of functional human organs provides new therapeutic strategies in drug-screening, disease modeling and inhibiting or preventing end point organ failure. Efficient stepwise differentiation methods from human embryonic stem cells (hESC) and human induced pluripotent stem cells (hiPSC) to insulin producing β-like cells have been demonstrated. For example, D'Amour et al. and Kroon E. et al. reported the efficient differentiation of hESCs into insulin producing cells which, after 4 to 5 months of in vivo maturation, were able to secrete insulin in response to glucose (D'Amour et al., 2006, Nature Biotechnology, 24, 1392-1401; Kroon et al., 2008, Nature Biotechnology, 26, 443-452). Recently, Rezania et al. and Pagliuca et al. reported in vitro differentiation methods that induced the formation of mature human beta-like cells that expressed the terminal β-cell markers MAFA and Nkx6-1, and exhibited partial functionality (e.g., insulin secretion) (Rezania et al., 2014, Nature Biotechnology, 32(11):1121-33; Pagliuca et al., 2014, Cell, 159, 428-439). However, in contrast to cadaveric human islets, those beta-like cells required in vivo functional maturation for a few months, and lacked the functionality provided by the other pancreatic islet cell types, such as glycemic control by a-cells (glucagon secretion) and δ-cells (somatostatin secretion). Further, the beta-like cells lacked both a mesenchyme and vascularized endothelial cells, which human islets naturally have. These crucial differences between hPSCs derived beta-like cells and human islets may compromise the ability of hPSCs-based therapies to treat insulin dependent diabetes (such as type 1 or late stage type 2 diabetes).

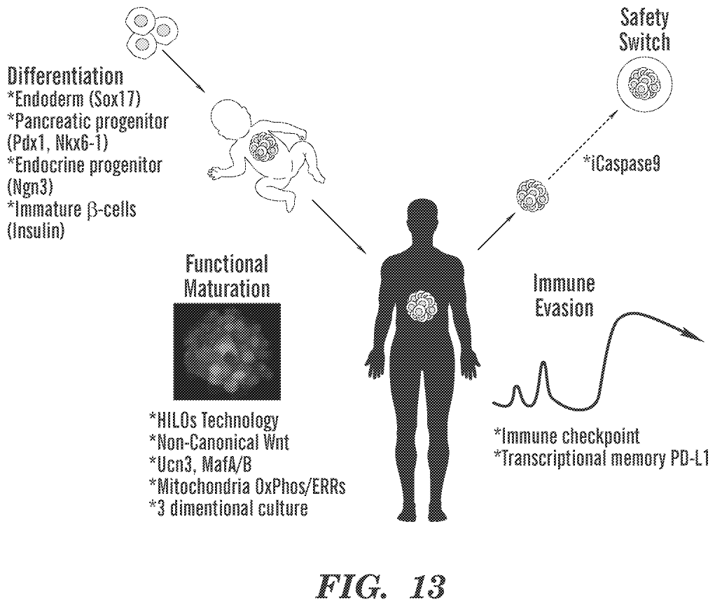

Previously, it was identified that a metabolic transition occurs during the neonatal to adult maturation of β-cells in which the orphan nuclear receptor Estrogen-related receptor γ (ERRγ) regulates an increase in oxidative metabolism required for fully functional β cells. Consistent with this result, human iPSC-derived β like cells expressing insulin, MAFA, and Nkx6-1 can be metabolically matured through the overexpression of ERRγ to increase their oxidative metabolism and thereby enhance their glucose stimulated insulin secretion (GSIS) functionality. These results indicated that, in addition to the expression of lineage determination factors such as PDX1, MAFA, Nkx6-1 and insulin, further cellular signaling which mature the β-cells' metabolism is required to generate fully functional β-cells. (FIG. 13).

During early pancreas organogenesis, newly specified pancreatic cells originate from the foregut endodermal sheet and form a pancreatic bud, a condensed tissue mass that is soon vascularized. A similar progression has been observed in liver organogenesis as well. Such large-scale morphogenetic changes depend on the exquisite orchestration of signals between endodermal epithelial, mesenchymal, and endothelial progenitors before blood perfusion. Takebe et al. successfully generated hepatic organ buds by culturing hepatic endoderm cells with endothelial and mesenchymal linages which rapidly vascularized and functionally matured in vivo (Takebe et al., 2013, Nature, 499:481-484).

Previous work did not reveal the possibility of generating in vitro other organoid tissue types, such as pancreas organoids, which were mature, functional, and vascularized. Further, previous work showed a lack of scalability because the organoids were generated using MATRIGEL® matrix, which is not efficient to use for scaled-up production.

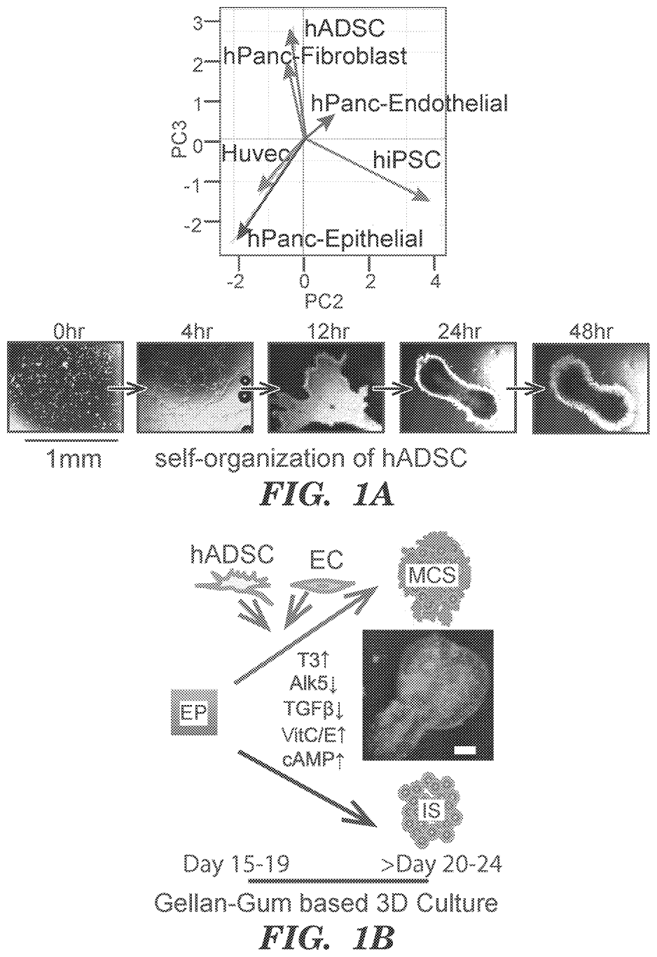

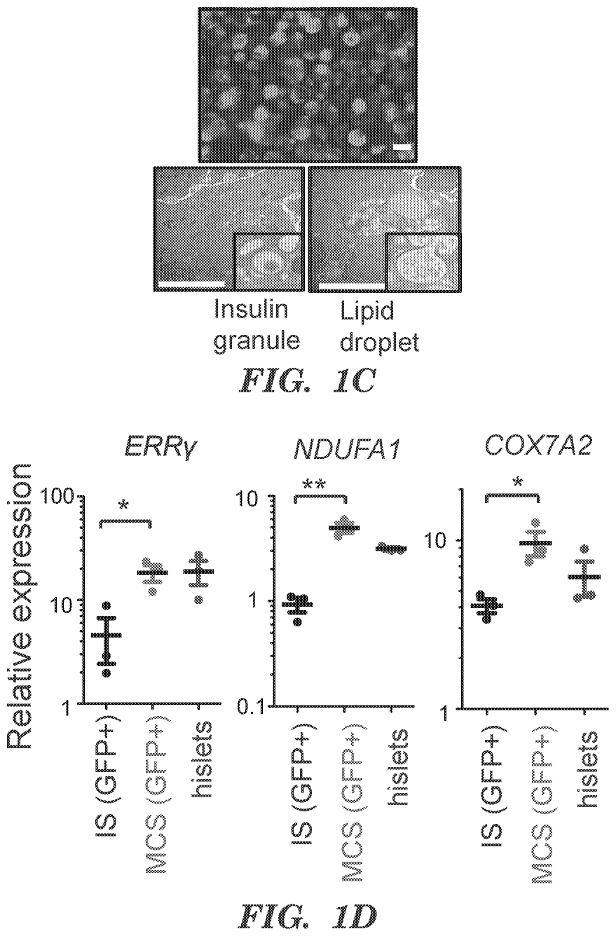

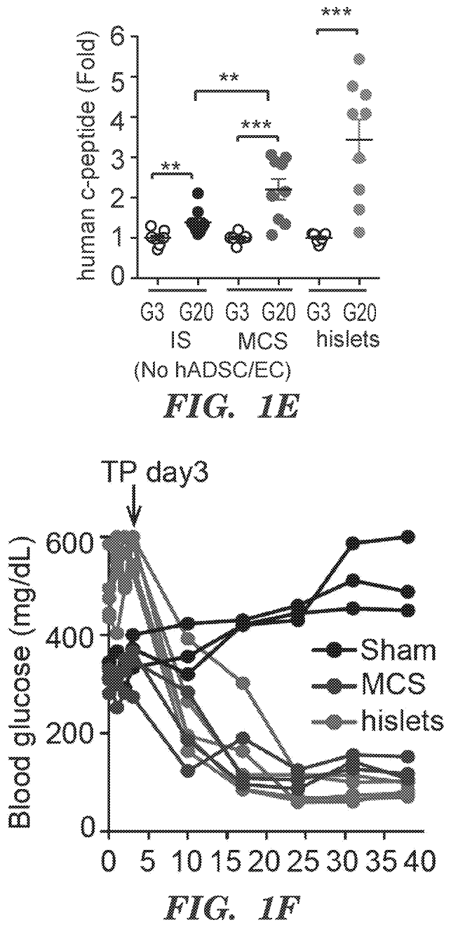

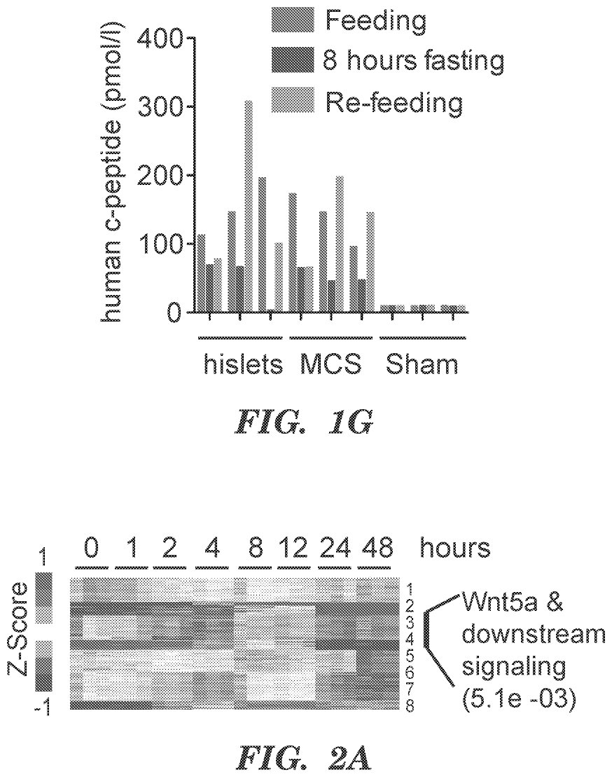

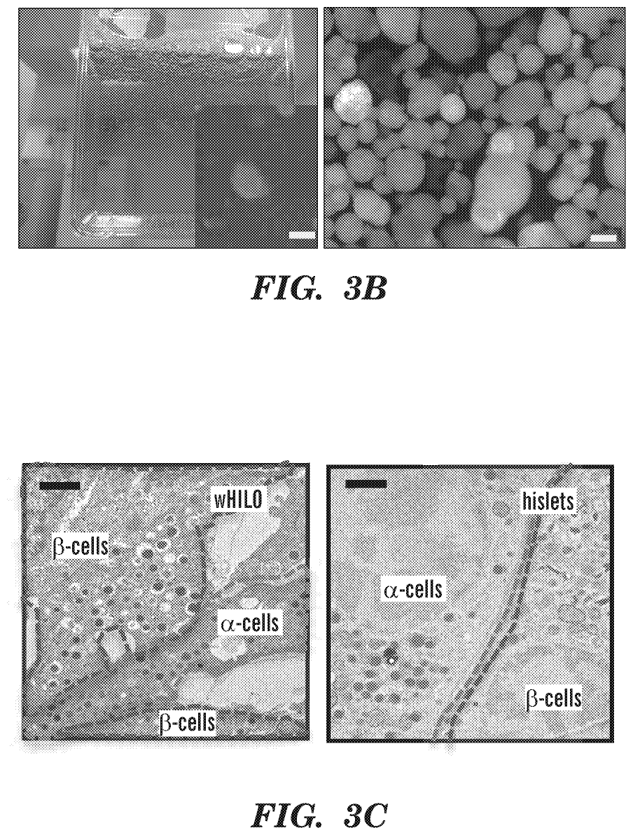

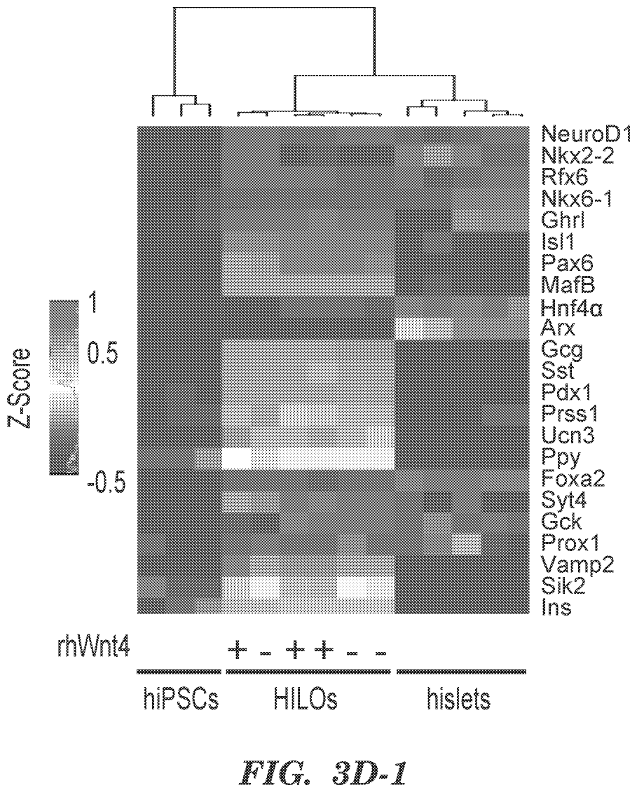

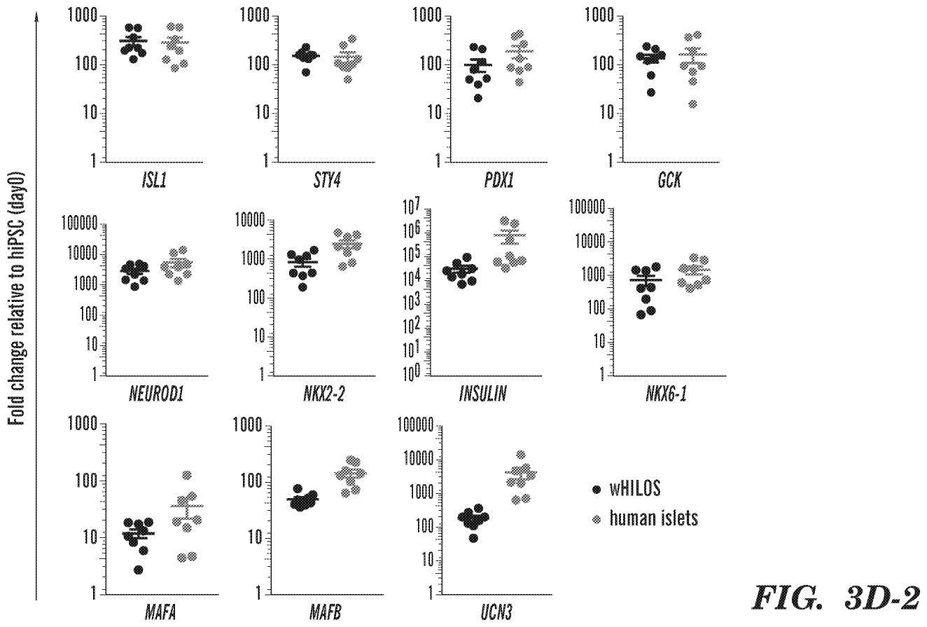

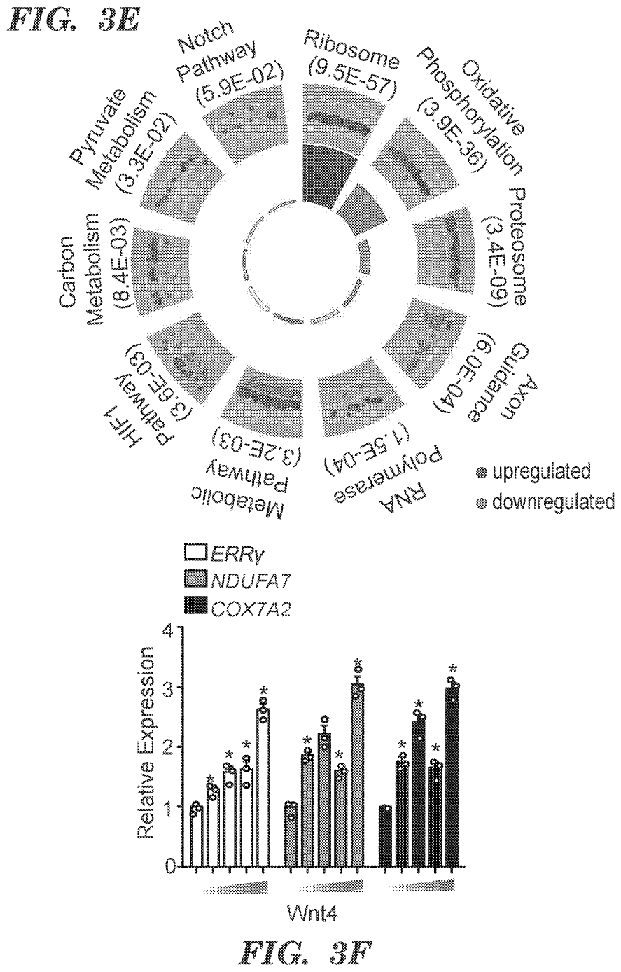

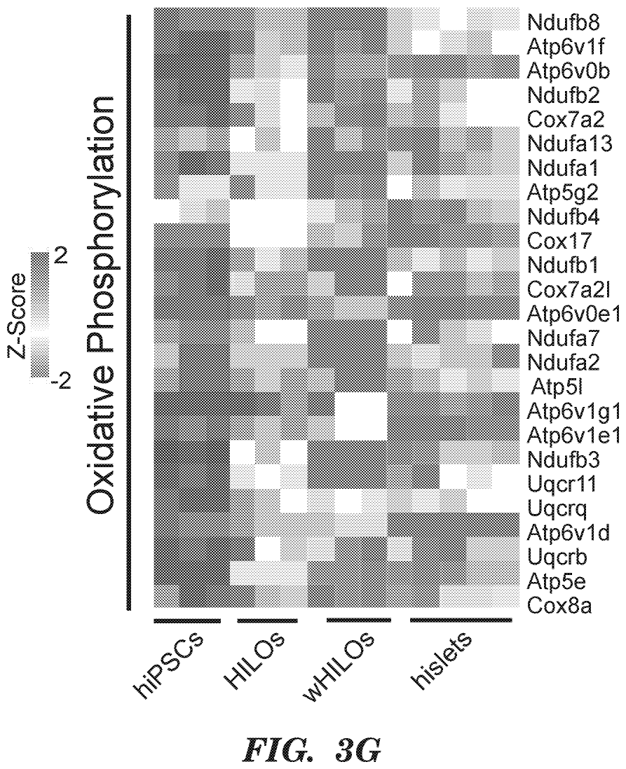

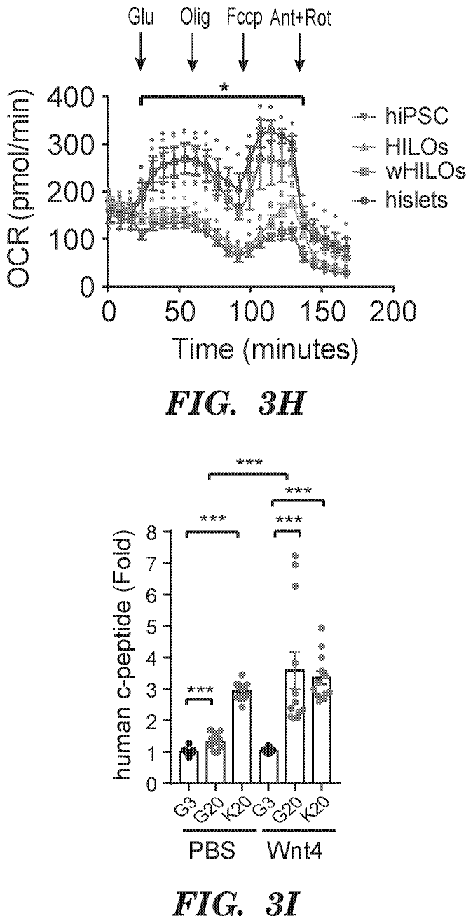

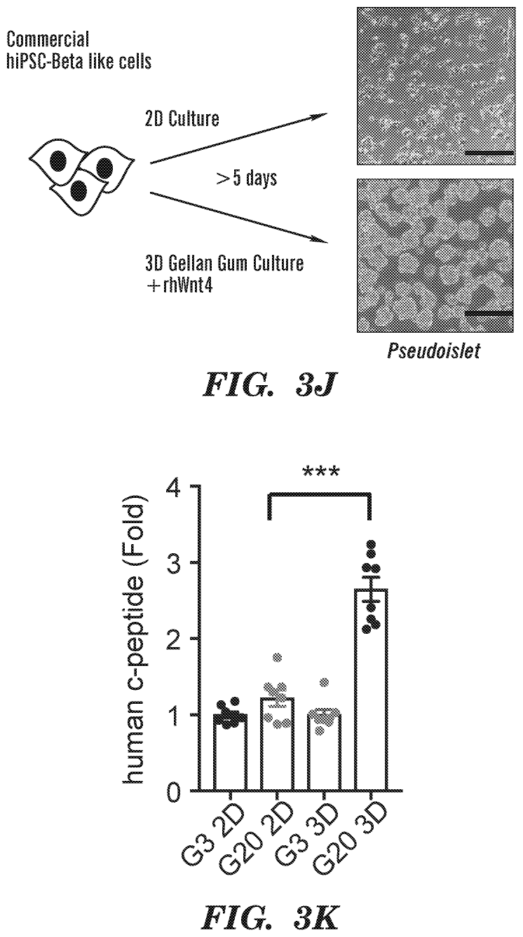

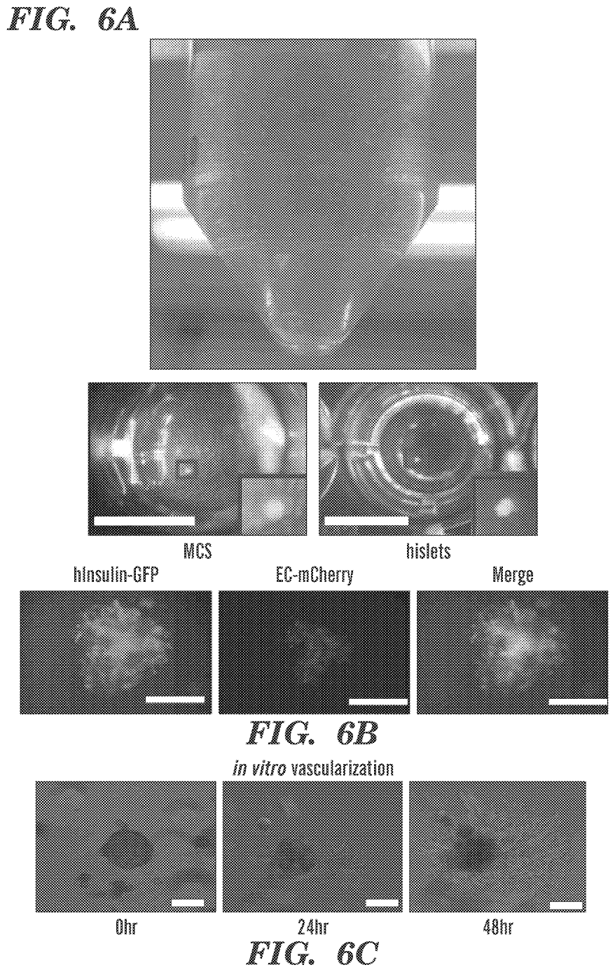

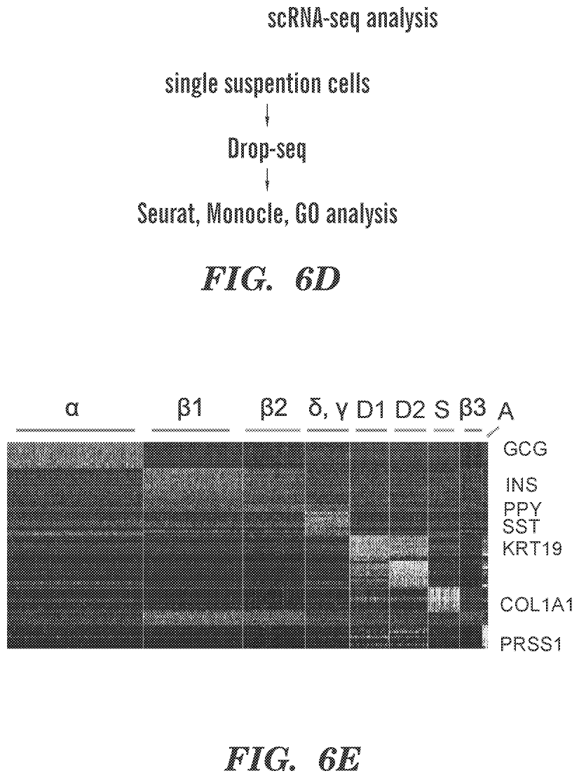

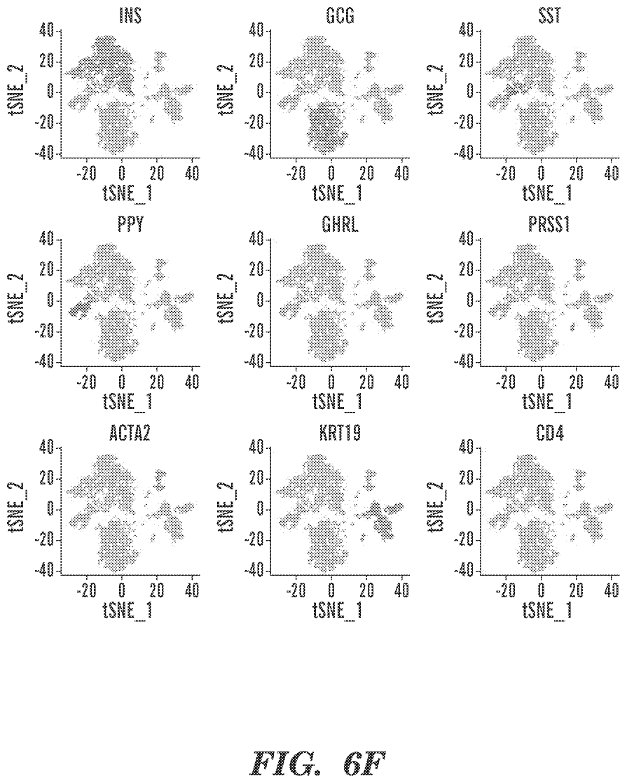

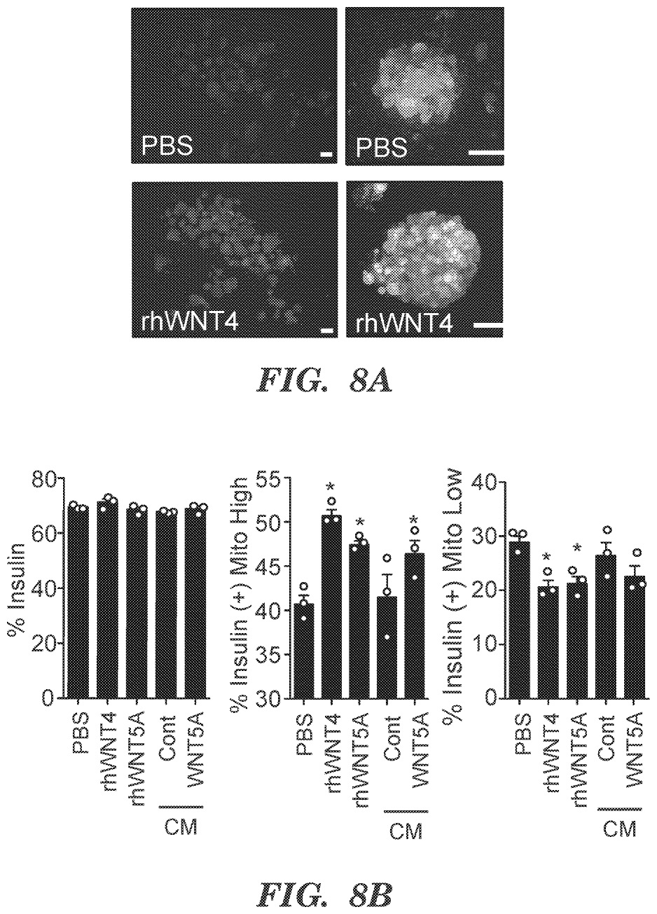

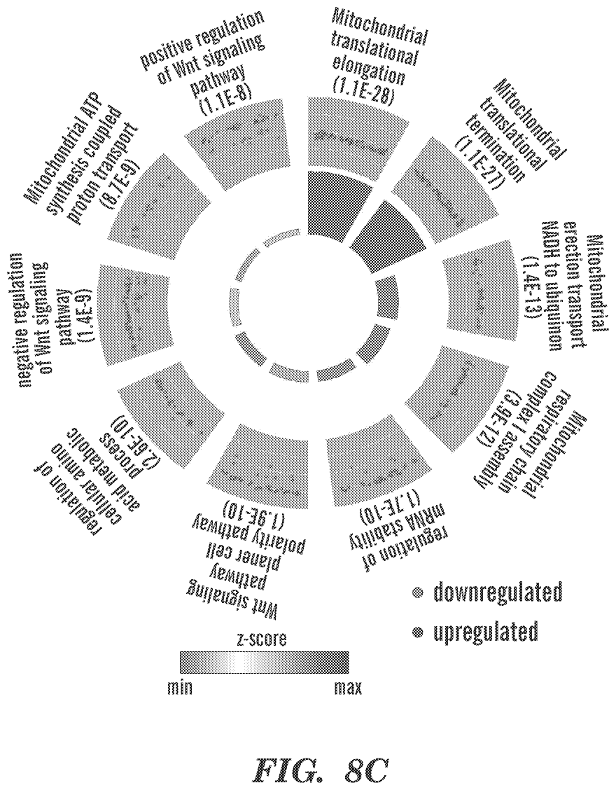

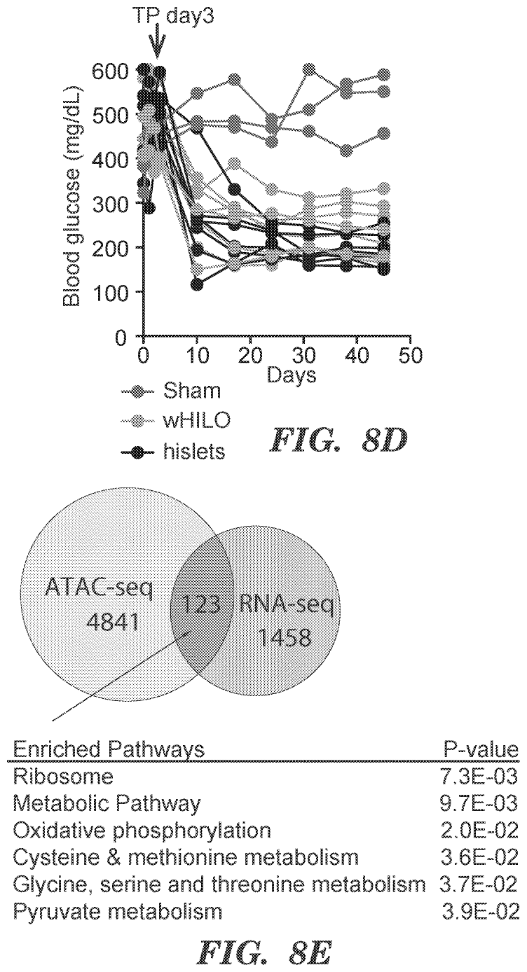

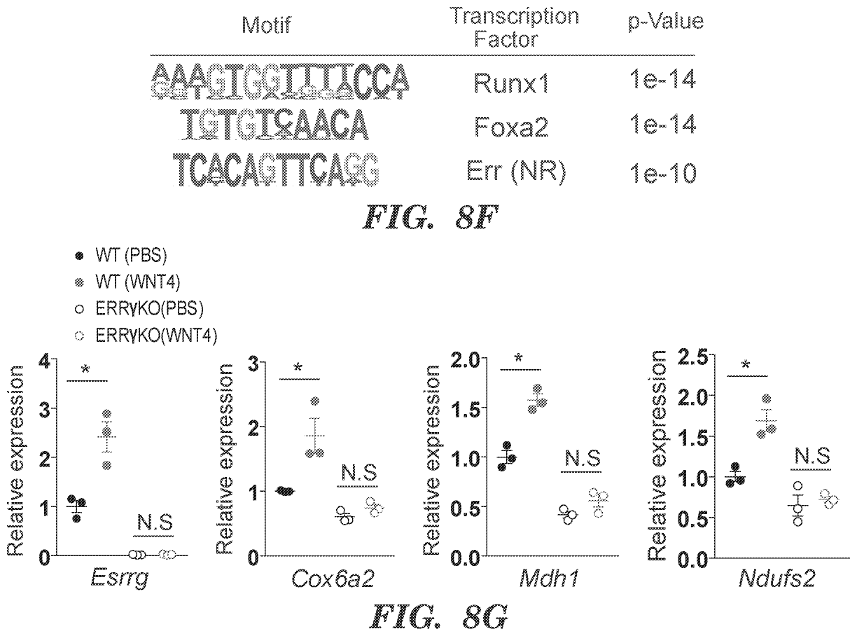

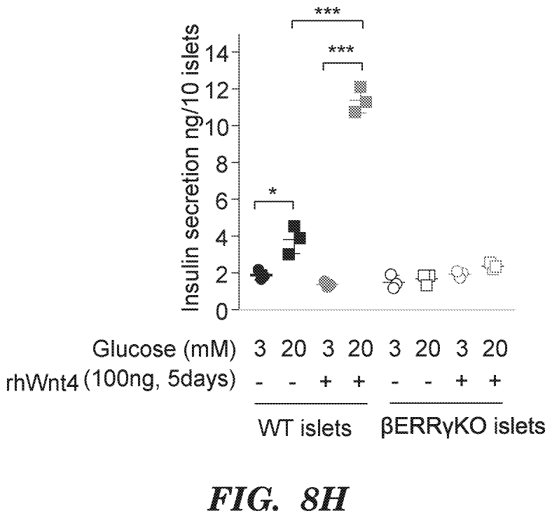

Described herein are studies demonstrating successful large-scale generation of human islet-like organoids (HILOs) that can secrete insulin and are vascularized, as seen in human islets, and that express one or more immune checkpoint proteins, thus affording the HILOs the ability to evade autoimmunity or immune detection by surveilling immune cells, e.g., T cells. It is demonstrated herein that (1) human adipose derived mesenchymal stem cells (hADSCs) have a self-organizing capacity (FIGS. 1A and 1B); (2) late stage pancreatic progenitors are capable of forming an islet-like cluster (organ buds) when co-cultured with HUVECs and hADSCs with comparable efficiency to beta-like cells; (FIGS. 1A-IC, FIG. 1E and FIGS. 3A-3C); (3) human islet-like organoids had improved expression of lineage determination factors, as well as metabolic regulatory genes including ERRγ; (4) islet insulin secretion assays revealed that human islet-like organoids contain functional cells capable of secreting insulin in response to glucose (e.g., Example 8); (5) human islet-like organoids (HILOs) exhibited vascularization (FIG. 6C); (6) human islet-like organoids derived from hiPSC as described herein recaptured human islet organogenesis and pathogenesis of type 1 and type 2 diabetes in a dish; (7) human islet-like organoids derived from hiPSC as described herein offered a new replaceable resource for human islet transplantation to treat type 1 and type 2 diabetes; (8) human islet-like organoids transplanted into an STZ-induced NODSCID mouse model of type 1 diabetes ameliorated type 1 diabetes in the recipient animals (FIGS. 1F and 1G); and (9) Wnt4 and Wnt5a increased the number of mitochondria-enriched β cells in HILOs (FIGS. 8A-8D), thus suggesting that both Wnt4 and Wnt5a (derived from pancreatic endocrine cells and supportive cells, respectively) enhance mitochondrial metabolic function to promote β cell maturation and sustainable GSIS function.

Also described herein are studies in which the role of certain Wnt (also “WNT” herein) proteins was assessed in developing human islet-like organoids which are capable of secreting insulin and which are vascularized, as seen in human islets. The WNT gene family consists of structurally related genes that encode secreted signaling proteins, which have been implicated in oncogenesis and in several developmental processes, including regulation of cell fate and patterning during embryogenesis. Wnt proteins comprise a major family of signaling molecules that orchestrate and influence a variety of cell biological and developmental processes. Wnt proteins undergo a complex set of posttranslational modifications involving several highly specialized processing enzymes. Upon release from the cell, the Wnt proteins interact with a number of molecules in the extracellular environment, such as glycans, protein-binding partners (e.g., WIF, Sfrp) and cell surface receptors. (Willert, K. et al., 2012, Cold Spring Harbor, Perspectives in Biology, 2012). From studies described herein, Wnt5a is the predominant Wnt protein that induces the self-organization of hADSCs; (2) Wnt5a, as well as Wnt4, activate the ERRγ-mitochondrial metabolic pathway; (3) Wnt4 is sufficient to induce in vitro functional maturation of hiPSC-derived islet-like organoids in the absence of additional cell types such as hADSC and HUVECs.

Generation of Mature HILOs that Evade Immune Detection

In vivo, β cells become functionally mature via a long, postnatal maturation process. To date, human induced pluripotent stem cells (hiPSCs) have not been successfully transformed into fully functional β cells by duplicating this process in vitro. Moreover, even though β cells derived from hiPSCs are immune-matched to the patient, life-long immune suppression may still be required to protect against transplant rejection after β cells are transplanted into a patient, particularly, patients with type 1 diabetes who generally have a hyper-reactive immune system. Thus, the generation of universal PSCs that resist immune rejection by expressing one or more checkpoint molecules is highly beneficial, as this would obviate a need for costly personalized therapies.

A self-organized, three-dimensional (3D) tissue architecture is required for organ formation and the terminal differentiation of organ-specific cell types. As described herein, 3D structured organoids comprising human pancreatic islet tissue were generated. The production of functional 3 cells requires cellular diversity within the developing islet, as well as cellular interactions that may influence the functional differentiation of islets from hiPSCs.

As described herein, a method for the scalable generation of human islet-like organoids (HILOs) from hiPSC is provided. The method utilizes a differentiation pathway that results in enhanced functional maturation and endows the resulting HILOs with immune evasive function. Advantageously, the described method does not require the use of instruments, such as a magnetic spinner or an air-liquid surface, thereby resulting in a simplified and highly reproducible procedure. The scalability of the system allows for both large- and small-scale production of mature HILOs. Tissue maturity is critical for recapitulating all aspects of pancreatic islet function. Since hiPSC-derived pancreatic progenitors or β-like cells reach functional maturation with physiological levels of insulin secretion in vivo within a few months, the in vitro differentiated β-like cells have the potential to be fully functional, mature β-like cells.

The scalable process for generating islet-like organoids from hiPSCs as described herein includes effective signals for functional maturation of the cells, and cellular heterogeneity. In an aspect, a functional, polymer-based, 3-dimensional (3D) culture system and activation of non-canonical Wnt (e.g., Wnt4) signaling are provided to generate 3D structured human islet-like organoids (HILOs) that contain critical pancreatic islet cell types, including beta (β) cells (insulin), alpha (α) cells (glucagon), delta (δ) cells (somatostatin), gamma (γ) cells (PPY), and F cells (ghrelin (GHRL)).

The scalable, 3D system for generating mature human islet-like organoids (HILOs) involves stimulating the non-canonical Wnt pathway to achieve mitochondrial OxPhos function and functional insulin secretion as described herein provides medically useful, therapeutic biological material for the treatment of diseases, such as diabetes. As described herein, the stem cell derived, mature islets or HILOs can express an immune check point molecule; therefore, they are capable of evading allogenic immune rejection and thus provide a fundamental cure for insulin dependent diabetes, without resorting to immunosuppressants. Such HILOs may serve as universal (allogeneic) pancreatic islets, instead of patient-specific or autologous islets, leading to greater availability of therapeutic biological materials and cost reductions in the treatment of insulin dependent diabetes.

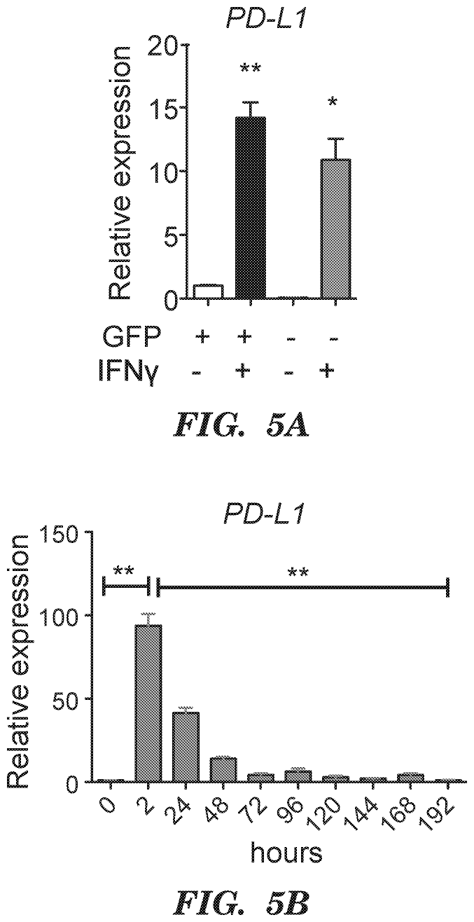

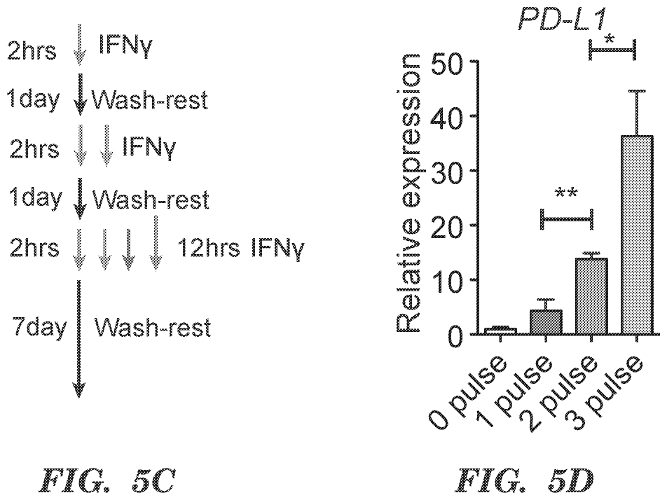

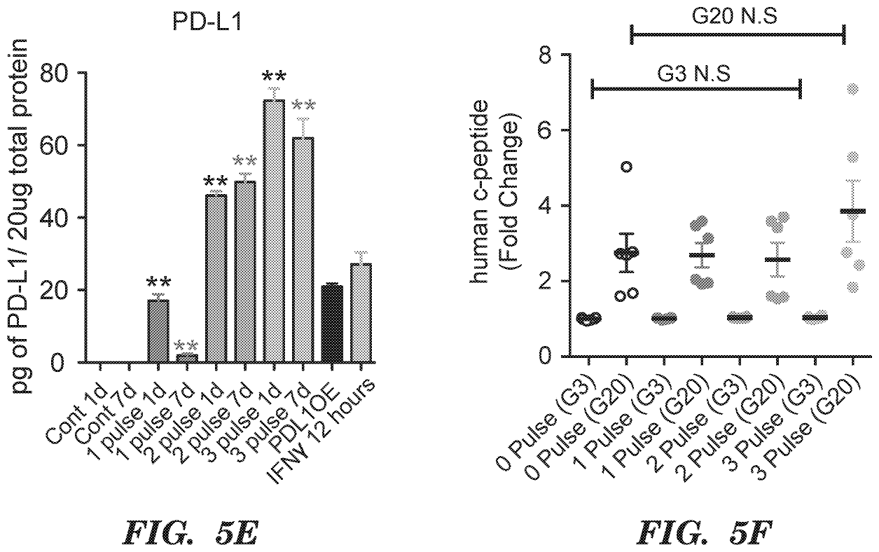

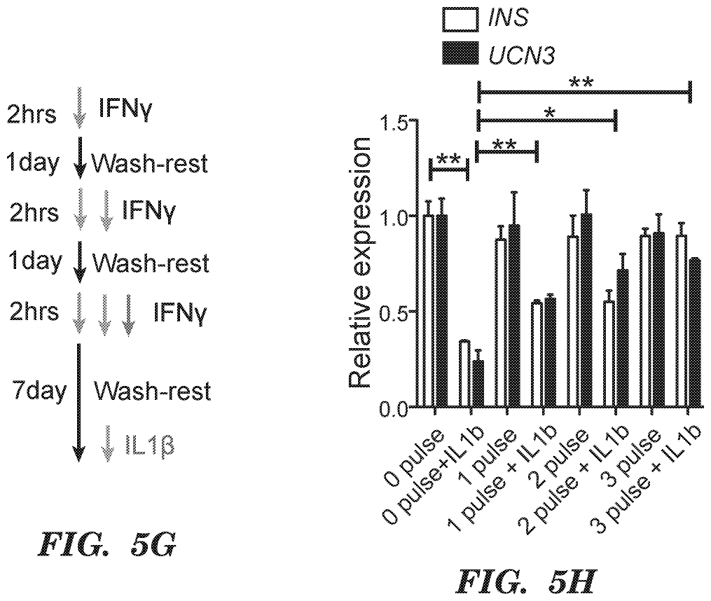

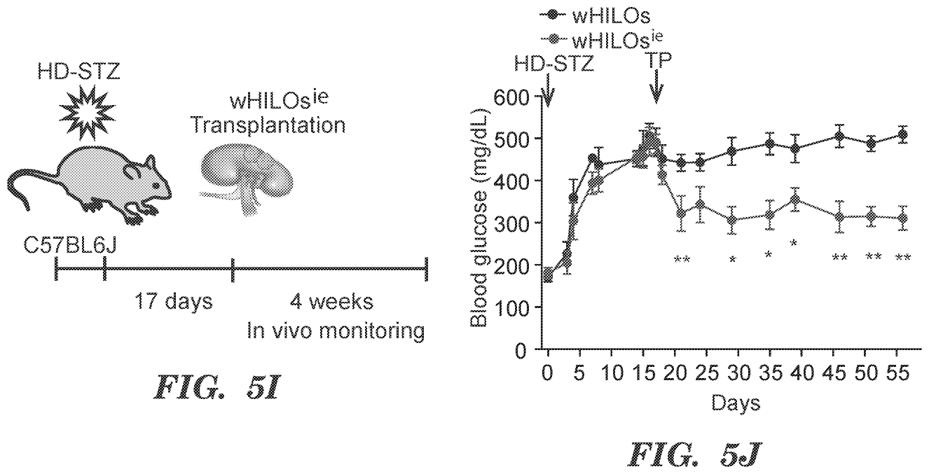

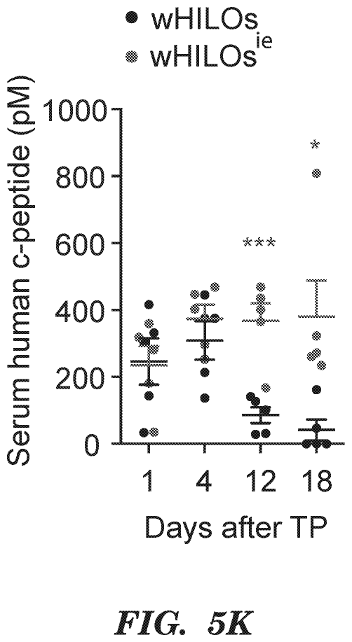

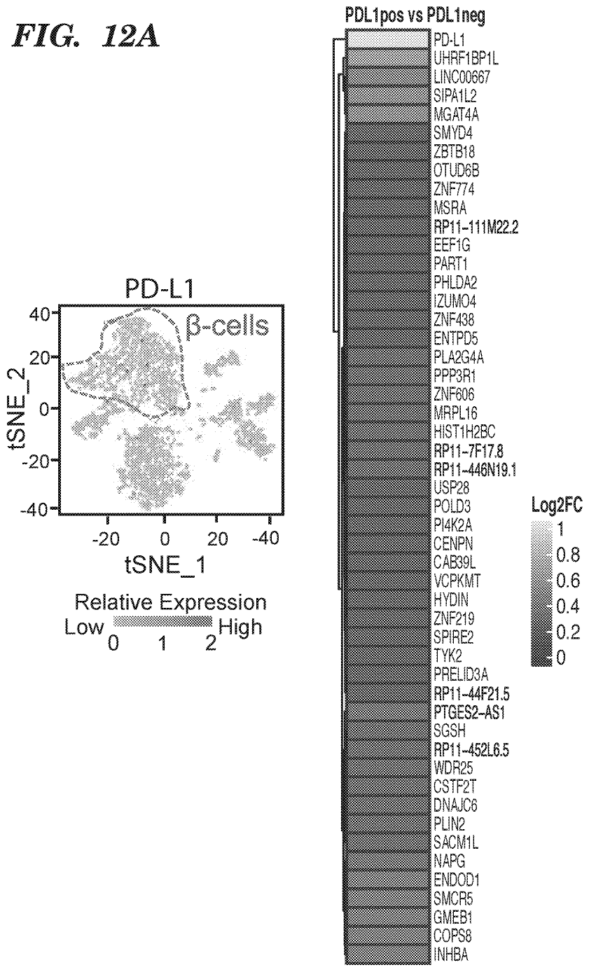

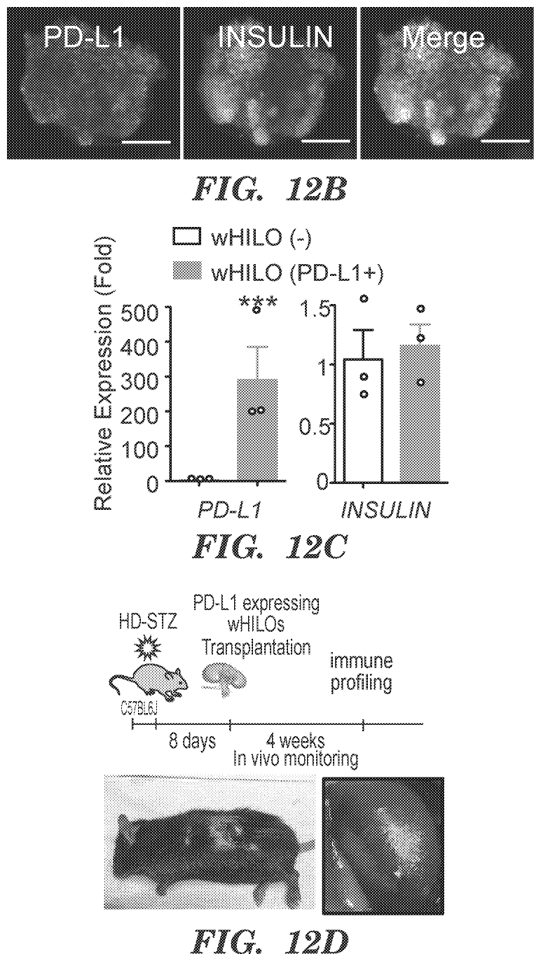

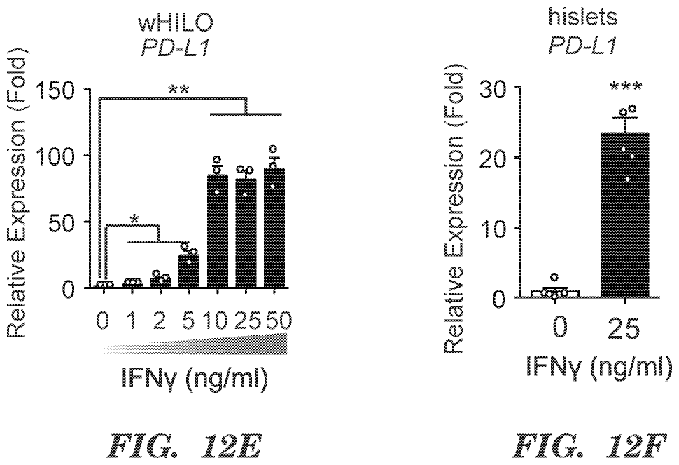

As described herein, the IFNγ pathway was assessed for the ability to minimize host immune responses against transplanted or implanted wHILOs. Following a short exposure of wHILOs to IFNγ stimulation, it was found that IFNγ rapidly and robustly induced PD-L1 expression in wHILOs (FIGS. 12E and 12F). Notably, IFNγ induced PD-L1 expression to levels similar to those in both insulin-expressing and insulin non-expressing cells (GFP+ and GFP-cells, respectively), (FIGS. 5A and 5B). Repeated exposure of HILOs to IFNγ (IFNγ stimulation) induced a similar effect in wHILOs, specifically, a sustained induction of PD-L1 in the HILOs. In an aspect, repeated short exposures to IFNγ (multiple pulse stimulation, MPS) led to sustained PD-L1 expression and concomitant increases in PD-L1 protein levels (FIGS. 5C, 5D and 5E). In embodiments, human islets or HILOs, e.g., mature islets or HILOs are exposed to (contacted with) IFNγ for at least 0.5-5 hours, at least 1-5 hours, at least 1-3 hours, at least 1-2.5 hours, or at least 1-2 hours. In particular embodiments, human islets or HILOs, e.g., mature islets or HILOs are exposed to (contacted with) IFNγ for greater than 1 hour, greater than 2 hours, for 1 hour, for 2 hours, or for 3 hours, prior to washing the islets or HILOs and allowing them to rest in medium without IFNγ. In embodiments, each exposure of the human islets or HILOs to IFNγ is termed a “pulse.” In embodiments, the human islets or HILOs are exposed to, contacted or pulsed with IFNγ at least one time, at least two times, at least three times, at least four times, at least five times, etc., or 1, 2, 3, 4, or 5 times, in a one-day or a multi-day (e.g., over a 72 hour time period, or a longer time period) protocol in which cells are allowed to recover (e.g., in medium or matrix without IFNγ) between IFNγ pulses for about 24 hours. In a particular embodiment, the human islets or HILOs are pulsed with IFNγ three times over 3 days, (72 hours), for 2 hours per pulse period, to achieve a constitutive level of PD-L1 expression in the islets or HILOs. Following this IFNγ MPS regimen, the IFNγ-stimulated human islets or HILOs showed high levels of PD-L1 protein expression at 7 days post MPS. In embodiments, the human islets or HILOs are exposed to (contacted or pulsed with) IFNγ in an amount of 1-100 ng/ml, 1-50 ng/ml, 1-25 ng/ml, 1-20 ng/ml, 1-10 ng/ml, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml. In a particular embodiment, IFNγ is in an amount of 10 ng/ml or 20 ng/ml for each exposure or pulse period. In a particular embodiment, the human islets or HILOs, including mature human islets or HILOs, are exposed to, contacted or pulsed with 2 pulses of IFNγ for 2 hours per pulse in a 2-day period. In a particular embodiment, the human islets or HILOs, including mature human islets or HILOs, are exposed to, contacted or pulsed with 3 pulses of IFNγ for 2 hours per pulse over a 3-day (day3) period.

GSIS functionality was not compromised by exposure of the wHILOs to MPS by IFNγ (FIG. 5F). Furthermore, IFNγ-treated wHILOs were protected against IL-1β-induced β cell dedifferentiation, as revealed by the expression of the B cell identity markers INS and UCN3 (FIG. 5H).

Normal, in utero development of a human pancreas takes more than 280 days, and full functional maturity is not reached until a few years after birth; therefore, gaining a complete understanding of the complex pathways involved in the development and maturation of human islets is a necessary step toward generating functional islets in vitro. A pivotal aspect for functional maturity of β cells is the activation of the mitochondrial metabolic pathway, which occurs naturally in postnatal maturation and is required for functional β cells nutritional sensing insulin secretion function. For HILOs, sustainable mitochondrial activation may be achieved through Wnt4 driven mitochondria metabolic regulation.

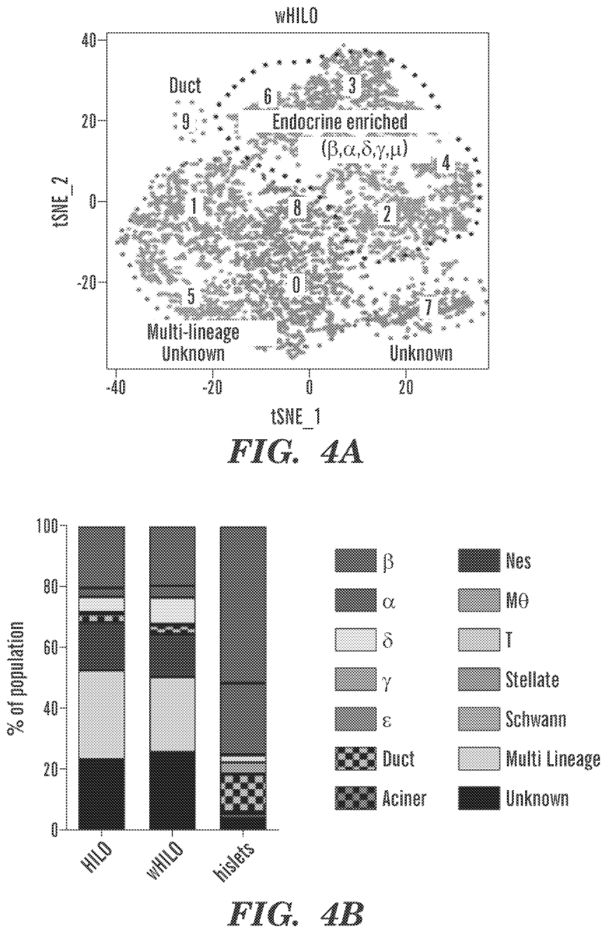

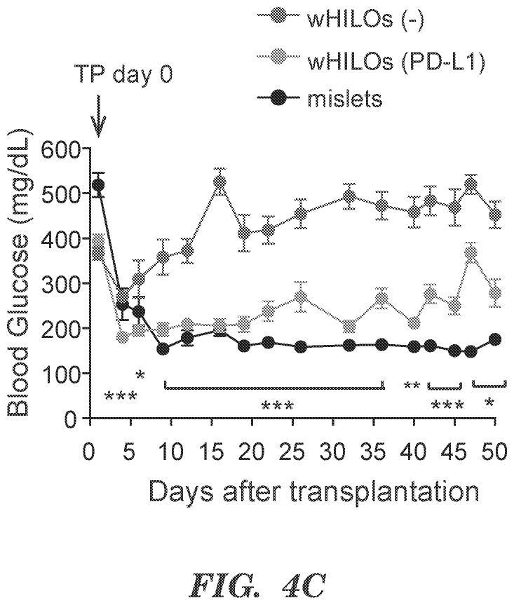

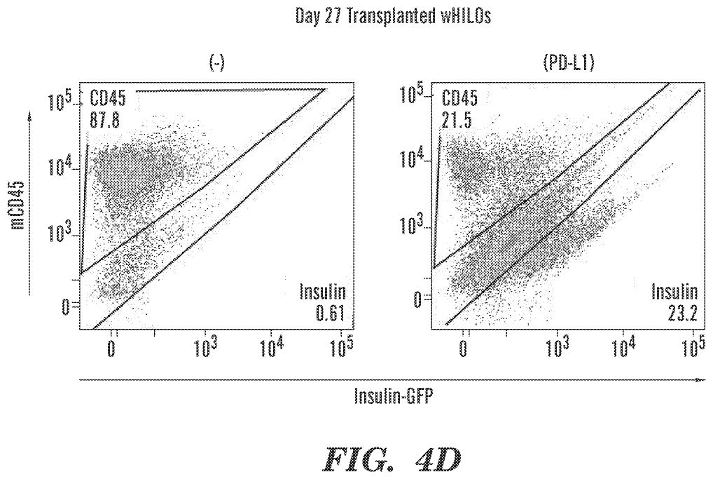

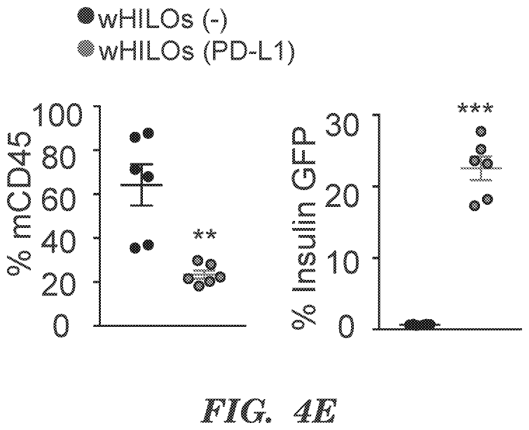

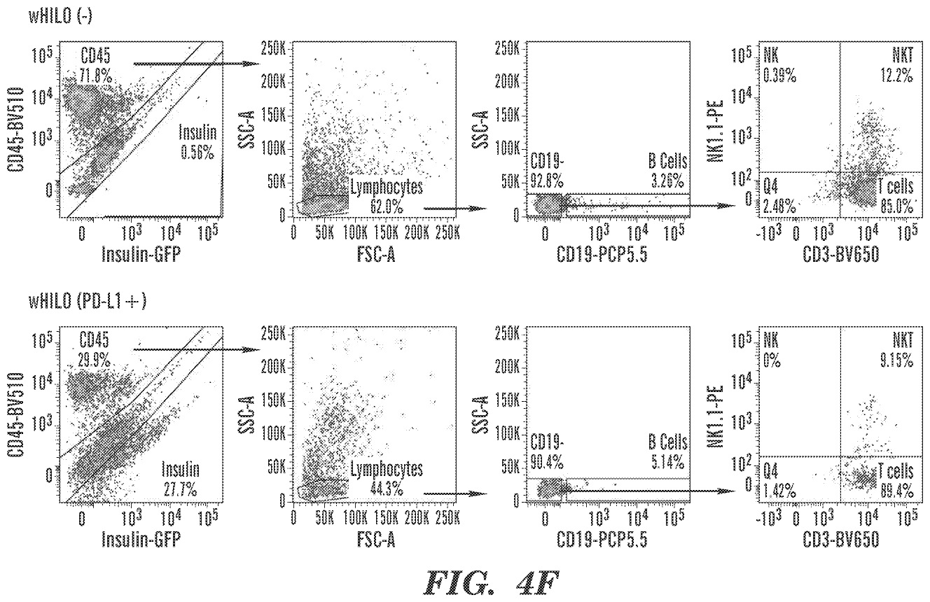

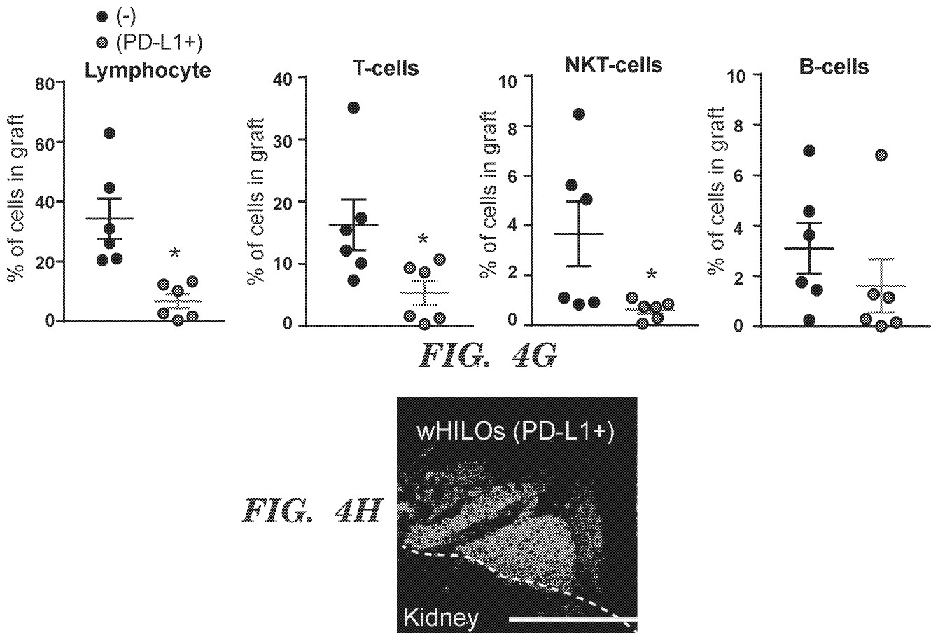

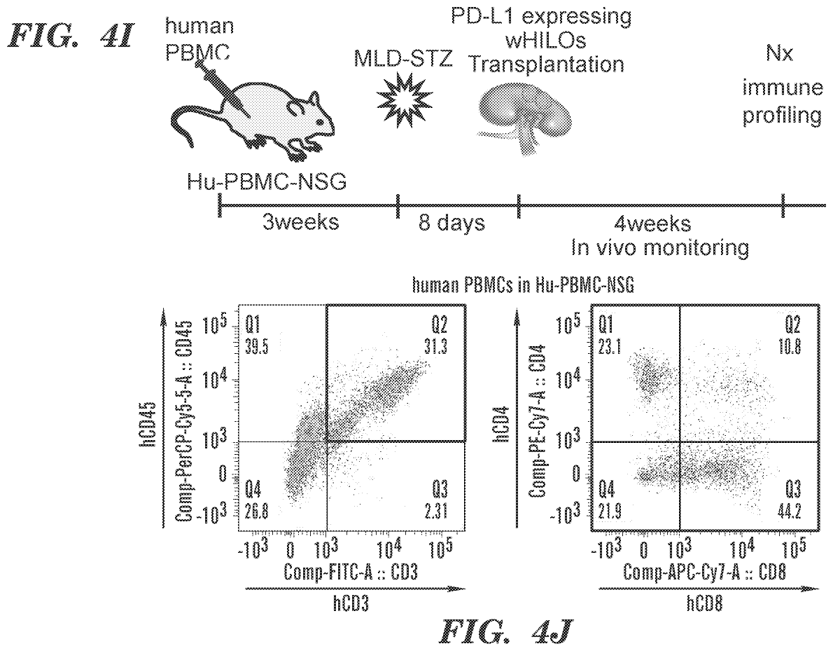

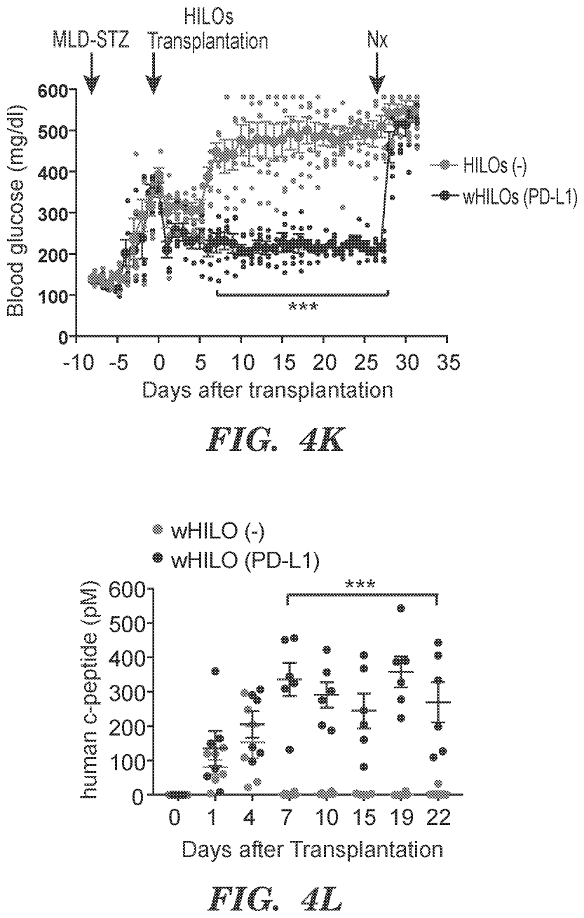

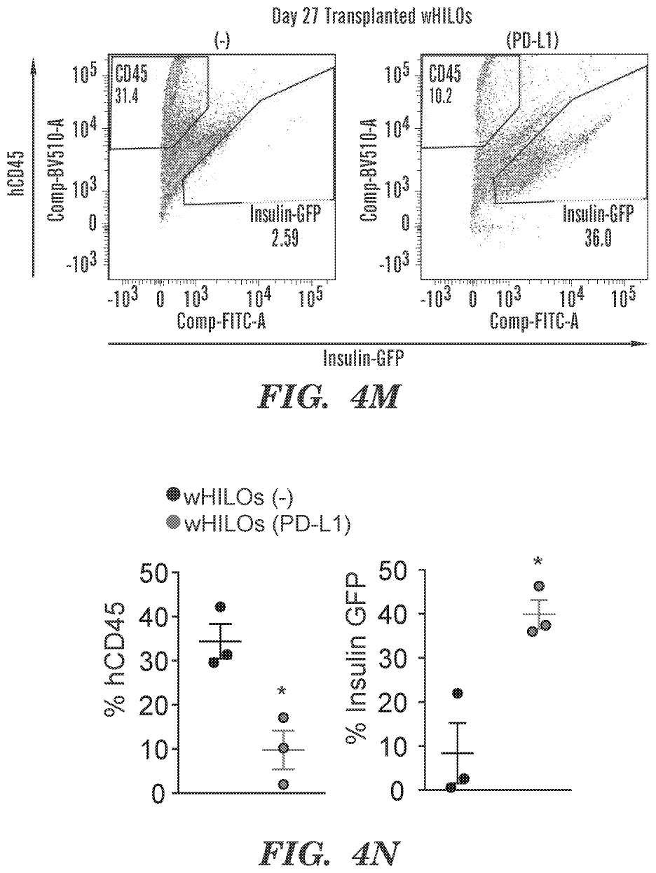

In an aspect, enhancing the ability of transplanted β cells to evade immune detection as described herein provides an alternative or adjunct strategy to MHC matching (A. Morizane et al., 2017, Nature communications, 8:385) for reducing the risk of autoimmune rejection of transplanted islet cells, pancreatic islets, organoids and HILOS. Stem cell-, islets- and organoid-based treatments for diabetes must achieve protection of the transplanted cells, islets and organoids from autoimmune rejection, in addition to their functional maturity. When PD-L1 negative mature HILOs were transplanted into diabetic immune-competent C57BL/6J mice, the xenograft was rejected and failed to produce detectable amounts of human c-peptide. In contrast, mature HILOs that expressed PD-L1 (either via molecular engineering or induction of expression of PD-L1 in organoid cells as described herein), successfully survived more than 50 days following transplantation into immune competent animals. (FIGS. 4D-4E and FIGS. 12A-12C). Moreover, acquisition of immune tolerance did not require the presence of Tregs. Thus, in an aspect, additional immune protection may be achieved by co-culturing Tregs in the gel-based system used to produce mature HILOs. During antigen presentation, interactions between cytotoxic T-lymphocyte antigen-4 (CTLA-4) and B7 molecules, as well as programed death 1 (PD-1) protein and its ligand PD-L1, negatively regulate immune responses in a non-redundant manner. As described herein, PD-L1 negative, control HILOs were rejected in T and B cell competent C57BL6J mice, but were not rejected in T and B cell-deficient NOD-SCID mice (e.g., Example 8), suggesting that allogenic rejection for PD-L1 negative control mature HILOs were mainly through T cells and β cells reaction in vivo.

The generation of iPSCs by somatic cell reprogramming provides a source of patient-specific cells (e.g., autologous cells) that may be differentiated into any lineage. Moreover, generating insulin-producing cells from iPSCs provides an invaluable tool for autologous transplantation, which would greatly reduce the risk for autoimmune rejection. While allogenic transplantation of MHC-matching grafts has proven effective in reducing immune responses and is useful, this technique may not result in complete evasion of the immune system and immune surveillance, even in less immunological sites, such as the brain. Thus, a combination of MHC matching and the induction of immune tolerance may provide a further approach to controlling immune responses against transplanted stem cells, islets and organoids. In some cases, such procedures may obviate a need for immunosuppressive drugs.

Because ongoing autoimmunity in patients with type 1 diabetes could still result in immunogenicity when patient-specific, stem cell-derived islets are transplanted, or stem cell-based islet cell replacement approaches are used, employing allogeneic hiPSCs together with immunosuppressive or tolerogenic treatments (for controlling both alloreactivity and autoreactivity) provide advantageous therapies for patients with type 1 diabetes. In addition, co-stimulation blockade procedures involving the expression of one or more checkpoint inhibitor molecules as well as a checkpoint protein to evade immune surveillance, e.g., CTLA4Ig- and PD-L1-expressing human stem cells, β cells, islets cells, or organoid cells, may provide clinically relevant materials for successful transplantation/implantation in subjects for diabetes treatment. By protecting HILOs via PD-L1 expression to promote graft/transplant/implant survival, HILO allografts can experience reduced immune cell infiltration, in the absence of immunosuppressive drugs. However, it will be appreciated that one or more immunosuppressive may be used if medically required or desired.

### Methods of Treatment

Islet transplantation is a therapy for treating insulin deficient diabetes such as type 1 and late stage type 2 diabetes. Thus, in an aspect, a method of treating a pancreatic disease such as type 1 or type 2 diabetes are provided, in which the method comprises administering a pancreatic or pancreatic islet organoid, in particular, a HILO expressing a checkpoint protein as described, to a subject (e.g., a mammalian subject, such as a human or human patient) by transplantation (or implantation). In an embodiment, the method treats a subject suffering from, susceptible to, or at risk of having, a pancreatic disease (e.g., type 1 diabetes), disorder, or symptom thereof. The method includes the step of transplanting a pancreatic or pancreatic islet organoid (HILO) in the mammal sufficient to treat the disease, disorder, or symptom thereof, under conditions such that the disease, disorder, or symptom is treated.

As used herein, the terms “treat,” treating,” “treatment,” and the like refer to reducing, diminishing, ameliorating, abrogating, or alleviating a disease, disorder and/or the symptoms associated therewith. It will be appreciated that, although not precluded, treating a disease, disorder, condition, or symptom thereof does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of, or susceptible to, developing or having a disorder or condition.

The therapeutic methods (which include prophylactic treatment) generally comprise administration, in particular, transplantation or implantation, of an effective amount of a pancreatic islet or pancreatic islet organoid (e.g., a HILO) to a subject (e.g., animal, mammal, human) in need thereof, including a mammal, particularly a human. In particular, the pancreatic islet or pancreatic islet organoid (e.g., HILO) is molecularly engineered to express one or more checkpoint proteins. In an embodiment, the checkpoint protein is PD-L1. In an embodiment, a cell, islet, or organoid is subjected to multiple intermittent exposures to interferon gamma (IFNγ), (multiple pulse stimulation or MPS), according to the methods described herein. The MPS methods yield cells, islets, or organoids in which the expression of a checkpoint protein such as PD-L1 is sustained over long time periods following transplantation or administration to a subject, thereby allowing the transplanted or administered cells, islets, or organoids to function while avoiding autoimmunity or immune detection. In an embodiment, the administration of a pancreatic islet or pancreatic islet organoid (e.g., HILO) may be by any suitable means that results in an amount of the organoid that, combined with other components, is effective in ameliorating, reducing, abrogating, diminishing, or stabilizing a pancreatic disease such as type 1 or type 2 diabetes.

In certain aspects, the subject may be further administered an immunosuppressant. The immunosuppressant can be administered to the subject before, during, or after the subject is administered (e.g., transplanted or implanted) with the organoid. The immunosuppressive agent can be an agent that inhibits or prevents rejection (e.g., acute rejection) of the transplanted organoid upon transplantation, or an agent that maintains immunosuppression after the transplantation. Immunosuppressants include, but are not limited to, basilizimab, antithymocyte globulin, alemtuzumab, prednisone, azathioprine, mycophenolate, cyclosporine, sirolimus, and tacrolimus.

In some embodiments, at least about 100,000, at least about 200,000, at least about 300,000, at least about 400,000, at least about 500,000, at least about 600,000, at least about 700,000, at least about 800,000, at least about 900,000 or at least about 1 million pancreatic islet organoids (HILOs) are transplanted or implanted into the subject. In some embodiments, islets of the subject are removed prior to transplanting or implanting the organoids of the invention. In some other embodiments, pancreatic islet organoids (HILOs) are transplanted or implanted into a subject by injection into the upper abdomen of the subjects. In some embodiments, the pancreatic islet organoids (HILOs) are injected into the liver. The pancreatic islet organoids can be injected into the subject using a catheter. In some other embodiments, the pancreatic organoid or pancreatic islet organoid (HILO) is administered to the subject by surgery, e.g., transplant surgery. In another embodiment, pancreatic islet organoids (HILOs) are transplanted onto the omentum. For omentum transplantation, a layering technique can be used in which the islet organoid (or cells thereof) are combined with autologous plasma and are laparoscopically layered onto the omentum. A solution (20 ml) containing recombinant thrombin (1000 U/ml) is next layered over the islet organoid, followed by another layer of autologous plasma to produce a biodegradable biologic scaffold that can survive and function in the patient for at least a year (See, e.g., Baidal, D. et al., 2017, N. Engl. J. Med., 376:19). In another embodiment, hydrogel biomaterials that mitigate an immune response by the recipient can be used for islet organoid transplantation. (See, e.g., Vegas, A. et al., 2016, Nature Biotechnology, 34:345-352).

While organoids, pancreatic organoids, or pancreatic islet organoids (e.g., HILOs) are preferably engineered to express one or more checkpoint proteins as described herein, an immune reaction to the transplanted organoid (e.g., HILO) may be further reduced in the subject by encapsulating the organoid, pancreatic organoid, or pancreatic islet organoid (HILO) in a hydrogel prior to transplanting in the subject. Such methods of transplantation are further described in Vegas et al., 2016, Nature Medicine. doi:10.1038/nm.4030; Vegas et al., 2016, Nature Biotechnology, doi:10.1038/nbt.3462. In some embodiments, the hydrogel contains an alginate or alginate derivative (e.g., triazole-thiomorpholine dioxide). Various modifications of alginate hydrogels that substantially reduce inflammatory or fibrotic effects of alginate hydrogels have also been identified (Vegas et al., 2016, Nature Biotechnology, doi:10.1038/nbt.3462). Thus, in some other embodiments, the hydrogel contains a chemical modification that reduces an inflammatory effect of the transplanted organoid in the subject.

Screening Assays Pancreatic islet organoids and pancreatic organoids (HILOs) as described herein can be employed to model diseases of the pancreas in vitro or in vivo. Such pancreas disease models can identify drugs that are useful for treatment of a pancreatic disease. Thus, in some aspects, the invention provides methods for identifying modulators, i.e., candidate or test compounds or agents (e.g., proteins, peptides, peptidomimetics, peptoids, polynucleotides, small molecules or other drugs) that can treat a pancreatic disease, particularly type 2 diabetes and/or pancreatic cancer. In one embodiment, the compound or agent modulates an activity of a pancreatic organoid or pancreatic islet organoid (HILO) as described herein.

The test compounds or agents can be obtained singly or using any of the numerous approaches in combinatorial library methods known in the art, including, but not limited to, biological libraries; peptoid libraries (libraries of molecules having the functionalities of peptides, but with a novel, non-peptide backbone which are resistant to enzymatic degradation and remain bioactive; see, e.g., Zuckermann, R. N. et al., 1994, J Med. Chem., 37:2678-85; spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des., 12:145).

Examples of methods for the synthesis of molecular libraries can be found in the art, for example in: DeWitt et al., 1993, Proc. Natl. Acad. Sci. U.S.A., 90:6909; Erb et al., 1994, Proc. Natl. Acad. Sci. USA, 91:11422; Zuckermann et al., 1994, J. Med. Chem., 37:2678; Cho et al., 1993, Science, 261:1303; Carrell et al., 1994, Angew. Chem. Int. Ed. Engl., 33:2059; Carell et al., 1994, Angew. Chem. Int. Ed. Engl., 33:2061; and Gallop et al., 1994, J. Med. Chem., 37:1233.

Libraries of compounds may be presented in solution (e.g., Houghten, 1992, Biotechniques, 13:412-421), or on beads (Lam, 1991, Nature, 354:82-84), chips (Fodor, 1993, Nature, 364:555-556), bacteria (Ladner, U.S. Pat. No. 5,223,409), spores (Ladner U.S. Pat. No. 5,223,409), plasmids (Cull et al., 1992, Proc Natl Acad Sci USA, 89:1865-1869) or on phage (Scott and Smith, 1990, Science, 249:386-390; Devlin, 1990, Science, 249:404-406; Cwirla et al., 1990, Proc. Natl. Acad. Sci. USA, 87:6378-6382; Felici, 1991, J Mol. Biol., 222:301-310; and Ladner, Ibid., supra).

Chemical compounds to be used as test agents (i.e., potential inhibitors, antagonists, agonists) can be obtained from commercial sources or can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, 1989, Comprehensive Organic Transformations, VCH Publishers; T. W. Greene and P. G. M. Wuts, Protective Groups in Organic Synthesis, 2nd ed., John Wiley and Sons (1991); L. Fieser and M. Fieser, Fieser and Fieser's Reagentsfor Organic Synthesis, John Wiley and Sons (1994); and L. Paquette, ed., Encyclopedia of Reagents for Organic Synthesis, John Wiley and Sons (1995), and subsequent editions thereof.

Combinations of substituents and variables in compounds encompassed by these methods are only those that result in the formation of stable compounds. The term “stable”, as used herein, refers to compounds that possess stability sufficient to allow manufacture and that maintain the integrity of the compound for a sufficient period of time to be useful for the purposes detailed herein (e.g., transport, storage, assaying, activity, therapeutic administration to a subject).

The compounds described herein can contain one or more asymmetric centers and thus occur as racemates and racemic mixtures, single enantiomers, individual diastereomers and diastereomeric mixtures. All such isomeric forms of these compounds are expressly included in the described methods. The compounds described herein can also be represented in multiple tautomeric forms, all of which are included herein. The compounds can also occur in cis- or trans- or E- or Z-double bond isomeric forms. All such isomeric forms of such compounds are expressly included.

Test agents, molecules and compounds can also be peptides (e.g., growth factors, cytokines, receptor ligands) or polynucleotides encoding such peptides, and the like.

Screening methods identify agents that increase or decrease a biological activity of pancreatic organoids and pancreatic islet organoids (e.g., HILOs) as described herein. In some embodiments, a pancreatic disease, such as diabetes, (e.g., type 2 diabetes) or pancreatic cancer, is induced or mimicked in the pancreatic islet organoid (e.g., HILO) or pancreatic organoid. Type 2 diabetes in the pancreatic organoid or pancreatic islet organoid (e.g., HILO) can be induced, for example, by contacting the organoid with free fatty acids (FFAs), glucose, and cytokines (in particular, high levels of glucose and/or high levels of FFAs). In one embodiment, a pancreatic organoid or pancreatic islet organoid (e.g., HILO) is co-cultured with pancreatic cancer cells, stellate cells and immune cells to create a human pancreatic cancer microenvironment in vitro.

In some embodiments, the organoid is contacted with a candidate agent, molecule, or compound, and an effect of the candidate agent, molecule, or compound on a biological activity, function, or event is assayed. In some embodiments, the candidate agent, molecule, or compound is a drug approved by the Food and Drug Administration (FDA). For example, biological activities of a pancreatic organoid or pancreatic islet organoid (e.g., HILO) assayed in the screening methods include insulin secretion (e.g., glucose-stimulated insulin secretion (GSIS)), beta cell apoptosis, LDHA activity, K(ATP) channel activity, mitochondrial function, level or activity of NDUFA4, ESRRG, KCNK3, or MAFA polypeptides or encoding polynucleotides, cell death, cell growth, and metastasis. In some embodiments, the agent, molecule, or compound increases GSIS.

In other embodiments, pancreatic islet cells, pancreatic organoid, or pancreatic islet organoid (e.g., HILO) is transplanted or implanted into a host to model pancreatic disease, such as type 2 diabetes or pancreatic cancer, in vivo. Methods of transplanting or implanting an organ or organoid are known in the art. The host can be any non-human mammal, such as a rat or mouse.

In addition to the expression of a checkpoint protein in cells, islets, organoids, pancreatic islet cells, pancreatic organoids, or pancreatic islet organoids (e.g., HILOs) for evading autoimmunity and immune detection, a recipient's immune reaction to the transplanted biological material, such as an organoid (e.g., HILO), can be further reduced, if desired, by encapsulating the organoid (e.g., HILO) in a hydrogel and then transplanting the encapsulated organoid (e.g., HILO) in the animal. Such methods of transplantation are described in Vegas et al., 2016, Nature Medicine, doi:10.1038/nm.4030; and Vegas et al., 2016, Nature Biotechnology, doi:10.1038/nbt.3462. In some embodiments, the hydrogel contains an alginate or alginate derivative (e.g., triazole-thiomorpholine dioxide). Various modifications of alginate hydrogels that substantially reduce inflammatory or fibrotic effects of alginate hydrogels have also been identified (Vegas et al., 2016, Nature Biotechnology, Ibid.). In still other embodiments, the hydrogel contains a chemical modification that reduces an inflammatory effect of the transplanted organoid in the host.

In some embodiments, a pancreatic organoid or pancreatic islet organoid (e.g., HILO) and liver organoid are co-transplanted or implanted in the animal. The liver is a major target organ for metastasis of pancreatic cancer. In mice, in vivo endothelial cells in the mini pancreas and in the mini liver are connected to each other and create a pancreas-liver vasculature network for pancreatic cancer metastasis. Therefore, an animal co-transplanted with a a pancreatic organoid or pancreatic islet organoid (e.g., HILO) and a liver organoid can be useful for studies of human pancreatic cancer metastasis into human liver. In some embodiments, the co-transplanted organoids are subjected to multiple intermittent exposures to IFNγ (MPS procedure) according to the methods as described herein.

In some embodiments, an animal transplanted with an organoid (e.g., HILO) as described herein is administered an environmental stress (e.g., a high fat/high glucose diet or is administered pancreatic cancer cells) to induce or mimic pancreatic disease in the animal. In some other embodiments, the animal is transplanted with a pancreatic islet, pancreatic organoid, or pancreatic islet organoid (e.g., HILO) and/or a liver organoid in which a disease (e.g., type 2 diabetes or pancreatic cancer) has been induced.

In some embodiments, a candidate agent, molecule, or compound is administered to an animal. In certain embodiments, the candidate agent, molecule, or compound is a drug approved by the Food and Drug Administration (FDA). In some embodiments, an effect of the candidate agent, molecule, or compound on a phenotype in the animal (such as biological activity or function associated with the pancreas, or activities associated with a disease such as pancreatic disease) is assayed. Exemplary, yet nonlimiting, biological activities include one or more of insulin secretion (e.g., glucose-stimulated insulin secretion (GSIS)), beta cell apoptosis, lactate dehydrogenase (LDHA) activity, K(ATP) channel activity, mitochondrial function, level or activity of NDUFA4 (Cytochrome c oxidase subunit NDUFA4), ESRRG, or MAFA (musculoaponeurotic fibrosarcoma oncogene family, protein A) polypeptide or encoding polynucleotide, cell death, cell growth, and metastasis. In some embodiments, the candidate agent, molecule, or compound increases GSIS.

In any one of the embodiments herein, the effect of the candidate agent, molecule, or compound (i.e., ability to modulate a pancreatic activity or function) is measured relative to a reference or control. The reference can be, for example, an untreated pancreatic organoid or pancreatic islet organoid. In some embodiments, the reference is a host transplanted with an organoid (e.g, HILO) as described herein, where the host is not administered a candidate agent, molecule, or compound.

Agents, molecules, or compounds useful in the methods as described herein can also be detected by identifying an increase in expression of a desirable marker (e.g., MAFA as a beta cell fate marker). The level of expression can be measured in a number of ways, including, but not limited to, measuring the mRNA encoded by the genetic markers; measuring the amount of protein encoded by the genetic markers; or measuring the activity of the protein encoded by the genetic markers.

The level of mRNA corresponding to a marker can be determined both by in situ and by in vitro formats. The isolated mRNA can be used in hybridization or amplification assays that include, but are not limited to, Southern or Northern analyses, polymerase chain reaction (PCR) analyses and probe arrays. In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. The skilled practitioner can adapt known mRNA detection methods for use in detecting the level of mRNA encoded by the markers described herein.

The level of mRNA in a sample can be evaluated with nucleic acid amplification, e.g., by rtPCR (C. Mullis, 1987, U.S. Pat. No. 4,683,202), ligase chain reaction (Barany, 1991, Proc. Natd. Acad. Sci. USA, 88:189-193), self-sustained sequence replication (Guatelli et al., 1990, Proc. Natd. Acad. Sci. USA, 87:1874-1878), transcriptional amplification system (Kwoh et al., 1989, Proc. Natd. Acad. Sci. USA, 86:1173-1177), Q-Beta Replicase (Lizardi et al., 1988, Bio Technology, 6:1197), rolling circle replication (Lizardi et al., U.S. Pat. No. 5,854,033), or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule (polynucleotide) comprising the nucleotide sequence flanked by the primers.

### Kits

Also provided are kits containing an immunoprotected cell, human islet-like organoid or pancreatic islet organoid as described herein, or a pharmaceutically acceptable composition (therapeutic composition) containing the immunoprotected cell, human islet-like organoid or pancreatic islet organoid and a pharmaceutically acceptable carrier, diluent, or excipient, for administering to, or transplanting into, a subject in need thereof. As will be appreciated by the skilled practitioner in the art, such a kit comprises a sterile container which contains the therapeutic composition; such containers can be boxes, ampoules, bottles, vials, tubes, bags, pouches, or other suitable container forms known in the art. The containers can be made of plastic, glass, or other materials suitable for holding biological medicaments. In some embodiments, a kit may include multiple containers that house the immunoprotected cell, human islet-like organoid or pancreatic islet organoid, a composition thereof, diluents, vehicles, or excipients, as necessary, and instructions for use. The instructions will generally include information about the use of the immunoprotected cell, human islet-like organoid or pancreatic islet organoid or composition thereof for treating a disease, such as a pancreatic disease or diabetes. In other embodiments, the instructions include at least one of the following: description of the therapeutic agent (immunoprotected cell, human islet-like organoid or pancreatic islet organoid); dosage schedule and administration for treatment of the disease, or transplantation; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Advantages and Applicability of the Embodiments

A combination of genetic and environmental factors underlies the autoimmune destruction of ß cells, and while exogenous insulin provides glycemic control, the long-term complications associated with Type 1 diabetes are a continuing concern. Thus, the ability to generate ß cells suitable for transplantation has the potential to significantly improve patients' lives. While cadaveric islet cell transplantation offers one mode of therapy, alternative stem cell-based approaches continue to face numerous challenges in generating GSIS competent ß cells on a large-scale and protecting transplanted cells from auto-immunity and allogenic rejection. For the latter, it is generally considered that self-contained transplantation devices, immune suppressive therapies, or both are required.

The methods and systems described herein provide useful protocols, such as 3D culturing conditions that systematically drive the differentiation of pluripotent stem cells (e.g., hiPSCs), stem cells, or embryonic stem (ES) cells, into insulin-positive, glucose-sensitive ß-like cells, and lead to the generation of metabolically mature, immune evasive human islet-like organoids (wHILO^{ie}) capable of secreting insulin in response to a glucose challenge. Furthermore, these functionally mature HILOs rapidly reestablish glucose homeostasis upon transplantation into diabetic, immune-competent mice. A feature of the described protocols furthers the inventors' discoveries that oxidative mitochondrial metabolism was central for postnatal ß cell maturation and that the transcription factor ERRγ was necessary and sufficient for this metabolic program. The identification of WNT4 as a potent maturation factor for inducing both ERRγ expression and for enhancing mitochondrial oxidative phosphorylation allowed for the production of wHILOs in fully chemically defined medium (FIGS. 3F and 3H).

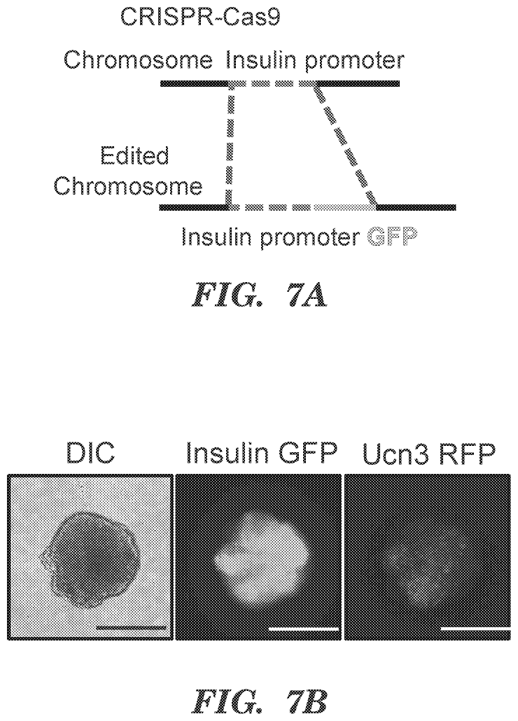

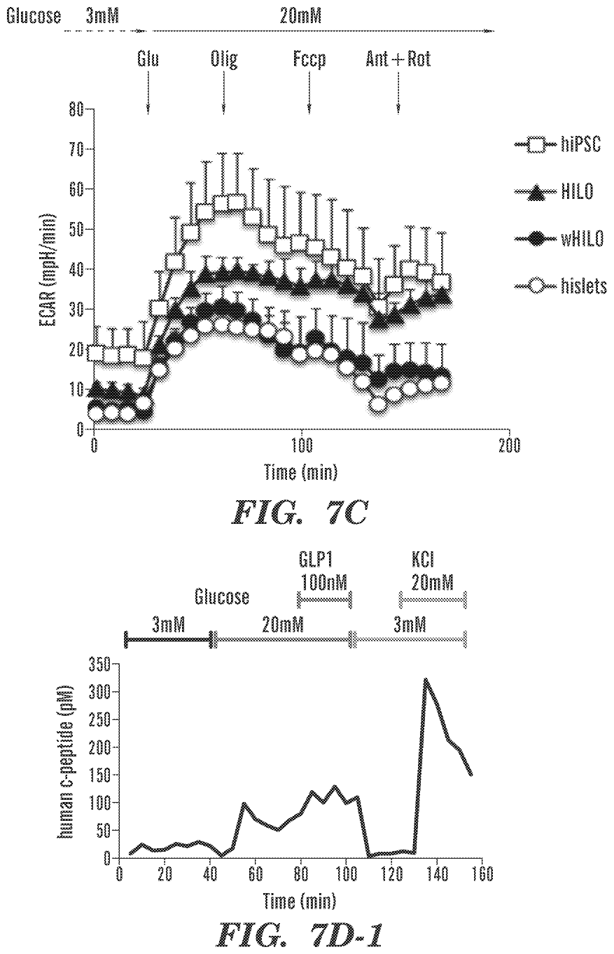

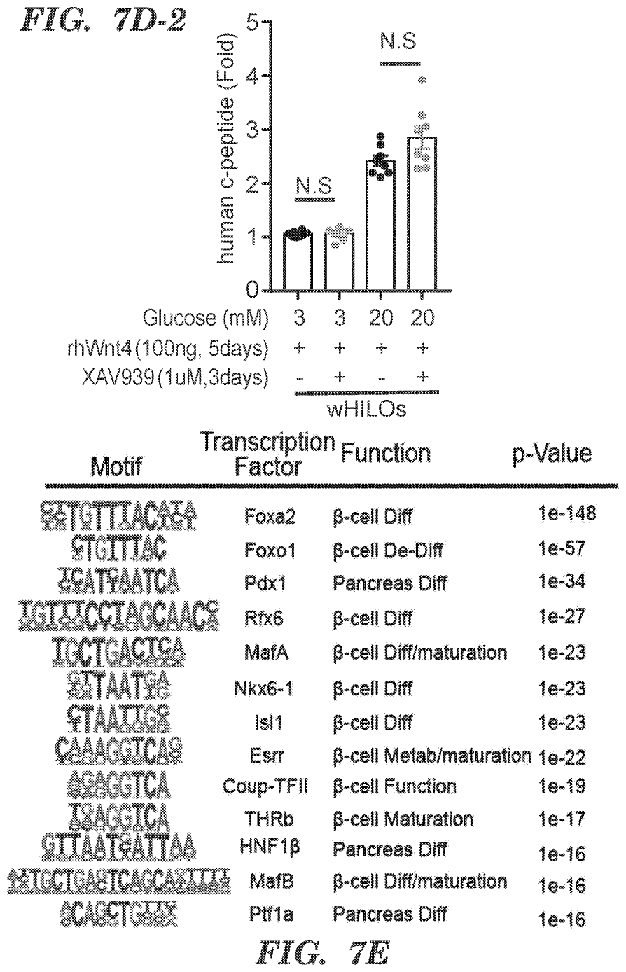

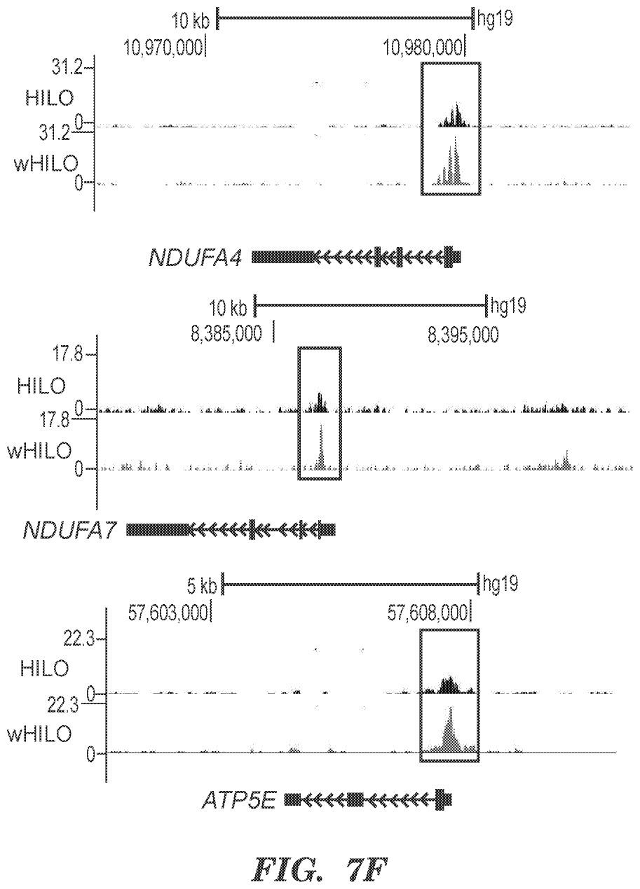

As would be appreciated by the skilled practitioner, challenges for stem cell-based therapeutics include autoimmune rejection of transplanted cells, in addition to metabolic and functional maturity of the cells. However, the methods, systems, and biological products generated and provided herein provide advantageous solutions to such challenges. By way of example, the finding that wHILOs maintained functionality in NOD-SCID but not in C57BL6J mice implicates T cells and β cells in the xenograft rejection (FIG. 3K and FIG. 7C). During antigen presentation, interactions between cytotoxic T-lymphocyte antigen-4 (CTLA-4) and B7 molecules, as well as programmed cell death protein 1 (PD1) receptor and its ligand PD-L1, negatively regulate immune responses in anon-redundant manner. As described and exemplified herein, HILOs, such as wHILOs, overexpressing PD-L1 are protected from xenograft (FIG. 4C) and allogenic (FIG. 4K) rejection. As further described and exemplified herein, methods and systems were developed in which multiple, repeated exposures to limited IFNγ concentrations (IFNγ MPS treatment method) over period of time led to sustained, endogenous PD-L1 expression without compromising the GSIS activity of the cells (e.g., β-cells), HILOs and the cells therein. Notably, the resultant immune evasive HILOs maintained glucose homeostasis in immune-competent as well as in humanized diabetic mice in the absence of a transplantation device.

The generation of iPSCs by somatic cell reprogramming provides a source of patient-specific syngeneic or autologous cells that can potentially be differentiated into any lineage. Thus, generating insulin-producing cells from iPSCs for autologous transplantation might dramatically reduce the risk for autoimmune rejection. However, in practical terms, generating clinical-grade autologous transplants that meet manufacturing standards, quality assurance, and regulatory compliance involves expensive and time-consuming procedures. Although the allogenic transplantation of MHC-matching grafts has proven effective in reducing immune responses, this technique generally does not result in complete evasion of the immune system, even in less immunological sites such as the brain. Furthermore, the possible destruction of the transplanted insulin-producing cells by autoreactive T cells remains. Thus, the present methods and their resulting cells and products (e.g., immune evasive HILOs and cells) provide beneficial and long-lasting therapeutics that maintain function (e.g., GSIS) and integrity for significant time periods after transplantation or administration to a subject in need. In embodiments, MHC matching and/or the induction of immune tolerance may further be employed to control immune responses, optimally without immunosuppressive drugs.

Provided and described in an embodiment herein are advantageous methods and culture systems (e.g., a 3D culture system) for the generation of human islet-like organoids (HILOs). The methods and systems incorporate non-canonical WNT signaling to promote metabolic maturation and glucose-sensitive insulin secretion in HILOs and the cells therein, and limited IFNγ exposure, namely, multiple pulse stimulation with IFNγ, to drive the sustained expression of endogenous PD-L1 in the HILOs and cells therein. The ability to generate functional immune evasive HILOs, e.g., wHILO^{ie}, that are capable of avoiding immune detection over a significant period of time (over 50 days or longer) represents a major advance that offers a viable alternative to current cadaveric islet use or device-dependent technologies.

The practice of the methods and protocols described herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as in “Molecular Cloning: A Laboratory Manual”, second edition (Sambrook, 1989), as well as subsequent editions; “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides described herein, and, as such, may be considered and employed in making and practicing the invention.

Particularly useful techniques for particular embodiments are discussed in the following examples, which are set forth to provide those of ordinary skill in the art with a complete disclosure and description of how to make and use the products, assays, procedures, screening, and therapeutic methods as described, without intending to limit the description and disclosure herein.

### EXAMPLES

### Example 1: Generation and Characterization of Pancreatic and Pancreatic Islet Organoids

Although an animal disease model can yield insight into the pathogenesis of diseases, drugs identified from screens using animal models often fail to be adopted in human patients. Generation of functional human organoids provides a new therapeutic strategy in drug-screening and disease modeling. Described herein is a technique to generate a 3D human pancreatic mini-organ, or organoid (e.g., HILO), in a dish. Using this technique, diseases such as human type 2 diabetes can be modeled in vitro to find effective drugs in genetic, patient or environmental specific diseases such as human type 2 diabetes.

### Developing Gellan Gum Based 3D Culture System for β-Like Cells Differentiation

It is known that 3 dimensional (3D) culture systems contribute to facilitating self-organization and integration of cells. Therefore, MATRIGEL® matrix containing extracellular matrix components such as collagen and fibronectin is often used as the basement of a 3D culture system. However, MATRIGEL® matrix-based 3D culture systems are not ideal for large-scale human organoid generation because of their cost and difficulties in scale up. Described hereinbelow are Gellan-gum based 3D culture systems and methods for β-like cell differentiation, which are cost effective and easily scalable. In an embodiment, using a fully chemically-defined stepwise differentiation protocol, human pluripotent cells (hPSCs) are differentiated into insulin producing islet-like spherical cell clusters with high efficiency and reproducibility in Gellan-gum based 3D culture systems. Single dissociated pluripotent stem cells (PSCs) successfully formed into spheres within 5 days in Gellan gum containing STEMCELL™ TeSR™ media. Fifteen (15) to 21 days after differentiation in Gellan gum-containing Custom TeSR™ with defined small molecule stimulation, insulin positive GFP clusters were observed. Global transcriptome analysis by RNA-seq revealed the stepwise differentiation of hiPSCs into insulin positive cells expressing R cell lineage specific marker genes including Pdx1, Nkx6-1, GATA6 and MAFB. The differentiation of hiPSCs, as well as the human ESC lines HuES8 and HIES, into islet-like cell clusters was further confirmed by the progressive loss of the pluripotent marker Nanog, the induction of the B cell specific marker Nkx6-1, and the progressive induction of the endocrine hormones insulin, somatostatin and glucagon, as determined by qPCR. These results demonstrated that the Gellan-gum based 3D culture systems is suitable for the generation of large-scale islet-like organoids from hPSCs.

### Generation of Scalable, Human Islet-Like Organoids In Vitro

β-like cells derived from human embryonic stem cells (hESC) or human induced pluripotent stem cells (hiPSC) have limited functionality and lack the morphological and functional feature of human islets. Previous studies revealed that co-culturing hiPSC derived hepatocyte with human umbilical vein endothelial cells (HUVECs) and human bone marrow-derived mesenchymal stem cells (hMSC) generates self-organized 3D liver-bud spheres in matrigel (Takebe et al., 2013, Nature, 499:481-484). This study found that the liver “organoids” had superior expression of lineage determinant factors compared to the differentiation of isolated hepatocytes and that these organoids rapidly vascularized and functionally matured in vivo.

Studies have found that hiPSC-derived pancreatic progenitor cells (hiPSC-PP) generated using a 2D differentiation protocol (Yoshihara et al, 2016, Cell Metab. 23, 622-634) did not self-organize in 3D MATRIGEL® matrix. (See, e.g., WO 2017/205511). In contrast, HUVEC cells rapidly formed a vasculature-like structure while human adipocyte-derived stem cells (hADSCs) self-organized in 3D MATRIGEL® matrix. In MATRIGEL® matrix, dispersed hADSC cells projected processes within 4 hours, formed a cloth-like wrapper within 12 hours, and adopted a sphere-like formation within 24 to 48 hours. Furthermore, a minimum cell density for self-organization was identified (i.e., ~10,000-20,000 cells in 300 μl of MATRIGEL® matrix in −2 cm² well. RNA-seq analysis identified dynamic transcriptional changes during hADSC 3D self-organization, suggesting that the ability to self-organize under 3D culture conditions is an inherent feature of naïve hADSCs. These results identify the mesenchymal hADSC as a resource for generating self-organizing organoids.

To explore pancreatic organogenesis, hiPSC-PP (1×10⁶ cells) cells were co-cultured with HUVECs (7×10⁵ cells) and hADSCs (1-2×10⁵ cells) (FIGS. 1A and 1B) in Matrigel matrix. This co-culture yielded macroscopically visible 3D cell clusters 48 hours after seeding. Furthermore, insulin expression, based on the expression of a GFP reporter, was detected 5 days after seeding and increased with time in culture in the human islet-like organoids. In addition, HUVECs-based endothelial cells are integrated inside the organoids as shown by fluorescence-labeled (mCherry) HUVECs. The limitations of MATRIGEL® matrix for organoid production include high cost, difficult organoid recovery, scaling restrictions, and batch to batch variabilities.

Methods to generate morphologically identical human islet-like organoids using gellan gum based 3D cultures are described herein below and in WO 2017/205511. Human induced pluripotent stem cells derived pancreatic progenitors (hiPSC-PPs) (1×10⁸ cells) were cultivated with a stromal cell population such as human umbilical vein endothelial cells (HUVECs) (2-7×10⁶ cells) and human adipose-derived stem cells (hADSCs) (2-7×10⁶) in 50 ml of gellan gum based 3D culture media. HiPSC-PP rapidly formed isle-like sphere formation with HUVECs and hADSCs within 5 days after seeding into the gellan gum based 3D culture media. Human islets like mini-organs expressed human insulin GFP reporter in 5 days after seeding with gradually enhancing GFP intensity. Co-culturing hiPSC-PP, hADSCs, and HUVECs according to this method, generated human islet-like organoids with high reproducibility that were morphologically similar to human islets. In addition, the generated human islet-like organoids contained insulin granules in β-like cells. Gene expression analyses revealed increased expression of β cell fate determinant genes (Insulin, Nkx6-1, PCSK1 and UCN3) and mitochondrial related metabolic genes (Esrrg, Ndufal, Ndufa 12, Cox7a2. Atp5b) in the insulin expressing cell population (GFP enriched (GFP+)) in islet-like organoids compared to those prepared without hADSC and HUVEC co-culture. Glucose-stimulated human c-peptide secretion assay revealed that islet-like organoids generated by this method are able to secrete human c-peptide in response to high (20 mM) glucose.

An in vitro functional vascularization test was performed. Islet-like mini organs generated in gellan gum were transferred to MATRIGEL® matrix and cultured in endothelial growth media (EGM). Green fluorescence indicates expression of insulin genes. Within 24 hours to 48 hours after stimulation by EGM, the outgrowth of HUVEC cells was observed, indicating that human islet-like organoids generated by the method possessed the ability to form vascular structures.

### Establishment of Single Islet Insulin Secretion Assay Using Proinsulin-NanoLuc Gaussia Luciferase Assay System

It was previously published that a reporter construct, in which the Gaussia luciferase is placed within the c-peptide portion of proinsulin accurately measures insulin secretion without affecting β-cell function (Burns et al., 2015, Cell metabolism, 21, 126-137). Using a lentiviral system, INS-1 cells stably expressing this Gaussia luciferase were generated. Luciferase secretion from INS-1 cells stably expressing Proinsulin-NanoLuc increased with high-glucose (20 mM), high glucose with Exendin-4 (G20 mM+Ex4), and the depolarizing agent, potassium chloride, confirming the utility of this reporter system. Next, the usefulness of this reporter to measure insulin secretion in mouse or human islets transiently infected with the Proinsulin-NanoLuc reporter was evaluated. Luciferase secretion in response to 20 mM high glucose was detected in both transiently infected mouse and human islets were detected. Importantly, the assay sensitivity was sufficient that insulin secretion could be qualified at the level of single islets. These results indicate that the Proinsulin-NanoLuc luciferase reporter based insulin secretion assay is applicable to not only the rat beta cell line INS-1 cells, but also to primary mouse and human primary β cells. (See, e.g., WO 2017/205511).

Establishment of hiPSC and hESC Cells Incorporating Dual Lineage and Functional Reporters

Human iPSCs and hESCs stably expressing reporters for Pcell lineage (human insulin reporter) and β cell function (proinsulin-NanoLuc reporter) were generated, hiPSC^{hINS-GFP/Sec-Luc} and hESC^{hINS-GFP/Sec-Luc}, respectively. First, a neomycin resistant construct of human insulin GFP reporter was generated by inserting human insulin promoter sequence of pGreenZeo lenti-reporter (SR10028PA-1, System Bioscience) into pGreenFire Lenti-Reporter plasmid (TR019PA-1, System Bioscience) (named as hINS-GFP-EF1a-Neo). hINS-GFP-EF1a-Neo lenti virus was infected into hiPSC and hESC by spin fection (800 g, 1 hour, 37° C.) followed by a medium changed to fresh STEMCELL™ TeSR™ medium. Three (3) days after the first infection, the cells were treated with 100 μg/ml G418 in STEMCELL™ TeSR™ medium for 7 days. Selected hiPSC and hESC cells stably expressing hINS-GFP-EF1a-Neo were subsequently infected with the Proinsulin-NanoLuc (Addgene, Plasmid #62057) lenti-virus by spin fection (800 g, 1 hour, 37° C.) followed by a medium change to fresh STEMCELL™ TeSR™ medium. Three (3) days after the second infection, the cells were treated with 5 μg/ml blasticysin and 100 μg/ml G418 in STEMCELL™ TeSR™ medium for 7 days. Subsequently, cells were maintained in STEMCELL™ TeSR™ medium. The generated stable cell lines incorporating the dual reporters maintained self-renewal and pluripotency capabilities, as well as the capacity to differentiate into insulin producing β like cells (see, e.g., WO 2017/205511).

### Pooled Human Islet-Like Organoid Cultures Display Consistent Insulin Secretion Despite Variable Functionality Seen in Individual Organoids

Recent studies have reported the generation of insulin producing β-like cells from hESC and hiPSC capable of secreting insulin in response to glucose (Pagliuca et al., 2014, Cell, 159, 428-439; Rezania et al., 2014, Nature Biotechnology, 32(11):1121-33; Russ et al., 2015, EMBO Journal, 34:1759-1772). However, fully functional human islet-like clusters able to appropriately secrete insulin in response to nutritional signals including glucose, amino acids, fatty acids and incretins such as GLP-1 have yet to be demonstrated. To date, efforts have focused on the independent generation of insulin producing β-like cells, glucagon producing u-like cells, and somatostatin producing δ-like cells from hPSC. However, these approaches lack the supporting cells important for regulation, such as mesenchymal cells, adipose cells, and vasculature cells. Since the 3D structure of islets naturally enhances their function, these missing cellular components may compromise the functionality of islet-like cells clusters. In addition, organogenesis of pancreatic islets involves clonal expansion of β-cells, suggesting that these cells may have multiple functions in islet-like organoids. To test this idea, single organoid proinsulin secretion assays were performed. Human islet-like organoids generated by methods described herein are morphologically identical with human islet. However, significant variability was seen in the glucose-stimulated insulin secretion (GSIS) capabilities of individual human islet-like organoids compared to human islets, as measured by proinsulin luciferase secretion assay. Consistent GSIS functionality was demonstrated in pooled organoids (10 to 100 organoids for assay). Furthermore, pooled human islet like organoids demonstrate enhanced GSIS when co-stimulation with GLP-1, as well as robust KCl-stimulated insulin secretion.

In vitro cultured iPSC-derived human pancreatic islet-like organoids generated herein retained their ability to respond to glucose, GLP1 and KCl after extended time (133 days) in culture.

### Example 2: Transplantation of Functional Pancreatic Islet Organoids Rescued Type 1 Diabetic Mice

Expression of specific functional islets markers such as MAFA, UCN3 and mitochondrial oxidative genes such as ERRγ (Esrrg), Ndufa 1, Ndufa 12, Cox7a2 and Atp5b in hiPSC-derived human islet-like organoids was observed, as further described in the below Examples. Notably, these islet-like organoids recapture in a dish both human islets development as well as the pathogenesis of diabetes. Transplantation of these functional islet-like organoids rescue type 1 diabetic mice with long survival, rapid vascularization, and reduced immune rejection.

### Example 3: Wnt Proteins in the Metabolic Maturation of iPSC-Derived Islet Organoids

Fltp and Esrrg genes were found to be expressed in iPSC-derived islet organoids (day 21, generated without co-culture with hADSCs or HUVECs) after treatment with PBS, WNT3a (500 ng/ml), recombinant human (rh)WNT4 (100 ng/ml), or rhWNT5a (400 ng/ml) for 5 days. Esrrg gene expression was induced in hiPSC-derived islet organoids that were generated in the absence of supporting hADSC or HUVECs, in response to increasing doses of rhWNT4 (0, 10, 25, 50, 100, 200 ng/ml) and rhWNT5a (0, 25, 50, 100, 200, 400 ng/ml). In addition, mitochondrial genes involved in oxidative phosphorylation (Cox7a2, Ndufa1, Ndufa7), lactate dehydrogenase (Ldha) and Fltp (a Wnt/planar cell polarity (PCP) effector and reporter gene) were induced in hiPSC-derived islet organoids that were generated in the absence of supporting hADSC or HUVECs, in response to increasing doses of rhWNT4 (0, 10, 25, 50, 100, 200 ng/ml) and rhWNT5a (0, 25, 50, 100, 200, 400 ng/ml). Mitochondrial (Mitotracker; Mito-Red) and insulin (Insulin-GFP) levels were increased in hiPSC-derived islet organoids (day 27) after 8 days treatment with PBS or WNT4 (100 ng/ml). Human iPSC-derived islet organoids (day 27) were generated after 8 days treatment with PBS or WNT4 (100 ng/ml). Insulin production was found in hiPSC-derived islet organoids (day 27) after 8 days treatment with rhWNT4 (100 ng/ml), rhWNT5a (400 ng/ml), or WNT5a secreting fibroblast conditioned media (50%), compared with PBS and control fibroblast conditioned media (50%). Human iPSC (hiPSC)-derived islet organoids (day 22) treated with rhWnt4 (100 ng/ml) for 12 days showed functional maturation based on their secretion of human c-peptide, as measured in response to low glucose (3 mM, “G3 mM”), high glucose (20 mM, “G20 mM”), or high KCl levels (20 mM, “KCL20 mM”), (see, e.g., WO 2017/205511).

### Example 4: Generation of Functional Human Islet-Like Organoids (HILOs) from Induced Pluripotent Stem Cells (iPSC) Using a Functional Polymer-Based 3D Culture System

Stem cell-derived human islets hold promise as a therapy for insulin dependent diabetes. This Example describes the generation of human islet-like organoids (HILOs) from induced pluripotent stem cells (iPSCs) and shows that activation of the non-canonical WNT pathway drives a metabolic maturation step necessary for robust glucose-stimulated insulin secretion. These functionally mature HILOs containing multiple endocrine cell types maintain glucose homeostasis upon transplantation into diabetic NOD-SCID mice. Furthermore, overexpression of PD-L1 generated immune evasive, immunologically protected HILOs that maintained glucose homeostasis in immune-competent type 1 diabetic mice for at least 50 days. The ability to generate, in a scalable fashion, functional islet-like organoids that avoid immune detection provides an advantageous and beneficial new therapy for diabetes.

Islet transplantation provides superior long-term blood glucose control for type 1 and late-stage type 2 diabetics; however, the availability and quality of cadaveric islets is currently limiting. While the differentiation of induced pluripotent stem cells (iPSCs) into insulin-producing β-like cells represents an advance in the field, the methods for generating functional β-like cells appropriate for human therapy and treatment provided herein provide biologically functional cell and HILO products suitable for use as therapeutics and in transplantation.

As described, an ERRγ-driven, postnatal metabolic maturation step is necessary for β cell glucose stimulated insulin secretion (GSIS). In addition, ERRγ overexpression in iPSC-derived β-like cells was sufficient for in vitro and in vivo functionality. To generate functional cells suitable for transplantation, culture conditions that replicate the cellular architecture, as well as the cell type complexity of islets, were developed. Accordingly, as transcriptionally-similar models of pancreatic fibroblast and epithelial cells, human adipose derived stem cells (hADSCs) and human umbilical vein endothelial cells (HUVECs) were used for their cell-intrinsic abilities of to form organ-like and vascular structures, respectively, when grown in 3 dimensional (3D) Matrigel cultures (FIG. 1A). Incorporating hADSCs and HUVECs during the differentiation of human induced pluripotent stem cell (hiPSC)-derived endocrine progenitors (EPs) in a 3-dimensional polysaccharide based gel (gellan gum) led to the formation of multicellular spheroids (MCSs), comparable in size to human islets. (FIG. 1B; FIGS. 6A-6F). These MCSs contain insulin-producing cells, as seen from the expression of GFP driven by the insulin promoter and the presence of insulin granules (FIG. 1C); the incorporation of hADSCs was confirmed by the presence of cells containing lipids in droplet-like structures. (FIG. 1E). Compared to endocrine progenitors (EPs) differentiated in the absence of hADSCs and HUVECS (IS), the expression of ERRγ and the mitochondrial genes NDUFA1 and COX7A2 were increased in MCSs, consistent with functional metabolic maturation (FIG. 1D). Consistent with their functional maturation, the MCSs displayed improved insulin secretion in response to a glucose challenge (measured by c-peptide secretion), (FIG. 1E). In addition, MCSs developed vascular-like structures when stimulated with endothelial growth media, suggesting the possibility of extended in vivo functionality (FIG. 6C). Indeed, MCSs transplanted into the kidney capsule were able to maintain glucose homeostasis for approximately 40 days in STZ-induced diabetic NOD-SCID mice (diabetic mouse model), displaying similar efficacy to human islet transplantations (FIG. 1F). Furthermore, transplanted MCSs remained glucose responsive, appropriately regulating insulin secretion in the fed, fasted, and refed states as indicated by c-peptide levels (FIG. 1G); (mouse insulin levels were <0.2 ng/ml, not shown).

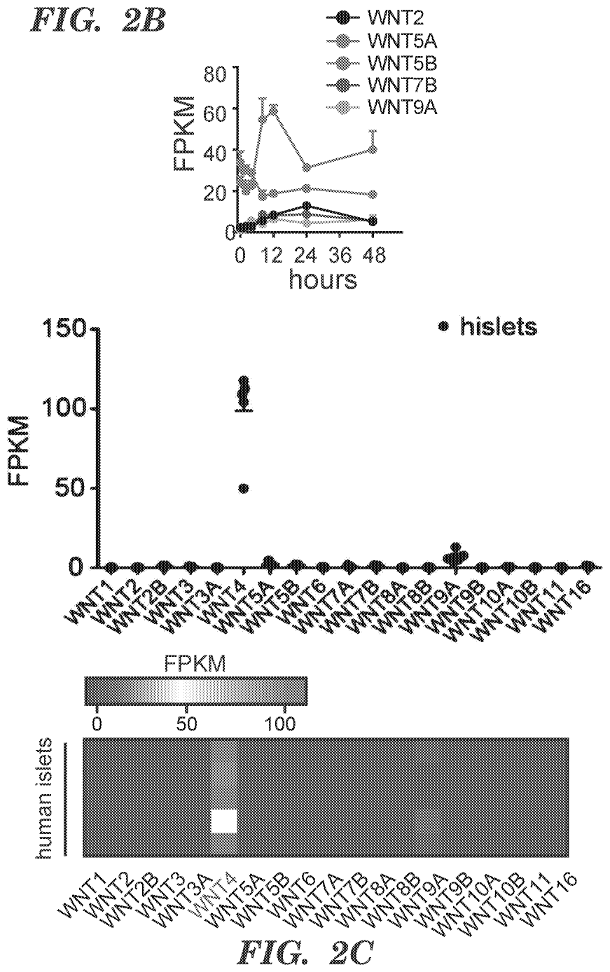

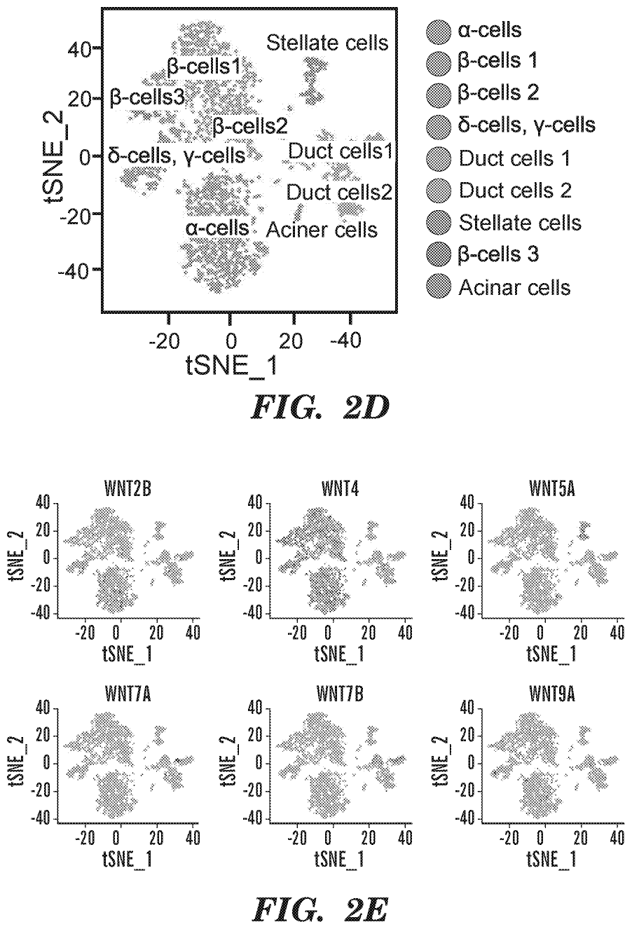

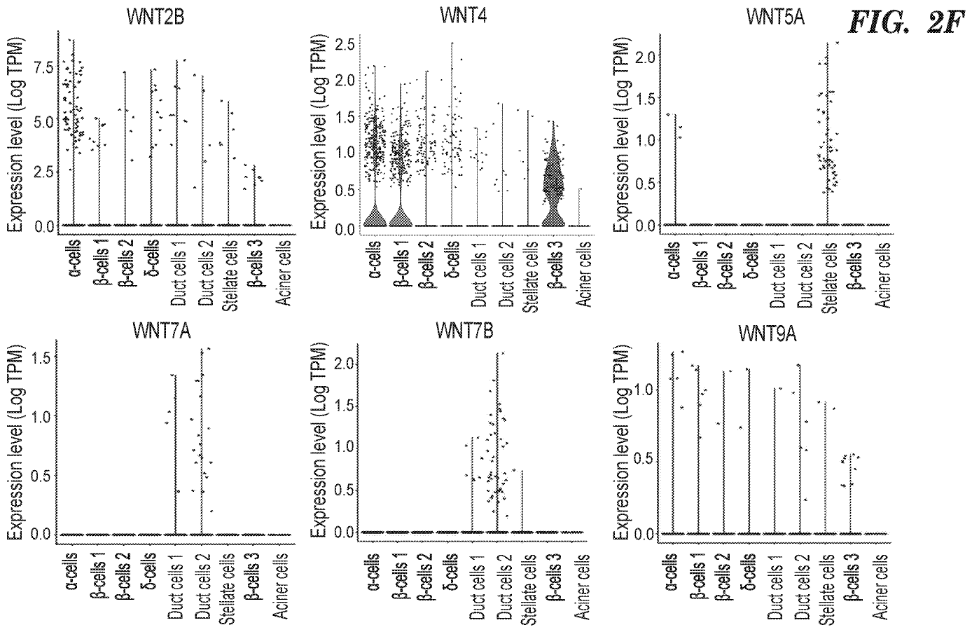

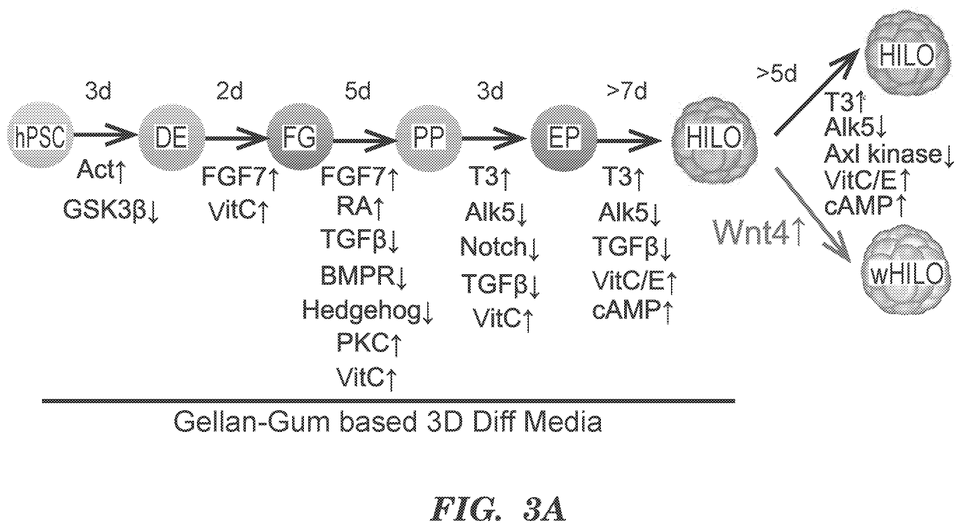

The results obtained support the role of 3D multicellular interactions in organogenesis, as previously shown for liver organoids. The transcriptional changes induced during the initial 48 hours of hADSC single cell type 3D culture were assessed to understand the molecular signals driving the cell-intrinsic ability to self-assemble (FIG. 2A). Gene ontology analysis identified metabolic and cytokine signaling pathways, as well as WNT signaling, enriched in the altered transcripts (FIG. 2A). Consistent with this, the temporal expressions of WNTs during hADSC self-assembly revealed a transient, approximately 2-fold increase in WNT5a expression that coincided with the initial cell-cell interactions observed in three dimensional (3D) cultures (FIG. 2B).

### Example 5: The Non-Canonical Wnt Pathway Regulates Gene Expression to Enable Oxidative Phosphorylation and Maturation of HILOs

The non-canonical WNT pathway is a marker for non-proliferative, mature β cells, and WNT4 expression is enhanced during the postnatal functional maturation of mouse islets. In experimental studies using human islets, WNT4 was discovered to be highly expressed in the human islets (FIG. 2C), in agreement with these findings. Moreover, single cell sequencing of human islets identified widespread expression of WNT4 in β and α cells, along with more restricted WNT5A expression predominantly in stellate cells (FIGS. 2D, 2E, 2F; FIGS. 6D-6F). To demonstrate that non-canonical WNT signaling was sufficient for the functional maturation of iPSC-derived β cells or β-like cells, CRISPR-Cas9 genome editing was used to insert the GFP coding sequences downstream of the insulin promoter in hiPSCs (FIG. 7A), to generate a reporter for endogenous insulin promoter activity and to allow endogenous insulin promoter activity to be visualized. These engineered hiPSCs were subsequently differentiated in a fully chemically-defined 3D culture system that incorporated WNT4 in the final endocrine progenitor (EP) maturation step (FIG. 3A). This optimized 3D differentiation protocol led to the formation of human islet-like organoids (HILOs) that expressed insulin (FIGS. 3A and 3B). In addition, expression of Urocortin-3, secreted from 3 cells to regulate 6 (delta) cell somatostatin secretion, co-localized with insulin in HILOs (FIG. 2B). The analysis of the HILOs by electron microscopy revealed structural similarity to human islets, most notably, by the presence of insulin and glucagon granules in the HILOs (FIG. 3C).

Comparative transcriptional analyses confirmed the induction of key islet cell markers in WNT4-treated HILOs (wHILOs) to levels comparable to those seen in human islets, including 1 cell specific genes (NKX2-2, NEUROD1, RFX6, GCK) and α cell-specific genes (ARX), (FIGS. 3D-1 and 3D-2). Importantly, the expression of β cell lineage specification markers, including INS, NKX6-1, UCN3, MAFB and SYT4, was not affected by the addition of WNT4, thus indicating that this non-canonical WNT signaling was not affecting cell fate determination. In contrast, WNT4 dose-dependently increased the expression of ERRγ (encoded by ESRRG), as well as components of the mitochondrial respiratory chain NDUFA7 and COX7A2 in HILOs (FIG. 3F). Consistent with these inductions, HILOs generated in the presence of WNT4 displayed increased oxidative metabolism, as measured by an increase in oxygen consumption rate (OCR) and decreased extracellular acidification rate (ECAR), replicating the metabolic characteristics of healthy human islets (FIG. 3H and FIG. 7C). WNT4 treated HILOs showed improved in vitro GSIS; an effect that was not blocked by the β-catenin inhibitor XAV939 (FIG. 3I; FIGS. 7D-1 and 7D-2). Similarly, culturing commercially-available hiPSC-derived β like cells in 3D differentiation medium containing WNT4 promoted pseudo-islet formation and GSIS functionality. (FIG. 3J and FIG. 3K). Importantly, wHILOs (i.e., HILOs cultured in culture or differentiation medium containing WNT4) restored glycemic control upon transplantation into STZ-induced NOD-SCID diabetic mice and maintained glucose homeostasis for more than 6 weeks (FIG. 8D). In combination, these results indicate that non-canonical WNT signaling is sufficient to induce a metabolic maturation of HILOs needed for robust GSIS, in a manner that mimics the postnatal maturation of human islets. Accordingly, culturing stem cells (e.g., hiPSCs, PSCs, or embryonic stem (ES) cells) in medium containing WNT (e.g., WNT4) generates islets and islet like organoids (wHILOs) which are functionally mature and islet-like and which express more mature ß-cell markers and produce insulin.

To understand the molecular transformations driving the maturation of HILOs, the transcriptional changes induced by WNT4 treatment of HILOs were assessed. The expression of 1581 and 1354 genes were increased and decreased, respectively, by WNT4 treatment (100 ng/ml for days 26-33). Gene ontology analysis identified metabolic pathways, most notably oxidative phosphorylation, enriched in this gene set FIG. 3E. Genes associated with the ribosome include mitochondrial translation and elongation gene clusters, as determined by GOTERM_BP analysis by DAVID, FIG. 8C). Consistent with an effect on cellular metabolism, WNT4 treatment comprehensively increased the expression of OxPhos genes in HILOs to levels similar to those seen in human islets, and increased mitochondrial number (FIG. 3G and FIG. 8A).

To examine the specific effects on the β-like cell population, insulin-expressing cells were sorted based on GFP expression from HILOs with and without WNT4 or WNT5a treatment. The proportion of insulin expressing cells was not affected by WNT treatment, in agreement with the invariant B cell lineage marker expression during HILO maturation (FIG. 8B). However, WNT4 and WNT5a treatment increased the mitochondrial content of the insulin-expressing cells, supporting the notion of a metabolic maturation of β cells (FIG. 8B). To identify genetic effectors of this maturation step, the WNT4-induced changes in chromatin accessibility were mapped in the sorted, GFP+ cells by ATAC-Seq. Widespread alterations in chromatin accessibility were seen with WNT4 treatment, in agreement with the extent of transcriptional changes. An overlap of the regions with increased chromatin accessibility with the HILO genes induced by WNT4 treatment identified 123 genes (FIG. 8E). Gene ontology identified metabolic pathways, including oxidative phosphorylation, enriched in this gene set. Furthermore, motif analysis in genes where increased chromatin accessibility corresponded with increased gene expression identified j cell maturation factors including Foxa2 and ERRs. (FIG. 8F). Consistent with this, WNT4-induced increases in chromatin accessibility were seen at oxidative phosphorylation genes including ERRγ target genes NDUFA4, NDUFA7 and ATP5E (FIG. 7F). Further supporting the essential role of ERRγ signaling, WNT4 (100 ng/ml for 5 days) induced the expression of mitochondrial metabolic genes and improved GSIS function in isolated neonatal islets from WT, but not from ERRγ B cell specific knockout (KO) mice (ERRγKO mice), (FIG. 8G and FIG. 8H). Without wishing to be bound by theory, these results, taken together, support the concept that non-canonical WNT4 signaling enhances mitochondrial function, in large part through the induction of ERRγ, to drive the metabolic maturation of 3-like cells.

### Example 6: Cellular Complexity of Mature HILOs

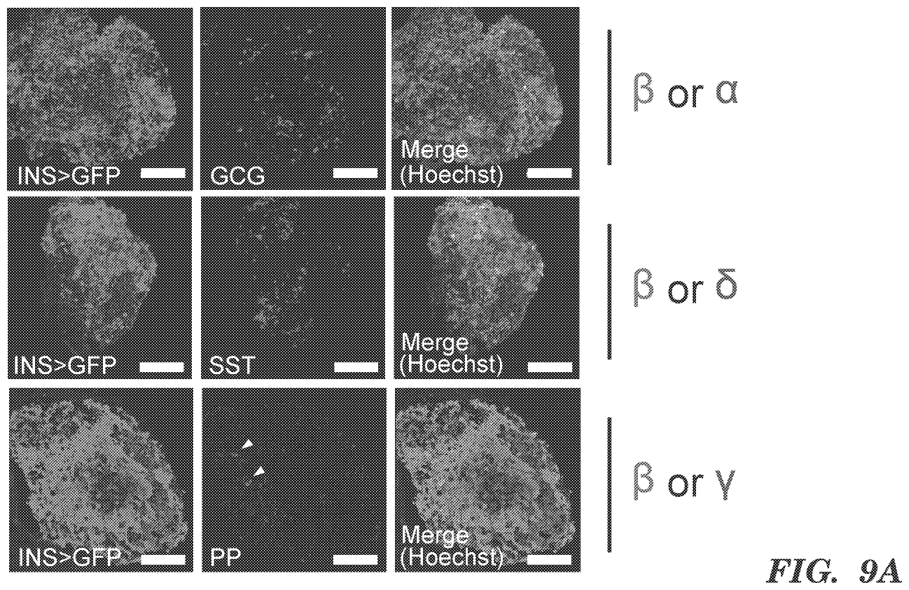

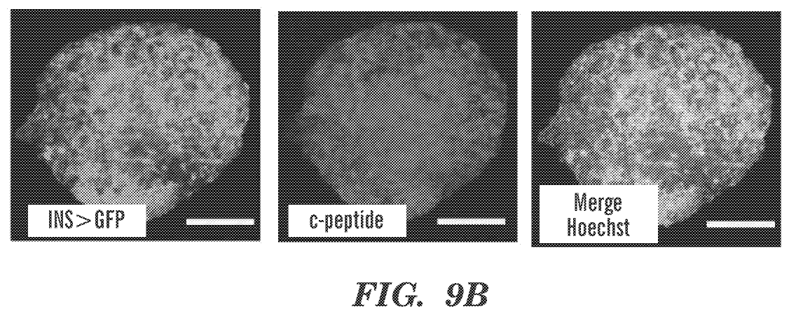

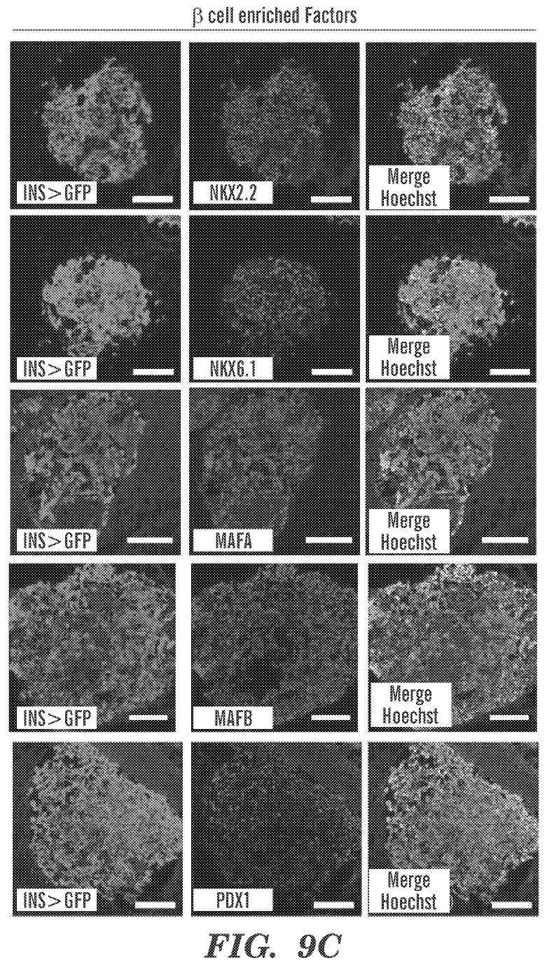

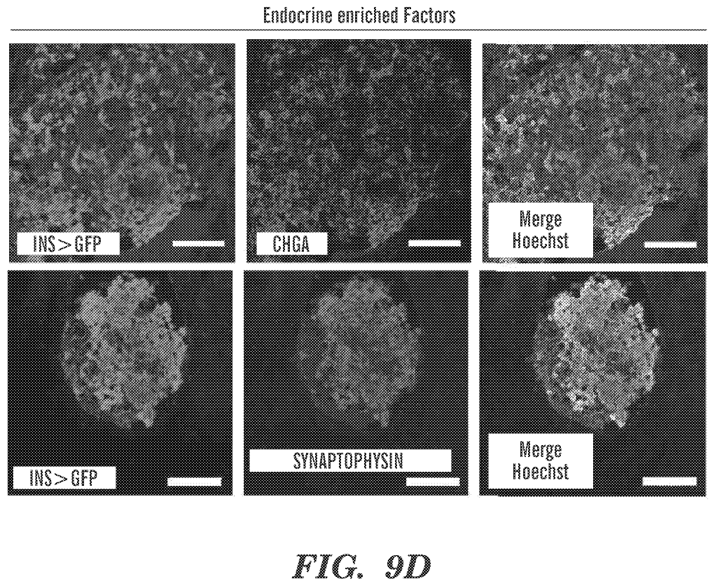

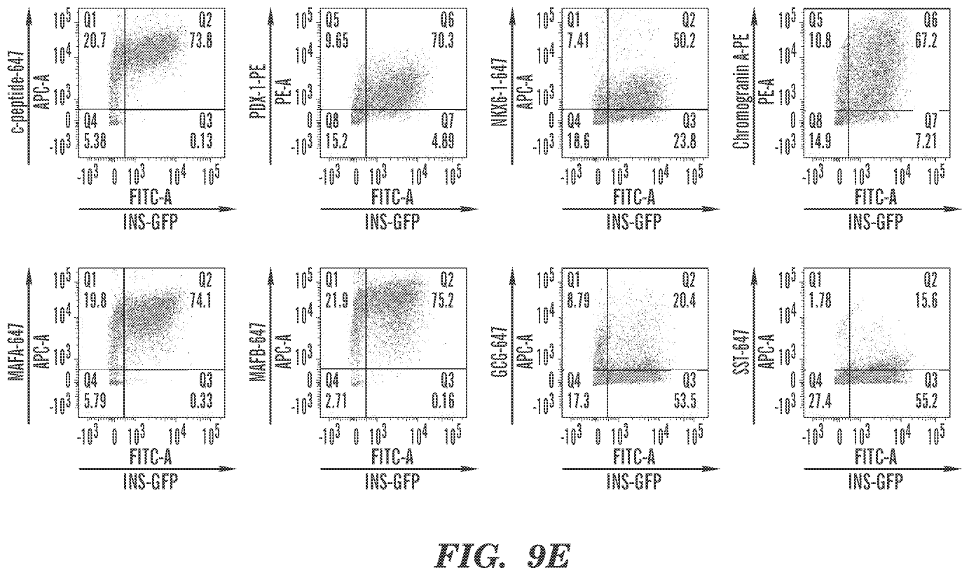

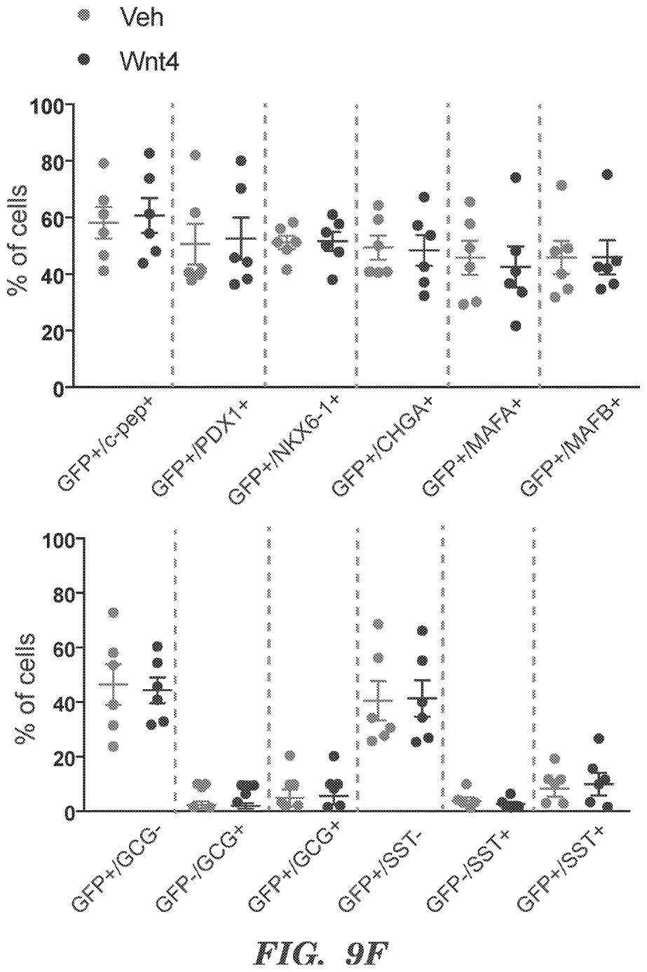

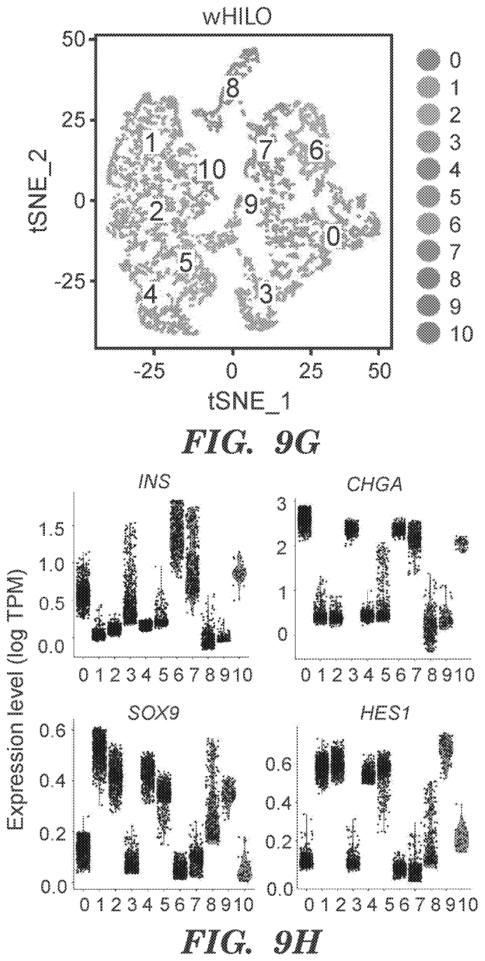

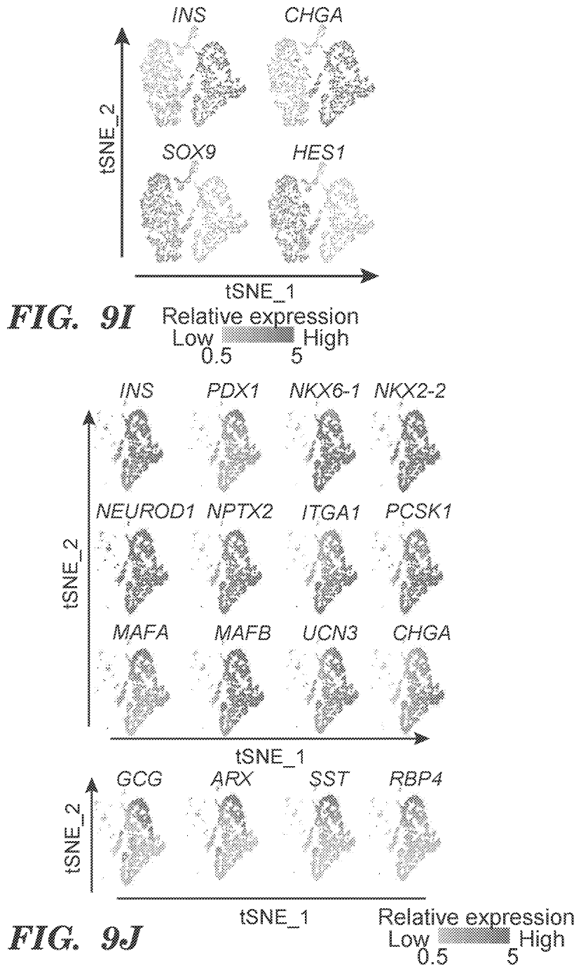

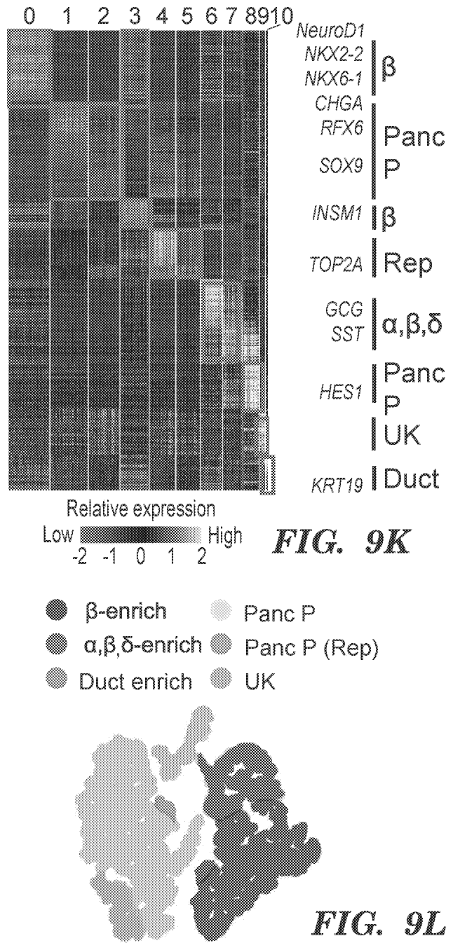

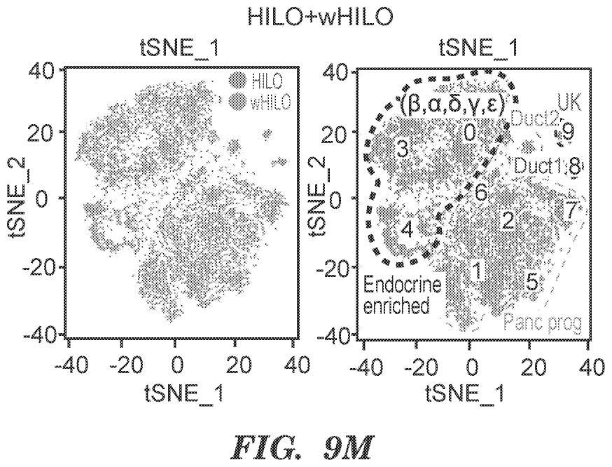

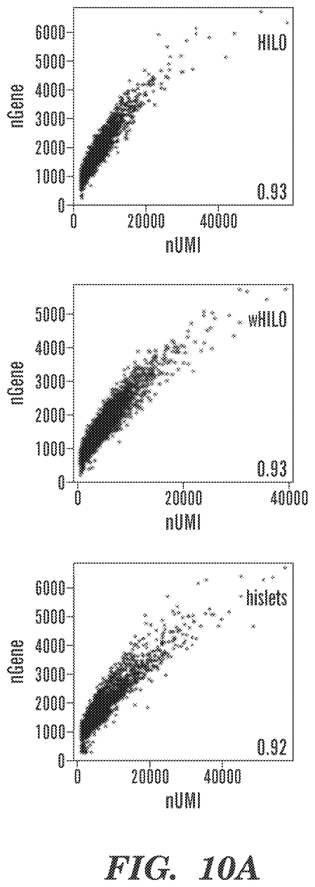

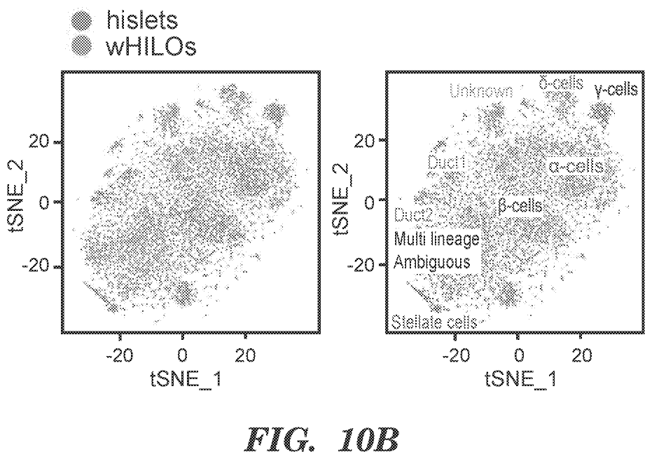

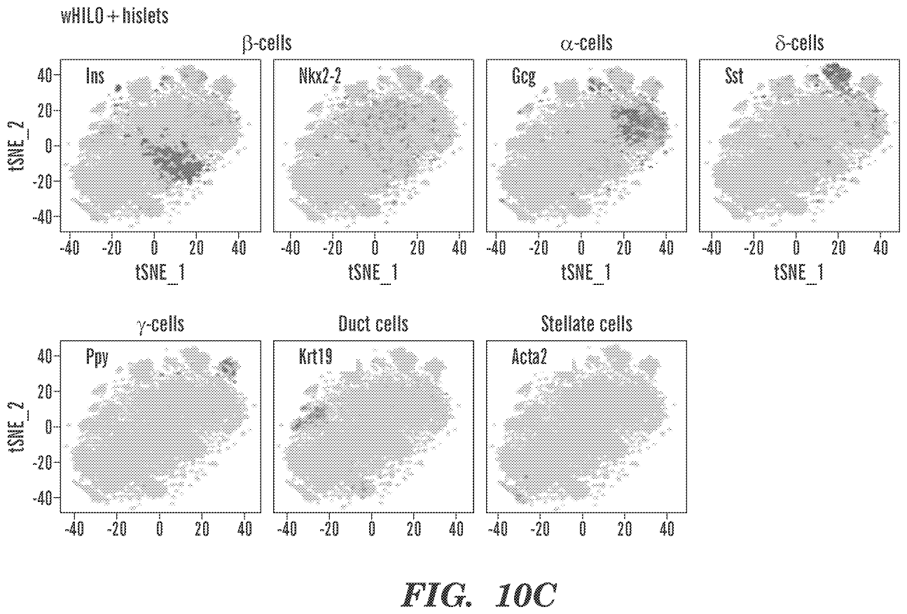

Immunohistochemical and flow cytometric analyses revealed that approximately 50-60% of wHILO cells co-expressed insulin and j cell markers, as well as low levels of additional endocrine cells (glucagon⁺, somatostatin⁺, pancreatic polypeptide⁺ (PP⁺)) (FIGS. 9A-9F). In agreement with the transcriptional comparisons, the cellular composition of HILOs was not altered by WNT4 treatment (FIG. 9F). To comprehensively characterize the cellular complexity of metabolically mature HILOs and gain insight into the in vitro maturation program, the single cell transcriptomes of HILOs (PBS-treated, n=4078) and wHILOs (WNT4-treated, n=4840) were compared with those of human islets (n=3245) (Table 1). Cellular transcriptomes in each analysis were clustered by principal component analysis of read counts with dimensionality reduction using t-distributed stochastic neighbor embedding (t-SNE). Clustering of wHILOs revealed populations enriched in β cell markers, as well as in Sox9⁺HES1⁺ pancreatic progenitor clusters (FIGS. 9G-9J). Signature gene expression analyses further distinguished non-replicating and replicating ductal-endocrine bipotent cells (+/−TOP2A), hormone positive endocrine enriched cells (GCG⁺, SST⁺), ductal-like cells (KRT19⁺) and a small population of cells with unknown function (UK). (FIG. 9K and FIG. 9L). Co-clustering of HILO and wHILO data sets provided additional evidence for the presence of multiple endocrine-like cell types (based on the highly expressed genes in each cluster) that were largely independent of WNT4 treatment (FIG. 9M). To confirm the presence of multiple endocrine-like cell types, an integrated analysis of the combined wHILO and human islet single cell data sets was performed (FIGS. 10A-10C). While differences were evident, wHILO cells were found clustering with islet endocrine cells including (β, α, δ and γ cells, indicating transcriptional similarities (FIG. 10B). Notably, a functional classification based on co-clustering with islet cell types revealed a predominance of β- and α-like cells in wHILOs (FIG. 10B).

TABLE 1

| Sample identification | HILO | wHILO | H-ISLETS |
|---|---|---|---|
| Estimated Number of Cells | 4,078 | 4,840 | 3,245 |
| Fraction Reads in Cells | 88.90% | 89.20% | 79.70% |
| Mean Reads per Cell | 16,482 | 13,496 | 22,195 |
| Median Genes per Cell | 1,582 | 1,455 | 1,486 |
| Total Genes Detected | 22,003 | 22,076 | 21,007 |
| Median UMI Counts per Cell | 4,754 | 4,220 | 5,618 |
| Number of Reads | 67,216,051 | 65,324,121 | 72,025,806 |
| Valid Barcodes | 98.50% | 98.50% | 98.60% |
| Reads Mapped Confidently | 58.30% | 58.10% | 64.40% |
| to Transcriptome | | | |
| Reads Mapped Confidently | 62.20% | 62.00% | 68.10% |
| to Exonic Regions | | | |
| Reads Mapped Confidently | 24% | 23.70% | 19.00% |
| to Intergenic Regions | | | |
| Reads Mapped Confidently | 4.70% | 4.70% | 4.20% |
| to Intergenic Regions | | | |
| Reads Mapped Antisense to Gene | 4.10% | 4.00% | 4.40% |
| Sequencing Saturation | 32.30% | 27.00% | 38.60% |
| Q30 Bases in Barcode | 96.80% | 96.80% | 96.80% |
| Q30 Bases in RNA Read | 80.50% | 79.40% | 80.40% |
| Q30 Bases in UMI | 96.40% | 96.40% | 96.40% |

| | | |
|---|---|---|
| Genomic Modification | CRISPR-InsulinGFP Reporter | None |

| | |
|---|---|
| Transcriptome | GRCh38 |
| Chemistry | Single Cell 3′ v2 |
| Cell Ranger Version | 2.0.2 |

### Example 7: PD-L1 Provides Immune Protection for HILOs

The clinical utility of transplanted islets is limited by both allogenic and autoimmune responses. Given the ability of checkpoint molecules to suppress immune responses, the endogenous expression of immune checkpoint proteins in human islets was investigated. A small subset of β cells in healthy islets showed a unique gene expression signature that included PD-L1 expression (FIG. 12A), a determinant of immune tolerance in β cells. To create wHILOs that exhibited exogenous PD-L1 expression to thereby protect them upon transplantation, PD-L1-expressing hiPSC clones were generated using a lentiviral system and subsequently differentiated into metabolically mature wHILOs, as delineated in FIG. 3A. PD-L1 over-expression in the HILOs did not affect insulin expression (FIGS. 12B and 12C). PD-L1-expressing wHILOs and those that did not express PD-L1 were transplanted into the kidney capsules of immune competent diabetic mice (STZ-treated C57BL6J mice), (FIG. 12D). wHILOs with and without PD-L1 overexpression were able to restore glycemic control within days of transplantation with similar efficacy (FIG. 4C). However, the functionality of wHILOs lacking PD-L1 expression was progressively lost over a period of weeks, as monitored by the increases in blood glucose levels. By contrast, the PD-L1⁺ wHILOs were able to maintain glucose homeostasis for >50 days in the absence of immunosuppressive drugs (FIG. 4C).

To confirm the immune-suppressive actions of PD-L1, transplanted wHILOs were recovered from recipient mice 27 days after transplantation, and the cellular compositions were compared by flow cytometry. The infiltration of CD45⁺ immune cells, including T and NKT cells, was markedly decreased in grafts that had received wHILOs that expressed PD-L1 (FIGS. 4D-4G). Furthermore, negligible numbers of insulin-expressing cells were found in grafts that had received wHILOs lacking PD-L1 expression, in agreement with the largely unregulated blood glucose levels observed 27 days after transplantation (FIG. 4D, FIG. 4F and FIG. 4H).

The persistence of wHILO (PD-L1) as xenografts led to an assessment of their functionality in a model incorporating a reconstituted human T cell repertoire. After confirming the presence of human T cells, HuPBMS-NSG-SGM3 mice were rendered diabetic by multi low dose STZ treatment (50 mg/kg/day for 5 days, MLD-STZ) and were subsequently transplanted with wHILO (FIG. 4I and FIG. 4J). Transplanted wHILOs (PD-L1) provided sustained blood glucose control compared to those lacking PD-L1 expression, with human c-peptide levels correlating with the extent of glycemic control (FIG. 4K and FIG. 4L). The rapid development of hyperglycemia upon surgical removal of the transplanted kidneys implicated graft-derived insulin as the primary effector (FIG. 4K). Subsequent analysis of the recovered grafts revealed a marked reduction in the number of insulin expressing cells in wHILOs and a corresponding increase in human lymphocytes (FIG. 4E and FIG. 4M).

### Example 8: Epigenetic Memory Drives Immune Tolerant wHILOs

PD-L1 expression is induced by IFNγ stimulation in multiple cancers; however, extended exposure to cytokines, including IFNγ, has been found to induce β-cell death and/or de-differentiation. In this Example, experiments were performed to assess whether the IFNγ pathway was capable of minimizing host immune responses against transplanted wHILOs. Following exposure of wHILOs to IFNγ stimulation, it was found that IFNγ rapidly and robustly induced PD-L1 expression in wHILOs (FIGS. 12E and 12F). In particular, an approximately 20-fold increase in PD-L1 expression was observed 12 hours after IFNγ treatment. (FIG. 12F). Notably, IFNγ induced PD-L1 expression in wHILOs to similar levels in both insulin-expressing and insulin non-expressing cells (GFP+ and GFP− cells, respectively), (FIG. 5A). Subsequent dose-escalating studies in wHILOs identified maximum PD-L1 induction after a 2-hour, 10 ng/ml IFNγ exposure. (FIG. 12E). However, the induction was transient, with PD-L1 expression rapidly decreasing in the days following exposure to IFNγ (FIG. 5B). Because tolerance to inflammatory stimuli such as lipopolysaccharide has been associated with epigenetic changes, experiments were performed to investigate whether sequential IFNγ stimulation induced longer term or sustained effects in wHILOs, specifically, a sustained induction of PD-L1 in the HILOs. Indeed, it was discovered that repeated short exposures (intermittent exposure) to IFNγ (multiple pulse stimulation, “MPS”) led to sustained PD-L1 expression and concomitant increases in PD-L1 protein levels (FIGS. 5C, 5D and 5E). Importantly, GSIS functionality was not compromised by exposure of the wHILOs to MPS IFNγ (FIG. 5F). Furthermore, MPS IFNγ-treated wHILOs were protected against IL-1β-induced β cell dedifferentiation, as revealed by the expression of the β cell identity markers INS and UCN3 (FIG. 5G and FIG. 5H).

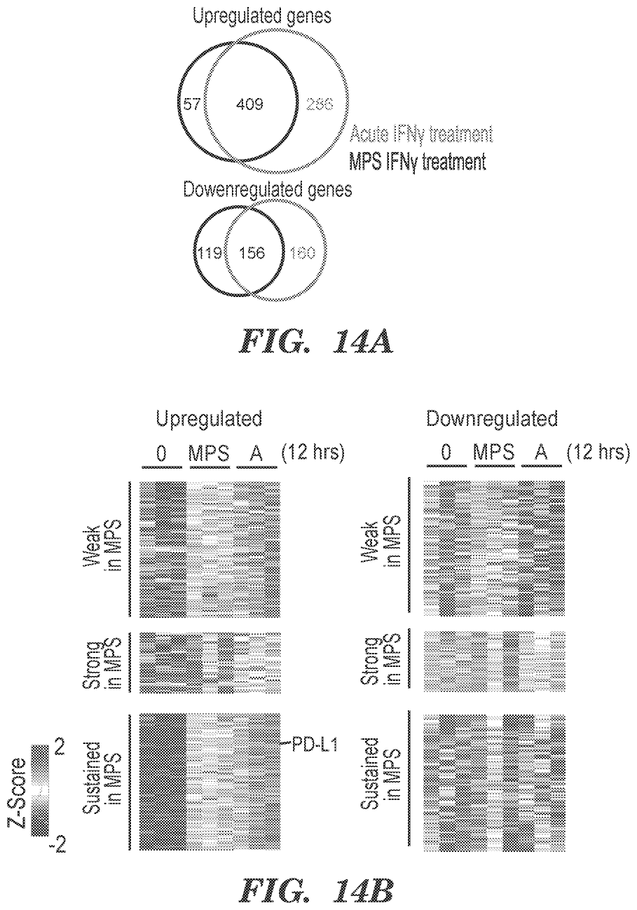

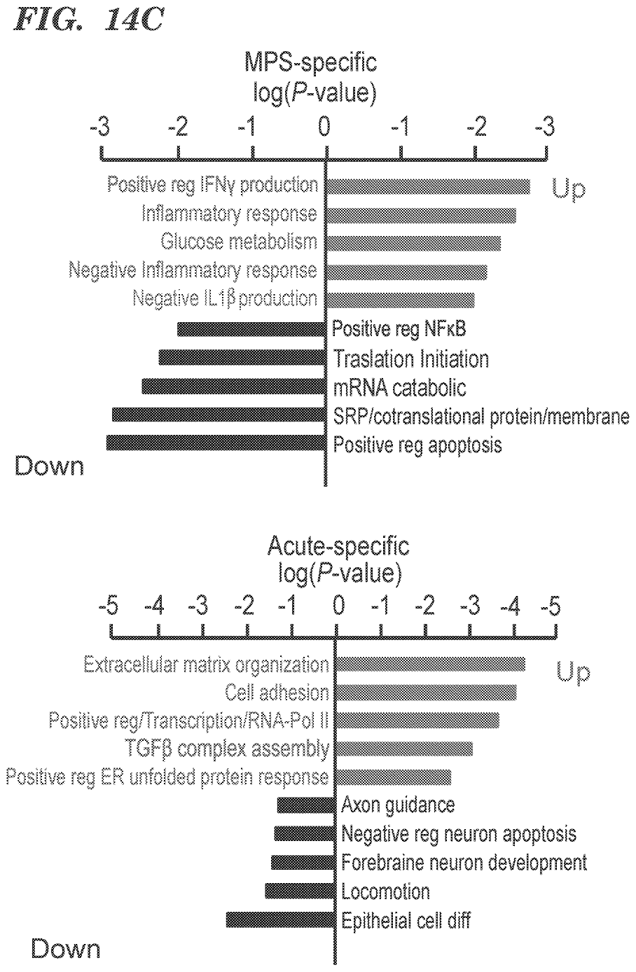

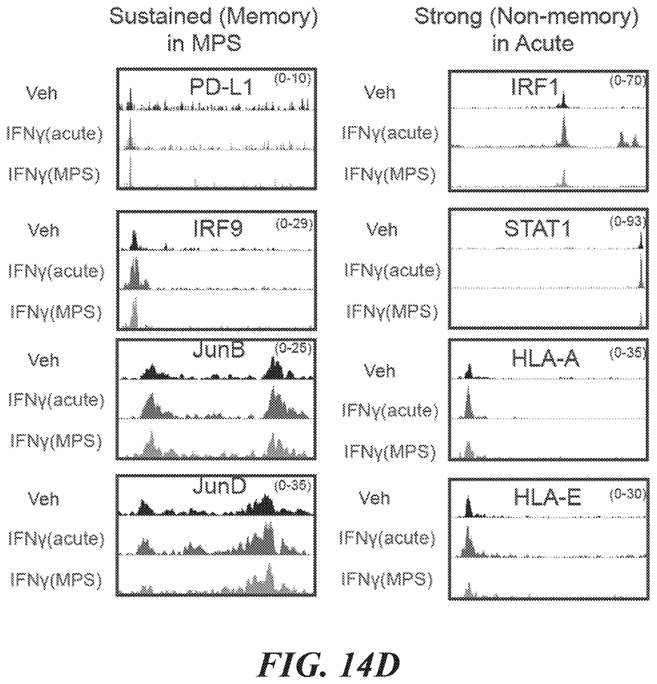

ATAC-Seq was used in studies to provide mechanistic insight into the IFNγ-driven changes in wHILOs. As measured by ATAC-Seq, the genome-wide transcriptional changes induced by acute (12 h exposure) and MPS treatments were associated with alterations in chromatin accessibility. Largely overlapping gene sets were induced by the IFNγ treatments that included PD-L1, while approximately half of the downregulated genes were commonly affected (FIG. 14A and FIG. 14B). Gene ontology of the commonly upregulated gene set identified IFNγ pathways (not shown). In contrast, pathways that reflect the cell inflammation status including negative regulation of IL-1ß production and inflammatory pathways were identified only in the MPS-upregulated gene set, while positive regulation of NFkB signaling and apoptosis were found selectively in the MPS-downregulated gene set (FIG. 14C). Overlaying changes in chromatin accessibility revealed persistent increases at gene loci including PD-L1, IRF9, JUNB, and JUND after MPS IFNγ treatment, in agreement with the sustained increases in gene transcript levels. In contrast, while increased accessibility was seen at known IFNγ-responsive genes, including IRF1 and STAT1, after the acute treatment, these increases were not sustained (FIG. 14D).

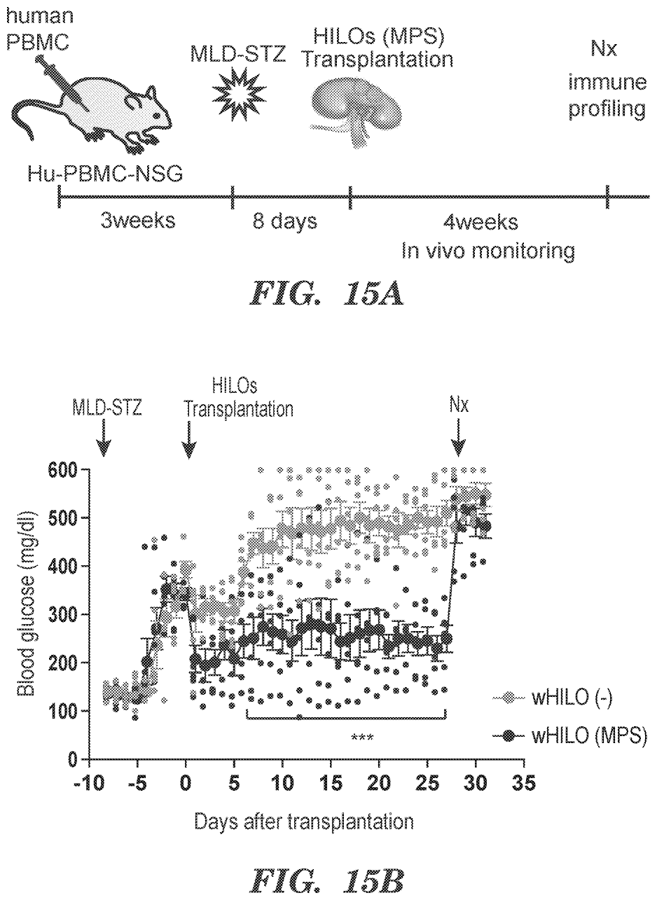

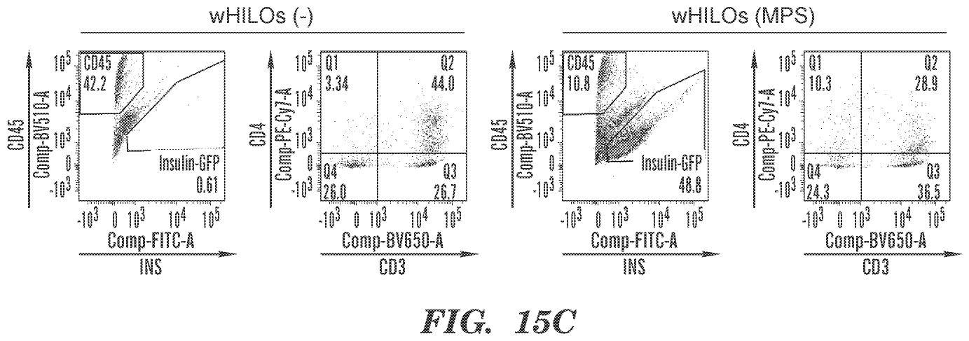

To confirm that IFNγ treatment generated immune evasive wHILOs (wHILO^{ie}), the ability of wHILO^{ie} to provide long term glucose regulation in immune competent mice was assessed. Transplantation of wHILO^{ie} into STZ-induced diabetic C56BL6J mice lowered blood glucose levels in the mice within days and maintained reduced levels for >40 days (FIG. 5I, FIG. 5J). In contrast, the efficacy of transplanted, naive wHILOs (no IFNγ exposure) progressively decreased, which was consistent with the reduced levels of human c-peptide observed in the serum of recipient mice (FIG. 5K). Similar results were found with transplantation into humanized diabetic mice. Notably, the reduced glucose levels achieved with wHILO (MPS treated) transplantation were lost upon surgical removal of the recipient kidney (FIGS. 15A and 15B). As support for the immunosuppressive role of IFNγ-induced PD-L1 in the transplanted wHILOs, reduced lymphocyte infiltration, as well as a decrease in the relative number of activated T helper cells (CD4⁺CD3⁺), were observed in the recovered grafts. Moreover, the number of insulin expressing cells was markedly increased in wHILO (MPS treated) grafts (FIG. 15C).

Without intending to be bound by theory, the results described herein suggest that prior IFNγ stimulation, namely, exposure of cells, such as wHILOs, to the MPS IFNγ protocol, induces an epigenetic memory that leads to cytokine tolerance and sustained de novo PD-L1 expression in wHILOs. Such IFNγ stimulated wHILOs (wHILO^{ie}) offer utility of as a therapy to alleviate diseases, such as pancreatic diseases, or insulin dependent diabetes, for example, type 1 or type 2 diabetes.

The findings, based on the above-described experiments, that wHILOs maintained functionality in NOD-SCID but not in C57BL6J mice implicates T cells and β cells in their allogenic rejection. During antigen presentation, interactions between cytotoxic T-lymphocyte antigen-4 (CTLA-4) and B7 molecules, as well as programmed cell death protein 1 (PD1) and its ligand PD-L1, negatively regulate immune responses in a non-redundant manner. The results of the experiments demonstrate that wHILOs that express PD-L1, such as by induction or overexpression as described herein, are protected from allogenic rejection. Furthermore, as described supra, a protocol is provided in which repeated exposure to limited IFNγ concentrations leads to sustained, endogenous PD-L1 expression without compromising glucose stimulated insulin secretion (GSIS) activity. Of note and unexpectedly, the resultant immune evasive HILOs described herein were able to maintain glucose homeostasis in immune-competent type 1 diabetic mice for ~50 days in the absence of a transplantation device. The immune evasive cells (such as in HILOs) that result from IFNγ exposure according to the method described herein not only exhibit metabolic and functional maturity, but they overcome autoimmune rejection of transplanted cells, which provides a solution to a general problem that exists for other stem cell-based therapeutics.

### Example 9: Methods Used in the Above-Described Examples

### Maintenance of Mouse Lines

Animals were maintained in a specific pathogen-free animal facility on a 12 hour light-dark cycle at an ambient temperature of 23° C. Water and food were provided ad libitum. Animal experiments used age- and background-matched male C57BL6J (Stock No 000664), NOD-SCID mice (NOD.Cg-Prkdcscid Il2rgtm1Wjl/SzJ, Stock No 005557), ß cell specific ERRγ knockout mice (Yoshihara, E. et al., 2016, Cell metabolism 23, 622-634, doi:10.1016/j.cmet.2016.03.005), hu-PBMC-SGM3 mice, called ‘humanized mice’. Female NSG™ mice were injected with human peripheral blood mononuclear cells (PBMCs) in NSG-SGM3 (Jackson 013062) strain) All procedures involving animals were performed in accordance with protocols approved by the IACUC and Animal Resources Department of the Salk Institute for Biological Studies.

Generation of Human Insulin Reporter and PD-L1 Overexpressing Human iPSC Lines

To mark β cell specification, human induced pluripotent stem cells (hiPSCs) derived from HUVECs were infected with a human insulin GFP reporter, as described by E. Yoshihara et al. (2016, Cell metabolism, 23:622-634). To visualize endogenous insulin promoter activity, CRISPR/Cas9 genome editing was used to knockin GFP into the insulin promoter (Tables 1 and 2).

TABLE 2

| NCBI or Primer | | | Primers | Primers |
|---|---|---|---|---|
| Bank (PB) ID | Genes | Species | (Forward) | (Reverse) |
| NM_206594.2 | ESRRG (ERRy) | hu* | gctaacactgtcgcagtttga | cgaacagctggaatcaatgtg |
| 316659406c1 | NDUFA7 | hu | tgcagctacgctaccagga | ggaggctgagttcgcttgg |
| (PB) | | | | |
| 103472000b1 | COX7A2 | hu | ctcggaggtagttccggttc | tctgcccaatctgacgaagag |
| (PB) | | | | |
| 316659406c1 | NDUFA1 | hu | atgctccgccagatcatcg | tgccagacgcaagagatacag |
| (PB) | | | | |
| NM_002509.4 | NKX2-2 | hu | ggccttcagtactccctgca | gggacttggagcttgagtcct |
| 115387113c1 | ISL1 | hu | gcggagtgtaatcag | gcatttgatcccgtacaacct |
| (PB) | | | tatttgga | |
| NM_005461.4 | MAFB | hu | gcctgcgctaattgtaggag | cgcacttgaaagttgcaaaa |
| NM_020783.3 | STY4 | hu | ttcaggacggggtgagttac | tttggcatggtacaggttca |
| NM_000162.3 | GlucoKinase | hu | gctggaatcaatttcccaga | ctccccacacaggatgagtt |
| NM_000207.2 | INSULIN | hu | agcctttgtgaaccaacacc | gctggtagagggagcagatg |
| NM_002054.4 | GLUCAGON | hu | aggcagacccactcagtga | aacaatggcgacctcttctg |
| NM_001048.3 | SOMATOSTATIN | hu | gtacttcttggcagagc | cagaagaaattcttgc |
| | | | tgctg | agccag |
| NM_000209.3 | PDX-1 | hu | ggatgaagtctaccaaa | ccagatcttgatgtgt |
| | | | gctcacgc | ctctcggtc |
| NM_201589 | MAFA | hu | cttcagcaaggaggag | ctcgtatttctccttg |
| | | | gtcatc | tacaggtcc |
| NM_006168.2 | NKX6-1 | hu | attcgttggggatgacagag | tcaacagctgcgtgattttc |
| NM_053049.3 | UCN3 | hu | gatgggcttggctttgtaga | ggagggaagtccactctgc |
| NM_002500.4 | NEUROD1 | hu | gttctcaggacgaggagcac | cttgggcttttgatcgtcat |
| NM_014143.3 | CD274 (PD-L1) | hu | tatggtggtgccgactacaa | tgcttgtccagatgacttcg |
| NM_001002.3 | U3664 (RPLP0) | hu/mo | gtgctgatgggcaagaac | aggtcctccttggtgaac |
| NM_021893.3 | CD274 (PD-L1) | mo | tgctgcataatcagctacgg | gctggtcacattgagaagca |
| NM_001243792.1 | Esrrg (ERR) | mo | gcaaggcattcttcaagagg | ggctgggcagctgtactcta |
| NM_009943.2 | COX6a2 | mo | ctctcgactgggtgaaggag | gaagagccagcacaaaggtc |
| NM_008618.3 | MDH1 | mo | gaagccctgaaagacgacag | tcgacacgaactctccctct |
| NM_153064.4 | NDUFS2 | mo | gatccgagtgctctttggag | atgtcatccagaagcccaag |

Species*: hu: human; mo: mouse

TABLE 3

| Sequence | | |
|---|---|---|
| Name | Sequence | Vector |
| Human | GTGGTTGACGC | pCas- |
| insulin | TGTCCGTCA | Guide- |
| guide 1 | | EF1a-GFP |
| | | vector |
| | | (Origene |
| | | 100018) |
| Human | CTGTTCGTCCT | pCas- |
| insulin | TCATCAAGA | Guide- |
| guide 2 | | EF1a-GFP |
| | | vector |
| | | (Origene |
| | | 100018) |
| Left Arm | ATAAGACACAGTTATGCTT | Luc- |
| | ATGGAAGCGTGCTGACAAA | LoxP- |
| | CAGTAATTACAGAGCTGAG | PGK- |
| | GATCATCTGTTCAGTCTTG | Puro- |
| | AAAATAAAAGTTTTATTCT | LoxP |
| | GCTCATAATAAAATGATTG | |
| | CAGCATCAGAATGAGGAAG | |
| | GAAAGGTAGAATGAGGATA | |
| | AATACAATTTTAGAAATGG | |
| | TATAGACTTTGCAAATCAC | |
| | CACCTCTTCCATTGATAAA | |
| | TTTAGAATCTAGAGTTGAG | |
| | TTAGATATTGACACTGGTT | |
| | CTCCAAGAGAAAGGTAAAA | |
| | TAAAAGCAATCGGACTCTT | |
| | TAGAGCTTTTGTTTATGGC | |
| | CTGTCTGGGCCCTTTGTTG | |
| | TAACCCTGTCATGCCCTTA | |
| | TGCTGATTACCTTCTTGTA | |
| | GAACAAGAAGTATTGACTA | |
| | GAGAATGAATGATGTGTAG | |
| | TCCCTAGCCCTTAGGAAAC | |
| | TCTCTCAAAGAGCAATGTC | |
| | TTTAACATATGAATTCTGT | |
| | TTTTTTCCTCCTTTTACCT | |
| | TTCCCTTTCCCTTTCTCTA | |
| | TTTTTCACCATCTCTTTTG | |
| | TTTCTACCTCTTTTGGTCT | |
| | CTGTGCTTGACACTCTCTC | |
| | CTCTTTCTGTCTCTCTTTG | |
| | TATCTCCTCAATCTCAGGC | |
| | TTCTCTGCAGA | |
| Right | CTGGTGGCTCTTCAGACGC | |
| Arm | CAGTGGGAGCTACAGTTCA | |
| | ACCATGAATGGCCATCAGA | |
| | ACGGACTTGACTCGCCACC | |
| | TCTCTACCCTTCTGCTCCT | |
| | ATCCTGGGAGGTAGTGGGC | |
| | CTGTCAGGAAACTGTATGA | |
| | TGACTGCTCCAGCACCATT | |
| | GTTGAAGATCCCCAGACCA | |
| | AGTGTGAATACATGCTCAA | |
| | CTCGATGCCCAAGAGACTG | |
| | TGTTTAGTGTGTGGTGACA | |
| | TCGCTTCTGGGTACCACTA | |
| | TGGGGTAGCATCATGTGAA | |
| | GCCTGCAAGGCATTCTTCA | |
| | AGAGGACAATTCAAGGTTA | |
| | GTGTCGGACCTGGGAATAC | |
| | TCTCCCCACTTCCAACCTC | |
| | ACATGATGGGTTTTTGTTT | |
| | TTCCTTATTCTTATTCTCA | |
| | TAAGTCAAGTATCATAGTT | |
| | TTAATTCTCTCTTGAGTAG | |
| | AAAATGGAAATAGATTACA | |
| | ATTGATAGTGGAAGATTTA | |
| | TAGAATAAAATCCCCCCAG | |
| | ATATACTCCATATCTATTA | |
| | ATTTTCCTCTTACTGTTAA | |
| | GCTTTAATGGTGCAAGGAT | |
| | AATAAACTTTGGGTAGAGT | |
| | TTACAAGAGCATAGTTATT | |
| | ATTAGAGCAATGTGGGTCT | |
| | ATATAGCAACT | |

PD-L1 expressing hiPSCs were generated by infecting hiPSCs with a lentivirus (abm, LV113090) encoding human CD274 (PD-L1) with puromycin selection (Table 4). The human UCN3 proximal promoter sequence (−1298/+103) was introduced by In-Fusion cloning (Clonetech) into the promoterless pLV-Cherry-Picker backbone (Clontech, 632574) using the ApaI/NotI restriction enzyme sites. Primer sequences for PCR amplification of the promoter sequence from genomic DNA were 5′-GTCCATGCTGATCCATCCTT-3′ (forward) (SEQ ID NO: 107) and 5′-TGCTTCTCCGGTATTGTTCC-3′ (reverse) (SEQ ID NO: 108). A dual reporter line for human UCN3 mcherry and human insulin GFP (hINS-GFP-EF1α-Neo), Yoshihara et al., Ibid., was generated in hiPSC.

TABLE 4
Plasmid Information

| | Name | | | Sequence |
|---|---|---|---|---|
| | (Donor)/ | Spe- | Char- | Primer |
| System | Catalog # | cies | acter | Fw/Rv |
| Lentivirus | CD274 | human | Over- | |
| | (PD-L1) | | expres- | |
| | Lentivirus | | sion | |
| | Vector/ | | | |
| | (abm | | | |
| | LV113090) | | | |
| Lentivirus | UCN3- | human | mCherry | 5′-GTCCA |
| | Cherry | | reporter | TGCTGATC |
| | reporter | | | CATCCTT-3′ |
| | | | | (forward) |
| | | | | 5′-TGCTTC |
| | | | | TCCGGTATT |
| | | | | GTTCC-3′ |
| | | | | (reverse) |

### Virus Production

Lentiviruses were produced using second- or third-generation lentiviral systems in HEK293T cell line using methods as described herein (e.g., Example 10 methods) and as known and practiced by those skilled in the art.

### 3D Gellan Gum (3DKG) Culture Medium

Aqueous solutions of low acyl gellan gum (Kelcogel F GG-LA), (Modernist pantry), 0.3% w/v, were sterilized by autoclaving prior to dilution in mTeSR1 or Custom TeSR medium (StemCell Technologies, final concentration 0.015%) and the addition of methylcellulose (R&D systems, final concentration 0.3%) and penicillin/streptozocin.

More specifically, by way of example, Kelcogel F low acyl GG GG-LA (Modernist pantry) was suspended in pure water 0.3% (w/v) and dissolved by stirring at 90° C. or by microwave. The aqueous solution was sterilized at 121° C. for 20 minutes in an autoclave. The solution was added to TeSR or Custam TeSR at a final concentration of 0.015%. Methylcellulose (MC) stock solution was added to a final concentration of 0.3% (R&D systems) (e.g., 0.3% Kelcogel stock; Kelcogel F low acyl GG GG-LA 300 mg+MilliQ water 100 ml: 3DKG Stem TeSR Base Medium; Stem TeSR 95 ml+0.3% Kelcogel 5 ml+MC stock solution 300 μl. A 1% final concentration of Penicillin/streptozocin was added for 3DKG Stem TeSR.

### Human Multicellular Spheroids (MCSs)

Pancreatic endocrine (PE) cells were prepared from human iPSC as described in the publication of Yoshihara, E. et al. (2016, Cell Metabolism, 23(4):622-634). In brief, HUVEC-derived hiPSC, obtained from the Salk Stem Cell Core Facility, were maintained on matrigel (BD)-coated dishes in complete Stem TeSR Medium at 37° C. in a humidified 5% CO₂ incubator. Prior to pancreatic differentiation, hiPSC were infected with a human insulin reporter lentivirus (pGreenZero lenti reporter human insulin, System Biosciences) by Spinfection (800 g, 1 hour), and then the cell medium was changed to 100 ng/ml human Activin (R&D Systems), 3 μM CHIR99021 (Selleckchem) in differentiation medium (800 ml DMEM/F12, 13.28 g BSA, 10 ml Glutamax, 560 mg NaHCO₃, 330 mg thiamine, 100 mg reduced glutathione, 3300 mg Vitamin C, 14 μg Selenium, 10 ml NEAA, 2 ml Trace Element B, 1 ml Trace Element C, 7μ 1 β-ME, 2 ml DLC, 2 ml GABA, 2 ml LiCl, 129.7 μg PA, Insulin 2 mg, made up to 1000 ml) for 2 days, and then the cells were maintained in 100 ng/ml human Activin in differentiation medium for another 2 days (Stage 1, Pancreatic Endoderm). Subsequently, this medium was replaced with differentiation medium containing 1 μM dorsomorphin (Calbiochem), 2 μM Retinoic Acid (Sigma), 10 μM SB431542 and 1% of B27 supplement for 7 days (Stage 2). The medium was then replaced with differentiation medium containing 10 μM forskolin (Sigma), 10 μM dexamethasone (Stemgent), 10 μM TGFβ RI Kinase inhibitor II/Alk5 inhibitor II (Calbiochem or Enzo), 10IM Nicotinamide (Sigma), 1 μM 3,3′,5-Triiodo-L-thyronine sodium salt (T3) and 1% of B27 supplement for 4-5 days (day15-day19, Pancreatic endocrine progenitors developed). The medium was replaced every day (stage 1), and then every other day (stage 2 and stage 3).

Primary HUVEC cells and human adipose-derived stem cells (hADSC) (Invitrogen or PromoCell) were cultured in 15 cm dishes with EBM Media (Lonza, cc-3121) or MesenProRS Media (GIBCO, 12747-010 or Preadipocyte Growth Medium Kit, C-27417), respectively, at 37° C. in a humidified 5% CO₂ incubator. For co-culturing experiments, pancreatic endocrine progenitors derived from human iPSC were treated with Accutase, while HUVECs and hADSC were treated with TrypLE (GIBCO, 12604-013). Cells were collected into 50 ml tubes. hiPSC-EP (1×10⁶ cells), HUVECs (7×10⁶ cells) and hADSCs (1-2×10⁵ cells) were co-cultured in a single well of a 24 well plate with 300 μl of matrigel.

For MCS generation, hiPSC-EP (day15-day21, 1×10⁶ cells), HUVECs (7×10⁶ cells) and hADSCs (1-2×10⁵ cells) were co-cultured in 3D Kelco Gel Custom TeSR with 10 μM forskolin (Sigma), 10 μM dexamethasone (Stemgent), 10 μM TGFβ RI Kinase inhibitor II/Alk5 inhibitor II (Calbiochem or Enzo), 10IM Nicotinamide (Sigma), 1 μM 3,3′,5-Triiodo-L-thyronine sodium salt (T3) and 1% of B27 supplement, R428 (2 μM), Zinc sulfate (10 μM) and N-Cys (1 mM). The medium was changed every other day, and islet-like clusters formed within a few days. (FIGS. 6A-6F).

### Human Pancreatic Islet-Like Organoid (HILO) Cultures

hiPSCs were cultured in matrigel-coated plates. Single cell suspensions were prepared using Accutase, washed in PBS, and collected by centrifugation (1000-1300 rpm for 5 min). Cells were re-suspended with 3D Kelco Gel Stem TeSR™ Base Medium in the presence of the ROCK inhibitor (10 μM Y-27632, StemCell) for 5 to 7 days until spheroids reached 50-100 μm diameter. The medium was then replaced with 0.015% Kelco gel containing 0.3% methylcellulose and supplemented with 100 ng/ml human Activin A (R&D Systems), 3 μM CHIR99021 (Axon or Selleckchem) in differentiation medium (S1) for 1 day, and then 100 ng/ml human Activin in differentiation medium (S1) for another 2 days (Stage 1, Definitive Endoderm). Subsequently, the medium was replaced with differentiation medium (S2) with 50 ng/ml FGF7 (R&D Systems) for 2 days, differentiation medium (S3) with 50 ng/ml FGF7, 0.25 μM SANT-1 (Sigma), 1 μM Retinoic Acid (Sigma), 100 nM LDN193189, 10 μM Alk5 inhibitor II and 200 nM of the B-Amyloid Precursor Protein modulator TPB for 3 days, then 50 ng/ml FGF7, 0.25 μM SANT-1 (Sigma), 1 μM Retinoic Acid (Sigma), 100 nM LDN193189, 10 μM Alk5 inhibitor II and 100 nM of the B-Amyloid Precursor Protein modulator TPB for 2 days. Subsequently the medium was replaced with differentiation medium (S4) with 0.25 μM SANT-1, 50 nM retinoic acid, 100 nM LDN193189, 10 μM Alk5 inhibitor II, 1 μM T3 for 3 days. Subsequently, the medium was replaced with differentiation medium (S5) with 100 nM LDN193189, 100 nM y-secretase inhibitor XX (GSiXX, Millipore), 10 μM Alk5 inhibitor II, 1 μM T3 for 7 days. Subsequently, the medium was replaced with differentiation media (S5) with 10 μM Trolox (Calbiochem), 2 μM R428 (Selleckchem), 1 mM N-acetyl cysteine, 10p M Alk5 inhibitor II, 1 μM T3 for an additional 7 to 20 days. After confirmation of insulin expression by qPCR or reporter activity (typically days 20-30), the medium was changed to differentiation medium (S5) with 10 μM Trolox (Calbiochem), 2 μM R428 (Selleckchem), 1 mM N-acetyl cysteine, 10 μM Alk5 inhibitor II, 1 μM T3 and 100 ng/ml rhWnt4 (R&D Systems) with or without the addition of laminins (LM-511/521 and LM-411/421) for 5-10 days.

### WNT5A Conditional Medium

WNT5A-producing fibroblasts (ATCC CRL-2814) and control fibroblasts (ATCC CRL-2648) were cultured in DMEM containing 10% FBS and 1% penicillin/Streptomycin (Complete Medium). Upon reaching confluency, cells were washed with PBS prior to incubation in Complete Medium for one week. Conditioned medium was subsequently collected, filtered through a 0.2 μm sterile filter, and frozen at −80° C. in 50 ml aliquots. Conditioned medium was mixed with Differentiation Medium (S5 with 10 μM Trolox, 2 μM R428, 1 mM N-acetyl cysteine, 10 μM Alk5 inhibitor II, 1 μM T3) at a 1:1 ratio, and then was used to treat HILOs for 5-10 days.

PD-L1 Induction in Human Islets and wHILOs

PD-L1 expression was induced by recombinant human IFNγ (R&D Systems, 285-IF, 2-12 hours treatment at 1-50 ng/ml final concentration). For acute treatment, wHILOs were treated with 10 ng/ml IFNγ in the differentiation medium (S5 with 10IM Trolox, 2 μM R428, 1 mM N-acetyl cysteine, 10IM Alk5 inhibitor II, 1 μM T3 and 100 ng/ml rhWnt4 (recombinant human Wnt4)) for 2 hours. Cells were then washed twice with PBS prior to culturing in differentiation medium (S5 with 10IM Trolox, 2 μM R428, 1 mM N-acetyl cysteine, 10 μM Alk5 inhibitor II, 1 μM T3 and 100 ng/ml rhWnt4) (single pulse stimulation). IFNγ exposure was repeated 3 times with washing and 24 hours resting time in differentiation medium (S5 with 10IM Trolox, 2 μM R428, 1 mM N-acetyl cysteine, 10 μM Alk5 inhibitor II, 1 μM T3 and 100 ng/ml rhWnt4) between each IFNγ exposure (MPS stimulation) to generate wHILO^{ie}. After the final IFNγ pulse, cells were cultured in the tissue culture incubator for a week prior to the RNA-seq analyses (FIGS. 14A-14C), ATAC-seq analyses (FIG. 14D) and transplantation into STZ-induced diabetic C57BL6J mice (FIG. 5J) or humanized mice (FIG. 15B).

### Isolation of Pancreatic Islets

Mouse pancreatic islets were isolated as previously described by E. Yoshihara et al., 2010, Nature communications, 1:127, with slight modifications. Briefly, 0.5 mg/ml collagenase P (Roche REF11213873001, diluted in HBSS buffer, GIBCO, 14170-112) was injected through the common bile duct, and the perfused pancreas was dissected and incubated at 37° C. for 21 minutes. Digested exocrine cells and intact islets were separated via centrifugation over Histopaque-1077 (Sigma, H8889) at 900×g for 15 minutes, and intact islets were manually selected. Human islets were provided by the Integrated Islets Distribution Program under an approved protocol.

### Insulin/c-Peptide Secretion Assays

Insulin release from intact islets was monitored using batch incubation methods as reported by E. Yoshihara et al., 2016, Cell metabolism, 23:622-634. Briefly, overnight-cultured, isolated pancreatic islets (RPMI-1640 medium supplemented with 10% (v/v) fetal bovine serum and 1% (v/v) Antibiotic-Antimycotic (Gibco)) were pre-cultured at 37° C. for 30 minutes in Krebs-Ringer bicarbonate buffer (KRBB) containing 129.4 mM NaCl, 3.7 mM KCl, 2.7 mM CaCl₂), 1.3 mM KH₂PO₄, 1.3 mM MgSO₄, 24.8 mM NaHCO₃ (equilibrated with 5% CO₂, 95% O₂, pH 7.4), 10 mM HEPES and 0.2% (v/v) BSA (fraction V, Sigma) (KRBH) with 3 mM glucose). Pancreatic islets were incubated in Krebs-Ringer bicarbonate HEPES (KRBH) buffer (500 μL/10 islets) with 3 mM or 20 mM glucose for 30 minutes to determine insulin secretion levels. After 30 minutes, the islets were pelleted by centrifugation and secreted insulin levels were determined in the medium by Enzyme Linked Immunosorbent Assay (ELISA), (Rat/mouse Insulin ELISA KIT (Millipore) and Human Insulin ELISA KIT or ultrasensitive human c-peptide ELISA Kit (Millipore) for mouse and human islets, respectively). For human iPSC derived cells, the cells (1×10⁶ cells/well in 24 well culture plates) were pre-cultured in 3 mM glucose KRBH buffer (500 μl/well). The cells were then incubated in KRBB (200 μl/well) with 3 mM or 20 mM glucose for 30 minutes to determine c-peptide secretion levels as an indicator of insulin secretion levels. After 30 minutes, the cells were pelleted by centrifugation and c-peptide levels were determined in the supernatant medium using the human c-peptide ELISA KIT (Millipore). (e.g., FIGS. 7D-1 and 7D-2).

### Oxygen Consumption and Extracellular Acidification Rates

Oxygen consumption rate (OCR) and extracellular acidification rate (ECAR) (e.g., of islets) were recorded in 24-well plates using an XF24 sea horse (Seahorse Biosciences). (FIG. 7C). Briefly, 70 size matched, human islets, hiPSC spheroids, or HILOs were pre-cultured in 3 mM glucose XF DMEM medium (pH 7.4) supplemented to contain 1 mM sodium pyruvate (Base Medium) for 1 hour prior to transfer to XF24 islet culture plates in Base Medium. OCRs (reported as percent change compared to 3 mM glucose) were recorded during the incremental addition of glucose, up to a final concentration of 20 mM glucose. Subsequently, mitochondrial stress reagents (oligomycin, Fccp, Rotenone, and Antimycin A), were added as instructed in the Mitostress Kit (Seahorse Biosciences).

### Islet and HILO Transplantation Studies

Immunodeficient NOD-SCID, C57BL6J and Hu-PBMC-SGM3 mice were purchased from Jackson Laboratory and maintained in autoclaved cages in a SPF facility at the Salk Institute. Mice were rendered diabetic by a single high dose (180 mg/kg) injection or 5 times with a multi low dose (MLD, 50 mg/kg) injection of streptozotocin (STZ; i.p., Sigma S0130-500MG). One week after the STZ injection, mice with blood glucose levels higher than 300 mg/dl were used as transplant recipients. Human and mouse islets (200-500 islets or 500-1,000 IEQ for mouse islets, 500-1,000 islets or 1,000-2,000 IEQ for human islets per animal) or HILOs (500 clusters) were resuspended in 200 μl RPMI-1640 medium, loaded into laboratory tubing (SiLastic, 508-004), and centrifuged (400×g for 1-2 minutes) to generate cell clusters in the center of the tubing. Cell clusters were transplanted (approximately 30-50 μl) under the kidney capsules in 8 to 16-week-old STZ-injected diabetic mice. Ketamine (80 mg/kg) and xylazine (10 mg/kg) were used as surgical anesthetics, and mice were placed on 37° C. heating pads to recover. Blood glucose levels were monitored by using a commercially available blood glucose/ketone monitor (Nova Max Plus). Nephrectomy (Nx) for graft removal experiments were carried out to confirm the efficacy for glucose regulation in the transplanted wHILOs. The kidney with graft was ligated at the renal hilum using 4-0 silk (LOOK, SP116), and then was resected. Removed grafts were processed for analyses of immune profiling.

### ATAC-seq

ATAC-seq was performed on 5×10⁴ GFP-positive (GFP+) cells isolated using Fluorescence Activated Cell Sorting (FACS) from HILOs treated with PBS or with 100 ng/ml rhWnt4 from day 27 to day 34 as described in J. D. Buenrostro et al., 2015, Current Protocols in Molecular Biology, 109:21-29. Reads were aligned by Bowtie to hg19, and peaks were called by HOMER using default settings. Differential peaks and motif analyses from 2 biological duplicates were identified using HOMER essentially as instructed (see, e.g., S. Heinz et al., 2010, J. Mol. Cell, 38:576-589). Detailed methods for HOMER are freely available, e.g., at http://http://homer.salk.edu/homer/. Briefly, the program searches against the target and background sequences for enrichment of known motifs, and returns motifs enriched with a threshold of 1.5-fold change and a p-value of less than 0.05. Promoter regions, defined as 1 kilobase (kB) upstream from the transcription start site, of genes with enhanced chromatin accessibility upon Wnt4 treatment, were interrogated for enriched motifs of 8-16 bp using HOMER motif analysis.

### Bulk RNA-Seq Library Generation

Total RNA was isolated from cell pellets treated with RNAlater (Invitrogen) using the RNeasy micro kit (Qiagen) and treated with DNaseI (Qiagen) for 30 minutes at room temperature. Sequencing libraries were prepared from 100-500 ng total RNA using the TruSeq RNA Sample Preparation Kit v2 (Illumina) according to the manufacturer's protocol. Briefly, mRNA was purified, fragmented, and used for first- and second-strand cDNA synthesis followed by adenylation of 3′ ends. Samples were ligated to unique adapters and PCR amplified. Libraries were then validated using the 2100 BioAnalyzer (Agilent), normalized and pooled for sequencing.

### High-Throughput Sequencing and Analysis

RNA-Seq libraries prepared from 3 biological replicates for each experimental condition were sequenced on the Illumina HiSeq 2500 using bar-coded multiplexing and a 100 bp read length. Image analysis and base calling were automatically generated with the Illumina HiSeq Real-Time Analysis Software. This yielded a median of 29.9M usable reads per sample. Short read sequences were mapped to a UCSC hg19 reference sequence using the RNA-Seq aligner STAR (A. Dobin et al., 2013, Bioinformatics, 29:15-21). Known splice junctions from hg19 were supplied to the aligner and de novo junction discovery was also permitted. Differential gene expression analysis, statistical testing and annotation were performed using Cuffdiff 2 (C. Trapnell et al., 2013, Nature Biotechnology, 31:46-53). Transcript expression was calculated as gene-level relative abundance in fragments per kilobase of exon model per million (fpkm) mapped fragments and employed correction for transcript abundance bias (A. Roberts et al., 2011, Bioinformatics, 27:2325-2329). RNA-Seq results for genes of interest were also explored visually using the UCSC Genome Browser. Heatmaps were generated by R-Script with heatmap.2 (gplot) software or Cluster with Javatree view software. Scale of heatmaps was determined by Z-score (FIG. 2A, FIG. 3D and FIG. 3G).

### Droplet-Based Single-Cell RNA Sequence

Three biological replicates (200 clusters per replicate) of hiPSC-derived endocrine progenitor cells (day15), HILOs, and WNT4-treated HILOs (100 ng/ml rhWNT4 for 5 days), as well as human islets (IIDP donor ID 1874), were dissociated into single cell suspensions using TrypLE. Single cells were processed through the Chromium Single Cell Platform using the GemCode Gel Bead, Chip and Library Kits (10×Genomics) as per the manufacturer's protocol. In brief, 8,800 single cells were sorted into 0.4% BSA in PBS for a targeted 5000 cell recovery. Cells were transferred into Gel Beads (Chromium Single Cell 3” v2) in Emulsion in the Chromium instrument, where cell lysis and barcoded reverse transcription of RNA was carried out, followed by amplification, shearing and 5′ adaptor and sample index attachment. Libraries were sequenced on an Illumina HiSeq 4000 instrument.

scRNA-seq Data Analysis

Initial data processing, including de-multiplexing, alignment to the GRCh38 transcriptome and unique molecular identifier (UMI)-collapsing, were performed using Cell Ranger software (10×Genomics, ver2.0.2). An overview of single cell sample information was generated from the results of Cell Ranger pipelines. R studio (https:www.rstudio.com), Cell Ranger R Kit, Seurat, monocle and other custom R scripts were used. For the identification of cell types, the cluster cell function of monocle was used. (FIG. 4B). Clustering of cells was performed using the Seurat R package in two iterative rounds of principal component analysis.

Cells having unique gene counts less than 200 were removed (FilterCells function) prior to normalization of digital gene expression matrices by total expression, multipled by a scale factor (default setting of 10,000) and log-transformed (NormalizeData function). A set of variable genes was then identified by binning the average expression of all genes and dispersion (variance divided by the mean) for each gene, placing these genes into bins, and then calculating z-score for dispersion within each bin (FindValiableGenes Function). Linear dimensional reduction was performed using the default setting of RunPCA, and the principal components were evaluated for statistically significant gene expression signals using the Jackstraw method (JackStraw function, not shown). At most, 12 principal components were used in this second round of clustering. t-distributed stochastic neighbor embedding (t-SNE) mapping was used to visualize scRNA-seq results.

Clustered cell populations were classified, and the top10 differentially expressed genes were identified (FindAllMarkers function). Cell types within the clustered cell populations were verified by examining the expression of canonical marker genes, including insulin (β-cells), glucagon (α-cells), somatostain (δ-cells), pancreatic polypeptide (γ-cells), ghrelin (ε-cells), Prss1 (aciner cells), Krt19 (duct cells) and Acta2 (stellate cells). (FIGS. 2D, 2E, FIG. 4A and FIGS. 6D-6F).

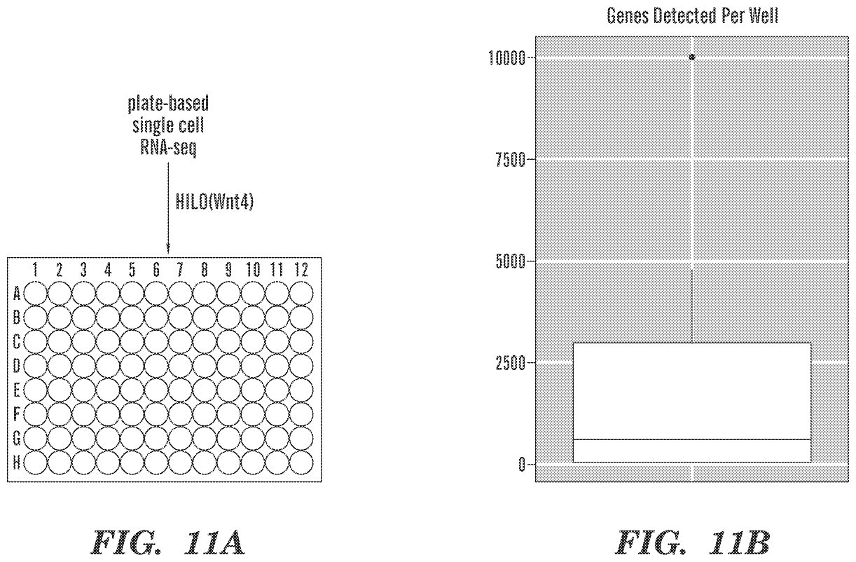

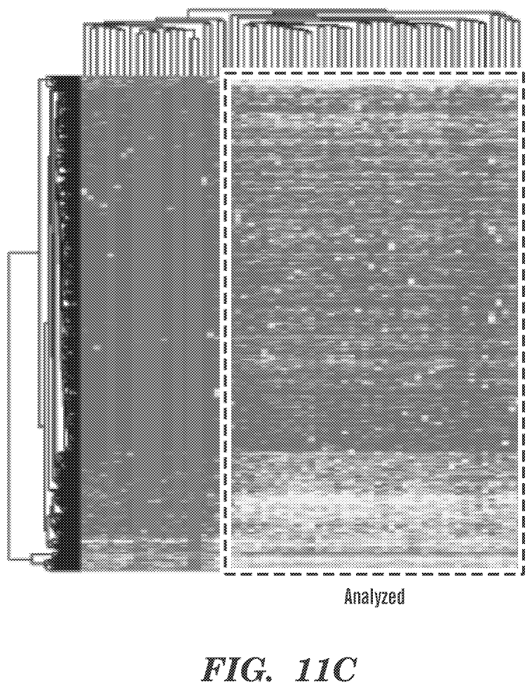

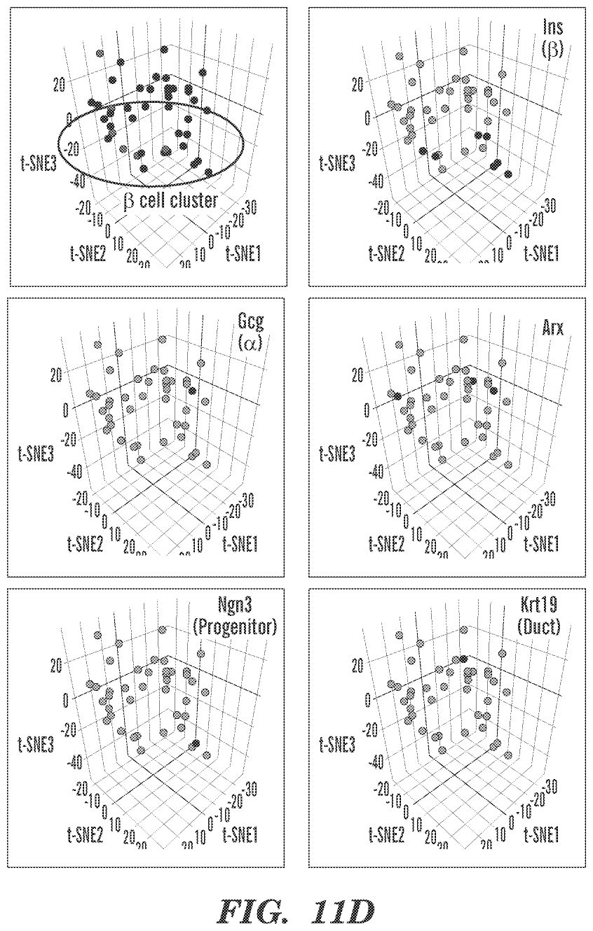

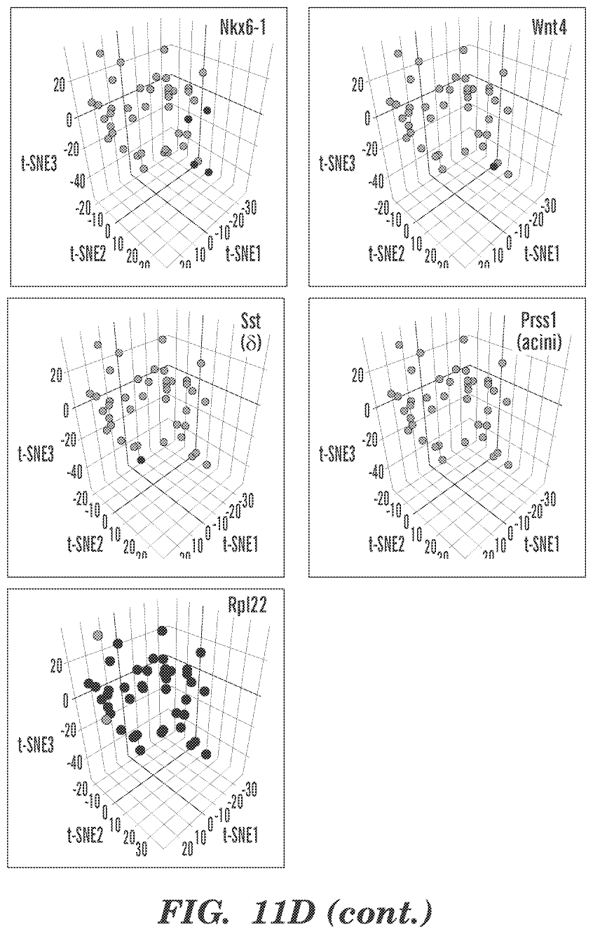

scRNA-seq data from WNT4-treated HILOs (4,840 cells) and human islets (7,248 cells) were combined in 1 Seurat object, and the highly variable genes were identified as described above. Cell types within the clustered populations were identified by reference to differentially expressed genes in human islet cells. The β-cell populations identified in WNT4-treated HILOs and human islets were compared to identify differentially expressed genes. (FIGS. 10A-10C; FIGS. 11A-11D).

### Software and Program for Bioinformatics Analysis

The following software or programs were used for genomic data analysis: R studio (https://www.rstudio.com/); Cell Ranger R Kit (https://support.10×genomics.com/single-cell-gene-expression/software/pipelines/latest/rkit); Seurat (https://satijalab.org/seurat/); Monocle (http://cole-trapnell-lab.github.io/monocle-release/); DAVID (https://david.ncifcrf.gov/home.jsp); GOplot (https://wencke.github.io); UCSC genome browser (http://genome.ucsc.edu); and Homer (http://homer.ucsd.edu/homer/).

### Immunohistochemistry (IHC)

Immunohistochemistry (IHC) of frozen or paraffin-embedded sections of pancreas and human islets or iβeta cells in the kidney capsule (4% PFA-fixed cells) was performed using antibodies to insulin (anti-Insulin antibody, 1/100, Abcam ab7842)), c-peptide (anti-c-peptide antibody, 1/100, Abcam ab30477), glucagon (anti-glucagon antibody, 1/100, Abcam ab10988), somatostatin (anti-somatostatin antibody, 1/100, Abcam ab103790), pancreatic polypeptide (anti-pancreatic polypeptide antibody, 1/100, Abcam, ab113694), NKX2-2 (anti-NKX2-2 antibody, 1/100, DSHB, 74.5A5), NKX6-1 (anti-NKX6-1 antibody, 1/100, DSHB, F55A12), MAFA (anti-MAFA antibody, 1/100, Abcam, ab26405), MAFB (anti-MAFB antibody, 1/100, Abcam, ab26405), PDX-1 (anti-PDX-1 antibody, 1/100, R&D, AF2419), CHGA (anti-CHGA antibody, 1/100, Abcam, ab15160), Synaptophysin (anti-Synaptophysin antibody, 1/100, Biogenex, MU363-UC) and PD-L1 (anti-PD-L1 antibody, 1/100, Abcam, ab20592), (Table 5). Secondary antibodies were coupled to Alexa 568, 647 (Life Technologies), and IHC staining was visualized by confocal microscopy (ZEISS) or fluorescence microscopy. Hoechst 33342 (Thermo Scientific, 62249, 1 μg/ml final concentration) was used for nuclear staining.

TABLE 5

| Antibody (Ab) | | | | | Source/ | |
|---|---|---|---|---|---|---|
| Name | Species* | Host | Ab Type | Applications | Company | Catalog ID |
| Insulin | H, M, R | Guinea pig | Polyclonal | IHC | abcam | ab7842 |
| c-peptide | H, M | Guinea pig | Polyclonal | IHC | abcam | ab30477 |
| Glucagon | H, M, R | Mouse | Monoclonal | IHC | abcam | ab10988 |
| Somatostatin | H, M, R | Rabbit | Polyclonal | IHC | abcam | ab103790 |
| Insulin | H, M, R | Guinea pig | Polyclonal | IHC | abcam | ab7842 |
| Pancreatic | H | Rabbit | Polycronal | IHC | abcam | ab113694 |
| Polypeptide | | | | | | |
| NKX2-2 | H, M, R, C | Chicken | Monoclonal | IHC | DSHB | 74.5A5 |
| NKX6-1 | H, M, R | Rat | Monoclonal | IHC | DSHB | F55A12 |
| MAFA | H, M | Rabbit | Polyclonal | IHC/Flow | Novus | NB400-137 |
| | | | | cytometry | Biologicals | |
| MAFB | H, M, R | Rabbit | Polyclonal | IHC/Flow | abcam | ab66506 |
| | | | | cytometry | | |
| PDX-1 | H, M | Goat | Polyclonal | IHC | R&D Systems | AF2419 |
| ChromograninA | H, M, Mon | Rabbit | Polyclonal | IHC | abcam | ab15160 |
| Synaptophysin | H | Mouse | Monoclonal/ | IHC | BioGenex | MU363-UC |
| | | | Polyclonal | | | |
| PD-L1 antybody | H | Rabbit | Monoclonal | IHC | abcam | ab205921 |
| ChromograninA-PE | H | Mouse | Monoclonal/ | Flow | BD | 564563 |
| | | | Polyclonal | cytometry | Bioscience | |
| NKX6-1- | H, M | Mouse | Monoclonal/ | Flow | BD | 563338 |
| Alexa647 | | | Polyclonal | cytometry | Bioscience | |
| PDX-1-PE | H, M | Mouse | Monoclonal/ | Flow | BD | 562161 |
| | | | Polyclonal | cytometry | Bioscience | |
| anti-mouse | M | Rat | Monoclonal | Flow | BioLegend | 103138 |
| CD45-510 | | | | cytometry | | |
| anti-mouse | M | Rat | Monoclonal | Flow | BioLegend | 100229 |
| CD3-650 | | | | cytometry | | |
| anti-mouse | M | Rat | Monoclonal | Flow | BioLegend | 115533 |
| CD19- | | | | cytometry | | |
| PerCP/Cy5.5 | | | | | | |
| anti-mouse | M | Mouse | Monoclonal | Flow | eBioscience | 12-5941-82 |
| NK1.1-PE | | | | cytometry | | |
| anti-mouse | M | Rat | Monoclonal | Flow | eBioscience | 17-5773-80 |
| FoxP3-APC | | | | cytometry | | |
| anti-human | H | Mouse | Monoclonal | Flow | BioLegend | 368526 |
| CD45-510 | | | | cytometry | | |
| anti-human | H | Mouse | Monoclonal | Flow | BioLegend | 317324 |
| CD3-650 | | | | cytometry | | |
| anti-human- | H | Rat | Monoclonal | Flow | BioLegend | 357410 |
| CD4-PE/Cy7 | | | | cytometry | | |
| anti-human- | H | Mouse | Monoclonal | Flow | BioLegend | 368524 |
| CD8-FITC | | | | cytometry | | |
| anti-human | H | Mouse | Monoclonal | Flow | BioLegend | 363016 |
| CD19- | | | | cytometry | | |
| PerCP/Cy5.5 | | | | | | |

Species*: H = Human; M = mouse; R = Rat; C = Chicken; Mon = Monkey

### Flow Cytometry

Clusters at indicated stages were dissociated with TrypLE (GIBCO) with 20 ug/ml DNase for 12 minutes at 37° C. and then were fixed with 4% PFA for 10 minutes at room temperature. Clusters were then permeabilized with 0.2% Triton X for 10 min, blocking with 1000 goat serum for 30 min and stained for various intracellular markers with antibodies, c-peptide, (1/100, abcam, ab30477), PDX-1 (1/100, BD, 562161), NKX6-1 (1/100, BD, 563338), Chromogranin A (1/100, BD, 564583), MAFA (1/100, abeam, ab264583), MAFB (1/100, abeam, ab66506), Glucagon (1/100, abeam, ab82270), Somatostatin (1/100, abeam, 108456) for analysis on a BD Biosciences LSRII instrument. Data were analysed by FlowJo software. Secondary antibodies for c-peptide, Glucagon and Somatostatin were coupled to Alexa 647 (Life Technologies).

### Electron Microscopy (EM) Analysis

Human islets and HILOs in suspension were pelleted in 2% low melting point agarose and subsequently fixed in 2.5% glutaraldehyde with 2% paraformaldehyde in 0.15M cacodylate buffer containing 2 mM calcium chloride (pH 7.4) for one hour at 4° C. Excess agarose was removed, and the pellet was washed in buffer prior to secondary fixing in 1% osmium tetroxide/0.3% potassium ferrocyanide in buffer. After washing in water, the pellet was en bloc stained with 2% uranyl acetate, followed by graded dehydration in ethanol (35%, 50%, 70%, 90%, 100%, 100%). Samples were then rapidly infiltrated in Spurr's resin using a Pelco BioWave microwave processing unit (Ted Pella, Redding, CA), embedded in Pelco Pyramid tip mold (Ted Pella, Redding, CA), and cured at 60° C. overnight. 70 nm ultrathin sections were cut on a Leica UC7 ultramicrotome (Leica, Vienna) and examined on a Libra120 (Zeiss, Oberkochen, Germany) at 120V.

### Immune Profiling of Transplanted HILOs

Transplanted HILOs were harvested at day 26 after transplantation and were dissociated into single cells using TrypLE. After blocking a common epitope found in extracellular regions of mouse Fc-receptors by Fc block (Anti-mouse CD16/CD32 (Fc Shield) (70-0161-U500) staining, antibodies (1:100 dilution) to the cell surface markers CD19 (PerCP/Cy5.5 anti-mouse CD19, BioLegend, 115533), Nk1.1 (anti-mouse Nk1.1 PE, eBioscience, 12-5941-81), CD45 (brilliant violet510 anti-mouse CD45, BioLegend, 103138), CD3 (brilliant violet650 anti-mouse CD3, BioLegend, 100229), Cd11b (anti-human/mouse APC-cyanine, TONBO, 25-0112U100) were used for FACS-based immune profiling. For flow cytometry analyses, data were collected using a BD Biosciences LSRII. For cell sorting, a BD Influx was used (100 micron nozzle tip and 1×PBS sheath fluid with sheath pressure set to 18.5PSI) with sample and collection cooling set to 4 degrees C. Viable (Zombie-UV dye negative) single cells were selected for FACS or analyses using Forward scatter (FSC) and Side scatter (SSC) gating, followed by pulse-width discrimination for FSC and SSC.

The described protocol assays infiltration of lymphocytes (T cells, β cells) into an organ or tissue, e.g., kidney or kidney capsule, following transplant, implant, or transfer of donor cells, islets, organoids (and cells therein). The reduced numbers of CD45+ T cells that infiltrate into tissue such as kidney following transplantation of insulin-producing PD-L1+ wHILOs versus insulin-producing PD-L1-wHILOs demonstrates that the HILOs (and cells therein) expressing PD-L1 are protected from recognition as foreign by T cells and from T cell killing after transplantation (e.g., 27 days after transplantation), (FIGS. 4D and 4E).

Detecting Immunoprotected Cells, Islets, or Organoids (and Cells Therein) Following Transplant, Implant, or Transfer into a Recipient Subject

Primary human cells, islets, and/or organoids derived from human tissues are labeled via infection with a lentiviral-mediated TYF-CMV-eGFP (green fluorescent protein. (Mao, Y et al., 2015. International Journal of Medical Sciences, 12(5), 407-15. doi:10.7150/ijms.11270), which has been shown to produce sustained high GFP expression. GFP-expressing cells/islets/organoids are then exposed to 2-3 IFN-γ, treatments (e.g., MPS IFNγ exposure as described supra), and the subsequent induction of PDL-1 expression is confirmed by q PCR. TFN-exposed cells. islets and/or organoids are transplanted into the kidney capsule of an immune-competent mouse, with naïve cells/islets//organoids (i.e., no IFNγ exposure) transplanted into the ipsilateral kidney capsule as controls. Mice are sacrificed 2-3 weeks after transplantation and kidney resident GFP-positive cells are quantified by fluorescence activated cell sorting (FACS) analysis. Increases in cells/islets/organoids that survive following IFNγ exposure are determined quantitatively, based on the numbers of GFP cells in each kidney as determined from individual mice.

### Quantitative RT-PCR Analysis

Total RNA was extracted using TRIzol reagent (Invitrogen) and RNeasy KIT (Qiagen). Reverse transcription was performed with a SuperScript III First-Strand Synthesis System kit (Invitrogen) or PrimeScript RT reagent kit (TAKARA). Real time quantitative RT-PCR (qPCR) was performed using SYBR Green (Bio-Rad). Primer information is listed in Table 2.

### In Vitro Vascularization

Human multicellular spheroids (MCSs) were embedded in 300 μl of Matrigel with EBM medium (Ronza, cc-3121) in 24 well tissue culture plates. Vascularization was observed over the following 24-72 hours.

Statistical Methods Results were expressed as the mean±SEM. Statistical comparisons were made using Student's t test. Statistically significant differences are indicated as *p<0.05, **p<0.01, ***p<0.001.

### Example 10: Human Islet-Like Organoids

The generation of functional human organs according to methods described herein provides new strategies for drug-screening and disease modeling. Specifically, functional organoids can be used as models of type 2 diabetes for drug screening. Human islet-like organoids responded to amyloid polypeptide (hIAPP) toxicity, an inducer of β cell loss in type 2 diabetic patients and islet dysfunction after transplantation in hyperglycemic patients, hIAPP dose-dependently induced G0/G1 arrest in 24 hours in human islet-like organoids (See, e.g., WO 2017/205511). Such human-like organoids may also be induced to express PD-L1 according to the methods and systems described herein, so as to avoid immune detection and destruction when used for transplantation, implantation, or administration to a subject in need thereof.

In an exemplary assay, 3D mini organs are exposed to stressors that induce type 2 diabetes, such as high levels of free fatty acids (FFAs) and/or, glucose and selected cytokines. The stressed 3D mini organs are then treated with various drugs. In some embodiments, the drug is approved by the Food and Drug Administration (FDA).

As output, the following are assayed in human pancreatic islet organoids: insulin secretion, beta cell apoptosis (PI stain), lactate dehydrogenase A (LDHA) expression via a luciferase reporter, and changes in expression of marker genes including NDUFA4 (Mitochondrial oxidative phosphorylation), ESRRG (Mitochondrial function), KCNK3 (Katp channel activity) and MAFA (beta cell fate marker). For the human pancreas organoid, amylase secretion and apoptosis of exocrine cells (PI stain) are assayed.

In an exemplary assay for modeling human pancreatic cancer tumorigenesis and metastasis in a dish and the potential to screen for drugs that target those diseases, a 3D mini human pancreas is co-cultured with pancreatic cancer cells, stellate cells, and immune cells to create human pancreatic cancer microenvironment in a dish. Various drugs (e.g., FDA-approved drugs) are then screened to find compounds which effectively suppress pancreatic cancer growth or metastasis in a mini human pancreas microenvironment. As output, the following are measured for the pancreas organoid: apoptosis of exocrine cells (PI stain), collagen synthesis (Trichrome stain) and stellate cells activation (GFAP-reporter). Potential candidate drugs identified in these assays are tested in pancreatic cancer tumorigenesis and metastasis mouse models. Genes expression and morphology as well as the degree of cell death, cell growth, and metastasis are investigated.

In an exemplary assay for modeling of human Type 2 diabetes in mice, human islet organoids and/or human liver organoids are transplanted into mice. The mice are then administered various stressors that induce type 2 diabetes, such as a high fat diet (HFD) or cytokines injection. The potential candidate drugs identified in this assay are further tested in human type 2 diabetic mouse model. Genes expression and morphology as well as the degree of diabetes are investigated.

In an exemplary assay for modeling of human pancreatic cancer tumorigenesis and metastasis in mice, human pancreas organoids and/or human liver organoids are transplanted into mice. Mice transplanted with a mini pancreas are used to study human pancreatic cancer growth in human pancreas microenvironment. In another exemplary assay, a mini pancreas and mini liver are co-transplanted in mice. The liver is a major site for metastasis of pancreatic cancer. In vivo, endothelial cells in the mini pancreas and in the mini liver create a pancreas-liver vasculature network for pancreatic cancer metastasis. Thus, mice co-transplanted with a mini pancreas and mini liver are used to study the metastasis of human pancreatic cancer into the human liver. The generation of functional organ-like clusters from pluripotent stem cells (PSC) and human islets and HILOs as described herein provides insight into the mechanisms underlying human diseases, as well as biological therapeutics that function following introduction or transplant into a recipient subject.

The results hereinabove were obtained using the following materials and methods: 3D KELCOGEL® (3DKG) culture medium

KELCOGEL® F low acyl gellan gum (GG-LA) obtained from Modernist Pantry was suspended in pure water 0.3% (w/v) and dissolved by stirring at 90° C. or by microwave. The aqueous solution was sterilized at 121° C. for 20 minutes in an autoclave. The solution was added to TeSR™ medium (Ludwid et al., Nature Methods, 3, 637-646) or custom TeSR™ medium (800 ml DMEM/F12, 13.28 g BSA. 10 ml Glutamax, 560 mg NaHCO₃, 330 mg thiamine, 100 mg reduced glutathione, 3300 mg Vitamin C, 14 μg Selenium, 10 ml NEAA, 2 ml Trace Element B, 1 ml Trace Element C, 7 μl β-ME, 2 ml DLC, 2 ml GABA, 2 ml LiCl, 129.7 μg pipecolic acid, Insulin 2 mg up to 1000 ml) at a final concentration of 0.015%. Methylcellulose (MC) stock solution was added to a final concentration of 0.3% (R&D systems) (e.g., 0.3% KELCOGEL® stock: KELCOGEL® F low acyl GG-LA 300 mg+MilliQ water 100 ml; 3D-KELCOGEL® (3DKG) Stem TeSR™ Base Medium: STEMCELL™ TeSR™ 95 ml+0.3% KELCOGEL® stock 5 ml+MC stock solution 300 ul; 3DKG Custom TeSR™ Base Medium: custom TeSR™ media 95 ml+0.3% KELCOGEL® stock 5 ml+MC stock solution 300 ul; 1% final concentration of Penicillin/streptozocin was added for 3DKG medium.

### Preparation of Human Pancreatic Endocrine Progenitors and β-Like Cells In Vitro

Pancreatic endocrine cells (hiPSC-PEs) were prepared from human iPSC using differentiation methods as previously described. Briefly, human induced pluripotent stem cells (hiPSC) derived from HUVECs were obtained from the Stem Cell Core (Salk Institute). Cells were maintained on MATRIGEL® (BD)-coated dishes in complete STEMCELL™ TeSR™ medium at 37° C. in a humidified 5% CO₂ incubator. For pancreatic differentiation, hiPSC were infected with a human insulin reporter lentivirus (pGreenZero lenti reporter human insulin, System Biosciences) by Spinfection (800 g, 1 hour). Methods 1: Medium was changed to 100 ng/ml human Activin (R&D Systems), 25 ng/ml recombinant human Wnt3a (R&D Systems) in custom TeSR™ medium (800 ml DMEM/F12, 13.28 g BSA, 10 ml Glutamax, 560 mg NaHCO₃, 330 mg thiamine, 100 mg reduced glutathione, 3300 mg Vitamin C, 14 μg Selenium, 10 ml NEAA, 2 ml Trace Element B, 1 ml Trace Element C, 7 μl β-ME, 2 ml DLC, 2 ml GABA, 2 ml LiCl, 129.7 μg PA, Insulin 2 mg up to 1000 ml) for 2 days and then 100 ng/ml human Activin in differentiation medium for another 2 days (Stage 1, Pancreatic Endoderm). Subsequently, the medium was replaced with custom TeSR™ medium with 1 μM dorsomorphin (Calbiochem), 2 μM Retinoic Acid (Sigma), 10 μM SB431542 and 1% of B27 supplement for 7 days (Stage 2). Medium was then replaced with custom TeSR™ medium with 10 uM forskolin (Sigma), 10 μM dexamethasone (Stemgent), 10 μM TGFβ RI Kinase inhibitor II/Alk5 inhibitor II (Calbiochem or Enzo), 10 μM Nicotinamide (Sigma), 1 μM 3,3′,5-Triiodo-L-thyronine sodium salt (T3) and 1% of B27 supplement for 4-5 days (day 15-day 21, Pancreatic endocrine progenitors). Medium was replaced every day (stage 1) or every other day (stage 2 & stage 3).

Methods 2: Medium was changed to 100 ng/ml human Activin (R&D Systems), 25 ng/ml recombinant human Wnt3a (R&D Systems) or 3 μM CHIR99021 (Axon or Selleckchem) in differentiation medium (S1) for 1 day and then 100 ng/ml human Activin in differentiation medium (S1) for another 2 days (Stage 1, Pancreatic Endoderm). Subsequently, medium was replaced with differentiation medium (S2) with 50 ng/ml FGF7 (R&D Systems) for 2 days and then differentiation medium (S3) with 50 ng/ml FGF7, 0.25 μM SANT-1 (Sigma), 1 μM Retinoic Acid (Sigma), 100 nM LDN193189 and 100 nM a-Amyloid Precursor Protein Modulator TPB for 3 days. Subsequently, medium was replaced with differentiation medium (S4) with 0.25 μM SANT-1, 50 nM Retinoic Acid, 10IM Alk5 inhibitor II, 1 μM T3 for 3 days. Subsequently, medium was replaced with differentiation medium (S5) with 100 nM LDN193189, 100 nM Gamma Secretase inhibitor XX GSiXX (Millipore), 10p M Alk5 inhibitor II, 1p M T3 for 7 days. Subsequently, medium was replaced with differentiation medium (S5) with 10IM Trolox (Calbiochem), 2 μM R428 (Selleckchem), 1 mM N-acetyl cysteine, 10IM Alk5 inhibitor II, 1 μM T3 for additional 7 to 20 days.

S1 Medium (MCDB131 Medium, 8 mM glucose, 2.46 g/L NaHCO₃, 2% Fatty acid free BSA, 0.25 mM L-Ascorbic acid 0.002% Insulin-Transferrin-Selenium ITS-X (GIBCO), 2 mM Glutamax, 1% Penicillin-Streptomycin), S2 Medium (MCDB131 Medium, 8 mM glucose, 1.23 g/L NaHCO₃, 2% Fatty acid free BSA, 0.25 mM L-Ascorbic acid, 0.002% Insulin-Transferrin-Selenium ITS-X (GIBCO), 2 mM Glutamax, 1% Penicillin-Streptomycin), S3 Medium (MCDB131 Medium, 8 mM glucose, 1.23 g/L NaHCO₃, 2% Fatty acid free BSA, 0.25 mM L-Ascorbic acid, 0.5% Insulin-Transferrin-Selenium ITS-X (GIBCO), 2 mM Glutamax, 1% Penicillin-Streptomycin), S4 Medium (MCDB131 Medium, 8 mM glucose, 1.23 g/L NaHCO₃, 2% Fatty acid free BSA, 0.25 mM L-Ascorbic acid, 0.002% Insulin-Transferrin-Selenium ITS-X (GIBCO), 2 mM Glutamax, 1% Penicillin-Streptomycin, 10 μg/ml Heparin, 10IM Zinc Sulfate), S5 Medium (MCDB131 Medium or BLAR Medium, 20 mM glucose, 1.754 g/L NaHCO₃, 2% Fatty acid free BSA, 0.25 mM L-Ascorbic acid, 0.002% Insulin-Transferrin-Selenium ITS-X (GIBCO), 2 mM Glutamax, 1% Penicillin-Streptomycin). For 3-dimensional (3D) culture, hiPSC or hESC were cultured in 3DKG Stem TeSR™ Base Medium with 10IM Y-27632 for 5 to 7 days and then the medium was replaced each Differentiation medium with 0.015% Kelcogel and 0.3% Methylcellulose.

Generation of Three-Dimensional Pancreatic Islet Bud In Vitro: Islet-Like Organoids in Matrigel Through Co-Culture with hADSCs and HUVECs

Primary HUVECs and human Adipose-derived stem cells (hADSC) (Invitrogen or PromoCell) were cultured in 15 cm dish with EBM Medium (Ronza, cc-3121) or MesenProRS™ Medium (GIBCO, 12747-010 or Preadipocyte Growth Medium Kit, C-27417), respectively, at 37° C. in a humidified 5% CO₂ incubator. For co-culturing experiments, pancreatic endocrine progenitors derived from human iPSC were treated with Accutase, while HUVECs and hADSC were treated with TrypLE (GIBCO, 12604-013) and cells collected into a 50 ml tube, respectively. After the cells were counted, 1×10⁶ cells of hiPS-PP, 7×10⁶ cells of HUVEC and 1-2×10⁵ cells of hADSC were co-cultured in 1 well of 24 well with 300 ul of MATRIGEL® matrix. For the purpose of scalable generation of human islets like organoids, 1×10⁶ cells of hiPS-PP (day 15-day 21), 7×10⁶ cells of HUVEC and 1-2×10⁵ cells of hADSC were co-cultured in 3DKG Custom TeSR® media with 10 μM forskolin (Sigma), 10 μM dexamethasone (Stemgent), 10IM TGFβ RI Kinase inhibitor II/Alk5 inhibitor II (Calbiochem or Enzo), 10IM Nicotinamide (Sigma), 1 uM 3,3′,5-Triiodo-L-thyronine sodium salt (T3) and 1% of B27 supplement, R428 (2 μM), Zinc sulfate (10 μM) and N-Cys (1 mM). (Methods 1) or co-cultured in differentiation medium (S5) with 100 nM LDN193189, 100 nM Gamma Secretase inhibitor XX GSiXX (Millipore), 10 μM Alk5 inhibitor II, 1 μM T3 for 7 days. Subsequently, medium was replaced with differentiation medium (S5) with 10IM Trolox (Calbiochem), 2 μM R428 (Selleckchem), 1 mM N-acetyl cysteine, 10 μM Alk5 inhibitor II, 1 μM T3 for an additional 7 to 20 days (Methods 2). Mixed cells formed spherical, islet-like clusters within a few days. The medium was changed every other day.

Generation of 3D (Three-Dimensional) Pancreatic Islet Buds In Vitro: Islet-Like Organoids in Scalable Gellan Gum Through Co-Culture with hADSCs and HUVECs

Cells were prepared as described above. Briefly, 1×10⁸ cells of hiPS-PP, 2-7×10⁷ cells of HUVECs and 5-7×10⁶ cells of hADSC were co-cultured in 60-100 ml of 3DKG Custom TeSR™ with 10 μM forskolin (Sigma), 10 μM dexamethasone (Stemgent), 10 μM TGFβ RI Kinase inhibitor II/Alk5 inhibitor II (Calbiochem or Enzo), 10p M Nicotinamide (Sigma), 1 μM 3,3′,5-Triiodo-L-thyronine sodium salt (T3) and 1% of B27 supplement, R428 (2 μM), Zinc sulfate (10IM) and N-Cys (1 mM) (Methods 1) or co-cultured in differentiation media (S5) with 100 LDN193189, 100 nM Gamma Secretase inhibitor XX GSiXX (Millipore), 10p M Alk5 inhibitor II, 1 μM T3 for 7 days. Subsequently, media was replaced with differentiation media (S5) with 10 μM Trolox (Calbiochem), 2 μM R428 (Selleckchem), 1 mM N-acetyl cysteine, 10 μM Alk5 inhibitor II, 1 μM T3 for additional 7 to 20 days (Methods 2). Mixed cells formed spherical, islet-like clusters within a few days. Media was changed every day or every other day.

Generation of 3D (Three-Dimensional) Pancreatic Islets Bud In Vitro: Islet-Like Organoids in Scalable Gellan Gum 3D Culture Methods without (w/o) Using hADSC and HUVECs

Human PSCs, including iPSC or ESC, were initially cultured in matrigel-coated plates (2 dimensional (2D) cultures. Cells were then treated with Accutase (Innovative Cell Technologies, Inc., San Diego, CA) to generate a single cell suspension, washed with PBS and centrifuged at 1000-1300 rpm for 5 minutes to pellet cells. Cells were resuspended with 3DKG Stem TeSR™ Base Medium (Stemcell Technologies, Cambridge, MA) with 10 μM Y-27632 (a RHO/ROCK pathway inhibitor compound) and cultured for an additional for 5 to 7 days until PSC sphere growth reached 50-100 μm diameter. Media was then replaced with differentiation media supplemented with 0.015% Kelcogel and 0.3% Methylcellulose. The culture medium was changed to differentiation medium (S1) containing 100 ng/ml human Activin (R&D Systems), 25 ng/ml recombinant human Wnt3a (R&D Systems) or 3 μM CHIR99021, a glycogen synthase kinase GSK-3 inhibitor (Axon Medchem, Reston, VA; or Selleckchem) for 1 day and then to differentiation medium (S1) containing 100 ng/ml human Activin for another 2 days (Stage 1, Pancreatic Endoderm). Subsequently, the medium was replaced with differentiation medium (S2) containing 50 ng/ml FGF7 (R&D Systems) for 2 days, and then with differentiation medium (S3) containing 50 ng/ml FGF7, 0.25 μM SANT-1 (Sigma), 1 μM Retinoic Acid (Sigma), 100 nM LDN193189 (an ALK2 and ALK3 inhibitor, Sigma) and 100 nM α-Amyloid Precursor Protein Modulator TPB for 3 days. Subsequently, this medium was replaced with differentiation medium (S4) containing 0.25 μM SANT-1, 50 nM Retinoic Acid, 10 μM Alk5 inhibitor II, 1 μM T3 for 3 days. Subsequently, the medium was replaced with differentiation medium (S5) containing 100 nM LDN193189, 100 nM Gamma Secretase inhibitor XX GSiXX (Millipore) 10 μM Alk5 inhibitor II, 1 μM T3 for 7 days. Subsequently, the medium was replaced with differentiation medium (S5) containing 10 μM Trolox (Calbiochem), 2 μM R428 (Selleckchem), 1 mM N-acetyl cysteine, 10 μM Alk5 inhibitor II, 1 μM T3 for an additional 7 to 20 days.

After confirmation of the insulin gene expression by either reporter expression or qPCR (typically on day 20-30), the medium was changed to differentiation medium (S5) containing 10 μM Trolox (Calbiochem), 2 μM R428 (Selleckchem), 1 mM N-acetyl cysteine, 10 μM Alk5 inhibitor II, 1 μM T3 and 100 ng/ml recombinant human (rh)Wnt4 (R&D Systems), 400 ng/ml rhWnt5a, or 50% Wnt5a conditioned medium for 1-20 days. Wnt5a conditioned medium was prepared by culturing an L-Wnt5a cell line (ATCC, CRL-2814) in DMEM with 10% FBS, 1% Penicillin-streptomycin for 4 days after cells had reached 70-100% confluence in T175-T225 cell culture flasks.

### Generation of 3D (Three-Dimensional) Liver Bud In Vitro: Organ Buds

Hepatocyte cells (hiPSC-HEs) from human iPSC were prepared using differentiation methods as previously described. Briefly, hiPSCs were maintained on MATRIGEL® (BD)-coated dishes in complete STEMCELL™ TeSR™ medium at 37° C. in a humidified 5% CO₂ incubator. For hepatic differentiation, hiPSC (90% confluence in 6 well) were cultured with 100 ng/ml human Activin (Sigma) and 25 ng/ml recombinant human Wnt3a (R&D systems) or 3 μM CHIR99021 and 1% B27 supplement minus Insulin in RPMI-1640 medium for 1 day and then 100 ng/ml human Activin and 1% B27 supplement minus Insulin in RPMI medium for another 4 days (Stage 1 Hepatic-Endoderm). Subsequently, the medium was replaced with differentiation medium with 10 ng/ml bFGF, 20 ng/ml BMP4 and 10% of B27 supplement in RPMI-1640 medium for 3 days (Stage 2). The medium was then replaced with differentiation medium with 0.1 μM Dexamethasone, 20 ng/ml OncostatinM (R&D Systems) and 10-20 ng/ml Hepatic Growth Factor (HGF, R&D Systems) and 1% of B27 supplement in Hepatocyte Culture Media (Lonza, MD, CC-3198, withdraw EGF and Gentamicin/Amphotericin-B) for 4-22 days (day15-day19, Pancreatic endocrine progenitors). The medium was replaced every day (stage 1) or every other day (stage 2 & stage 3). Primary HUVECs cells and human Adipose-derived stem cells (hADSC) (InVitrogen or PromoCell) were cultured in 15 cm dish with EBM Medium (Ronza, cc-3121) or MesenProRS Medium (GIBCO, 12747-010 or Preadipocyte Growth Medium Kit, C-27417), respectively, at 37° C. in a humidified 5% CO₂ incubator. For co-culturing experiments, day 10-hepatocytes derived from human iPSC were treated with Accutase, while HUVECs and hADSC were treated with TrypLE (GIBCO, 12604-013) and cells were collected into 50 ml tubes, respectively. After the cells were counted, 1×10⁶ cells of hiPS-PP, 7×10⁶ cells of HUVEC and 1-2×10⁵ cells of hADSC were co-cultured in 1 well of 24 well with 300 ul of matrigel. Liver-like organoids were formed within 1 to 2 days. Then, liver-like organoids were taken out from MATRIGEL® matrix and cultured in in 3DKG Custom TeSR™. In an embodiment, cells (hepatocytes) of the liver-like organoids were molecularly engineered to express one or more checkpoint proteins.

### Generation of 3D (Three-Dimensional) Heart Bud In Vitro: Organ Buds

Cardiomyocyte cells (hiPSC-CDs) were prepared from human iPSC using differentiation methods as previously described. Briefly, hiPSCs were maintained on MATRIGEL® (BD)-coated dishes in complete Stemcell™ TeSR™ media at 37° C. in a humidified 5% CO₂ incubator. For cardiac differentiation, hiPSC (90% confluence in 6 well) were cultured with 100 ng/ml human Activin (R&D Systems) and 10 μM CHIR99021 and 1% B27 supplement minus Insulin in RPMI1640 media for 1 days and then 1% B27 supplement 5 minus Insulin in RPMI media for another 2 days (Stage 1 cardiac-Mesoderm). Subsequently, medium was replaced with RPMI1640 with 5 μM IWP-2 and 1% B27 supplement minus Insulin in RPMI medium for 1 days (Stage 2). The medium was then replaced with 1% B27 supplement minus Insulin in RPMI Medium for 6 days or more (Stage 3). Cardiac contraction started around day 13. The medium was replaced every day (stage 1) or every other day (stage 2 & stage 3). Primary HUVECs cells and human Adipose-derived stem cells (hADSC) (Invitrogen or PromoCell) were cultured in 15 cm dish with EBM Medium (Ronza, cc-3121) or MesenProRS™ Media (GIBCO, 12747-010 or Preadipocyte Growth Medium Kit, C-27417), respectively, at 37° C. in a humidified 5% CO₂ incubator. For co-culturing experiments, day 13 to day 15 cardiomyocytes derived from human iPSC were treated with Dispase, while HUVECs and hADSC were treated with TrypLE (GIBCO, 12604-013) and cells collected into 50 ml tubes, respectively. After the cells were counted, 1×10⁶ cells of hiPS-PP, 7×10⁶ cells of HUVEC and 1-2×10⁵ cells of hADSC were co-cultured in 3DKG Custom TeSR™ medium. Mini heart like organs capable of contracting were formed within a few days. In an embodiment, cells (cardiomyocytes) of the mini-heart-like organoids were molecularly engineered to express one or more checkpoint proteins.

### Generation of 3D (Three-Dimensional) Intestine Bud In Vitro: Organ Buds

Intestinal cells (hiPSC-ITs) were prepared from human iPSC using differentiation methods as previously described. Briefly, hiPSCs were maintained on Matrigel® (BD)-coated dishes in complete Stemcell™ TeSR™ Medium at 37° C. in a humidified 5% CO₂ incubator. For intestinal cell differentiation, hiPSC (90% confluence in 6 well plates) were cultured with 100 ng/ml human Activin (R&D Systems), 3 μM CHIR99021, 2 mM Glutamax and 1% B27 supplement minus Insulin in RPMI1640 medium for 1 day and then 100 ng/ml human Activin (R&D Systems), 2 mM Glutamax and 1% B27 supplement minus Insulin in RPMI1640 medium for another 3 days (Stage 1 Forgut-Endoderm). Subsequently, medium was replaced with 500 ng/ml Wnt3a, 500 ng/ml FGF4 and 1% B27 supplement in RPMI 1640 medium for 4 days (Stage 2). Cells were transferred to Matrigel® matrix and then a 3D-spheroid Matrigel® dorm was made in the bottom of 24 well. The medium was then replaced with 1% B27 supplement, 1% N2 supplement, 500 ng/ml R-spondin, 100 ng/ml Noggin, 50 ng/ml EGF, 2 mM Glutamax™ supplement, 10 μM HEPES in DMEM/F12 Medium for 7 days or more (stage3). Intestinal-like organoid spheroids were observed within a week. The medium was replaced every day (stage 1) and every other day (stage 2 & stage 3). Primary HUVECs cells and human Adipose-derived stem cells (hADSC) (Invitrogen or PromoCell) were cultured in a 15 cm dish with EBM Media (Ronza, cc-3121) or MesenProRS™ Medium (GIBCO®, 12747-010 or Preadipocyte Growth Medium Kit, C-27417), respectively, at 37° C. in a humidified 5% CO₂ incubator. For co-culturing experiments, intestinal progenitors (day 7) derived from human iPSC were treated with Accutase, while HUVECs and hADSC were treated with TrypLE (GIBCO®, 12604-013) and cells were collected into 50 ml tubes, respectively. After counting the cells, 1×10⁶ cells of hiPS-PP, 7×10⁶ HUVEC cells and 1-2×10⁵ hADSC cells were co-cultured in 3DKG Custom TeSR™ medium. In an embodiment, intestinal cells of the intestine-like organoids were molecularly engineered to express one or more checkpoint proteins.

Insulin Secretion Assay (Primary Mouse and Human Pancreatic Islets and Human iPSC-Derived Cells)

Insulin release from intact islets was monitored using batch incubation methods (Yoshihara et al., 2010, Nat. Commun. 1:127). Briefly, overnight-cultured isolated pancreatic islets (RPMI-1640 supplemented with 10% (v/v) fetal bovine serum and 1% (v/v) Antibiotic-Antimycotic (Gibco)) were pre-cultured at 37° C. for 30 min (Krebs-Ringer bicarbonate buffer (KRBB) containing 129.4 mM NaCl, 3.7 mM KCl, 2.7 mM CaCl₂), 1.3 mM KH₂PO₄, 1.3 mM MgSO₄, 24.8 mM NaHCO₃ (equilibrated with 5% CO₂, 95% O₂, pH7.4), 10 mM HEPES and 0.2% (v/v) BSA (fraction V, Sigma) (KRBH) with 3 mM glucose). Pancreatic islets were then incubated in KRBH buffer (500 μl/10 islets) with 3 mM or 20 mM glucose to determine insulin secretion levels. After 30 min, islets were pelleted by centrifugation and insulin levels determined by ELISA (Rat/mouse Insulin ELISA KIT (Millipore) and Human Insulin ELISA KIT (Millipore) for mouse and human islets, respectively). For human iPSC derived cells, the cells (1×10⁶ cells/well in 24 well) were pre-cultured in 3 mM glucose KRBH buffer (500 μl/well). The cells were then incubated in KRBB (200 μl/well) with 3 mM or 20 mM glucose to determine c-peptide secretion levels as indicator of insulin secretion levels. After 30 min, the cells were pelleted by centrifugation and c-peptide levels were determined by human c-peptide ELISA KIT (Millipore).

### Example 10 Methods

### Quantitative RT-PCR Analysis

Total RNA was extracted using TRIzol reagent (Invitrogen) and RNeasy KIT (Qiagen). Reverse transcription was performed with a SuperScript III First-Strand Synthesis System kit (Invitrogen) or PrimeScript RT reagent kit (TAKARA). Real time quantitative RT-PCR (qPCR) was performed using SYBR Green (Bio-Rad).

### Lentivirus Production for Proinsulin-NanoLuc

Proinsulin-NanoLuc in pLX304 (Addgene, #62057) was obtained from Addgene. Proinsulin-NanoLuc lentivirus was produced using a second-generation viral packaging system. Briefly, 14 μg of Proinsulin-NanoLuc, 6.6 μg of PsPAX2 packaging plasmid (Addgene 12260), 5.4 μg of pMD2.G envelope plasmid (Addgene 12259) and 54 μl Lipofectamin2000 (Invitrogen) were used to transfect a T75 flask of HEK293LTV packaging cells. Twenty-four (24) hours after transfection, media was changed to fresh DMEM with 10% FBS and 1% Penicillin/Streptozocine. Forty-eight (48) hours and 96 hours after transfection, viruses were collected as day 1 and day 3, respectively and passed through 0.2 μm cellulose acetate filters (VWR). Viruses were aliquoted and frozen at −80° C. until use.

### Gaussia Luciferase Assay for Insulin Secretion Measurement

Mouse islets, human islets and human islets like organoids were plated in their respective growth media with 10 μg/ml Polybrene® polymer (Santacruz). Viruses were then added. After overnight culture, cells were placed in fresh growth media. Forty-eight (48) to 72 hours after infection, mouse islets, human islets and human islet-like organoids were picked up by hand and then placed into 96 wells with single islet or organoid. Then, insulin secretion assays were performed. Briefly, a single islet or organoid was pre-incubated with 3 mM glucose KRBB at 37° C. for 30 min to 1 hour. The cells were then incubated in KRBB (100 μl/well) with 3 mM for 30 min and then sequentially incubated with 20 mM glucose with or without 100 nM Exendin-4 or 3 mM glucose with 20 mM KCl (100 μl/well). To determine Gaussia Luciferase activity as indicator of insulin secretion levels, 10 μl of samples are used for Luciferase assay using Pierce Gaussia Luciferase Flash Assay Kit (Prod #16159, Thermo Scientific).

INS-1 cells were infected with the virus by spinfection (800 g, 1 hour at 37° C. and then changed to fresh INS-1 growth media. Seventy-two (72) hours after transfection, INS-1 cells were treated with 5 μg/ml Blasticidin (Invitrogen) for 7 days to select for Proinsulin-NanoLuc expressing cells. For insulin secretion assay, the cells (5×10⁴-1×10⁵ cells/well in 96 well) were pre-cultured in 3 mM glucose KRBB (100 μl/well). The cells were then incubated in KRBB (100 μl/well) with 3 mM and then sequentially incubated with 20 mM glucose with or without 100 nM Exendine-4 or 3 mM glucose with 20 mM KCl (100 μl/well). To determine Gaussia Luciferase activity as indicator of insulin secretion levels, 10 μl of samples are used for Luciferase assay using Pierce Gaussia Luciferase Flash Assay Kit (Prod #16159, Thermo Scientific).

### Vascularization Test In Vitro

Human islet-like organoids were embedded in 1 well of 24 well plate with 300 μl of Matrigel® matrix with EBM Media (Ronza, cc-3121). Vascularization was observed within 24-72 hours.

3D Culture of hADSCs and WNT Protein Expression

hADSCs undergo changes in the expression of Wnt genes, in particular genes in the Wnt5a pathway, during the spontaneous self-organization that occurs in 3D culture. Wnt5a was found to be the predominant protein expressed among the Wnt proteins in hADSC 3D culture over time.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-24. (canceled)

25. An immunoprotected cell, human islet-like organoid or pancreatic islet organoid having sustained expression of an immune checkpoint protein, said cell or organoid produced following contact with multiple intermittent exposures to interferon gamma (IFNγ) to increase survival or reduce cell or organoid death following transplantation.

26. The immunoprotected cell, human islet-like organoid or pancreatic islet organoid of claim 25, wherein the donor cell is an organoid cell, an islet cell, an islet-like organoid cell, a β-like islet cell.)

27. The immunoprotected cell, human islet-like organoid or pancreatic islet organoid of claim 25, wherein the islet-like organoid or a pancreatic islet organoid comprises endocrine progenitor cell-derived, multi-lineage cells including beta, alpha, delta, epsilon cells, duct-like cells, or a combination thereof, is vascularized, and exhibits glucose-stimulated insulin secretion (GSIS).

28. The immunoprotected cell, human islet-like organoid or pancreatic islet organoid of claim 25, wherein IFNγ is in an amount of about 1 ng/ml to 25 ng/ml; wherein the cell or organoid is contacted for greater than 1 hour at a first time point during a time period of at least about or equal to 24-hours; wherein, following the first time point, the cell or organoid is washed and rested in medium in the absence of IFNγ between being contacted with IFNγ; and wherein the cell or organoid is contacted for at least about 0.5 hour at two or more additional time points thereafter.

29. The human islet-like organoid or pancreatic islet organoid of claim 25, which exhibits sustained expression of the immune checkpoint protein PD-L1.

30. A human islet-like organoid (HILO) derived from endocrine progenitor cells cultured in a three-dimensional matrix comprising Wnt4 or Wnt5 protein and comprising multi-lineage cells comprising at least two of beta (β) cells, alpha (α) cells, delta (δ) cells, epsilon (ε) cells and duct-like cells, wherein the HILO is vascularized, exhibits glucose-stimulated insulin secretion (GSIS) and exhibits sustained expression of an immune checkpoint protein.

31. The human islet-like organoid (HILO) of claim 30, which is a pancreatic islet-like organoid or a pancreatic organoid.

32. The human islet-like organoid (HILO) of claim 30, wherein the organoid further exhibits KCl-stimulated insulin secretion or glucose stimulated insulin secretion.

33. The human islet-like organoid (HILO) of claim 30, wherein the three-dimensional matrix comprises gellan gum.

34. The human islet-like organoid (HILO) of claim 30, wherein the three-dimensional matrix comprises recombinant human Wnt4 protein and/or recombinant human Wnt5 protein, or conditioned cell medium containing Wnt4 and/or Wnt5 protein.

35. The human islet-like organoid (HILO) of claim 30, wherein the endocrine progenitor cells are selected from induced pluripotent stem cells (iPSCs), embryonic pluripotent stem cells (ePSCs), and/or pancreatic progenitor cells.

36. The human islet-like organoid (HILO) of claim 30, wherein the endocrine progenitor cells express at least one of neurogenin 3, neurod1, Nkx2.2 and Pax4 biomarkers.

37. The human islet-like organoid (HILO) of claim 30, which expresses FLTP and ESRR gamma genes.

38. The human islet-like organoid (HILO) of claim 30, further comprising an adipose-derived stem cell and/or an endothelial cell.

39. The human islet-like organoid (HILO) of claim 38, wherein the adipose-derived stem cell is a human adipose-derived stem cell (hADSC) and/or the endothelial cell is a human umbilical vein endothelial cell (HUVEC).

40. The human islet-like organoid (HILO) of claim 30, which further exhibits KCl-stimulated insulin secretion, GLP-1 stimulated insulin secretion, somatostatin secretion, or glucagon secretion.

41. The human islet-like organoid (HILO) of claim 30, wherein the human islet-like organoid (HILO) expresses a beta cell lineage marker selected from the group consisting of NKX2-2, NEUROD1, RFX6, GCK, INS, NKX6-1, UCN3, MAFB and SYT4 and an ARX alpha cell lineage marker.

42. The human islet-like organoid (HILO) of claim 30, wherein the pancreatic HILO expresses a beta cell transcription factor selected from the group consisting of Pdx1, MafA, Pax4, Pax6, NeuroD1, Nkx6-1, Gata6, and Foxa2.

43. The human islet-like organoid (HILO) of claim 30, wherein the immune checkpoint protein binds to an immune cell-expressed cognate ligand selected from programmed cell-death protein 1 (PD-1); cytotoxic T-lymphocyte protein 4 (CTLA-4); lymphocyte activation gene 3 protein (LAG-3); killer cell immunoglobulin-like receptor (KIR); indoleamine 2,3-dioxygenase 1 (IDO1); tumor necrosis factor receptor superfamily member 9 (4-1BB); glucocorticoid-induced TNFR family related gene (GITR); T-cell immunoglobulin domain and mucin domain (TIM-3); tumor necrosis factor receptor superfamily member 4, (OX40); adenosine A2A receptor (A2AR); B7-H3; B7-H4; B7-1/B7-2; BTLA; V-domain Ig suppressor of T cell activation (VISTA); or a combination of any of the foregoing.

44. A kit comprising an immunoprotected cell, human islet-like organoid or pancreatic islet organoid of claim 25, or a pharmaceutically acceptable composition comprising said immunoprotected cell, human islet-like organoid or pancreatic islet organoid.

45. A kit comprising the human islet-like organoid (HILO) of claim 30, or a pharmaceutically acceptable composition comprising said HILO.


SEQUENCE LISTING (110 sequences)

SEQ ID NO: 1 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - DNA, 2057 bp, Homo sapiens
  source 1..2057: mol_type = other DNA
atattgtgcttccaccactgccaataacaaaataactagcaaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtttaaattacttcaggggaagagaagacaaaacgagtctttcattcggtgtgaacttttctctttaattttaactgatttaacactttttgtgaattaatgaaatgataaagacttttatgtgagatttccttatcacagaaataaaatatctccaaatgtttccttttcaaaaaaaaaaaaaaaaaa

SEQ ID NO: 3 - protein, 609 aa, Homo sapiens [sequence omitted]

SEQ ID NO: 4 - DNA, 2264 bp, Homo sapiens
  source 1..2264: mol_type = other DNA
agtatattagtgctaatttccctccgtttgtcctagcttttctcttctgtcaaccccacacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaaaaaaaaaaaaaaaaaaa

SEQ ID NO: 5 - protein, 313 aa, Homo sapiens
MYVSYLLDKDVSMYPSSVRHSGGLNLAPQNFVSPPQYPDYGGYHVAAAAAAAANLDSAQSPGPSWPAAYGAPLREDWNGYAPGGAAAAANAVAHGLNGGSPAAAMGYSSPADYHPHHHPHHHPHHPAAAPSCASGLLQTLNPGPPGPAATAAAEQLSPGGQRRNLCEWMRKPAQQSLGSQVKTRTKDKYRVVYTDHQRLELEKEFHYSRYITIRRKAELAATLGLSERQVKIWFQNRRAKERKINKKKLQQQQQQQPPQPPPPPPQPPQPQPGPLRSVPEPLSPVSSLQASVSGSVPGVLGPTGGVLNPTVTQ

SEQ ID NO: 6 - DNA, 2370 bp, Homo sapiens
  source 1..2370: mol_type = other DNA
ctccaaccattggtgtctgtgtcattactaatagagtcttgtaaacactcgttaatcacggaaggccgccggcctggggctccgcacgccagcctgtggcgggtcttccccgcctctgcagcctagtgggaaggaggtgggaggaaagaaggaagaaagggagggagggaggaggcaggccagagggagggaccgcctcggaggcagaagagccgcgaggagccagcggagcaccgcgggctggggcgcagccacccgccgctcctcgagtcccctcgcccctttcccttcgtgccccccggcagcctccagcgtcggtccccaggcagcatggtgaggtctgctcccggaccctcgccaccatgtacgtgagctacctcctggacaaggacgtgagcatgtaccctagctccgtgcgccactctggcggcctcaacctggcgccgcagaacttcgtcagccccccgcagtacccggactacggcggttaccacgtggcggccgcagctgcagcggcagcgaacttggacagcgcgcagtccccggggccatcctggccggcagcgtatggcgccccactccgggaggactggaatggctacgcgcccggaggcgccgcggccgccgccaacgccgtggctcacggcctcaacggtggctccccggccgcagccatgggctacagcagccccgcagactaccatccgcaccaccacccgcatcaccacccgcaccacccggccgccgcgccttcctgcgcttctgggctgctgcaaacgctcaaccccggccctcctgggcccgccgccaccgctgccgccgagcagctgtctcccggcggccagcggcggaacctgtgcgagtggatgcggaagccggcgcagcagtccctcggcagccaagtgaaaaccaggacgaaagacaaatatcgagtggtgtacacggaccaccagcggctggagctggagaaggagtttcactacagtcgctacatcaccatccggaggaaagccgagctagccgccacgctggggctctctgagaggcaggttaaaatctggtttcagaaccgcagagcaaaggagaggaaaatcaacaagaagaagttgcagcagcaacagcagcagcagccaccacagccgcctccgccgccaccacagcctccccagcctcagccaggtcctctgagaagtgtcccagagcccttgagtccggtgtcttccctgcaagcctcagtgtctggctctgtccctggggttctggggccaactgggggggtgctaaaccccaccgtcacccagtgacccaccgggttctgcagcggcagagcaattccaggctgagccatgaggagcgtggactctgctagactcctcaggagagacccctcccctcccacccacagccatagacctacagacctggctctcagaggaaaaatgggagccaggagtaagacaagtgggatttggggcctcaagaaatatactctcccagatttttactttttcccatctggctttttctgccactgaggagacagaaagcctccgctgggcttcattccggactggcagaagcattgcctggactgaccacaccaaccaggccttcatcctcctccccagctcttctcttcctagatctgcaggctgcacctctggctagagccgaggggagagagggactcaagggaaaggcaagcttgaggccaagatggctgctgcctgctcatggccctcggaggtccagctgggcctcctgcctccgggcaggcaaggtttacactgcggaagccaaaggcagctaagatagaaagctggactgaccaaagactgcagaacccccaggtggcctgcgtcttttttctcttcccttcccagaccaggaaaggcttggctggtgtatgcacagggtgtggtatgagggggtggttattggactccaggcctgaccagggggcccgaacagggacttgtttagagagcctgtcaccagagcttctctgggctgaatgtatgtcagtgctataaatgccagagccaacctggacttcctgtcattttcacaatcttggggctgatgaagaagggggtggggggagtttgtgttgttgttgctgctgtttgggttgttggtctgtgtaacatccaagccagagtttttaaagccttctggatccatggggggagaagtgatatggtgaagggaagtggggagtatttgaacacagttgaattttttctaaaaagaaaaagagataaatgagctttccagatttcagattctgtatttatcttcagattttgtctgcaactattttttattttttaaagaaatgaaatatcttcaaaaaaaaaaaaaaaaaaa

SEQ ID NO: 7 - protein, 503 aa, Homo sapiens
MDLIPNLAVETWLLLAVSLILLYLYGTRTHGLFKKLGIPGPTPLPFLGNALSFRKGYWTFDMECYKKYRKVWGIYDCQQPMLAITDPDMIKTVLVKECYSVFTNRRPFGPVGFMKNAISIAEDEEWKRIRSLLSPTFTSGKLKEMVPIIAQYGDVLVRNLRREAETGKPVTLKHVFGAYSMDVITSTSFGVSIDSLNNPQDPFVENTKKLLRFNPLDPFVLSIKVFPFLTPILEALNITVFPRKVISFLTKSVKQIKEGRLKETQKHRVDFLQLMIDSQNSKDSETHKALSDLELMAQSIIFIFAGYETTSSVLSFIIYELATHPDVQQKVQKEIDTVLPNKAPPTYDTVLQLEYLDMVVNETLRLFPVAMRLERVCKKDVEINGMFIPKGVVVMIPSYVLHHDPKYWTEPEKFLPERFSKKNKDNIDPYIYTPFGSGPRNCIGMRFALVNMKLALVRVLQNFSFKPCKETQIPLKLRFGGLLLTEKPIVLKAESRDETVSGA

SEQ ID NO: 8 - DNA, 2099 bp, Homo sapiens
  source 1..2099: mol_type = other DNA
aatcactgctgtgcagggcaggaaagctccacacacacagcccagcaaacagcagcacgctgctgaaaaaaagactcagaggagagagataaggaaggaaagtagtgatggatctcatcccaaacttggccgtggaaacctggcttctcctggctgtcagcctgatactcctctatctatatggaacccgtacacatggactttttaagaagcttggaattccagggcccacacctctgccttttttgggaaatgctttgtccttccgtaagggctattggacgtttgacatggaatgttataaaaagtatagaaaagtctggggtatttatgactgtcaacagcctatgctggctatcacagatcccgacatgatcaaaacagtgctagtgaaagaatgttattctgtcttcacaaaccggaggcctttcgggccagtgggatttatgaaaaatgccatctctatagctgaggatgaagaatggaagagaatacgatcattgctgtctccaacattcaccagcggaaaactcaaggagatggtccctatcattgcccagtatggagatgtgttggtgagaaatctgaggcgggaagcagagacaggcaagcctgtcaccttgaaacacgtctttggggcctacagcatggatgtgatcactagcacatcatttggagtgagcatcgactctctcaacaatccacaagacccctttgtggaaaacaccaagaagcttttaagatttaatccattagatccattcgttctctcaataaaagtctttccattccttaccccaattcttgaagcattaaatatcactgtgtttccaagaaaagttataagttttctaacaaaatctgtaaaacagataaaagaaggtcgcctcaaagagacacaaaagcaccgagtggatttccttcagctgatgattgactctcagaattcaaaagactctgagacccacaaagctctgtctgatctggagctcatggcccaatcaattatctttatttttgctggctatgaaaccacgagcagtgttctctccttcattatatatgaactggccactcaccctgatgtccagcagaaagtgcagaaggaaattgatacagttttacccaataaggcaccacccacctatgatactgtgctacagttggagtatcttgacatggtggtgaatgaaacactcagattattcccagttgctatgagacttgagagggtctgcaaaaaagatgttgaaatcaatgggatgtttattcccaaaggggtggtggtgatgattccaagctatgttcttcatcatgacccaaagtactggacagagcctgagaagttcctccctgaaaggttcagtaaaaagaacaaggacaacatagatccttacatatacacaccctttggaagtggacccagaaactgcattggcatgaggtttgctctcgtgaacatgaaacttgctctagtcagagtccttcagaacttctccttcaaaccttgtaaagaaacacagatccccctgaaattacgctttggaggacttcttctaacagaaaaacccattgttctaaaggctgagtcaagggatgagaccgtaagtggagcctgatttccctaaggacttctggtttgctctttaagaaagctgtgccccagaacaccagagacctcaaattactttacaaatagaaccctgaaatgaagacgggcttcatccaatgtgctgcataaataatcagggattctgtacgtgcattgtgctctctcatggtctgtatagagtgttatacttggtaatatagaggagatgaccaaatcagtgctggggaagtagatttggcttctctgcttctcataggactatctccaccacccccagttagcaccattaactcctcctgagctctgataacataattaacatttctcaataatttcaaccacaatcattaataaaaataggaattattttgatggctctaacagtgacatttatatcatgtgttatatctgtagtattctatagtaagctttatattaagcaaatcaataaaaacctctttacaaaagtaaaaaaaaaaaaaaaaaa

SEQ ID NO: 9 - protein, 435 aa, Homo sapiens
MSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 10 - DNA, 5559 bp, Homo sapiens
  source 1..5559: mol_type = other DNA
aagctccaatcggggctttaagtccttgattaggagagtgtgagagctttggtcccaactggctgtgcctataggcttgtcactaggagaacatttgtgttaattgcactgtgctctgtcaaggaaactttgatttatagctggggtgcacaaataatggttgccggtcgcacatggattcggtagaactttgccttcctgaatctttttccctgcactacgaggaagagtagacttgaatgagacctgcctcatcagtcatgggatcatagtgtcacagatggaaaagcaactatcagctgaattgtactgaactacacacttggctaattcatcttattgctctacacatctaaaggaaggctcattctgttcttggagtctagacagcatcaggagttgggctcagtgaacaaaactttaatgtctagagcatttatgagggttttaatgattggaaaatctatcctgagaatgtggtcaccatatgtgacagccttgctttctatcttgtcttcagtttctggggcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagtttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccatactgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaagaggcaataaggcaaatgccagaattaaaaaaaaaaatcatcaaagacagaaaatgcctgaccaaattctaaaacctaatccatataagtttattcatttaggaatgttcgtttaaattaatctgcagtttttaccaagagctaagccaatatatgtgcttttcaaccagtattgtcacagcatgaaagtcaagtcaggttccagactgttaagaggtgtaatctaatgaagaaatcaattagatgccccgaaatctacagtcgctgaataaccaataaacagtaacctccatcaaatgctataccaatggaccagtgttagtagctgctccctgtattatgtgaacagtcttattctatgtacacagatgtaattaaaattgtaatcctaacaaacaaaagaaatgtagttcagcttttcaatgtttcatgtttgctgtgcttttctgaattttatgttgcattcaaagactgttgtcttgttcttgtggtgtttggattcttgtggtgtgtgcttttagacacagggtagaattagagacaatattggatgtacaattcctcaggagactacagtagtatattctattccttaccagtaataaggttcttcctaataataattaagagattgaaactccaaacaagtattcattatgaacagatacacatcaaaatcataataatattttcaaaacaaggaataatttctctaatggtttattatagaataccaatgtatagcttagaaataaaactttgaatatttcaagaatatagataagtctaatttttaaatgctgtatatatggctttcactcaatcatctctcagatgttgttattaactcgctctgtgttgttgcaaaactttttggtgcagattcgtttccaaaactattgctactttgtgtgctttaaacaaaataccttgggttgatgaaacatcaacccagtgctaggaatactgtgtatctatcattagctatatgggactatattgtagattgtggtttctcagtagagaagtgactgtagtgtgattctagataaatcatcattagcaattcattcagatggtcaataacttgaaatttatagctgtgataggagttcagaaattggcacatccctttaaaaataacaacagaaaatacaactcctgggaaaaaaggtgctgattctataagattatttatatatgtaagtgtttaaaaagattattttccagaaagtttgtgcagggtttaagttgctactattcaactacactatatataaataaaatatatacaatatatacattgttttcactgtatcacattaaagtacttgggcttcagaagtaagagccaaccaactgaaaacctgagatggagatatgttcaaagaatgagatacaattttttagttttcagtttaagtaactctcagcattacaaaagagtaagtatctcacaaataggaaataaaactaaaacgtggatttaaaaagaactgcacgggctttagggtaaatgctcatcttaaacctcactagagggaagtcttctcaagtttcaagcaagaccatttacttaatgtgaagttttggaaagttataaaggtgtatgttttagccatatgattttaattttaattttgcttcttttaggttcgttcttatttaaagcaatatgattgtgtgactccttgtagttacacttgtgtttcaatcagatcagattgttgtatttattccactattttgcatttaaatgataacataaaagatataaaaaatttaaaactgctatttttcttatagaagagaaaatgggtgttggtgattgtattttaattatttaagcgtctctgtttacctgcctaggaaaacattttatggcagtcttatgtgcaaagatcgtaaaaggacaaaaaatttaaactgcttataataatccaggagttgcattatagccagtagtaaaaataataataataataataaaaccatgtctatagctgtagatgggcttcacatctgtaaagcaatcaattgtatatttttgtgatgtgtaccatactgtgtgctccagcaaatgtccatttgtgtaaatgtatttattttatattgtatatattgttaaatgcaaaaaggagatatgattctgtaactccaatcagttcagatgtgtaactcaaattattatgcctttcaggatgatggtagagcaatattaaacaagcttccacttttgactgctaaaaaaaaaaaaaaaaa

SEQ ID NO: 11 - protein, 463 aa, Homo sapiens
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 12 - DNA, 2428 bp, Homo sapiens
  source 1..2428: mol_type = other DNA
cccgcccacttccaactaccgcctccggcctgcccagggagagagagggagtggagcccagggagagggagcgcgagagagggagggaggaggggacggtgctttggctgacttttttttaaaagagggtgggggtggggggtgattgctggtcgtttgttgtggctgttaaattttaaactgccatgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggttttatcccttgaatcttttcttccagatttttcttttctttacttggcttacaaaatatacaggcttggaaattatttcaagaaggagggagggataccctgtctggttgcaggttgtattttattttggcccagggagtgttgctgttttcccaacattttattaataaaattttcagacataaaaaa

SEQ ID NO: 13 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 14 - DNA, 3785 bp, Homo sapiens
  source 1..3785: mol_type = other DNA
agttccgacccacagcctggcacccttcggcgagcgctgtttgtttagggctcggtgagtccaatcaggagcccaggctgcagttttccggcagagcagtaagaggcgcctcctctctcctttttattcaccagcagcgcggcgcagaccccggactcgcgctcgcccgctggcgccctcggcttctctccgcgcctgggagcaccctccgccgcggccgttctccatgcgcagcgcccgcccgaggagctagacgtcagcttggagcggcgccggaccgtggatggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcctgagcccacgccgccaggaggcagggagggctccgccgcgggcctcactccactcgtgtctgcttttgtgcagcggtccagacagtggcgactgcgctgacagaacgtgattctcgtgcctttattttgaaagagatgtttttcccaagaggcttgctgaaagagtgagagaagatggaagggaagggccagtgcaactgggcgcttgggccactccagccagcccgcctccggggcggaccctgctccacttccagaagccaggactaggacctgggccttgcctgctatggaatattgagagagattttttaaaaaagattttgcattttgtccaaaatcatgtgcttcttctgatcaattttggttgttccagaatttcttcataccttttccacatccagatttcatgtgcgttcatggagaagatcacttgaggccatttggtacacatctctggaggctgagtcggttcatgaggtctcttatcaaaaatattactcagtttgcaagactgcattgtaactttaacatacactgtgactgacgtttctcaaagttcatattgtgtggctgatctgaagtcagtcggaatttgtaaacagggtagcaaacaagatatttttcttccatgtatacaataatttttttaaaaagtgcaatttgcgttgcagcaatcagtgttaaatcatttgcataagatttaacagcattttttataatgaatgtaaacattttaacttaatggtacttaaaataatttaaaagaaaaatgttaacttagacattcttatgcttcttttacaactacatcccattttatatttccaattgttaaagaaaaatatttcaagaacaaatcttctctcaggaaaattgcctttctctatttgttaagaatttttatacaagaacaccaatataccccctttattttactgtggaatatgtgctggaaaaattgcaacaacactttactacctaacggatagcatttgtaaatactctaggtatctgtaaacactctgatgaagtctgtatagtgtgactaacccacaggcaggttggtttacattaatttttttttttgaatgggatgtcctatggaaacctatttcaccagagttttaaaaataaaaagggtattgttttgtcttctgtacagtgagttccttcccttttcaaagctttctttttatgctgtatgtgactatagatattcatataaaacaagtgcacgtgaagtttgcaaaatgctttaaggccttcctttcaaagcatagtccttttggagccgttttgtaccttttataccttggcttatttgaagttgacacatggggttagttactactctccatgtgcattggggacagtttttataagtgggaaggactcagtattattatatttgagatgataagcattttgtttgggaacaatgcttaaaaatattccagaaagttcagattttttttctttgtgaatgaaatatattctggcccacgaacagggcgatttcctttcagttttttccttttgcaacgtgccttgaagtctcaaagctcacctgaggttgcagacgttacccccaacagaagataggtagaaatgattccagtggcctctttgtattttcttcattgttgagtagatttcaggaaatcaggaggtgtttcacaatacagaatgatggcctttaactgtgaaaaaaaaa

SEQ ID NO: 15 - protein, 394 aa, Homo sapiens
MKRQNVRTLALIVCTFTYLLVGAAVFDALESEPELIERQRLELRQQELRARYNLSQGGYEELERVVLRLKPHKAGVQWRFAGSFYFAITVITTIGYGHAAPSTDGGKVFCMFYALLGIPLTLVMFQSLGERINTLVRYLLHRAKKGLGMRRADVSMANMVLIGFFSCISTLCIGAAAFSHYEHWTFFQAYYYCFITLTTIGFGDYVALQKDQALQTQPQYVAFSFVYILTGLTVIGAFLNLVVLRFMTMNAEDEKRDAEHRALLTRNGQAGGGGGGGSAHTTDTASSTAAAGGGGFRNVYAEVLHFQSMCSCLWYKSREKLQYSIPMIIPRDLSTSDTCVEQSHSSPGGGGRYSDTPSRRCLCSGAPRSAISSVSTGLHSLSTFRGLMKRRSSV

SEQ ID NO: 16 - DNA, 3978 bp, Homo sapiens
  source 1..3978: mol_type = other DNA
ggcggcggcggcggcggcggccccgggcgctgagcgggtgcccggcgcggagagcggcgagcgcagccatgccccaggccgcctccggggcagcagcagcggcggccggggccgaggcgcgggccgggggcgccggggggccggcggcggcccgggcgggacgatgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagcctgtccaccttccgcggcctcatgaagcgcaggagctccgtgtgactgccccgaggggcctggagcacctgggggcgcgggcgggggacccctgctgggaggccaggagactgcccctgctgccttctgcccagtgggaccccgcacaacatccctcaccactctcccccagcacccccatctccgactgtgcctgcttgcaccagccggcaggaggccgggctctgaggacccctggggcccccatcggagccctgcaaattccgagaaatgtgaaacttggtggggtcagggaggaaaggcagaagctgggagcctcccttccctttgaaaatctaagaagctcccagtcctcagagaccctgctggtacccagacccccaccttcggaggggacttcatgttccgtgtacgtttgcatctctatttatacctctgtcctgctaggtctcccaccttcccttggttccaaaagccagggtgtctatgtccaagtcacccctactcagccccactccccttcctcatccccagctgtgtctcccaacctcccttcgtgttgttttgcatggctttgcagttatggagaaagtggaaacccagcagtccctaaagctggtccccagaaagcaggacagaaagaaggagggacaggcaggcagcaggaggggcgagctgggaggcaggaggcagcggcctgtcagtctgcagaatggtcgcactggaggttcaagctaactggcctccagccacattctcatagcaggtaggacttcagccttccagacactgcccttagaatctggaacagaagacttcagactcaccataattgctgataattacccactcttaaatttgtcgagtgatttttagcctctgaaaactctatgctggccactgattcctttgagtctcacaaaaccctacttaggtcatcagggcaggagttctcactcccattttacagatgagaatactgaggcctggacaggtgaagtgaccagagagcaaaaggcaaaggggtgggggctgggtgcagtggctcacacctgtattcccaacacttttggaggctgaggttggaggattgcttgagcccaggaatttgagaccagcctaggtgacatagtgagaccccatctctacaaaaaataaaaaattaaccaggtgtggtggcacgtgcctgggagtcccagcgacttgggaggctgaggtgggaggattgtttgagcctgggaggtcgaggctgtagtgagccctgattgcaccactgtactccagcctgggtgacagggcaagaccctgtctcaaaaaaaaaaaaaaaaatggcaaagggagacaagagcccagcctgcttgttgctagccaaagtgttctttccttccagcttggcctgctcttaaaagcaaagctcctgcagtgtacatcctggcattgtgtggctacctgggttttaaaccagaatcagaagtcccggatcagagggcactgctgaggttcagcctcttctcttcttggccaggaggcagcagctctgaatgggcccctgaggctgcacaggggcctttgtcactggggcgcatgcttacaaacagtgcagttcttgggaccgaggtaagcagggctgggtctcatggcagaaaggccaggatctggggctctaggaatttgggaattgggcagagtggccaagaaagctggcaggcatatcctatgggacatcacacctggcaccattgtcattgttggtgcctgtgtcccaagtagctagtgataagctgaggctgcagcaagaaacacccttcccaggtgggggagtttggaccagaggtgccctctgcccaccacacctgcaacccagaagcccagatggaacgcagctgacgaaggtgatgcttgaggctcacttttggggccccacagctggagccggtataatgactgggacaacatcaaggggtggatgaggggcctctcctcccgcaacactgccttcccatgctgttcccctgccagctccttaacactgccgaccaaggccagccctggcattcagggaaattggagggcagcacccgtagggtggccagcctcaggccccaccccagctgtgtcctctagtctctggggacccctggggggaagaagtctaccctgcttgtgagtcccgtctcagtgtggaggaactggctgcacgtgggacctgaaggtgccctctgtgtttatgttgggggtgggggggcagtgctggctgcctctgtcctgtgtgtgaccctgccctcgaagggtcctgtcctgtcagtcccgagggagccacaaccaaagctgcggagagaaggtggggaagggtgcagaatggccgtggggcacagcgtggcagactgttcagtctctgctgggtctttcctagggacctggaaggccagtgttgcttccccctcactccctttcactgcaggcagcctctctgcttccccaatgccttatgcctgggcacactgccacagaatatgcaatatgtgtgggtgaccatgccctcacgaccacacccccaccccgggcagcccccggactccaaaggtcgtggctgccacagcctccctcagctcttcctgcctatctgtcttcacactgagaatggcgcccaataaatgctatccacggagaccagg

SEQ ID NO: 17 - protein, 676 aa, Homo sapiens [sequence omitted]

SEQ ID NO: 18 - DNA, 3262 bp, Homo sapiens
  source 1..3262: mol_type = other DNA
gcggcggggctggcagcagtggctgcccgcactgcgcccgggcgctcgccttcgctgcagctcccggtgccgccgctcgggccggccccccggcaggccctcctcgttatggccgcggcctcctccccgcccagggccgagaggaagcgctggggttggggccgcctgccaggcgcccggcggggcagcgcgggcctggccaagaagtgccccttctcgctggagctggcggagggcggcccggcgggcggcgcgctctacgcgcccatcgcgcccggcgccccaggtcccgcgccccctgcgtccccggccgcgcccgccgcgcccccagttgcctccgaccttggcccgcggccgccggtgagcctagacccgcgcgtctccatctacagcacgcgccgcccggtgttggcgcgcacccacgtccagggccgcgtctacaacttcctcgagcgtcccaccggctggaaatgcttcgtttaccacttcgccgtcttcctcatcgtcctggtctgcctcatcttcagcgtgctgtccaccatcgagcagtatgccgccctggccacggggactctcttctggatggagatcgtgctggtggtgttcttcgggacggagtacgtggtccgcctctggtccgccggctgccgcagcaagtacgtgggcctctgggggcggctgcgctttgcccggaagcccatttccatcatcgacctcatcgtggtcgtggcctccatggtggtcctctgcgtgggctccaaggggcaggtgtttgccacgtcggccatcaggggcatccgcttcctgcagatcctgaggatgctacacgtcgaccgccagggaggcacctggaggctcctgggctccgtggtcttcatccaccgccaggagctgataaccaccctgtacatcggcttcctgggcctcatcttctcctcgtactttgtgtacctggctgagaaggacgcggtgaacgagtcaggccgcgtggagttcggcagctacgcagatgcgctgtggtggggggtggtcacagtcaccaccatcggctatggggacaaggtgccccagacgtgggtcgggaagaccatcgcctcctgcttctctgtctttgccatctccttctttgcgctcccagcggggattcttggctcggggtttgccctgaaggtgcagcagaagcagaggcagaagcacttcaaccggcagatcccggcggcagcctcactcattcagaccgcatggaggtgctatgctgccgagaaccccgactcctccacctggaagatctacatccggaaggccccccggagccacactctgctgtcacccagccccaaacccaagaagtctgtggtggtaaagaaaaaaaagttcaagctggacaaagacaatggggtgactcctggagagaagatgctcacagtcccccatatcacgtgcgaccccccagaagagcggcggctggaccacttctctgtcgacggctatgacagttctgtaaggaagagcccaacactgctggaagtgagcatgccccatttcatgagaaccaacagcttcgccgaggacctggacctggaaggggagactctgctgacacccatcacccacatctcacagctgcgggaacaccatcgggccaccattaaggtcattcgacgcatgcagtactttgtggccaagaagaaattccagcaagcgcggaagccttacgatgtgcgggacgtcattgagcagtactcgcagggccacctcaacctcatggtgcgcatcaaggagctgcagaggaggctggaccagtccattgggaagccctcactgttcatctccgtctcagaaaagagcaaggatcgcggcagcaacacgatcggcgcccgcctgaaccgagtagaagacaaggtgacgcagctggaccagaggctggcactcatcaccgacatgcttcaccagctgctctccttgcacggtggcagcacccccggcagcggcggcccccccagagagggcggggcccacatcacccagccctgcggcagtggcggctccgtcgaccctgagctcttcctgcccagcaacaccctgcccacctacgagcagctgaccgtgcccaggaggggccccgatgaggggtcctgaggaggggatggggctgggggatgggcctgagtgagaggggaggccaagagtggccccacctggccctctctgaaggaggccacctcctaaaaggcccagagagaagagccccactctcagaggccccaataccccatggaccatgctgtctggcacagcctgcacttgggggctcagcaaggccacctcttcctggccggtgtgggggccccgtctcaggtctgagttgttaccccaagcgccctggcccccacatggtgatgttgacatcactggcatggtggttgggacccagtggcagggcacagggcctggcccatgtatggccaggaagtagcacaggctgagtgcaggcccaccctgcttggcccagggggcttcctgaggggagacagagcaacccctggaccccagcctcaaatccaggaccctgccaggcacaggcagggcaggaccagcccacgctgactacagggccgccggcaataaaagcccaggagcccatttggagggcctgggcctggctccctcactctcaggaaatgctgacccatgggcaggagactgtggagactgctcctgagcccccagcttccagcaggagggacagtctcaccatttccccagggcacgtggttgagtggggggaacgcccacttccctgggttagactgccagctcttcctagctggagaggagccctgcctctccgcccctgagcccactgtgcgtggggctcccgcctccaacccctcgcccagtcccagcagccagccaaacacacagaaggggactgccacctccccttgccagctgctgagccgcagagaagtgacggttcctacacaggacaggggttccttctgggcattacatcgcatagaaatcaataatttgtggtgatttggatctgtgttttaatgagtttcacagtgtgattttgattattaattgtgcaagcttttcctaataaacgtggagaatcacaggctgggctgggcactgctctcaccttggttcctggggcatccatggggtctctcacagacaggacccctgcagttcccctggaagcagtgcccaggtggctgtggaataggaacgctaaaaaaaaaaaaaaaaa

SEQ ID NO: 19 - protein, 907 aa, Homo sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 20 - DNA, 4625 bp, Homo sapiens
  source 1..4625: mol_type = other DNA
aaaaaacgagcgtgcaagcagagatgctgctccacaccgctcaggccgcgagcagcagcaaggcgcaccgccactgtcgccgctgcagccagggctgctccgaaggccggcgtggcggcaaccggcacctctgtccccgccgcgcttctcctcgccgcccacgccgtggggtcaggaacgcggcgtctggcgctgcagacgcccgctgagttgcagaagcccacggagcggcgcccggcgcgccacggcccgtagcagtccggtgctgctctccgcccgcgtccggctcgtggccccctacttcgggcaccatggacacctcccggctcggtgtgctcctgtccttgcctgtgctgctgcagctggcgaccgggggcagctctcccaggtctggtgtgttgctgaggggctgccccacacactgtcattgcgagcccgacggcaggatgttgctcagggtggactgctccgacctggggctctcggagctgccttccaacctcagcgtcttcacctcctacctagacctcagtatgaacaacatcagtcagctgctcccgaatcccctgcccagtctccgcttcctggaggagttacgtcttgcgggaaacgctctgacatacattcccaagggagcattcactggcctttacagtcttaaagttcttatgctgcagaataatcagctaagacacgtacccacagaagctctgcagaatttgcgaagccttcaatccctgcgtctggatgctaaccacatcagctatgtgcccccaagctgtttcagtggcctgcattccctgaggcacctgtggctggatgacaatgcgttaacagaaatccccgtccaggcttttagaagtttatcggcattgcaagccatgaccttggccctgaacaaaatacaccacataccagactatgcctttggaaacctctccagcttggtagttctacatctccataacaatagaatccactccctgggaaagaaatgctttgatgggctccacagcctagagactttagatttaaattacaataaccttgatgaattccccactgcaattaggacactctccaaccttaaagaactaggatttcatagcaacaatatcaggtcgatacctgagaaagcatttgtaggcaacccttctcttattacaatacatttctatgacaatcccatccagtttgttgggagatctgcttttcaacatttacctgaactaagaacactgactctgaatggtgcctcacaaataactgaatttcctgatttaactggaactgcaaacctggagagtctgactttaactggagcacagatctcatctcttcctcaaaccgtctgcaatcagttacctaatctccaagtgctagatctgtcttacaacctattagaagatttacccagtttttcagtctgccaaaagcttcagaaaattgacctaagacataatgaaatctacgaaattaaagttgacactttccagcagttgcttagcctccgatcgctgaatttggcttggaacaaaattgctattattcaccccaatgcattttccactttgccatccctaataaagctggacctatcgtccaacctcctgtcgtcttttcctataactgggttacatggtttaactcacttaaaattaacaggaaatcatgccttacagagcttgatatcatctgaaaactttccagaactcaaggttatagaaatgccttatgcttaccagtgctgtgcatttggagtgtgtgagaatgcctataagatttctaatcaatggaataaaggtgacaacagcagtatggacgaccttcataagaaagatgctggaatgtttcaggctcaagatgaacgtgaccttgaagatttcctgcttgactttgaggaagacctgaaagcccttcattcagtgcagtgttcaccttccccaggccccttcaaaccctgtgaacacctgcttgatggctggctgatcagaattggagtgtggaccatagcagttctggcacttacttgtaatgctttggtgacttcaacagttttcagatcccctctgtacatttcccccattaaactgttaattggggtcatcgcagcagtgaacatgctcacgggagtctccagtgccgtgctggctggtgtggatgcgttcacttttggcagctttgcacgacatggtgcctggtgggagaatggggttggttgccatgtcattggttttttgtccatttttgcttcagaatcatctgttttcctgcttactctggcagccctggagcgtgggttctctgtgaaatattctgcaaaatttgaaacgaaagctccattttctagcctgaaagtaatcattttgctctgtgccctgctggccttgaccatggccgcagttcccctgctgggtggcagcaagtatggcgcctcccctctctgcctgcctttgccttttggggagcccagcaccatgggctacatggtcgctctcatcttgctcaattccctttgcttcctcatgatgaccattgcctacaccaagctctactgcaatttggacaagggagacctggagaatatttgggactgctctatggtaaaacacattgccctgttgctcttcaccaactgcatcctaaactgccctgtggctttcttgtccttctcctctttaataaaccttacatttatcagtcctgaagtaattaagtttatccttctggtggtagtcccacttcctgcatgtctcaatccccttctctacatcttgttcaatcctcactttaaggaggatctggtgagcctgagaaagcaaacctacgtctggacaagatcaaaacacccaagcttgatgtcaattaactctgatgatgtcgaaaaacagtcctgtgactcaactcaagccttggtaacctttaccagctccagcatcacttatgacctgcctcccagttccgtgccatcaccagcttatccagtgactgagagctgccatctttcctctgtggcatttgtcccatgtctctaattaatatgtgaaggaaaatgttttcaaaggttgagaacctgaaaatgtgagattgagtatatcagagcagtaattaataagaagagctgaggtgaaactcggtttaaaaaccaaaaaagaatctctcagttagtaagaaaaggctgaaaacctcttgatacttgagagtgaatataagtctaaatgctgctttgtataatttgttcagctaagggatagatcgatcacactatttaagtgagcccagatcaaaaaagcagattgaaattttctttagaaaagattctccatgatttgaattgcattctctttaaactcaccaatgtaatcattttgggaggagggagaacccacttgctttccaaatgggtttatttaaacccacaaactcaagaggttgttgggggaattaggaaaataagggttttcaatgacctacattgctaggtagaggctgtgatccatgggatttcattctaatgaccatgtgaagatgtttgagtcctcctttgcctttcctcagaaagaatccttctaaggcacaaatcccttagatggataatgtaaggtattgttaactcactcatattgagatcatttttagagataccaggttttatgtatcagcactagatggttccaccctcatgggataaaactgcttacaagtattttgaaagaaaaactgaccaaaattcttaaattgttactaaggcaatcatgcacaggtgacgtatgtcttatctgatttgtttttaactccttggtgcccaaagctcagaagggaattccactgccagcaatgaacatacctggaaaagaaagtaagcaatctgggattttttttctgggttagtaaagaatttttgcaataagttttatcagttgattcaaactgatgtgcatcttaatgatcaaatgtgcacattacataaattaagtccactgatacaacttcttacacatgtatctctagtagctctggcaaacccaatatctgacaccactttggactcaagagactcagtaacgtattatcctgtttatttagcttggttttagctgtgttctctctggataacccacttgatgttaggaacattacttctctgcttattccatattaatactgtgttaggtattttaagaagcaagttattaaataagaaaagtcaaagtattaattcttaccttctattatcctatattagcttcaatacatccaaaccaaatggctgttaggtagatttatttttatataagcatgtttattttgatcagatgttttaacttggatttgaaaaaatacatttatgagatgttttataagatgtgtaaatatagaactgtatttattactatagtaaaggttcagtaacattaaggaccatgataatgataataaaccttgtacagtggcatattctttgatttatattgtgtttctctgcccattttctttaaattcattaactgtatatatgtaaatatatagtacttgtaaatagattccaaatttgcttttctattgggtaaaaaataaatttgtaataaaatgtgtgactatgaaacaaaaaaaaaaaaaaaaaa

SEQ ID NO: 21 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 22 - DNA, 2226 bp, Homo sapiens
  source 1..2226: mol_type = other DNA
gtctgccggtcggttgtctggctgcgcgcgccacccgggcctctccagtgccccgcctggctcggcatccacccccagcccgactcacacgtgggttcccgcacgtccgccggccccccccgctgacgtcagcatagctgttccacttaaggcccctcccgcgcccagctcagagtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 23 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 24 - DNA, 2373 bp, Homo sapiens
  source 1..2373: mol_type = other DNA
gcgcggccgggcgcgggccccgggcgatggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccatcaccacgccgcccaccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctgtaggcgccggaccccgagcccgcgccgccgtcgccggggacaagttcgcgcaggcctctcggggcctcggctcggactccgcggtacaggacgtggacaccaggcccggcccggccgtgctggccccggtgccaagtctgcgggcgcggggctggaggccccttcgctcccggtccccgttcgcgcgcgtcggcccgggtcgccgtcctgaggttgagcggagaacggtgatttctaaggaaacttgagccaggtctaacttctttccaagcgtccgcttgtacatacgttgaacgtggttctccgttcccaccttcgccctgccagcctagagggaccgcgctgccgtcccttcccgggtggcccctgcctgcccccgccctccttcgttctcttctcagcctccctttccttgccttttttaacttcccctccccgttttaaaatcggtcttattttcgaagtatttataattattatgcttggtgattagaaaagaaaaccttggaggaagccccttctttccccagccggggtccgccctcagtcgcgagtcacagcatgagtcgctcgccaggaggggcccggcccctgcctgccccctccccgcttgcccccgaccctgctaccggcgttccttggaggtcgaagccagggacgtcacccgtgctgtgtccaggcctgctgtcctactatgctcaaccgggggtggggggaggggggtgagtcctgtgctcagtcgggtgggggctggcccggatcccgagctgctgtctctctatgcaccagaacatatctgtaactcctggggaaatacatcttgttttaaccttcaagagaagtgaaagaaaaaagtaatgcacagtatttctagcagaaaatttttttttttaagaggaggcttgggccagagccttctggcatggggcgggtggagaaagtgtttttattttaatttaaattgtgtttcgttttgtttgtggaatctttctttaatgcttcgtcgctctttggactagccgggagagagggcgaggaggcgggtgctccaggccctgtaggctgggccaggcgcctgggggatctgcccgttttcggaggccctcaggggccatcagtgggattccagccgctccacacccctcccctgagcactcggagtggaaggcgcgccgactcgttgaaagttttgttgtgtagttggttttcgttgagttcttttttcatttgctacgaaactgagaaaaagaaaaaaatacacaaaataaatctgttcagatccaagtca

SEQ ID NO: 25 - protein, 1939 aa, Homo sapiens
MTDAQMADFGAAAQYLRKSEKERLEAQTRPFDIRTECFVPDDKEEFVKAKILSREGGKVIAETENGKTVTVKEDQVLQQNPPKFDKIEDMAMLTFLHEPAVLFNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRGKKDNANANKGTLEDQIIQANPALEAFGNAKTVRNDNSSRFGKFIRIHFGATGKLASADIETYLLEKSRVIFQLKAERNYHIFYQILSNKKPELLDMLLVTNNPYDYAFVSQGEVSVASIDDSEELMATDSAFDVLGFTSEEKAGVYKLTGAIMHYGNMKFKQKQREEQAEPDGTEDADKSAYLMGLNSADLLKGLCHPRVKVGNEYVTKGQSVQQVYYSIGALAKAVYEKMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLYDNHLGKSNNFQKPRNIKGKQEAHFSLIHYAGTVDYNILGWLEKNKDPLNETVVALYQKSSLKLMATLFSSYATADTGDSGKSKGGKKKGSSFQTVSALHRENLNKLMTNLRTTHPHFVRCIIPNERKAPGVMDNPLVMHQLRCNGVLEGIRICRKGFPNRILYGDFRQRYRILNPVAIPEGQFIDSRKGTEKLLSSLDIDHNQYKFGHTKVFFKAGLLGLLEEMRDERLSRIITRMQAQARGQLMRIEFKKIVERRDALLVIQWNIRAFMGVKNWPWMKLYFKIKPLLKSAETEKEMATMKEEFGRIKETLEKSEARRKELEEKMVSLLQEKNDLQLQVQAEQDNLNDAEERCDQLIKNKIQLEAKVKEMNERLEDEEEMNAELTAKKRKLEDECSELKKDIDDLELTLAKVEKEKHATENKVKNLTEEMAGLDEIIAKLTKEKKALQEAHQQALDDLQVEEDKVNSLSKSKVKLEQQVDDLEGSLEQEKKVRMDLERAKRKLEGDLKLTQESIMDLENDKLQLEEKLKKKEFDINQQNSKIEDEQVLALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRVKLEEAQRSLNDFTTQRAKLQTENGELARQLEEKEALISQLTRGKLSYTQQMEDLKRQLEEEGKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNVHELEKVRKQLEVEKLELQSALEEAEASLEHEEGKILRAQLEFNQIKAEIERKLAEKDEEMEQAKRNHQRVVDSLQTSLDAETRSRNEVLRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMESDLTQLQSEVEEAVQECRNAEEKAKKAITDAAMMAEELKKEQDTSAHLERMKKNMEQTIKDLQHRLDEAEQIALKGGKKQLQKLEARVRELEGELEAEQKRNAESVKGMRKSERRIKELTYQTEEDKKNLLRLQDLVDKLQLKVKAYKRQAEEAEEQANTNLSKFRKVQHELDEAEERADIAESQVNKLRAKSRDIGAKQKMHDEE

SEQ ID NO: 26 - DNA, 5941 bp, Homo sapiens
  source 1..5941: mol_type = other DNA
agatagagagactcctgcggcccagattcttcaggattctccgtgaagggataaccaggggaagcaccaagatgaccgatgcccagatggctgactttggggcagcggcccagtacctccgcaagtcagagaaggagcgtctagaggcccagacccggccctttgacattcgcactgagtgcttcgtgcccgatgacaaggaagagtttgtcaaagccaagattttgtcccgggagggaggcaaggtcattgctgaaaccgagaatgggaagacggtgactgtgaaggaggaccaggtgttgcagcagaacccacccaagttcgacaagattgaggacatggccatgctgaccttcctgcacgagcccgcggtgcttttcaacctcaaggagcgctacgcggcctggatgatatatacctactcgggcctcttctgtgtcactgtcaacccctacaagtggctgccggtgtacaatgccgaggtggtggccgcctaccggggcaagaagaggagtgaggccccgccccacatcttctccatctccgacaacgcctatcagtacatgctgacagatcgggagaaccagtccatcctcatcacgggagaatccggggcggggaagactgtgaacaccaagcgtgtcatccagtactttgccagcattgcagccataggtgaccgtggcaagaaggacaatgccaatgcgaacaagggcaccctggaggaccagatcatccaggccaaccccgctctggaggccttcggcaatgccaagactgtccggaacgacaactcctcccgctttgggaaattcattaggatccactttggggccactggaaagctggcttctgcagacatagagacctacctgctggagaagtcccgggtgatcttccagctgaaagctgagagaaactaccacatcttctaccagattctgtccaacaagaagccggagttgctggacatgctgctggtcaccaacaatccctacgactacgccttcgtgtctcagggagaggtgtccgtggcctccattgatgactccgaggagctcatggccaccgatagtgcctttgacgtgctgggcttcacttcagaggagaaagctggcgtctacaagctgacgggagccatcatgcactacgggaacatgaagttcaagcagaagcagcgggaggagcaggcggagccagacggcaccgaagatgctgacaagtcggcctacctcatggggctgaactcagctgacctgctcaaggggctgtgccaccctcgggtgaaagtgggcaacgagtatgtcaccaaggggcagagcgtgcagcaggtgtactactccatcggggctctggccaaggcagtgtatgagaagatgttcaactggatggtgacgcgcatcaacgccaccctggagaccaagcagccacgccagtacttcataggagtcctggacatcgctggcttcgagatcttcgacttcaacagctttgagcagctctgcatcaacttcaccaacgagaagctgcagcagttcttcaaccaccacatgttcgtgctggagcaggaggagtacaagaaggagggcattgagtggacattcattgactttggcatggacctgcaggcctgcattgacctcatcgagaagcccatgggcatcatgtccatcctggaggaggagtgcatgttccccaaggccactgacatgaccttcaaggccaagctgtacgacaaccacctgggcaagtccaacaatttccagaagccacgcaacatcaaggggaagcaggaagcccacttctccctgatccactacgccggcactgtggactacaacatcctgggctggctggaaaaaaacaaggatcctctcaacgagactgttgtggccctgtaccagaagtcctccctcaagctcatggccactctcttctcctcctacgcaactgccgatactggggacagtggtaaaagcaaaggaggcaagaaaaagggctcatccttccagacggtgtcggctctccaccgggaaaatctcaacaagctaatgaccaacctgaggaccacccatcctcactttgtgcgttgcatcatccccaatgagcggaaggctccaggggtgatggacaaccccctggtcatgcaccagctgcgctgcaatggcgtgctggagggcatccgcatctgcaggaagggcttccccaaccgcatcctctacggggacttccggcagaggtatcgcatcctgaacccagtggccatccctgagggacagttcattgatagcaggaaggggacagagaagctgctcagctctctggacattgatcacaaccagtacaagtttggccacaccaaggtgttcttcaaggcagggctgcttgggctgctggaggagatgcgggatgagaggctgagccgcatcatcacgcgcatgcaggcccaagcccggggccagctcatgcgcattgagttcaagaagatagtggaacgcagggatgccctgctggtaatccagtggaacattcgggccttcatgggggtcaagaattggccctggatgaagctctacttcaagatcaagccgctgctgaagagcgcagagacggagaaggagatggccaccatgaaggaagagttcgggcgcatcaaagagacgctggagaagtccgaggctcgccgcaaggagctggaggagaagatggtgtccctgctgcaggagaagaatgacctgcagctccaagtgcaggcggaacaagacaacctcaatgatgctgaggagcgctgcgaccagctgatcaaaaacaagattcagctggaggccaaagtaaaggagatgaatgagaggctggaggatgaggaggagatgaacgcggagctcactgccaagaagcgcaagctggaagacgagtgctcagagctcaagaaggacattgatgacctggagctgacactggccaaggtggagaaggagaagcatgcaacagagaacaaggtgaagaacctaacagaggagatggctgggctggatgaaatcatcgctaagctgaccaaggagaagaaagctctacaagaggcccatcagcaggccctggatgaccttcaggttgaggaagacaaggtcaacagcctgtccaagtctaaggtcaagctggagcagcaggtggatgatctggagggatccctagagcaagagaagaaggtgcgcatggacctggagcgagcaaagcggaaactggagggcgacctgaagctgacccaggagagcatcatggacctggaaaatgataaactgcagctggaagaaaagcttaagaagaaggagtttgacattaatcagcagaacagtaagattgaggatgagcaggtgctggcccttcaactacagaagaaactgaaggaaaaccaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggcaaacctggagaaagtgtctcggacgctggaggaccaggccaatgagtaccgcgtgaagctagaagaggcccaacgctccctcaatgatttcaccacccagcgagccaagctgcagaccgagaatggagagttggcccggcagctagaggaaaaggaggcgctaatctcgcagctgacccgggggaagctctcttatacccagcaaatggaggacctcaaaaggcagctggaggaggagggcaaggcgaagaacgccctggcccatgcactgcagtcggcccggcatgactgcgacctgctgcgggagcagtacgaggaggagacagaggccaaggccgagctgcagcgcgtcctgtccaaggccaactcggaggtggcccagtggaggaccaagtatgagacggacgccattcagcggactgaggagctcgaagaggccaaaaagaagctggcccagcggctgcaggatgccgaggaggccgtggaggctgttaatgccaagtgctcctcactggagaagaccaagcaccggctacagaatgagatagaggacttgatggtggacgtagagcgctccaatgctgctgctgcagccctggacaagaagcagagaaactttgacaagatcctggccgagtggaagcagaagtatgaggagtcgcagtctgagctggagtcctcacagaaggaggctcgctccctcagcacagagctcttcaagctcaagaacgcctacgaggagtccctggagcacctagagaccttcaagcgggagaacaagaaccttcaggaggaaatctcggaccttactgagcagctaggagaaggaggaaagaatgtgcatgagctggagaaggtccgcaaacagctggaggtggagaagctggagctgcagtcagccctggaggaggcagaggcctccctggagcacgaggagggcaagatcctccgggcccagctagagttcaaccagatcaaggcagagatcgagcggaagctggcagagaaggacgaggagatggaacaggccaagcgcaaccaccagcgggtggtggactcgctgcagacctccctggatgcagagacacgcagccgcaacgaggtcctgagggtgaagaagaagatggaaggagacctcaatgagatggagatccagctcagccacgccaaccgcatggctgccgaggcccagaagcaagtcaagagcctccagagcttgctgaaggacacccagatccagctggacgatgcggtccgtgccaacgacgacctgaaggagaacatcgccatcgtggagcggcgcaacaacctgctgcaggctgagctggaggagctgcgtgccgtggtggagcagacagagcggtcccggaagctggcggagcaggagctgattgagaccagcgagcgggtgcagctgctgcattcccagaacaccagcctcatcaaccagaagaagaagatggagtcggatctgacccagctccagtcggaagtggaggaggcagtgcaggagtgcagaaacgccgaggagaaggccaagaaggccatcacggatgccgccatgatggcagaggagctgaagaaggagcaggacaccagcgcccacctggagcgcatgaagaagaacatggagcagaccattaaggacctgcagcaccggctggacgaggccgagcagatcgccctcaagggaggcaagaagcagctgcagaagctggaagcgcgggtgcgggagctggagggtgagctggaggccgagcagaagcgcaacgcagagtcggtgaagggcatgaggaagagcgagcggcgcatcaaggagctcacctaccagacagaggaagacaaaaagaacctgctgcggctacaggacctggtggacaagctgcaactgaaggtcaaggcctacaagcgccaggccgaggaggcggaggagcaagccaacaccaacctgtccaagttccgcaaggtgcagcatgagctggatgaggcagaggagcgggcggacatcgctgagtcccaggtcaacaagcttcgagccaagagccgtgacattggtgccaagcaaaaaatgcacgatgaggagtgacactgcctcgggaacctcactcttgccaacctgtaataaatatgagtgcc

SEQ ID NO: 27 - protein, 175 aa, Homo sapiens
MASRKAGTRGKVAATKQAQRGSSNVFSMFEQAQIQEFKEAFSCIDQNRDGIICKADLRETYSQLGKVSVPEEELDAMLQEGKGPINFTVFLTLFGEKLNGTDPEEAILSAFRMFDPSGKGVVNKDEFKQLLLTQADKFSPAEVEQMFALTPMDLAGNIDYKSLCYIITHGDEKEE

SEQ ID NO: 28 - DNA, 619 bp, Homo sapiens
  source 1..619: mol_type = other DNA
tctgcagagagaatggccagcaggaaggcggggacccggggcaaggtggcagccaccaagcaggcccaacgtggttcttccaacgtcttttccatgtttgaacaagcccagatacaggagttcaaagaagccttcagctgtatcgaccagaatcgtgatggcatcatctgcaaggcagacctgagggagacctactcccagctggggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgagttcaagcagcttctcctgacccaggcagacaagttctctccagctgaggtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattggaaaaaaaaaaaaaaaaaaaaaaaa

SEQ ID NO: 29 - protein, 5179 aa, Homo sapiens
MGLPLARLAAVCLALSLAGGSELQTEGRTRNHGHNVCSTWGNFHYKTFDGDVFRFPGPCDYNFASDCRGSYKEFAVHLKRGPGQAEAPAGVESILLTIKDDTIYLTRHLAVLNGAVVSTPHYSPGLLIEKSDAYTKVYSRAGLTLMWNREDALMLELDTKFRNHTCGLCGDYNGLQSYSEFLSDGVLFSPLEFGNMQKINQPDVVCEDPEEEVAPASCSEHRAECERLLTAEAFADCQDLVPLEPYLRACQQDRCRCPGGDTCVCSTVAEFSRQCSHAGGRPGNWRTATLCPKTCPGNLVYLESGSPCMDTCSHLEVSSLCEEHRMDGCFCPEGTVYDDIGDSGCVPVSQCHCRLHGHLYTPGQEITNDCEQCVCNAGRWVCKDLPCPGTCALEGGSHITTFDGKTYTFHGDCYYVLAKGDHNDSYALLGELAPCGSTDKQTCLKTVVLLADKKKNVVVFKSDGSVLLNELQVNLPHVTASFSVFRPSSYHIMVSMAIGVRLQVQLAPVMQLFVTLDQASQGQVQGLCGNFNGLEGDDFKTASGLVEATGAGFANTWKAQSTCHDKLDWLDDPCSLNIESANYAEHWCSLLKKTETPFGRCHSAVDPAEYYKRCKYDTCNCQNNEDCLCAALSSYARACTAKGVMLWGWREHVCNKDVGSCPNSQVFLYNLTTCQQTCRSLSEADSHCLEGFAPVDGCGCPDHTFLDEKGRCVPLAKCSCYHRGLYLEAGDVVVRQEERCVCRDGRLHCRQIRLIGQSCTAPKIHMDCSNLTALATSKPRALSCQTLAAGYYHTECVSGCVCPDGLMDDGRGGCVVEKECPCVHNNDLYSSGAKIKVDCNTCTCKRGRWVCTQAVCHGTCSIYGSGHYITFDGKYYDFDGHCSYVAVQDYCGQNSSLGSFSIITENVPCGTTGVTCSKAIKIFMGRTELKLEDKHRVVIQRDEGHHVAYTTREVGQYLVVESSTGIIVIWDKRTTVFIKLAPSYKGTVCGLCGNFDHRSNNDFTTRDHMVVSSELDFGNSWKEAPTCPDVSTNPEPCSLNPHRRSWAEKQCSILKSSVFSICHSKVDPKPFYEACVHDSCSCDTGGDCECFCSAVASYAQECTKEGACVFWRTPDLCPIFCDYYNPPHECEWHYEPCGNRSFETCRTINGIHSNISVSYLEGCYPRCPKDRPIYEEDLKKCVTADKCGCYVEDTHYPPGASVPTEETCKSCVCTNSSQVVCRPEEGKILNQTQDGAFCYWEICGPNGTVEKHFNICSITTRPSTLTTFTTITLPTTPTTFTTTTTTTTPTSSTVLSTTPKLCCLWSDWINEDHPSSGSDDGDRETFDGVCGAPEDIECRSVKDPHLSLEQLGQKVQCDVSVGFICKNEDQFGNGPFGLCYDYKIRVNCCWPMDKCITTPSPPTTTPSPPPTSTTTLPPTTTPSPPTTTTTTPPPTTTPSPPITTTTTPPPTTTPSPPISTTTTPPPTTTPSPPTTTPSPPTTTPSPPTTTTTTPPPTTTPSPPTTTPITPPASTTTLPPTTTPSPPTTTTTTPPPTTTPSPPTTTPITPPTSTTTLPPTTTPSPPPTTTTTPPPTTTPSPPTTTTPSPPTITTTTPPPTTTPSPPTTTTTTPPPTTTPSPPTTTPITPPTSTTTLPPTTTPSPPPTTTTTPPPTTTPSPPTTTTPSPPITTTTTPPPTTTPSSPITTTPSPPTTTMTTPSPTTTPSSPITTTTTPSSTTTPSPPPTTMTTPSPTTTPSPPTTTMTTLPPTTTSSPLTTTPLPPSITPPTFSPFSTTTPTTPCVPLCNWTGWLDSGKPNFHKPGGDTELIGDVCGPGWAANISCRATMYPDVPIGQLGQTVVCDVSVGLICKNEDQKPGGVIPMAFCLNYEINVQCCECVTQPTTMTTTTTENPTPPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTGPPTHTSTAPIAELTTSNPPPESSTPQTSRSTSSPLTESTTLLSTLPPAIEMTSTAPPSTPTAPTTTSGGHTLSPPPSTTTSPPGTPTRGTTTGSSSAPTPSTVQTTTTSAWTPTPTPLSTPSIIRTTGLRPYPSSVLICCVLNDTYYAPGEEVYNGTYGDTCYFVNCSLSCTLEFYNWSCPSTPSPTPTPSKSTPTPSKPSSTPSKPTPGTKPPECPDFDPPRQENETWWLCDCFMATCKYNNTVEIVKVECEPPPMPTCSNGLQPVRVEDPDGCCWHWECDCYCTGWGDPHYVTFDGLYYSYQGNCTYVLVEEISPSVDNFGVYIDNYHCDPNDKVSCPRTLIVRHETQEVLIKTVHMMPMQVQVQVNRQAVALPYKKYGLEVYQSGINYVVDIPELGVLVSYNGLSFSVRLPYHRFGNNTKGQCGTCTNTTSDDCILPSGEIVSNCEAAADQWLVNDPSKPHCPHSSSTTKRPAVTVPGGGKTTPHKDCTPSPLCQLIKDSLFAQCHALVPPQHYYDACVFDSCFMPGSSLECASLQAYAALCAQQNICLDWRNHTHGACLVECPSHREYQACGPAEEPTCKSSSSQQNNTVLVEGCFCPEGTMNYAPGFDVCVKTCGCVGPDNVPREFGEHFEFDCKNCVCLEGGSGIICQPKRCSQKPVTHCVEDGTYLATEVNPADTCCNITVCKCNTSLCKEKPSVCPLGFEVKSKMVPGRCCPFYWCESKGVCVHGNAEYQPGSPVYSSKCQDCVCTDKVDNNTLLNVIACTHVPCNTSCSPGFELMEAPGECCKKCEQTHCIIKRPDNQHVILKPGDFKSDPKNNCTFFSCVKIHNQLISSVSNITCPNFDASICIPGSITFMPNGCCKTCTPRNETRVPCSTVPVTTEVSYAGCTKTVLMNHCSGSCGTFVMYSAKAQALDHSCSCCKEEKTSQREVVLSCPNGGSLTHTYTHIESCQCQDTVCGLPTGTSRRARRSPRHLGSG

SEQ ID NO: 30 - DNA, 15720 bp, Homo sapiens
  source 1..15720: mol_type = other DNA
caacccacaccgcccctgccagccaccatggggctgccactagcccgcctggcggctgtgtgcctggccctgtctttggcagggggctcggagctccagacagagggcagaacccgaaaccacggccacaacgtctgcagcacctggggcaacttccactacaagaccttcgacggggacgtcttccgcttccccggcccctgcgactacaacttcgcctccgactgccgaggctcctacaaggaatttgctgtgcacctgaagcggggtccgggccaggctgaggcccccgccggggtggagtccatcctgctgaccatcaaggatgacaccatctacctcacccgccacctggctgtgcttaacggggccgtggtcagcaccccgcactacagccccgggctgctcattgagaagagcgatgcctacaccaaagtctactcccgcgccggcctcaccctcatgtggaaccgggaggatgcactcatgctggagctggacactaagttccggaaccacacctgtggcctctgcggggactacaacggcctgcagagctattcagaattcctctctgacggcgtgctcttcagtcccctggagtttgggaacatgcagaagatcaaccagcccgatgtggtgtgtgaggatcccgaggaggaggtggcccccgcatcctgctccgagcaccgcgccgagtgtgagaggctgctgaccgccgaggccttcgcggactgtcaggacctggtgccgctggagccgtatctgcgcgcctgccagcaggaccgctgccggtgcccgggcggtgacacctgcgtctgcagcaccgtggccgagttctcccgccagtgctcccacgccggcggccggcccgggaactggaggaccgccacgctctgccccaagacctgccccgggaacctggtgtacctggagagcggctcgccctgcatggacacctgctcacacctggaggtgagcagcctgtgcgaggagcaccgcatggacggctgtttctgcccagaaggcaccgtatatgacgacatcggggacagtggctgcgttcctgtgagccagtgccactgcaggctgcacggacacctgtacacaccgggccaggagatcaccaatgactgcgagcagtgtgtctgtaacgctggccgctgggtgtgcaaagacctgccctgccccggcacctgtgccctggaaggcggctcccacatcaccaccttcgatgggaagacgtacaccttccacggggactgctactatgtcctggccaagggtgaccacaacgattcctacgctctcctgggcgagctggccccctgtggctccacagacaagcagacctgcctgaagacggtggtgctgctggctgacaagaagaagaatgtggtggtcttcaagtccgatggcagtgtactgctcaacgagctgcaggtgaacctgccccacgtgaccgcgagcttctctgtcttccgcccgtcttcctaccacatcatggtgagcatggccattggcgtccggctgcaggtgcagctggccccagtcatgcaactctttgtgacactggaccaggcctcccaggggcaggtgcagggcctctgcgggaacttcaacggcctggaaggtgacgacttcaagacggccagcgggctggtggaggccacgggggccggctttgccaacacctggaaggcacagtcaacctgccatgacaagctggactggttggacgatccctgctccctgaacatcgagagcgccaactacgccgagcactggtgctccctcctgaagaagacagagaccccctttggcaggtgccactcggctgtggaccctgctgagtattacaagaggtgcaaatatgacacgtgtaactgtcagaacaatgaggactgcctgtgcgccgccctgtcctcctacgcgcgcgcctgcaccgccaagggcgtcatgctgtggggctggcgggagcatgtctgcaacaaggatgtgggctcctgccccaactcgcaggtcttcctgtacaacctgaccacctgccagcagacctgccgctccctctccgaggccgacagccactgtctcgagggctttgcgcctgtggacggctgcggctgccctgaccacaccttcctggacgagaagggccgctgcgtacccctggccaagtgctcctgttaccaccgcggtctctacctggaggcgggggacgtggtcgtcaggcaggaagaacgatgtgtgtgccgggatgggcggctgcactgtaggcagatccggctgatcggccagagctgcacggccccaaagatccacatggactgcagcaacctgactgcactggccacctcgaagccccgagccctcagctgccagacgctggccgccggctattaccacacagagtgtgtcagtggctgtgtgtgccccgacgggctgatggatgacggccggggtggctgcgtggtggagaaggaatgcccttgcgtccataacaacgacctgtattcttccggcgccaagatcaaggtggactgcaatacctgcacctgcaagagaggacgctgggtgtgcacccaggctgtgtgccatggcacctgctccatttacgggagtggccactacatcacctttgacgggaagtactacgactttgacggacactgctcctacgtggctgttcaggactactgcggccagaactcctcactgggctcattcagcatcatcaccgagaacgtcccctgtggcactacgggcgtcacctgctccaaggccatcaagatcttcatggggaggacggagctgaagttggaagacaagcaccgtgtggtgatccagcgtgatgagggtcaccacgtggcctacaccacgcgggaggtgggccagtacctggtggtggagtccagcacgggcatcatcgtcatctgggacaagaggaccaccgtgttcatcaagctggctccctcctacaagggcaccgtgtgtggcctgtgtgggaactttgaccaccgctccaacaacgacttcaccacgcgggaccacatggtggtgagcagcgagctggacttcgggaacagctggaaggaggcccccacctgcccagatgtgagcaccaaccccgagccctgcagcctgaacccgcaccgccgctcctgggccgagaagcagtgcagcatcctcaaaagcagcgtgttcagcatctgccacagcaaggtggaccccaagcccttctacgaggcctgtgtgcacgactcgtgctcctgtgacacgggtggggactgtgagtgcttctgctctgccgtggcctcctacgcccaggagtgtaccaaagagggggcctgcgtgttctggaggacgccggacctgtgccccatattctgcgactactacaaccctccgcatgagtgtgagtggcactatgagccatgtgggaaccggagcttcgagacctgcaggaccatcaatggcatccactccaacatctccgtgtcctacctggagggctgctacccccggtgccccaaggacaggcccatctatgaggaggatctgaagaagtgtgtcactgcagacaagtgtggctgctatgtcgaggacacccactacccacctggagcatcggttcccaccgaggagacctgcaagtcctgcgtgtgtaccaactcctcccaagtcgtctgcaggccggaggaaggaaagattcttaaccagacccaggatggcgccttctgctactgggagatctgtggccccaacgggacggtggagaagcacttcaacatctgttccattacgacacgcccgtccaccctgaccaccttcaccaccatcaccctccccaccacccccaccaccttcaccactaccaccaccaccaccaccccgacctccagcacagttttatcaacaactccgaagctgtgctgcctctggtctgactggatcaatgaggaccaccccagcagtggcagcgacgacggtgaccgagaaacatttgatggggtctgcggggcccctgaggacatcgagtgcaggtcggtcaaggatccccacctcagcttggagcagctaggccagaaggtgcagtgtgatgtctctgttgggttcatttgcaagaatgaagaccagtttggaaatggaccatttggactgtgttacgactacaagatacgtgtcaattgttgctggcccatggataagtgtatcaccactcccagccctccaactaccactcccagccctccaccaaccagcacgaccacccttccaccaaccaccacccccagccctccaaccaccaccacaaccacccctccaccaaccaccacccccagccctccaataaccaccacgaccacccctccaccaaccaccactcccagccctccaataagcaccacaaccacccctccaccaaccaccactcccagccctccaaccaccactcccagccctccaaccaccactcccagccctccaacaaccaccacaaccacccctccaccaaccaccactcccagccctccaacgactacgcccatcactccaccagccagcactaccacccttccaccaaccaccactcccagccctccaacaaccaccacaaccacccctccaccaaccaccactcccagtcctccaacgactacgcccatcactccaccaaccagcactactacccttccaccaaccaccactcccagccctccaccaaccaccacaaccacccctccaccaaccaccactcccagccctccaacaaccaccactcccagtcctccaacaatcaccacaaccacccctccaccaaccaccactcccagccctccaacaacgaccacaaccacccctccaccaaccaccactcccagccctccaacgactacacccatcactccaccaaccagcactaccacccttccaccaaccaccactcccagccctccaccaaccaccacaaccacccctccaccaaccaccactcccagccctccaacaaccaccactcccagccctccaataaccaccacaaccacccctccaccaaccaccactcccagctctccaataaccaccactcccagccctccaacaaccaccatgaccaccccttcaccaaccaccacccccagctctccaataaccaccacaaccaccccttcctcaactaccactcccagccctccaccaaccaccatgaccaccccttcaccaaccaccactcccagccctccaacaaccaccatgaccacccttccaccaaccaccacttccagccctctaacaactactcctctacctccatcaataactcctcctacattttcaccattctcaacgacaacccctactaccccatgcgtgcctctctgcaattggactggctggctggattctggaaaacccaactttcacaaaccaggtggagacacagaattgattggagacgtctgtggaccaggctgggcagctaacatctcttgcagagccaccatgtatcctgatgttcccattggacagcttggacaaacagtggtgtgtgatgtctctgtggggctgatatgcaaaaatgaagaccaaaagccaggtggggtcatccctatggccttctgcctcaactacgagatcaacgttcagtgctgtgagtgtgtcacccaacccaccaccatgacaaccaccaccacagagaacccaactccgccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccgggccccccacccacacaagcacagcaccgattgctgagttgaccacatccaatcctccgcctgagtcctcaacccctcagacctctcggtccacctcttcccctctcacggagtcaaccacccttctgagtaccctaccacctgccattgagatgaccagcacggccccaccctccacacccacggcacccacgaccacgagcggaggccacacactgtctccaccgcccagcaccaccacgtcccctccaggcacccccactcgcggtaccacgactgggtcatcttcagcccccacccccagcactgtgcagacgaccaccaccagtgcctggacccccacgccgaccccactctccacacccagcatcatcaggaccacaggcctgaggccctacccttcctctgtgcttatctgctgtgtcctgaacgacacctactacgcaccaggtgaggaggtgtacaacggcacatacggagacacctgttatttcgtcaactgctcactgagctgtacgttggagttctataactggtcctgcccatccacgccctccccaacacccacgccctccaagtcgacgcccacgccttccaagccatcgtccacgccctccaagccgacgcccggcaccaagccccccgagtgcccagactttgatcctcccagacaggagaacgagacttggtggctgtgcgactgcttcatggccacgtgcaagtacaacaacacggtggagatcgtgaaggtggagtgtgagccgccgcccatgcccacctgctccaacggcctccaacccgtgcgcgtcgaggaccccgacggctgctgctggcactgggagtgcgactgctactgcacgggctggggcgacccgcactatgtcaccttcgacggactctactacagctaccagggcaactgcacctacgtgctggtggaggagatcagcccctccgtggacaacttcggagtttacatcgacaactaccactgcgatcccaacgacaaggtgtcctgcccccgcaccctcatcgtgcgccacgagacccaggaggtgctgatcaagaccgtgcatatgatgcccatgcaggtgcaggtgcaggtgaacaggcaggcggtggcactgccctacaagaagtacgggctggaggtgtaccagtctggcatcaactacgtggtggacatccccgagctgggtgtcctcgtctcctacaatggcctgtccttctccgtcaggctgccctaccaccggtttggcaacaacaccaagggccagtgtggcacctgcaccaacaccacctccgacgactgcattctgcccagcggggagatcgtctccaactgtgaggctgcggctgaccagtggctggtgaacgacccctccaagccacactgcccccacagcagctccacgaccaagcgcccggccgtcactgtgcccgggggcggtaaaacgaccccacacaaggactgcaccccatctcccctctgccagctcatcaaggacagcctgtttgcccagtgccacgcactggtgcccccgcagcactactacgatgcctgcgtgttcgacagctgcttcatgccgggctcgagcctggagtgcgccagtctgcaggcctacgcagccctctgtgcccagcagaacatctgcctcgactggcggaaccacacgcatggggcctgcttggtggagtgcccatctcacagggagtaccaggcctgtggccctgcagaagagcccacgtgcaaatccagctcctcccagcagaacaacacagtcctggtggaaggctgcttctgtcctgagggcaccatgaactacgctcctggctttgatgtctgcgtgaagacctgcggctgtgtgggacctgacaatgtgcccagagagtttggggagcacttcgagttcgactgcaagaactgtgtctgcctggagggtggaagtggcatcatctgccaacccaagaggtgcagccagaagcccgttacccactgcgtggaagacggcacctacctcgccacggaggtcaaccctgccgacacctgctgcaacattaccgtctgcaagtgcaacaccagcctgtgcaaagagaagccctccgtgtgcccgctgggattcgaagtgaagagcaagatggtgcctggaaggtgctgtcctttctactggtgtgagtccaagggggtgtgtgttcacgggaatgctgagtaccagcccggttctccagtttattcctccaagtgccaggactgcgtgtgcacggacaaggtggacaacaacaccctgctcaacgtcatcgcctgcacccacgtgccctgcaacacctcctgcagccctggcttcgaactcatggaggcccccggggagtgctgtaagaagtgtgaacagacgcactgtatcatcaaacggcccgacaaccagcacgtcatcctgaagcccggggacttcaagagcgacccgaagaacaactgcacattcttcagctgcgtgaagatccacaaccagctcatctcgtccgtctccaacatcacctgccccaactttgatgccagcatttgcatcccgggctccatcacattcatgcccaatggatgctgcaagacctgcacccctcgcaatgagaccagggtgccctgctccaccgtccccgtcaccacggaggtttcgtacgccggctgcaccaagaccgtcctcatgaatcattgctccgggtcctgcgggacatttgtcatgtactcggccaaggcccaggccctggaccacagctgctcctgctgcaaagaggagaaaaccagccagcgtgaggtggtcctgagctgccccaatggcggctcgctgacacacacctacacccacatcgagagctgccagtgccaggacaccgtctgcgggctccccaccggcacctcccgccgggcccggcgctcccctaggcatctggggagcgggtgagcggggtgggcacagcccccttcactgccctcgacagctttacctcccccggaccctctgagcctcctaagctcggcttcctctcttcagatatttattgtctgagtctttgttcagtccttgctttccaataataaactcagggggacatgc

SEQ ID NO: 31 - protein, 324 aa, Homo sapiens
MFPSPALTPTPFSVKDILNLEQQQRSLAAAGELSARLEATLAPSSCMLAAFKPEAYAGPEAAAPGLPELRAELGRAPSPAKCASAFPAAPAFYPRAYSDPDPAKDPRAEKKELCALQKAVELEKTEADNAERPRARRRRKPRVLFSQAQVYELERRFKQQRYLSAPERDQLASVLKLTSTQVKIWFQNRRYKCKRQRQDQTLELVGLPPPPPPPARRIAVPVLVRDGKPCLGDSAPYAPAYGVGLNPYGYNAYPAYPGYGGAACSPGYSCTAAYPAGPSPAQPATAAANNNFVNFGVGDLNAVQSPGIPQSNSGVSTLHGIRAW

SEQ ID NO: 32 - DNA, 1669 bp, Homo sapiens
  source 1..1669: mol_type = other DNA
gctcctgtcatcgaggcccctggcccaatggcaggctgagtccccctcctctggcctggtcccgcctctcctgccccttgtgctcagcgctacctgctgcccggacacatccagagctggccgacgggtgcgcgggcgggcggcggcaccatgcagggaagctgccaggggccgtgggcagcgccgctttctgccgcccacctggcgctgtgagactggcgctgccaccatgttccccagccctgctctcacgcccacgcccttctcagtcaaagacatcctaaacctggaacagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgagctggagcggcgcttcaagcagcagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtagggaagggacccgcgtggcgcgaccctgaccgatcccacctcaacagctccctgactctcggggggagaaggggctcccaacatgaccctgagtcccctggattttgcattcactcctgcggagacctaggaactttttctgtcccacgcgcgtttgttcttgcgcacgggagagtttgtggcggcgattatgcagcgtgcaatgagtgatcctgcagcctggtgtcttagctgtccccccaggagtgccctccgagagtccatgggcacccccggttggaactgggactgagctcgggcacgcagggcctgagatctggccgcccattccgcgagccagggccgggcgcccgggcctttgctatctcgccgtcgcccgcccacgcacccacccgtatttatgtttttacctattgctgtaagaaatgacgatccccttcccattaaagagagtgcgttgaccccg

SEQ ID NO: 33 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 34 - DNA, 3002 bp, Homo sapiens
  source 1..3002: mol_type = other DNA
ggggaggaggggagaacggggagcgcacagcctggacgcgtgcgcaggcgtcaggcgcatagacctgctagcccctcagctagcggccccgcccgcgcttagcatcactaactgggctatataacctgagcgcccgcgcggccacgacacgaggaattcgcccacgcaggaggcgcggcgtccggaggccccagggttatgagactatcactgctcaggacctactaacaacaaaggaaatcgaaacatgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattagaggcacgccagtttcaccatttccgggaaacgaacccactgtgcttacagtgactgtcgtgtttacaaaaggcagccctttgggtactactgctgcaaagtgcaaatactccaagcttcaagtgatatatgtatttattgtcattactgcctttggaagaaacaggggatcaaagttcctgttcaccttatgtattattttctatagctcttctatttaaaaaataaaaaaatacagtaaagtttaaaaaatacaccacgaatttggtgtggctgtattcagatcgtattaattatctgatcgggataacaaaatcacaagcaataattaggatctatgcaatttttaaactagtaatgggccaattaaaatatatataaatatatatttttcaaccagcattttactacttgttacctttcccatgctgaattattttgttgtgattttgtacagaatttttaatgactttttataatgtggatttcctattttaaaaccatgcagcttcatcaatttttatacatatcagaaaagtagaattatatctaatttatacaaaataatttaactaatttaaaccagcagaaaagtgcttagaaagttattgtgttgccttagcacttctttcctctccaattgtaaaaaaaaaaaaaaaaaaaaaaaaaaaaaaaaaattgcacaatttgagcaattcatttcactttaaagtctttccgtctccctaaaataaaaaccagaatcataattttcaagagaagaaaaaattaagagatacattccctatcaaaacatatcaattcaacacattacttgcacaagcttgtatatacatattataaataaatgccaacatacccttctttaaatcaaaagctgcttgactatcacatacaatttgcactgttactttttagtcttttactcctttgcattccatgattttacagagaatctgaagctattgatgtttccagaaaatataaatgcatgattttatacatagtcacaaaaatggtggtttgtcatatattcatgtaataaatctgagcctaaatctaatcaggttgttaatgttgggatttatatctatagtagtcaattagtacagtagcttaaataaattcaaaccatttaattcataattagaacaatagctattgcatgtaaaatgcagtccagaataagtgctgtttgagatgtgatgctggtaccactggaatcgatctgtactgtaattttgtttgtaatcctgtatattatggtgtaatgcacaatttagaaaacattcatccagttgcaataaaatagtattgaaagtgagagcaattgttgcatttcttcttaaagggattctgtttttatttttggggaaagtagttgcttttttgctgagttaaaaaatactaaacactatatgtagaataaaagaaaagaaaaaagtttaccttggcatatgctcttgtctgtttatcttgcacagggagtcaccagttctatgtagataatgaaaagacctaactgatatttcattatttggaatatgggactggacggcagtacaaacagtgtgtttttttctttgttttaagtggcttagcctttaggttttttatttccatttttaaaaatgattgttacatgttttcttctatttctttttttaaaaggtggattttaataa

SEQ ID NO: 35 - protein, 367 aa, Homo sapiens
MLAVGAMEGTRQSAFLLSSPPLAALHSMAEMKTPLYPAAYPPLPAGPPSSSSSSSSSSSPSPPLGTHNPGGLKPPATGGLSSLGSPPQQLSAATPHGINDILSRPSMPVASGAALPSASPSGSSSSSSSSASASSASAAAAAAAAAAAAASSPAGLLAGLPRFSSLSPPPPPPGLYFSPSAAAVAAVGRYPKPLAELPGRTPIFWPGVMQSPPWRDARLACTPHQGSILLDKDGKRKHTRPTFSGQQIFALEKTFEQTKYLAGPERARLAYSLGMTESQVKVWFQNRRTKWRKKHAAEMATAKKKQDSETERLKGASENEEEDDDYNKPLDPNSDDEKITQLLKKHKSSSGGGGGLLLHASEPESSS

SEQ ID NO: 36 - DNA, 1116 bp, Homo sapiens
  source 1..1116: mol_type = other DNA
cgtgggatgttagcggtgggggcaatggagggcacccggcagagcgcattcctgctcagcagccctcccctggccgccctgcacagcatggccgagatgaagaccccgctgtaccctgccgcgtatcccccgctgcctgccggccccccctcctcctcgtcctcgtcgtcgtcctcctcgtcgccctccccgcctctgggcacccacaacccaggcggcctgaagcccccggccacgggggggctctcatccctcggcagccccccgcagcagctctcggccgccaccccacacggcatcaacgatatcctgagccggccctccatgcccgtggcctcgggggccgccctgccctccgcctcgccctccggttcctcctcctcctcttcctcgtccgcctctgcctcctccgcctctgccgccgccgcggctgctgccgcggccgcagccgccgcctcatccccggcggggctgctggccggactgccacgctttagcagcctgagcccgccgccgccgccgcccgggctctacttcagccccagcgccgcggccgtggccgccgtgggccggtaccccaagccgctggctgagctgcctggccggacgcccatcttctggcccggagtgatgcagagcccgccctggagggacgcacgcctggcctgtacccctcatcaaggatccattttgttggacaaagacgggaagagaaaacacacgagacccactttttccggacagcagatcttcgccctggagaagactttcgaacaaacaaaatacttggcggggcccgagagggctcgtttggcctattcgttggggatgacagagagtcaggtcaaggtctggttccagaaccgccggaccaagtggaggaagaagcacgctgccgagatggccacggccaagaagaagcaggactcggagacagagcgcctcaagggggcctcggagaacgaggaagaggacgacgactacaataagcctctggatcccaactcggacgacgagaaaatcacgcagctgttgaagaagcacaagtccagcagcggcggcggcggcggcctcctactgcacgcgtccgagccggagagctcatcctgaacgccg

SEQ ID NO: 37 - protein, 353 aa, Homo sapiens; identical to SEQ ID NO: 23

SEQ ID NO: 38 - DNA, 2058 bp, Homo sapiens
  source 1..2058: mol_type = other DNA
gggtccttcaggtaggaggtcctgggtgactttggaagtccgtagtgtctcattgcagataatttttagcttagggcctggtggctaggtcggttctctcctttccagtcggagacctctgccgcaaacatgctccgccagatcatcggtcaggccaagaagcatccgagcttgatccccctctttgtatttattggaactggagctactggagcaacactgtatctcttgcgtctggcattgttcaatccagatgtttgttgggacagaaataacccagagccctggaacaaactgggtcccaatgatcaatacaagttctactcagtgaatgtggattacagcaagctgaagaaggaacgtccagatttctaaatgaaatgtttcactataacgctgctttagaatgaaggtcttccagaagccacatccgcacaattttccacttaaccaggaaatatttctcctctaaatgcatgaaatcatgttggagatctctattgtaatctctattggagattacaatgattaaatcaataaataactgaaacttgatatgtgtcacttttttatgctgaaagtatgctctgaactttagagtataggaaattaactattagaatttaaagaatttcttgaatttctgtagtttgaaaatacgactttaagctgctttagtaaaacacttccattttgtgtatagactgttggtaacttcactagagcatacataacaactggaactggaaattatacaaaagtaaattgggaaggatactccagcatctgacactggcaaaatggaaacctttgagtttctcttactggctgttgaagtgtgtgcagtttttaacaatggtttttacttggcatctctttgttgtgattttcaaggttataagttgctttggtcctaggattgaagttgaaatctgagtttatcagtgctaaccatggtgctagtagtcaagagatcttgagaattttggctgctgagtcttggtgcagggtgcaggttttcttttcttttttcttttttttttttttgagatagtctctgtcacccaggctggagtgcagtggtacaaacatggatcactgcagcctctacctcccgggcttaagtgatcctcctgcctcagcccctaagtagccgggactacaggtatgtgccaccatgcccagttaatttttgtaattttttttagagacagggttttgccatgttgcccaggctggtctcaaactcttgagctcaagcgatccattctcctcagcctcccagggtgctgggattacaggcgtgagccattgcgcttagccatggtgcaggttttcaaaggccaggaagtatattcataattttaagatggggaatatagcaagttttcacataggtgtgtgtaagtcatcacatcatagaaacttgaggaattcagtgacattaattttggattttcatacgtaagtatacaattaaatgtttacagggtagtagaagcacattttaaatgtcaggaactgaactaagtatttgaattacgtggattatctcaaaaattttgaaattgttaaacgagttgaattacttgaattcattctgttagtcaaatggtggatatttacacccatgtagttttgaatttagagtgtgtagagtgttttcagttaccagactccatgcttttacctcctatgtgtcaggtataatttgaacctctaagaacagggtttctcaaccttgccactgttgactatttctgaaagacagtttggtttagcagaccatcccatgcgctttagcttgtttagtagctaacttgggctctgccactacagacaaaaagcactctttccctccaattcccacaggctatgagaagaatggagacattaccaaatgtccattggtgggcaaaattgcttcattcctacctctgttgagaattactctagatcctttggcacaaattacctcaaagtttaaaattgtgtaaacaaacagtgtgtcatgtaattgaaaaacattaagcaactccaaataaatgctacattaag

SEQ ID NO: 39 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 40 - DNA, 531 bp, Homo sapiens
  source 1..531: mol_type = other DNA
atgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 41 - protein, 343 aa, Homo sapiens
MNQLGGLFVNGRPLPLDTRQQIVRLAVSGMRPCDISRILKVSNGCVSKILGRYYRTGVLEPKGIGGSKPRLATPPVVARIAQLKGECPALFAWEIQRQLCAEGLCTQDKTPSVSSINRVLRALQEDQGLPCTRLRSPAVLAPAVLTPHSGSETPRGTHPGTGHRNRTIFSPSQAEALEKEFQRGQYPDSVARGKLATATSLPEDTVRVWFSNRRAKWRRQEKLKWEMQLPGASQGLTVPRVAPGIISAQQSPGSVPTAALPALEPLGPSCYQLCWATAPERCLSDTPPKACLKPCWGHLPPQPNSLDSGLLCLPCPSSHCHLASLSGSQALLWPGCPLLYGLE

SEQ ID NO: 42 - DNA, 2010 bp, Homo sapiens
  source 1..2010: mol_type = other DNA
caaagactcacccgtgagccagctctcaaagaaagcagcttgcgttgacagcctgggggcagcaaggatgcagtctcccaggagaggatgcactcggtggtgggaagccaggctggaggggcctgagtgaccctctccacaggcgggcagggcagtgggagaggtggtgtgtggatacctctgtctcacgcccagggatcagcagcatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaagcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcacctggccagtcttagtggctctcaggccctgctctggcctggctgcccactactgtatggcttggaatgaggcaggagtgggaaggagatggcatagagaagatctaataccatcctgcccattgtccttaccgtcctgcccatacagactgtggctccttcctccttcctgtgattgctccctcctgtgtggacgttgcctggccctgcctcgatgcctctctggcgcatcacctgattggaggggctggtaaagcaacacccacccacttctcacactagccttaagaggcctccactcagcagtaataaaagctgtttttattagcagtagttctgttgtccatcatgttttccctatgagcacccctatgcccactctaatattcaacaattatagacaatttgccctatcatttatttacatctatgtatctaccatctaatctatgcatgtatgtaggcaatacatgtatctaaacaatgtatttgtcaatgcatcaatttacctactctatgtatgcatctatatgtgtattatgtatgcgtgcatgcgtgcgcgcacacacacacacacacacacacactgacattatatcatggcattttattcctaaatcttccagcatgcatccccaaaaaacaagaaacttgtcttacataatcacaataatatatccacatctaagaaaatttactgtaacttcttaatctaagaaaattatgtatttttgtcatatgtattttgtcatatgtattttgtatttgcatatgtattttgtatttgcatatgtatttttgtcatagcagcaaacagagtgaaatgccatttttcatattct

SEQ ID NO: 43 - protein, 286 aa, Homo sapiens
MGADGMYDKLRMLNGQTGSWGTRPGWYPGTSVPGQPTQDGCQQQEGGGENTNSISSNGEDSDEAQMRLQLKRKLQRNRTSFTQEQIEALEKEFERTHYPDVFARERLAAKIDLPEARIQVWFSNRRAKWRREEKLRNQRRQASNTPSHIPISSSFSTSVYQPIPQPTTPVSSFTSGSMLGRTDTALTNTYSALPPMPSFTMANNLPMQPPVPSQTSSYSCMLPTSPSVNGRSYDTYTPPHMQTHMNSQPMGTSGTTSTGLISPGVSVPVQVPGSEPDMSQYWPRLQ

SEQ ID NO: 44 - DNA, 6732 bp, Homo sapiens
  source 1..6732: mol_type = other DNA
cttttcaattagccttccatgcatgatccggagcgacttccgcctatttccagaaattaagctcaaacttgacgtgcagctagttttattttaaagacaaatgtcagagaggctcatcatattttcccccctcttctatatttggagcttatttattgctaagaagctcaggctcctggcgtcaatttatcagtaggctccaaggagaagagaggagaggagaggagagctgaacagggagccacgtcttttcctgggagggctgctatctaagtcggggctgcaggtcacagcggagtgaatcagctcggtggtgtctttgtcaacgggcggccactgccggactccacccggcagaagattgtagagctagctcacagcggggcccggccgtgcgacatttcccgaattctgcagacccatgcagatgcaaaagtccaagtgctggacaatcaaaacgtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaacttaagtgttttgaagttgttcacaactacatatcaaaattaaccattgttgattgtaaaaaaccatgccaaagcctttgtatttcctttattatacagttttctttttaaccttatagtgtggtgttacaaattttatttccatgttagatcaacattctaaaccaatggttactttcacacacactctgttttacatcctgatgatccttaaaaaataatccttatagataccataaatcaaaaacgtgttagaaaaaaattccacttacagcagggtgtagatctgtgcccatttatacccacaacatatatacaaaatggtaacatttcccagttagccatttaattctaaagctcaaagtctagaaataatttaaaaatgcaacaagcgattagctaggaattgttttttgaattaggactggcattttcaatctgggcagatttccattgtcagcctatttcaacaatgatttcactgaagtatattcaaaagtagatttcttaaaggagactttctgaaagctgttgcctttttcaaataggccctctcccttttctgtctccctcccctttgcacaagaggcatcatttcccattgaaccactacagctgttcccatttgaatcttgctttctgtgcggttgtggatggttggagggtggaggggggatgttgcatgtcaaggaataatgagcacagacacatcaacagacaacaacaaagcagactgtgactggccggtgggaattaaaggccttcagtcattggcagcttaagccaaacattcccaaatctatgaagcagggcccattgttggtcagttgttatttgcaatgaagcacagttctgatcatgtttaaagtggaggcacgcagggcaggagtgcttgagcccaagcaaaggatggaaaaaaataagcctttgttgggtaaaaaaggactgtctgagactttcatttgttctgtgcaacatataagtcaatacagataagtcttcctctgcaaacttcactaaaaagcctgggggttctggcagtctagattaaaatgcttgcacatgcagaaacctctggggacaaagacacacttccactgaattatactctgctttaaaaaaatccccaaaagcaaatgatcagaaatgtagaaattaatggaaggatttaaacatgaccttctcgttcaatatctactgttttttagttaaggaattacttgtgaacagataattgagattcattgctccggcatgaaatatactaataattttattccaccagagttgctgcacatttggagacaccttcctaagttgcagtttttgtatgtgtgcatgtagttttgttcagtgtcagcctgcactgcacagcagcacatttctgcaggggagtgagcacacatacgcactgttggtacaattgccggtgcagacatttctacctcctgacattttgcagcctacattccctgagggctgtgtgctgagggaactgtcagagaagggctatgtgggagtgcatgccacagctgctggctggcttacttcttccttctcgctggctgtaatttccaccacggtcaggcagccagttccggcccacggttctgttgtgtagacagcagagactttggagacccggatgtcgcacgccaggtgcaagaggtgggaatgggagaaaaggagtgacgtgggagcggagggtctgtatgtgtgcacttgggcacgtatatgtgtgctctgaaggtcaggattgccagggcaaagtagcacagtctggtatagtctgaagaagcggctgctcagctgcagaagccctctggtccggcaggatgggaacggctgccttgccttctgcccacaccctagggacatgagctgtccttccaaacagagctccaggcactctcttggggacagcatggcaggctctgtgtggtagcagtgcctgggagttggccttttactcattgttgaaataatttttgtttattatttatttaacgatacatatatttatatatttatcaatggggtatctgcagggatgttttgacaccatcttccaggatggagattatttgtgaagacttcagtagaatcccaggactaaacgtctaaattttttctccaaacttgactgacttgggaaaaccaggtgaatagaataagagctgaatgttttaagtaataaacgttcaaactgctctaagtaaaaaaatgcattttactgcaatgaatttctagaatatttttcccccaaagctatgcctcctaacccttaaatggtgaacaactggtttcttgctacagctcactgccatttcttcttactatcatcactaggtttcctaagattcactcatacagtattatttgaagattcagctttgttctgtgaatgtcatcttaggattgtgtctatattcttttgcttatttctttttactctgggcctctcatactagtaagattttaaaaagccttttcttctctgtatgtttggctcaccaaggcgaaatatatattcttctctttttcatttctcaagaataaacctcatctgcttttttgtttttctgtgttttggcttggtactgaatgactcaactgctcggttttaaagttcaaagtgtaagtacttagggttagtactgcttatttcaataatgttgacggtgactatctttggaaagcagtaacatgctgtcttagaaatgacattaataatgggcttaaacaaatgaataggggggtccccccactctccttttgtatgcctatgtgtgtctgatttgttaaaagatggacagggaattgattgcagagtgtcgcttccttctaaagtagttttattttgtctactgttagtatttaaagatcctggaggtggacataaggaataaatggaagagaaaagtagatattgtatggtggctactaaaaggaaattcaaaaagtcttagaacccgagcacctgagcaaactgcagtagtcaaaatatttatctcatgttaaagaaaggcaaatctagtgtaagaaatgagtaccatatagggttttgaagttcatatactagaaacacttaaaagatatcatttcagatattacgtttggcattgttcttaagtatttatatctttgagtcaagctgataattaaaaaaaatctgttaatggagtgtatatttcataatgtatcaaaatggtgtctatacctaaggtagcattattgaagagagatatgtttatgtagtaagttattaacataatgagtaacaaataatgtttccagaagaaaggaaaacacattttcagagtgcgtttttatcagaggaagacaaaaatacacacccctctccagtagcttatttttacaaagccggcccagtgaattagaaaaacaaagcacttggatatgatttttggaaagcccaggtacacttattattcaaaatgcacttttactgagtttgaaaagtttcttttatatttaaaataagggttcaaatatgcatattcaatttttatagtagttatctatttgcaaagcatatattaactagtaattggctgttaattttatagacatggtagccagggaagtatatcaatgacctattaagtattttgacaagcaatttacatatctgatgacctcgtatctctttttcagcaagtcaaatgctatgtaattgttccattgtgtgttgtataaaatgaatcaacacggtaagaaaaaggttagagttattaaaataataaactgactaaaatactcatttgaatttattcagaatgttcataatgctttcaaaggacatagcagagcttttgtggagtatccgcacaacattatttattatctatggactaaatcaattttttgaagttgctttaaaatttaaaagcacctttgcttaatataaagccctttaattttaactgacagatcaattctgaaactttattttgaaaagaaaatggggaagaatctgtgtctttagaattaaaagaaatgaaaaaaataaacccgacattctaaaaaaatagaataagaaacctgatttttagtactaatgaaatagcgggtgacaaaatagttgtctttttgattttgatcacaaaaaataaactggtagtgacaggatatgatggagagatttgacatcctggcaaatcactgtcattgattcaattattctaattctgaataaaagctgtatacagtaaaa

SEQ ID NO: 45 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 46 - DNA, 2573 bp, Homo sapiens
  source 1..2573: mol_type = other DNA
gggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaacctttctggcagtgggatgattcgaattcacttttaaaattaaattagcgtgttttgttttg

SEQ ID NO: 47 - protein, 328 aa, Homo sapiens
MDAVLLEHFPGGLDAFPSSYFDEDDFFTDQSSRDPLEDGDELLADEQAEVEFLSHQLHEYCYRDGACLLLQPAPPAAPLALAPPSSGGLGEPDDGGGGGYCCETGAPPGGFPYSPGSPPSCLAYPCAGAAVLSPGARLRGLSGAAAAAARRRRRVRSEAELQQLRQAANVRERRRMQSINDAFEGLRSHIPTLPYEKRLSKVDTLRLAIGYINFLSELVQADLPLRGGGAGGCGGPGGGGRLGGDSPGSQAQKVIICHRGTRSPSPSDPDYGLPPLAGHSLSWTDEKQLKEQNIIRTAKVWTPEDPRKLNSKSSFNNIENEPPFEFVS

SEQ ID NO: 48 - DNA, 1339 bp, Homo sapiens
  source 1..1339: mol_type = other DNA
atggacgcggtgttgctggagcacttccccgggggcctagacgcctttccttcttcgtacttcgacgaggacgacttcttcaccgaccagtcttcacgggaccccctggaggacggcgatgagctgctggcggacgagcaggccgaggtggagttccttagccaccagctccacgagtactgctaccgcgacggggcgtgcctgctgctgcagcccgcgcccccggccgccccgctagcgctcgccccgccgtcctcggggggcctcggtgagccagacgacggcggcggcggcggctactgctgcgagacgggggcgcccccaggcggcttcccctactcgcccggctcgccgccctcgtgcctggcctacccgtgcgccggggcggcagtactgtctcccggggcgcggctgcgcggcctgagcggagcggcggctgcggcggcgcggcgccggcggcgggtgcgctccgaggcggagctgcagcagctgcggcaggcggccaacgtgcgcgagcggcggcgcatgcagtccatcaacgacgccttcgaggggctgcgctcgcacatccccacgctgccctacgagaagcgcctctccaaggtggacacgctgcgcctggccatcggctacatcaacttcctcagcgagctcgtgcaggccgacctgcccttgcgcggcggtggcgcgggcggctgcggggggccgggcggcggcgggcgcctgggcggggacagcccgggcagccaggcccagaaggtcatcatctgccatcggggcacccggtccccctcccccagcgaccctgattatggcctccctcccctagcaggacactctctctcatggactgatgaaaaacaactcaaggaacaaaatattatccgaacagccaaagtctggaccccagaggaccccagaaaactcaacagcaaatcttccttcaacaacatagaaaacgaaccaccatttgagtttgtgtcctgagaagtcccagactcggctgaagatctgattatgtctctgtgcatattgtacatgtaaatatctataatgtaaatgtaatttaagaatcaaatttttcgaatggcaatcaactgtttattatttatctatttattatcctgttgagttgatgaaatagatgatttctttttaaatatataatttatataacttatcctgattttctgaaaatatgcaatagcctatgattttcctgaactctgtgttgttgggagaactctggccagaaaacgtcctgcttatttattgccagatatggtttatttctaagcgttgtcaataaatgctatttacaccttttcctgaaaaaaaa

SEQ ID NO: 49 - DNA, 1555 bp, Homo sapiens
  source 1..1555: mol_type = other DNA
cggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcg

SEQ ID NO: 50 - protein, 385 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCKNPGSRAGNSAHQPPHPQPPVRFHPPLRRAGKVP

SEQ ID NO: 51 - DNA, 1056 bp, Homo sapiens
  source 1..1056: mol_type = other DNA
atgagtccccgctcgtgcctgcgttcgctgcgcctcctcgtcttcgccgtcttctcagccgccgcgagcaactggctgtacctggccaagctgtcgtcggtggggagcatctcagaggaggagacgtgcgagaaactcaagggcctgatccagaggcaggtgcagatgtgcaagcggaacctggaagtcatggactcggtgcgccgcggtgcccagctggccattgaggagtgccagtaccagttccggaaccggcgctggaactgctccacactcgactccttgcccgtcttcggcaaggtggtgacgcaagggattcgggaggcggccttggtgtacgccatctcttcggcaggtgtggcctttgcagtgacgcgggcgtgcagcagtggggagctggagaagtgcggctgtgacaggacagtgcatggggtcagcccacagggcttccagtggtcaggatgctctgacaacatcgcctacggtgtggccttctcacagtcgtttgtggatgtgcgggagagaagcaagggggcctcgtccagcagagccctcatgaacctccacaacaatgaggccggcaggaaggccatcctgacacacatgcgggtggaatgcaagtgccacggggtgtcaggctcctgtgaggtaaagacgtgctggcgagccgtgccgcccttccgccaggtgggtcacgcactgaaggagaagtttgatggtgccactgaggtggagccacgccgcgtgggctcctccagggcactggtgccacgcaacgcacagttcaagccgcacacagatgaggacttggtgtacttggagcctagccccgacttctgtgagcaggacatgcgcagcggcgtgctgggcacgaggggccgcacatgcaacaagacgtccaaggccatcgacggctgtgagctgctgtgctgtggccgcggcttccacacggcgcaggtggagctggctgaacgctgcagctgcaaattccactggtgctgcttcgtcaagtgccggcagtgccagcggctcgtggagttgcacacgtgccgatga

SEQ ID NO: 52 - protein, 351 aa, Homo sapiens
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGIREAALVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 53 - DNA, 1143 bp, Homo sapiens
  source 1..1143: mol_type = other DNA
atgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtag

SEQ ID NO: 54 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 55 - DNA, 21 bp, synthetic construct
  misc_feature 1..21: note = Description of Artificial Sequence: Synthetic primer
  source 1..21: mol_type = other DNA
gctaacactgtcgcagtttga

SEQ ID NO: 56 - DNA, 21 bp, synthetic construct
  misc_feature 1..21: note = Description of Artificial Sequence: Synthetic primer
  source 1..21: mol_type = other DNA
cgaacagctggaatcaatgtg

SEQ ID NO: 57 - DNA, 19 bp, synthetic construct
  misc_feature 1..19: note = Description of Artificial Sequence: Synthetic primer
  source 1..19: mol_type = other DNA
tgcagctacgctaccagga

SEQ ID NO: 58 - DNA, 19 bp, synthetic construct
  misc_feature 1..19: note = Description of Artificial Sequence: Synthetic primer
  source 1..19: mol_type = other DNA
ggaggctgagttcgcttgg

SEQ ID NO: 59 - DNA, 20 bp, synthetic construct
  misc_feature 1..20: note = Description of Artificial Sequence: Synthetic primer
  source 1..20: mol_type = other DNA
ctcggaggtagttccggttc

SEQ ID NO: 60 - DNA, 21 bp, synthetic construct
  misc_feature 1..21: note = Description of Artificial Sequence: Synthetic primer
  source 1..21: mol_type = other DNA
tctgcccaatctgacgaagag

SEQ ID NO: 61 - DNA, 19 bp, synthetic construct
  misc_feature 1..19: note = Description of Artificial Sequence: Synthetic primer
  source 1..19: mol_type = other DNA
atgctccgccagatcatcg

SEQ ID NO: 62 - DNA, 21 bp, synthetic construct
  misc_feature 1..21: note = Description of Artificial Sequence: Synthetic primer
  source 1..21: mol_type = other DNA
tgccagacgcaagagatacag

SEQ ID NO: 63 - DNA, 20 bp, synthetic construct
  misc_feature 1..20: note = Description of Artificial Sequence: Synthetic primer
  source 1..20: mol_type = other DNA
ggccttcagtactccctgca

SEQ ID NO: 64 - DNA, 21 bp, synthetic construct
  misc_feature 1..21: note = Description of Artificial Sequence: Synthetic primer
  source 1..21: mol_type = other DNA
gggacttggagcttgagtcct

SEQ ID NO: 65 - DNA, 23 bp, synthetic construct
  misc_feature 1..23: note = Description of Artificial Sequence: Synthetic primer
  source 1..23: mol_type = other DNA
gcggagtgtaatcagtatttgga

SEQ ID NO: 66 - DNA, 21 bp, synthetic construct
  misc_feature 1..21: note = Description of Artificial Sequence: Synthetic primer
  source 1..21: mol_type = other DNA
gcatttgatcccgtacaacct

SEQ ID NO: 67 - DNA, 20 bp, synthetic construct
  misc_feature 1..20: note = Description of Artificial Sequence: Synthetic primer
  source 1..20: mol_type = other DNA
gcctgcgctaattgtaggag

SEQ ID NO: 68 - DNA, 20 bp, synthetic construct
  misc_feature 1..20: note = Description of Artificial Sequence: Synthetic primer
  source 1..20: mol_type = other DNA
cgcacttgaaagttgcaaaa

SEQ ID NO: 69 - DNA, 20 bp, synthetic construct
  misc_feature 1..20: note = Description of Artificial Sequence: Synthetic primer
  source 1..20: mol_type = other DNA
ttcaggacggggtgagttac

SEQ ID NO: 70 - DNA, 20 bp, synthetic construct
  misc_feature 1..20: note = Description of Artificial Sequence: Synthetic primer
  source 1..20: mol_type = other DNA
tttggcatggtacaggttca

SEQ ID NO: 71 - DNA, 20 bp, synthetic construct
  misc_feature 1..20: note = Description of Artificial Sequence: Synthetic primer
  source 1..20: mol_type = other DNA
gctggaatcaatttcccaga

SEQ ID NO: 72 - DNA, 20 bp, synthetic construct
  misc_feature 1..20: note = Description of Artificial Sequence: Synthetic primer
  source 1..20: mol_type = other DNA
ctccccacacaggatgagtt

SEQ ID NO: 73 - DNA, 20 bp, synthetic construct
  misc_feature 1..20: note = Description of Artificial Sequence: Synthetic primer
  source 1..20: mol_type = other DNA
agcctttgtgaaccaacacc

SEQ ID NO: 74 - DNA, 20 bp, synthetic construct
  misc_feature 1..20: note = Description of Artificial Sequence: Synthetic primer
  source 1..20: mol_type = other DNA
gctggtagagggagcagatg

SEQ ID NO: 75 - DNA, 19 bp, synthetic construct
  misc_feature 1..19: note = Description of Artificial Sequence: Synthetic primer
  source 1..19: mol_type = other DNA
aggcagacccactcagtga

SEQ ID NO: 76 - DNA, 20 bp, synthetic construct
  misc_feature 1..20: note = Description of Artificial Sequence: Synthetic primer
  source 1..20: mol_type = other DNA
aacaatggcgacctcttctg

SEQ ID NO: 77 - DNA, 22 bp, synthetic construct
  misc_feature 1..22: note = Description of Artificial Sequence: Synthetic primer
  source 1..22: mol_type = other DNA
gtacttcttggcagagctgctg

SEQ ID NO: 78 - DNA, 22 bp, synthetic construct
  misc_feature 1..22: note = Description of Artificial Sequence: Synthetic primer
  source 1..22: mol_type = other DNA
cagaagaaattcttgcagccag

SEQ ID NO: 79 - DNA, 25 bp, synthetic construct
  misc_feature 1..25: note = Description of Artificial Sequence: Synthetic primer
  source 1..25: mol_type = other DNA
ggatgaagtctaccaaagctcacgc

SEQ ID NO: 80 - DNA, 25 bp, synthetic construct
  misc_feature 1..25: note = Description of Artificial Sequence: Synthetic primer
  source 1..25: mol_type = other DNA
ccagatcttgatgtgtctctcggtc

SEQ ID NO: 81 - DNA, 22 bp, synthetic construct
  misc_feature 1..22: note = Description of Artificial Sequence: Synthetic primer
  source 1..22: mol_type = other DNA
cttcagcaaggaggaggtcatc

SEQ ID NO: 82 - DNA, 25 bp, synthetic construct
  misc_feature 1..25: note = Description of Artificial Sequence: Synthetic primer
  source 1..25: mol_type = other DNA
ctcgtatttctccttgtacaggtcc

SEQ ID NO: 83 - DNA, 20 bp, synthetic construct
  misc_feature 1..20: note = Description of Artificial Sequence: Synthetic primer
  source 1..20: mol_type = other DNA
attcgttggggatgacagag

SEQ ID NO: 84 - DNA, 20 bp, synthetic construct
  misc_feature 1..20: note = Description of Artificial Sequence: Synthetic primer
  source 1..20: mol_type = other DNA
tcaacagctgcgtgattttc

SEQ ID NO: 85 - DNA, 20 bp, synthetic construct
  misc_feature 1..20: note = Description of Artificial Sequence: Synthetic primer
  source 1..20: mol_type = other DNA
gatgggcttggctttgtaga

SEQ ID NO: 86 - DNA, 19 bp, synthetic construct
  misc_feature 1..19: note = Description of Artificial Sequence: Synthetic primer
  source 1..19: mol_type = other DNA
ggagggaagtccactctgc

SEQ ID NO: 87 - DNA, 20 bp, synthetic construct
  misc_feature 1..20: note = Description of Artificial Sequence: Synthetic primer
  source 1..20: mol_type = other DNA
gttctcaggacgaggagcac

SEQ ID NO: 88 - DNA, 20 bp, synthetic construct
  misc_feature 1..20: note = Description of Artificial Sequence: Synthetic primer
  source 1..20: mol_type = other DNA
cttgggcttttgatcgtcat

SEQ ID NO: 89 - DNA, 20 bp, synthetic construct
  misc_feature 1..20: note = Description of Artificial Sequence: Synthetic primer
  source 1..20: mol_type = other DNA
tatggtggtgccgactacaa

SEQ ID NO: 90 - DNA, 20 bp, synthetic construct
  misc_feature 1..20: note = Description of Artificial Sequence: Synthetic primer
  source 1..20: mol_type = other DNA
tgcttgtccagatgacttcg

SEQ ID NO: 91 - DNA, 18 bp, synthetic construct
  misc_feature 1..18: note = Description of Artificial Sequence: Synthetic primer
  source 1..18: mol_type = other DNA
gtgctgatgggcaagaac

SEQ ID NO: 92 - DNA, 18 bp, synthetic construct
  misc_feature 1..18: note = Description of Artificial Sequence: Synthetic primer
  source 1..18: mol_type = other DNA
aggtcctccttggtgaac

SEQ ID NO: 93 - DNA, 20 bp, synthetic construct
  misc_feature 1..20: note = Description of Artificial Sequence: Synthetic primer
  source 1..20: mol_type = other DNA
tgctgcataatcagctacgg

SEQ ID NO: 94 - DNA, 20 bp, synthetic construct
  misc_feature 1..20: note = Description of Artificial Sequence: Synthetic primer
  source 1..20: mol_type = other DNA
gctggtcacattgagaagca

SEQ ID NO: 95 - DNA, 20 bp, synthetic construct
  misc_feature 1..20: note = Description of Artificial Sequence: Synthetic primer
  source 1..20: mol_type = other DNA
gcaaggcattcttcaagagg

SEQ ID NO: 96 - DNA, 20 bp, synthetic construct
  misc_feature 1..20: note = Description of Artificial Sequence: Synthetic primer
  source 1..20: mol_type = other DNA
ggctgggcagctgtactcta

SEQ ID NO: 97 - DNA, 20 bp, synthetic construct
  misc_feature 1..20: note = Description of Artificial Sequence: Synthetic primer
  source 1..20: mol_type = other DNA
ctctcgactgggtgaaggag

SEQ ID NO: 98 - DNA, 20 bp, synthetic construct
  misc_feature 1..20: note = Description of Artificial Sequence: Synthetic primer
  source 1..20: mol_type = other DNA
gaagagccagcacaaaggtc

SEQ ID NO: 99 - DNA, 20 bp, synthetic construct
  misc_feature 1..20: note = Description of Artificial Sequence: Synthetic primer
  source 1..20: mol_type = other DNA
gaagccctgaaagacgacag

SEQ ID NO: 100 - DNA, 20 bp, synthetic construct
  misc_feature 1..20: note = Description of Artificial Sequence: Synthetic primer
  source 1..20: mol_type = other DNA
tcgacacgaactctccctct

SEQ ID NO: 101 - DNA, 20 bp, synthetic construct
  misc_feature 1..20: note = Description of Artificial Sequence: Synthetic primer
  source 1..20: mol_type = other DNA
gatccgagtgctctttggag

SEQ ID NO: 102 - DNA, 20 bp, synthetic construct
  misc_feature 1..20: note = Description of Artificial Sequence: Synthetic primer
  source 1..20: mol_type = other DNA
atgtcatccagaagcccaag

SEQ ID NO: 103 - DNA, 20 bp, synthetic construct
  misc_feature 1..20: note = Description of Artificial Sequence: Synthetic oligonucleotide
  source 1..20: mol_type = other DNA
gtggttgacgctgtccgtca

SEQ ID NO: 104 - DNA, 20 bp, synthetic construct
  misc_feature 1..20: note = Description of Artificial Sequence: Synthetic oligonucleotide
  source 1..20: mol_type = other DNA
ctgttcgtccttcatcaaga

SEQ ID NO: 105 - DNA, 600 bp, synthetic construct
  misc_feature 1..600: note = Description of Artificial Sequence: Synthetic polynucleotide
  source 1..600: mol_type = other DNA
ataagacacagttatgcttatggaagcgtgctgacaaacagtaattacagagctgaggatcatctgttcagtcttgaaaataaaagttttattctgctcataataaaatgattgcagcatcagaatgaggaaggaaaggtagaatgaggataaatacaattttagaaatggtatagactttgcaaatcaccacctcttccattgataaatttagaatctagagttgagttagatattgacactggttctccaagagaaaggtaaaataaaagcaatcggactctttagagcttttgtttatggcctgtctgggccctttgttgtaaccctgtcatgcccttatgctgattaccttcttgtagaacaagaagtattgactagagaatgaatgatgtgtagtccctagcccttaggaaactctctcaaagagcaatgtctttaacatatgaattctgtttttttcctccttttacctttccctttccctttctctatttttcaccatctcttttgtttctacctcttttggtctctgtgcttgacactctctcctctttctgtctctctttgtatctcctcaatctcaggcttctctgcaga

SEQ ID NO: 106 - DNA, 600 bp, synthetic construct
  misc_feature 1..600: note = Description of Artificial Sequence: Synthetic polynucleotide
  source 1..600: mol_type = other DNA
ctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggttagtgtcggacctgggaatactctccccacttccaacctcacatgatgggtttttgtttttccttattcttattctcataagtcaagtatcatagttttaattctctcttgagtagaaaatggaaatagattacaattgatagtggaagatttatagaataaaatccccccagatatactccatatctattaattttcctcttactgttaagctttaatggtgcaaggataataaactttgggtagagtttacaagagcatagttattattagagcaatgtgggtctatatagcaact

SEQ ID NO: 107 - DNA, 20 bp, synthetic construct
  misc_feature 1..20: note = Description of Artificial Sequence: Synthetic primer
  source 1..20: mol_type = other DNA
gtccatgctgatccatcctt

SEQ ID NO: 108 - DNA, 20 bp, synthetic construct
  misc_feature 1..20: note = Description of Artificial Sequence: Synthetic primer
  source 1..20: mol_type = other DNA
tgcttctccggtattgttcc

SEQ ID NO: 109 - DNA, 20 bp, synthetic construct
  misc_feature 1..20: note = Description of Artificial Sequence: Synthetic primer
  source 1..20: mol_type = other DNA
gtccatgctgatccatcctt

SEQ ID NO: 110 - DNA, 20 bp, synthetic construct
  misc_feature 1..20: note = Description of Artificial Sequence: Synthetic primer
  source 1..20: mol_type = other DNA
tgcttctccggtattgttcc